(19) United States
(12) Patent Application Publication
(10) Pub. No.: US 2026/0209848 A1
(43) Pub. Date: Jul. 23, 2026

(54) METHODS AND COMPOSITIONS FOR TARGETING PRC2 GENE TARGETS

(71) Applicant: The Johns Hopkins University, Baltimore, MD (US)

(72) Inventors: Andrew P. Feinberg, Baltimore, MD (US); Michael A. Koldobskiy, Baltimore, MD (US); Oscar Camacho, Baltimore, MD (US)

(21) Appl. No.: 19/142,928

(22) PCT Filed: Dec. 20, 2023
(86) PCT No.: PCT/US2023/085165

§ 371 Date: Jun. 24, 2025

Related U.S. Application Data
(60) Provisional application No. 63/436,272, filed on Dec. 30, 2022.

(51) Int. Cl. C12Q 1/6876

(52) U.S. Cl. CPC C12Q 1/6876; C12Q 2600/136; C12Q 2600/154; C12Q 2600/158

(57) ABSTRACT

The invention provides herein epigenomic changes identified in old mice compared to young mice or old mice treated with Oct4, Sox2, Klf4 and c-Myc (OSKM). The invention further provides methods of identifying a compound by analyzing one or more PRC2-associated epigenomic change in a sample before and after contacting the sample with one or more compounds. Also provided herein are methods of using compounds identified using the methods described herein for improving proliferative capacity, cell and tissue regeneration, and/or reducing fibrosis.

### CROSS-REFERENCE TO RELATED APPLICATIONS

This application claims the benefit of priority under 35 U.S.C. § 119 (e) of U.S. Provisional Application Ser. No. 63/436,272, filed Dec. 30, 2022. The disclosure of the prior application is considered part of and is herein incorporated by reference in the disclosure of this application in its entirety.

### BACKGROUND OF THE INVENTION

### Field of the Invention

The present disclosure relates generally to epigenomic changes associated with aging and more specifically to epigenomic changes in the Polycomb repressive complex 2 (PRC2) gene targets.

### Background Information

Rejuvenation of tissues in physiologically aging mice can be accomplished by long-term partial reprogramming via expression of reprogramming factors (Oct4, Sox2, Klf4 and c-Myc). In skin, partial reprogramming results in histologic and functional rejuvenation with increased epidermal thickness, improved wound healing, transcriptomic changes and reversal of a chronological epigenetic clock. To investigate the epigenetic determinants of reprogramming-mediated rejuvenation, whole genome bisulfite sequencing was used to carry out unbiased comprehensive profiling of DNA methylation alterations in skin from mice subjected to reprogramming, as well as young and old controls. The inventors of the disclosure identified a striking convergence of age- and rejuvenation-related epigenetic alterations on targets of the Polycomb repressive complex 2 (PRC2). These results are also supported by a likewise prominent enrichment of PRC2 targets in gene expression data, suggesting that PRC2 activity can modulate aging and mediate tissue rejuvenation.

### SUMMARY OF THE INVENTION

The present disclosure provides a method of identifying compounds or agents useful for reversing epigenomic changes. Such methods include, analyzing one or more PRC2-associated epigenomic change in a sample before and after contacting the sample with one or more compounds. In some embodiments, the PRC2-associated epigenomic change includes a decrease in mean methylation level (MML) or a reduction in normalized methylation entropy (NME) in one or more PRC2 gene targets in the sample after contacting with the compound. In some embodiments, aging is associated with an increase in MML or NME in one or more PRC2 gene targets. In some embodiments, the compounds described herein reverse the epigenomic changes associated with aging.

In some embodiments, the PRC2-associated epigenomic change includes a decrease in MML or NME in a promoter of a PRC2 gene target; a H3K27me3 methylation site of a PRC2 gene target and/or a binding site of enhancer of zeste homolog 2 (EZH2), suppressor of zest 12 (SUZ12), embryonic ectoderm development (EED), jumonji and AT-rich interaction domain containing 2 (JARID2), metal response element binding transcription factor 2 (MTF2) or a combination thereof in the PRC2 gene target. As a non-limiting example, the promoter has H3K27me3, H3K4me3 or a combination thereof. In some embodiments, the compounds increase gene expression of one or more PRC2 gene targets. In some embodiments, the PRC2 gene targets are CREB-regulated transcription coactivator 1 (CRTC1), SMAD family member 3 (SMAD3), Wnt family member 3A (WNT3A), nuclear receptor subfamily 4 group A member 2 (NR4A2), homeobox containing 1 (HMBOX1), forkhead box A1 (FOXA1), calcium/calmodulin dependent protein kinase II beta (CAMK2B), early growth response (EGR), nerve growth factor (NGF), PIM-1 proto-oncogene, serine/threonine kinase (PIM1), T-Box transcription factor 3 (TBX3), twist family BHLH transcription factor 1 (TWIST1), methionine sulfoxide reductase B3 (MSRB3), ETS variant transcription factor 6 (ETV6), or a combination thereof.

The present disclosure also provides compounds identified by the methods of the disclosure.

In some embodiments, the present disclosure provides a method of rejuvenating a population of aging cells by contacting the aging cells with one or more compounds described herein.

In some embodiments, the present disclosure provides a method of improving proliferative capacity of a population of cells comprising contacting the population of cells with the compounds described herein.

In some embodiments, the present disclosure provides a method of reducing fibrosis comprising contacting the compounds of the disclosure with the cells of a subject.

In some embodiments, the present disclosure provides a method of tissue regeneration comprising contacting tissue with the compounds of the disclosure.

In some embodiments, the present disclosure provides a method of identifying a genomic region of differential DNA methylation. Such methods include (a) obtaining a DNA sample from one or more of a young cohort, an old cohort and an octamer-binding transcription factor 4 (Oct4), SRY-box transcription factor 2 (Sox2), KLF transcription factor 4 (Klf4) and c-Myc, (OSKM) treated old cohort; (b) performing whole-genome bisulfite sequencing (WGBS) on the DNA sample; (c) calculating DNA methylation potential energy landscape across the genome of the DNA sample; (d) measuring a mean methylation level (MML) and a normalized methylation entropy (NME) in an analysis region of the DNA sample; and/or (e) identifying the genomic region of differential DNA methylation in the young cohort and the OSKM treated old cohort as compared to the DNA sample from the old cohort. In some embodiments, the method further includes identifying the genomic region of differential DNA methylation by the Jensen-Shannon distance (JSD) of information theory.

### BRIEF DESCRIPTION OF THE DRAWINGS

FIGS. 1A-1E are graphs depicting mean methylation level (MML) and normalized methylation entropy (NME). The graphs show that age-associated genome-wide epigenetic shift is reversed in OSKM-mediated rejuvenation. FIG. 1A is a plot of principal components (PC1 and PC2) obtained from the principal components analysis of the WGBS data corresponding to the cohorts included in the study. FIG. 1B, is a graphic representation of two boxplots of genome-wide distributions of mean methylation level (MML) and normalized methylation entropy (NME) values of the cohorts included in the study. FIG. 1C, FIG. 1D, and FIG. 1E are graphic representations of density depicting densities of genome-wide distributions of MML and NME values in young, old control and old OSKM-treated representative samples.

FIGS. 2A-2D are graphs of methylation showing that DNA methylation discordance identifies PRC2 targets as major convergence elements in aging and OSKM-mediated rejuvenation. FIG. 2A is a set of graphs showing Gene Set Enrichment Analysis of the top 500 differentially methylated genes in old OSKM-treated vs old control and young vs old control comparisons, using the Chromatin Enrichment Analysis (ChEA) 2022 and Molecular Signatures Database (MSigDB) databases. FIG. 2B is a set of graphs showing boxplots of distributions of MML and NME values from the genomic regions annotated as EZH2 binding sites, regions harboring H3K27me3 histone mark and poised promoters of the cohorts included in the study. FIGS. 2C and 2D are graphs showing a representation of the JSD and differences in MML and NME values of a comparison between representative old OSKM-treated (O+OSKM-2) vs old control (O-2) samples along a differentially methylated region harboring the cluster HOXA7-HOXA9.

FIGS. 3A and 3B are graphs showing that PRC2 targets are differentially expressed in aging and OSKM-mediated rejuvenation. Gene Set Enrichment Analysis of the upregulated and downregulated differentially expressed genes in old OSKM-treated vs old control and young vs old control comparisons, using the Chromatin Enrichment Analysis (ChEA) 2022 database.

FIGS. 4A-4D are graphs showing MML and NME distributions within selected genomic regions, specifically, the boxplots of distributions of MML and NME values within selected genomic regions of the cohorts included in the study.

FIGS. 5A and 5B are graphs showing boxplots of distributions of JSD and differences in MML and NME within selected genomic regions defined as chromatin states by available chromHMM annotations in representative OSKM-treated old (O+OSKM-2) vs old control (O−2) and young (Y−3) vs old control (O−2) comparisons.

### DETAILED DESCRIPTION OF THE INVENTION

Before the present compositions and methods are described, it is to be understood that this invention is not limited to the particular composition, method, and experimental conditions described, as such composition, method, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, certain methods and materials are now described.

Cellular reprogramming using enforced expression of the transcription factors Oct4, Sox2, Klf4 and c-Myc (OSKM) has emerged as a powerful strategy to reverse phenotypes associated with aging. Expression of OSKM for short and repeated intervals in vivo has been shown to ameliorate physiologic aging phenotypes in tissues and extends life span in an accelerated mouse aging model. Long-term in vivo reprogramming protocols to investigate the effects of partial reprogramming in normal physiologically aging mice using mice carrying a single copy of an OSKM polycistronic cassette and a reverse tetracycline transactivator (rtTA) in a C57BL/6 (B6) genetic background (4F mice) have been established. Browder et al. used long-term reprogrammed 4F mice subjected to 7 months of cyclic partial reprogramming starting at 15 months of age and continued until 22 months of age, as well as control aged 4F mice subjected to mock treatment during the same aging interval, and a control cohort of untreated 4F young (3 months) mice (Browder, K. C. et al. Nature Aging 2022 2:3 2, 243-253 (2022); the contents of which are herein incorporated by reference in its entirety). Browder et al, showed that the skin of mice subjected to long-term reprogramming revealed changes consistent with histologic and functional rejuvenation when compared to old control mice, including increased epidermal thickness, higher proliferative capacity and decreased fibrosis after injury, reversal of age-related metabolic changes and downregulation of genes involved in inflammation and epidermal differentiation. This suggests that long-term reprogramming preserves a more plastic, less differentiated state in aged skin cells. Given the dramatic reversal of aging-related, metabolic, transcriptional and phenotypic alterations by long-term reprogramming, the inventors sought to understand epigenetic changes that underlie the rejuvenation process. The application of a DNA methylation array-based aging clock involving less than 700 mostly non-functional CpG sites has previously demonstrated a reversal of age-related DNA methylation changes following long-term reprogramming. However, this provides only a static assessment of the methylation state of a limited number of CpG sites, without a direct connection to biological aging.

In the present disclosure, the inventors have fully characterized the genome-wide DNA methylation landscape comprehensively in a model of controlled tissue rejuvenation induced by reprogramming factors. The skin was chosen for this analysis because of the dramatic histological and functional reversal of aging manifest in this tissue under OSKM, as well as the very strong human data showing highly reproducible superficial and histological metrics of skin aging compared to other tissue types. For analysis, whole-genome bisulfite sequencing (WGBS), that discriminates quantitatively and on a single-read basis methylation status of—15 million CpG dinucleotides and is >4 orders of magnitude more than the aging clock CpG site array was used. OSKM-mediated rejuvenation was also compared to old control and young cohorts.

Numerous methods for analyzing methylation status of a gene are known in the art and are used in the methods of the present invention to identify either hypomethylation or hypermethylation of a genomic region. In some embodiments, the determining of methylation status is performed by one or more techniques selected from the group consisting of a nucleic acid amplification, polymerase chain reaction (PCR), methylation specific PCR, bisulfite pyrosequencing, single-strand conformation polymorphism (SSCP) analysis, restriction analysis, and microarray technology. As illustrated in the Examples herein, analysis of methylation are performed by bisulfite genomic sequencing. Bisulfite treatment modifies DNA converting unmethylated, but not methylated, cytosines to uracil. Bisulfite treatment is carried out using the METHYLEASY bisulfite modification kit (Human Genetic Signatures).

In some embodiments, bisulfite pyrosequencing, which is a sequencing-based analysis of DNA methylation that quantitatively measures multiple, consecutive CpG sites individually with high accuracy and reproducibility, is used. Nucleic acid primers or probes for such analysis are derived from any of the Tables included herein or any other known genomic sequence.

It will be recognized that depending on the site bound by the primer and the direction of extension from a primer, that the primers listed above are used in different pairs. Furthermore, it will be recognized that additional primers are identified within the genomic regions identified in the Tables, especially primers that allow analysis of the same methylation sites as those analyzed with primers that correspond to the primers disclosed herein or any other known genomic sequence.

Altered methylation is identified by identifying a detectable difference in methylation. For example, hypomethylation is determined by identifying whether after bisulfite treatment a uracil or a cytosine is present a particular location. If uracil is present after bisulfite treatment, then the residue is unmethylated. Hypomethylation is present when there is a measurable decrease in methylation.

The term “nucleic acid molecule” is used broadly herein to mean a sequence of deoxyribonucleotides or ribonucleotides that are linked together by a phosphodiester bond. As such, the term “nucleic acid molecule” is meant to include DNA and RNA, which is single stranded or double stranded, as well as DNA/RNA hybrids. Furthermore, the term “nucleic acid molecule” as used herein includes naturally occurring nucleic acid molecules, which are isolated from a cell, as well as synthetic molecules, which are prepared, for example, by methods of chemical synthesis or by enzymatic methods such as by the polymerase chain reaction (PCR), and, in various embodiments, contains nucleotide analogs or a backbone bond other than a phosphodiester bond.

The terms “polynucleotide” and “oligonucleotide” also are used herein to refer to nucleic acid molecules. Although no specific distinction from each other or from “nucleic acid molecule” is intended by the use of these terms, the term “polynucleotide” is used generally in reference to a nucleic acid molecule that encodes a polypeptide, or a peptide portion thereof, whereas the term “oligonucleotide” is used generally in reference to a nucleotide sequence useful as a probe, a PCR primer, an antisense molecule, or the like. Of course, it will be recognized that an “oligonucleotide” also encodes a peptide. As such, the different terms are used primarily for convenience of discussion.

A polynucleotide or oligonucleotide comprising naturally occurring nucleotides and phosphodiester bonds are chemically synthesized or are produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase incorporates certain types of nucleotide analogs into a polynucleotide and, therefore, is used to produce such a polynucleotide recombinantly from an appropriate template.

In another aspect, the present invention includes kits that are useful for carrying out the methods of the present invention. The components contained in the kit depend on a number of factors, including: the particular analytical technique used to detect methylation or measure the degree of methylation or a change in methylation, and the one or more genomic regions being assayed for methylation status.

The present disclosure provides results related to biological aging as opposed to chronological aging because of the convergence of PRC2 targets among both differentially methylated and differentially expressed genes, and the relevance of some of the top epigenetically altered genes in aging and regeneration. The use of the targets described here as biomarkers for epigenetic rejuvenation serves in high throughput screening or evaluation of age-reversal interventions. Since PRC2 activity is modified pharmacologically, this opens the door for further studies to address to what extent PRC2 modulation recapitulates the rejuvenating influence of OSKM reprogramming.

The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1: Convergence of Aging- and Rejuvenation-Related Epigenetic Alterations on PRC2 Targets

Whole-genome bisulfite sequencing (WGBS) was carried out on skin from five mice subjected to long-term OSKM reprogramming from 15 months of age until 22 months, four control-treated 4F mice, and three young 4F (3 months old) mice. WGBS data was analyzed using informME, a powerful tool for quantifying epigenetic variability by computing DNA methylation potential energy landscapes (PELs) across the genome, capturing both mean methylation level (MML) and methylation variability (stochasticity) as encapsulated by normalized methylation entropy (NME), a version of Shannon entropy that quantifies the disorder of methylation within an analysis region. This method permits identification of genomic regions of significant DNA methylation discordance using the Jensen-Shannon distance (JSD) of information theory, based on differences in probability distributions of methylation rather than conventional differential methylation analysis that is based solely on mean methylation differences.

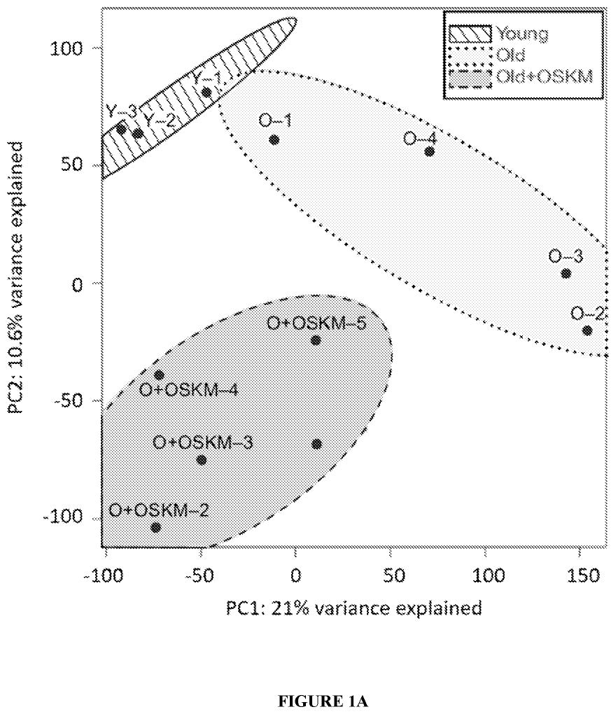

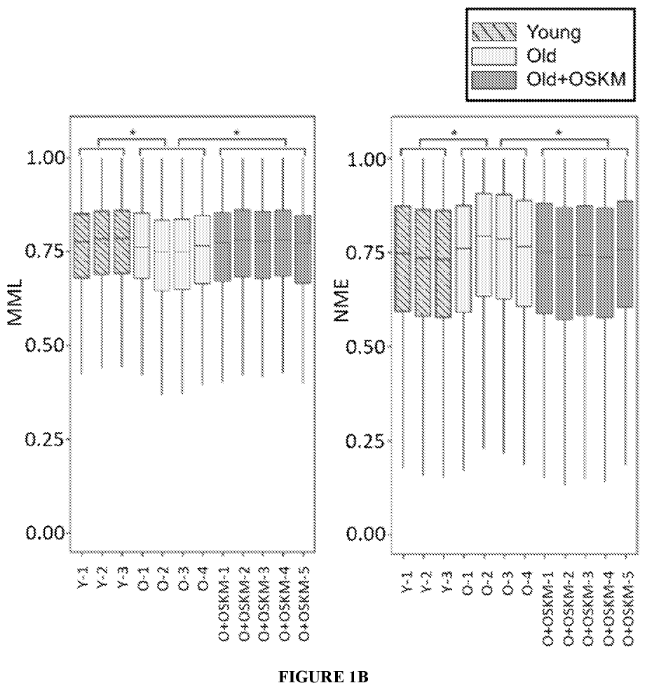

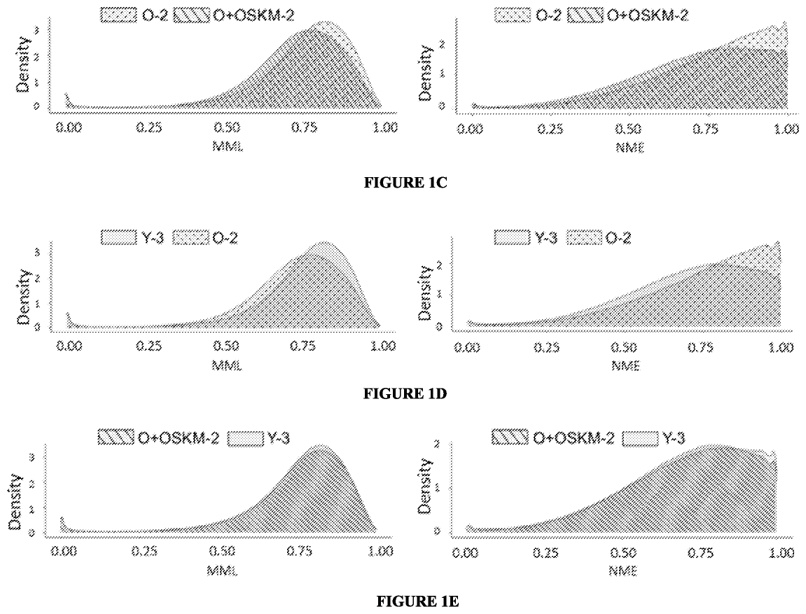

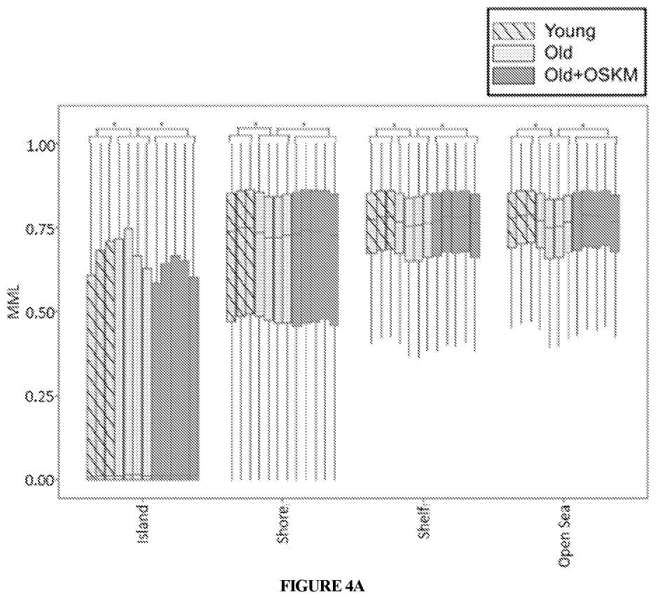

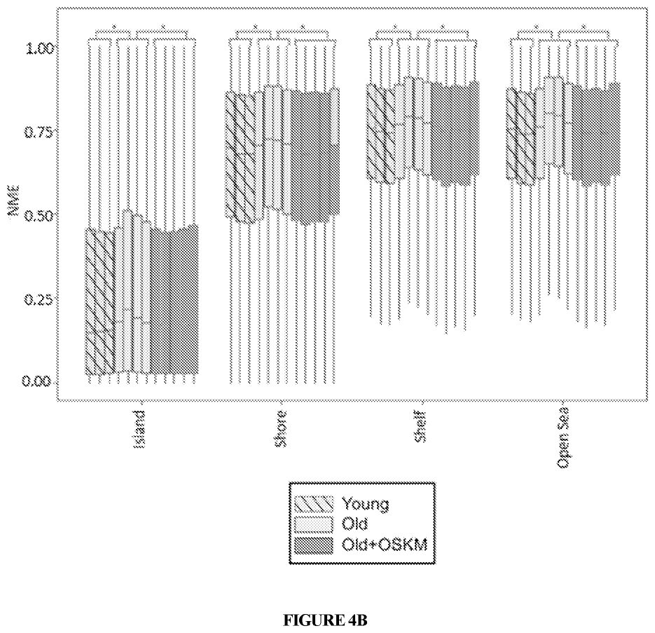

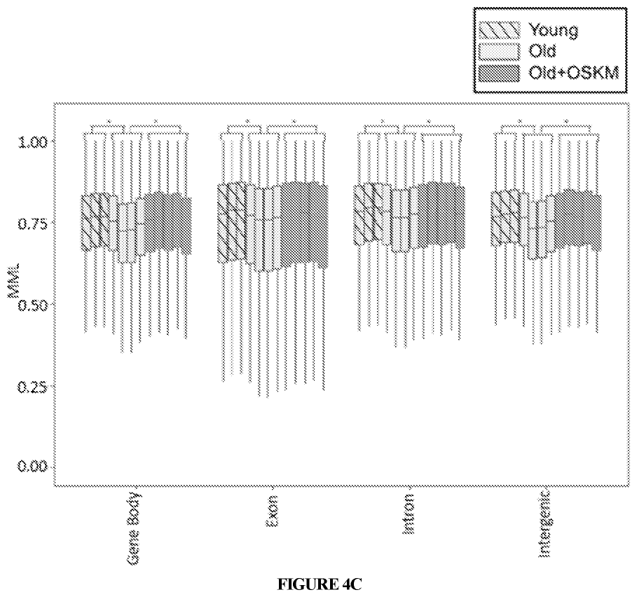

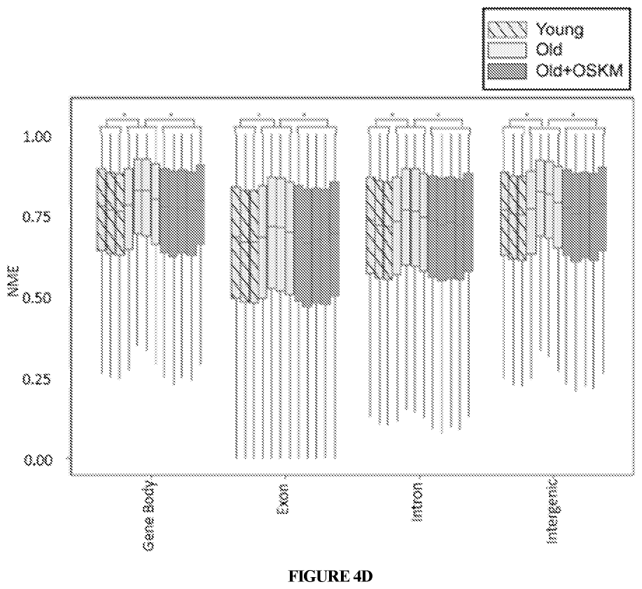

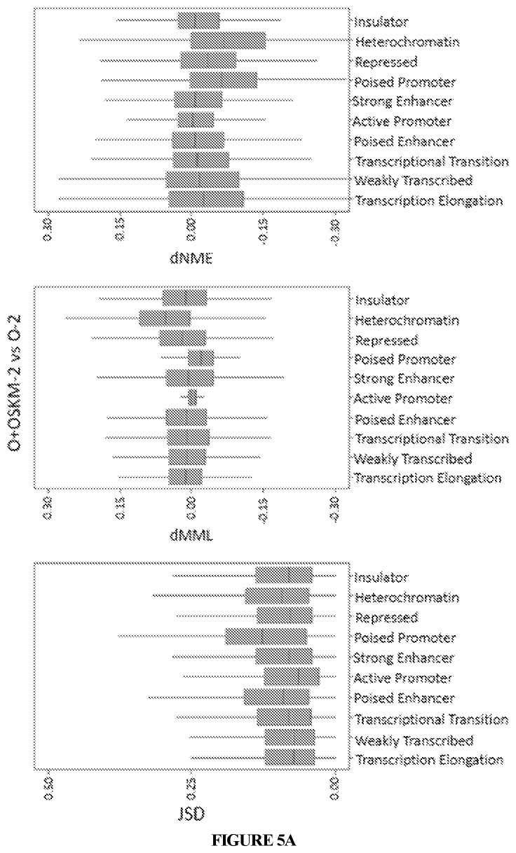

Principal component (PC) analysis revealed a clear separation between the different groups, PC1 capturing age and rejuvenation-related differences and PC2 capturing OSKM treatment-specific effects (FIG. 1A). Genome-wide distributions of the MML and NME showed a substantial shift towards hypomethylation and a more stochastic (disordered) epigenome in old control skin when compared to young skin (FIG. 1B, FIG. 1C). Conversely, old skin subjected to long-term reprogramming showed an increase in global methylation levels and a reduction of methylation entropy in comparison to old control samples, resembling young skin (FIG. 1B, FIG. 1D, FIG. 1E). The same trend was consistent when examining the MML and NME distributions over selected genomic features (FIGS. 4A-4D).

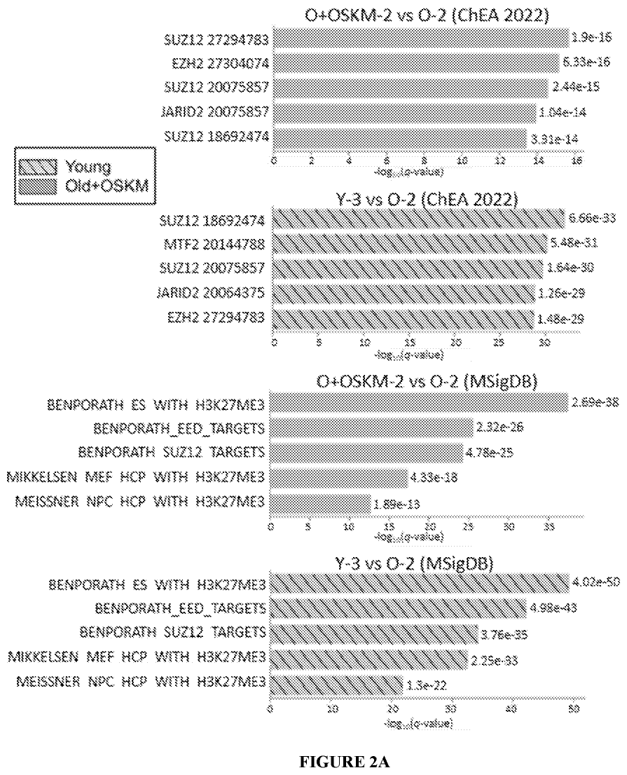

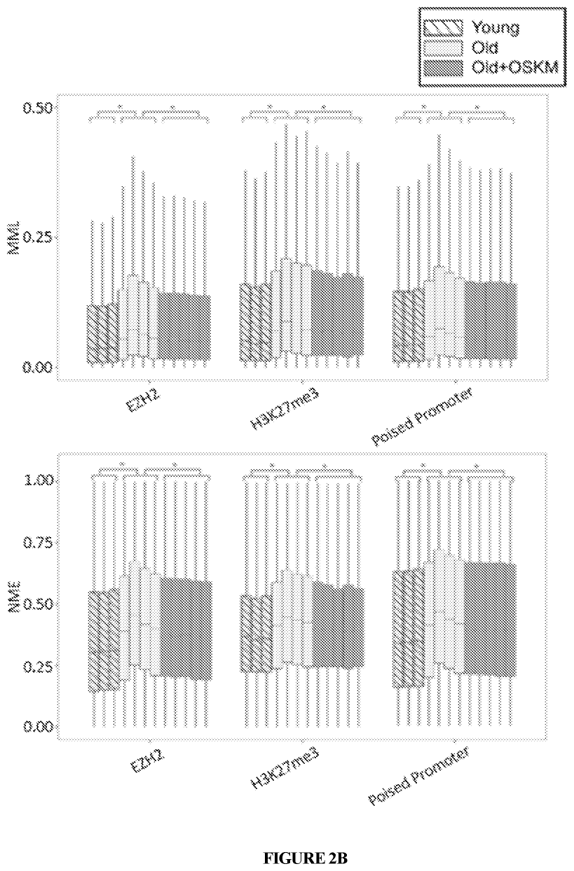

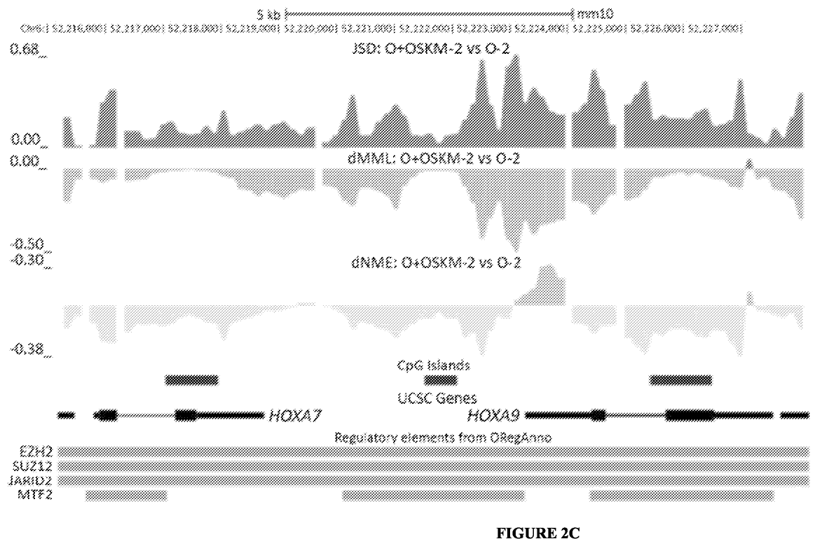

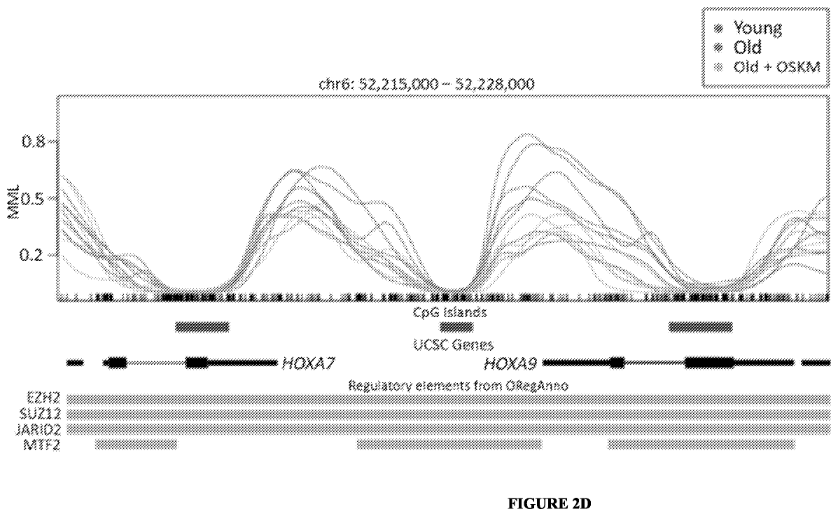

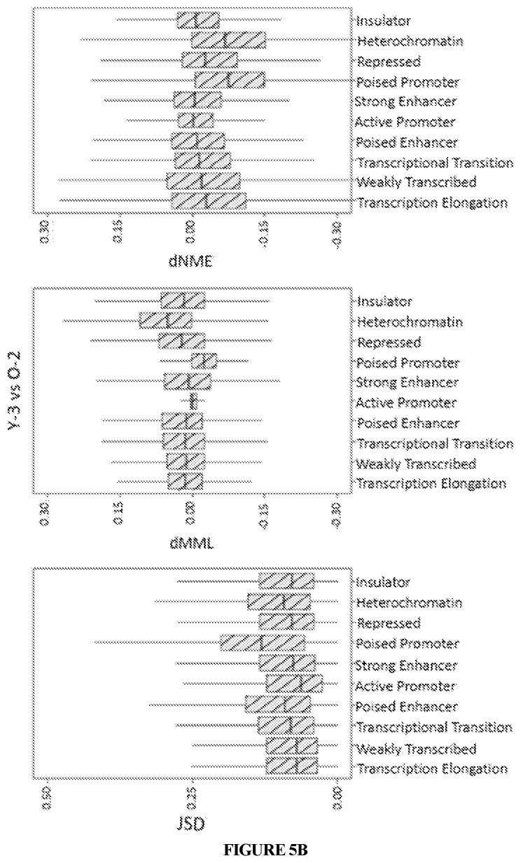

Given that aging and OSKM long-term reprogramming are associated with altered DNA methylation, the genomic targets of epigenetic disruption in skin aging and rejuvenation were identified next. Genes were ranked based on the potential of their DNA methylation states within their promoter regions to distinguish between the young, old control, and old OSKM-treated groups, using the mutual information between the methylation state and the phenotype within a gene feature, computed as the average of the sum of squares of all JSD values within analysis regions that overlap the gene feature groups. The most epigenetically discordant genes were identified and evaluated for enrichments among the top 500 genes in each group-wise comparison using Gene Set Enrichment Analysis (GSEA). Remarkably, in both old OSKM-treated vs old control comparisons and young vs old control comparisons, the top GSEA enrichments were consistently those genes sets related to Polycomb repressive complex 2 (PRC2) gene targets (EZH2, SUZ12, EED, JARID2 and MTF2 targets, and genes possessing H3K27me3 marks) (FIG. 2A). The dramatic enrichment observed in both comparisons suggested a convergence of aging- and rejuvenation-related epigenetic changes specifically to genes that are targets of PRC2 activity. When assessing the changes in MML and NME over genomic regions annotated as EZH2 binding sites, regions harboring H3K27me3 histone mark and poised promoters (defined as promoters possessing both H3K27me3 and H3K4me3) by employing available ChIP-seq data and chromHMM annotations, a significant shift of the methylation levels towards hypermethylation and a dramatic increase in entropy in the old control samples compared to young skin was identified. OSKM-treated old skin exhibited a specific reversal of methylation changes in these genomic regions (FIG. 2B). Interestingly, among the different chromatin states defined by chromHMM, poised promoters showed the greatest JSD and among the most elevated differences in NME comparing both OSKM-treated old and young with old control, indicating such domains are the most epigenetically dysregulated regions, gain significant entropy with age and such epigenetic disorder is reversed upon OSKM treatment. Furthermore, most features exhibited hypomethylation in old control samples, with the notable exception of poised promoters which showed aging-related hypermethylation, also partially reversed with OSKM treatment (FIGS. 5A and 5B). An example of a differentially methylated region is shown in FIGS. 2C and 2D, including the changes in JSD values and differences of MML and NME of the comparison OSKM-treated old vs old control along the cluster Hoxa7-Hoxa9, relevant in embryonic development, regeneration and aging.

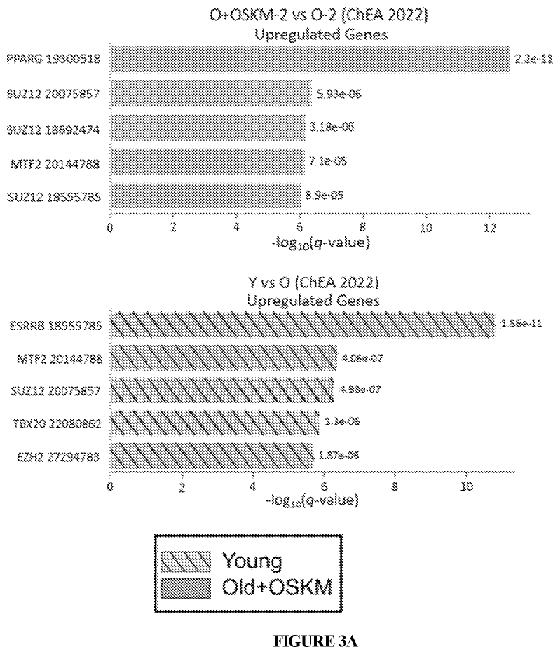

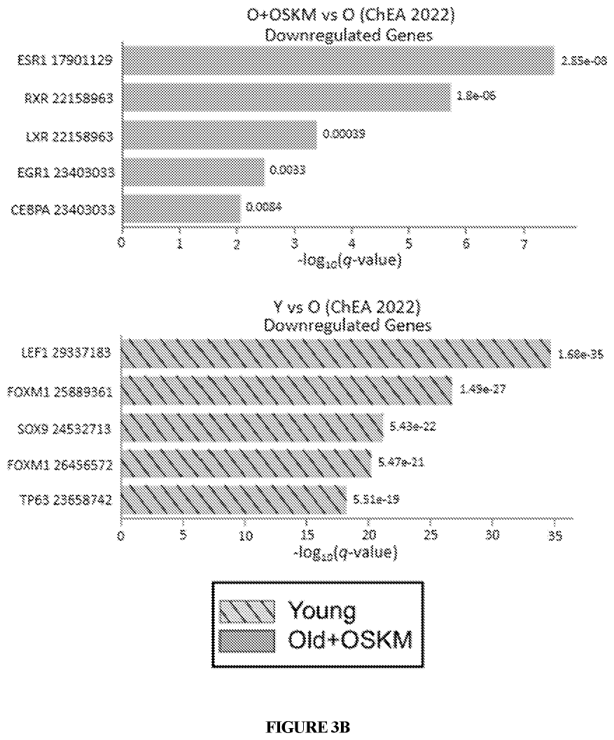

To evaluate the relationship of these findings to gene expression alterations during aging and reprogramming, and in order to identify potential epigenetic regulators driving such alterations, differential gene expression (DEG) analysis was carried out using RNA-seq data from young, old control, and OSKM-treated old skin samples. Using GSEA enrichment analysis, it was found that among overexpressed DEG when comparing both young and OSKM-treated old to control old skin there was likewise a prominent enrichment of PRC2 targets (FIGS. 3A and 3B), suggesting PRC2 drives age-related gene expression changes. Among the genes found to be both differentially methylated by the OSKM-treatment when compared to old control and differentially expressed in aging or OSKM reprogramming, several genes relevant in aging and regeneration were identified, including CRTC1, SMAD3, WNT3A, NR4A2, HMBOX1, FOXA1, CAMK2B, EGR, NGF, PIM1, TBX3, TWIST1, MSRB3, ETV6 and multiple members of the HOX and SOX families, as well as different genes that mediate the age-associated changes in metabolism, remodeling of extracellular matrix and inflammatory response. Among the epigenetically dysregulated genes without apparent change in gene expression FOXO3 and FOXO6, relevant in aging, and JARID2 and KDM2B, epigenetic regulators were identified.

Taken together, these results point to a central and previously unappreciated role for PRC2 signaling in the regulation of tissue aging and rejuvenation. The analysis of the aging epigenome based on epigenetic discordance described herein as captured by the JSD, identified PRC2 domains as the most epigenetically disrupted regions during aging due to hypermethylation and significant gain of entropy. Importantly, the data show that long-term OSKM treatment reverses such epigenetic disruption, decreasing both the mean methylation and entropy levels.

### EXPERIMENTAL METHODS

### WGBS Library Preparation and Sequencing

Genomic DNA was isolated using the MasterPure DNA Purification kit (Epicentre). Integrity of genomic DNA was confirmed by gel electrophoresis. WGBS single indexed libraries were generated using NEBNext Ultra DNA library Prep kit for Illumina (New England BioLabs) according to the manufacturer's instructions with the following modifications: 500 ng input gDNA was quantified by Qubit dsDNA BR assay (Invitrogen) and spiked with 1% unmethylated Lambda DNA (Promega, cat #D1521) to monitor bisulfite conversion efficiency. Input gDNA was fragmented by Covaris S220 Focused-ultrasonicator to an average insert size of 350 bp. Samples were sheared for 60 sec using Covaris microTUBEs, with instrument settings of duty cycle 10%, intensity 5 and cycles per burst 200. Size selection was performed using AMPure XP beads and insert sizes of 300-400 bp were isolated. Samples were bisulfite converted after size selection using EZ DNA Methylation-Gold Kit or EZ DNA Methylation-Lightning Kit (Zymo cat #D5005, cat #D5030) following the manufacturer's instructions. After bisulfite conversion, amplification was performed using Kapa Hifi Uracil+(Kapa Biosystems, cat #KK282) polymerase based on the following cycling conditions: 98° C. 45 s/8 cycles: 98° C. 15 s, 65° C. 30 s, 72° C. 30 s/72° C. 1 min. AMPure cleaned-up libraries were run on the 2100 Bioanalyzer (Agilent) High-Sensitivity DNA assay and samples were also run on the Bioanalyzer after shearing and size selection for quality control purposes. Libraries were quantified by qPCR using the Library Quantification Kit for Illumina sequencing platforms (Kapa Biosystems, cat #KK4824) and the 79001IT Real Time PCR System (Applied Biosystems). WGBS libraries were sequenced on an Illumina HiSeq4000 instrument using 150 bp paired-end indexed reads and 25% of non-indexed PhiX library control (Illumina). The bisulfite conversion rate of unmethylated Lambda DNA was 99.5% on average.

### Mapping and Quality Control of Whole Genome Bisulfite Sequencing

FASTQ files were processed using Trim Galore! v.0.4.0 (Babraham Institute) to perform single-pass adapter- and quality-trimming of reads. FastQC v.0.11.2 was employed for quality control of reads. Reads were aligned to the GRCm38 genome using Bismark v.01.14.5. Separate M-bias plots for read 1 and read 2 were generated by running the Bismark methylation extractor using the ‘mbias only’ flag, and these plots were used to determine how many bases to remove from the 5′ end of reads. The number was generally higher for read 2, which is known to exhibit a lower quality. The amount of 5′ trimming ranged from 5 bp to 20 bp. BAM files were subsequently processed with Samtools v.0.1.19 for sorting, merging, duplicate removal and indexing.

### Genomic Features and Annotations

Files and tracks bear genomic coordinates for mm 10. CGIs annotations were obtained from the UCSD Genome Browser. CGI shores were defined as sequences flanking 2-kb on either side of CGIs, shelves as sequences flanking 2-kb beyond the shores, and open seas as everything else. The R package ‘TxDb.Mmusculus. UCSC.mm10.knownGene’ was used to define genes, exons, and introns. The promoter region of a gene was defined as the 4-kb window centered at its transcription start site (TSS) and determined the gene body region to be the remainder of the gene. ChIP-seq and chromHMM data from mouse embryonic stem cells were obtained from the Mouse Encode Project.

### PEL Computation

DNA methylation was computed using potential energy landscapes (PELs) from WGBS data using informME (v0.3.2), a freely available information-theoretic pipeline for methylation analysis based on the 1D Ising model of statistical physics 1,2. For these computations, the entire genome was partitioned into consecutive non-overlapping genomic windows of 3-kb each and a PEL was estimated within each window from available WGBS reads using a maximum-likelihood approach 1,2.

### Methylation Level and Entropy

Each 3-kb estimation window was further partitioned into 20 non-overlapping analysis regions of size 150 bp each. Within each analysis region, the probability distribution of the methylation level was computed, the mean methylation level (MML), and the normalized methylation entropy (NME) directly from the associated PEL using informME. The methylation level within an analysis region with N CpG sites is given by L=EVEn=MXn, where Xn is a binary random variable that takes values 0 or 1 with a certain probability, indicating that the n-th CpG site in the analysis region is unmethylated or methylated, respectively. The MML is the expected value of L, given by EL=Eil×PW, where P (1), 1=0, 1N, 2N, . . . , 1, is the associated probability distribution of the methylation level, whereas the NME is a normalized version of the Shannon entropy of L, given by h-fifiog2:f910V+ME/PHog2:f91P(/).

### Jensen-Shannon Distance

The Jensen-Shannon distance (JSD) was computed between two probability distributions, P1 and P2, of the methylation level in a test (AML) and a reference (normal) sample within an analysis region by 12DKLP1, P−+DKLP2,P−, where P−=P1+P22 and DKLQ1,Q2=E1Q11 log 2Q11Q21 is the relative entropy of a probability distribution Q1 with respect to a probability distribution Q2. This quantity ranges between 0 and 1, taking its minimum value only when the probability distributions P1 and P2 are identical (no statistical discordance in methylation level) and its maximum value of 1 only when the supports of the two probability distributions do not intersect each other (maximum statistical discordance).

### Differential Methylation Analysis

Differential analysis was performed between test (AML) and reference (normal) WGBS samples using informME. Jensen-Shannon distances (JSDs) was computed, within analysis regions between the corresponding methylation level probability distributions, as well as differences between mean methylation levels (dMMLs), normalized methylation entropies (dNMEs).

### Hypothesis Testing and Gene Ranking

In a single test/reference comparison, within the promoter and body regions of each gene in the genome, the magnitude of the Jensen-Shannon distance (JSD) was computed, which was calculated as the square root of the average of the squared JSD values within all analysis regions that overlap each feature (promoter or body). By following a previous statistical methodology, hypothesis testing was performed to test against the null hypothesis that the JSD magnitude within a particular genomic feature (promoter or body) is explained by normal technical, statistical, or biological variability. This was done by empirically constructing a null distribution for the values of all JSD magnitudes genome-wide, which was obtained by comparing our three young 4F samples. To account for variability in the number of analysis regions overlapping each genomic feature, generalized additive models were employed for location scale and shape (GAMLSS) with a logit skewed Student's t-distribution. Hypothesis testing was performed simultaneously for methylation discordance within a gene's promoter and body using Fisher's summary statistic to test the null hypothesis that epigenetic discordance observed within a gene's promoter or body in a test/reference comparison is only associated with biological, statistical, or technical variability in the reference samples, against the alternative hypothesis that this discordance is due to other factors within at least one of the two features considered (promoter or body). To evaluate genes in multiple test/reference comparisons, Fisher's summary test statistic was used to test the null hypothesis that epigenetic discordance observed within a gene's promoter and body in the test/reference comparisons is only associated with biological, statistical, or technical variability in the reference samples, against the alternative hypothesis that this discordance is due to other factors within at least one of the two genomic features considered (promoter or body) in at least one of the test/reference comparisons, and followed a similar approach to evaluate genes using only their promoters or bodies. Finally each gene was scored by using the computed p-value for rejecting the null hypothesis and produced a ranked list of genes with increasing p-values, breaking possible ties by combining the p-value rankings obtained from each single test/reference comparison using the method of rank products. Finally, the statistical significance was evaluated for each ranking while controlling for the false-discovery rate (FDR) at 0.05 using q-values computed by the Benjamini-Hochberg (BH) procedure.

### Differentially Gene Expression Analysis

RNA-seq reads were mapped to the GRCm38 genome and transcript-level quantification was performed using Salmon v.1.9.0. Tximport v.1.2.0 was used to compute normalized gene-level counts from the transcript-level abundance estimates (scaling these using the average transcript length over samples and the library size). Only genes with at least 1 cpm in at least 3 samples were retained for downstream analysis. Differential gene expression was calculated using DESeq2 v.3.15, with trimmed mean of M-values normalization and multiple hypothesis correction of p-values performed using the Benjamini-Hochberg method. Differential expression of genes was tested in two comparisons: (1) old+OSKM vs old control and (2) young vs. old. For a gene to be called a differentially expressed gene (DEG) it had to have a Benjamini-Hochberg adjusted P-value<0.05 with no minimum log₂ fold change cutoff.

### Gene Set Enrichment Analysis (GSEA)

GSEA was performed using two different databases of gene sets; (1) Chromatin Enrichment Analysis (ChEA) 2022 as part of Enrichr and (2) Molecular Signatures Database (MSigDB) v.7.0, available by the Broad Institute's GSEA tool.

TABLE 1
(Supplementary Table S1a. WGBS samples)
WGBS data

| | | | Referenced | | Average | | | Age at the |
|---|---|---|---|---|---|---|---|---|
| | | Sample | in the | Data | CpG | | | start of the |
| Group | Type | ID | manuscript as | source | depth | Age | Treatment | treatment |
| Young | PRIMARY | Y4F-1 | Y-1 | This | 8.3 | 3 | Control (PBS | — |
| | | | | paper | | months | used as vehicle) | |
| Young | PRIMARY | Y4F-3 | Y-2 | This | 9.4 | 3 | Control (PBS | — |
| | | | | paper | | months | used as vehicle) | |
| Young | PRIMARY | Y4F-4 | Y-3 | This | 9.6 | 3 | Control (PBS | — |
| | | | | paper | | months | used as vehicle) | |
| Old | PRIMARY | 5760-con | O-1 | This | 10 | 22 | Control (PBS | — |
| | | | | paper | | months | used as vehicle) | |
| Old | PRIMARY | 5761-con | O-2 | This | 11.8 | 22 | Control (PBS | — |
| | | | | paper | | months | used as vehicle) | |
| Old | PRIMARY | 5762-con | O-3 | This | 8.9 | 22 | Control (PBS | — |
| | | | | paper | | months | used as vehicle) | |
| Old | PRIMARY | 5762-con | O-4 | This | 8.3 | 22 | Control (PBS | — |
| | | | | paper | | months | used as vehicle) | |
| Old + | PRIMARY | 253-dox | O + | This | 7.6 | 22 | Cyclic | 15 |
| OSKM | | | OSKM-1 | paper | | months | induction of | months |
| | | | | | | | OSKM via | |
| | | | | | | | doxycycline* | |
| Old + | PRIMARY | 254-dox | O + | This | 8.4 | 22 | Cyclic | 15 |
| OSKM | | | OSKM-2 | paper | | months | induction of | months |
| | | | | | | | OSKM via | |
| | | | | | | | doxycycline* | |
| Old + | PRIMARY | 255-dox | O + | This | 8.9 | 22 | Cyclic | 15 |
| OSKM | | | OSKM-3 | paper | | months | induction of | months |
| | | | | | | | OSKM via | |
| | | | | | | | doxycycline* | |
| Old + | PRIMARY | 284-dox | O + | This | 8.5 | 22 | Cyclic | 15 |
| OSKM | | | OSKM-4 | paper | | months | induction of | months |
| | | | | | | | OSKM via | |
| | | | | | | | doxycycline* | |
| Old + | PRIMARY | 904-dox | O + | This | 7.9 | 22 | Cyclic | 15 |
| OSKM | | | OSKM-5 | paper | | months | induction of | months |
| | | | | | | | OSKM via | |
| | | | | | | | doxycycline* | |

TABLE 2
(Supplementary Table S1b. RNA-seq data)

| | | | | | | Age at the |
|---|---|---|---|---|---|---|
| | | Sample | Data | | | start of the |
| Group | Type | ID | source ** | Age | Treatment | treatment |
| Young | PRIMARY | Young_Skin_non- | GEO accession | 3 | Control (PBS | — |
| | | dox_5269 | number: | months | used as vehicle) | |
| | | | GSM5737119 | | | |
| Young | PRIMARY | Young_Skin_non- | GEO accession | 3 | Control (PBS | — |
| | | dox_5275 | number: | months | used as vehicle) | |
| | | | GSM5737120 | | | |
| Young | PRIMARY | Young_Skin_non- | GEO accession | 3 | Control (PBS | — |
| | | dox_5281 | number: | months | used as vehicle) | |
| | | | GSM5737121 | | | |
| Young | PRIMARY | Young_Skin_non- | GEO accession | 3 | Control (PBS | — |
| | | dox_5287 | number: | months | used as vehicle) | |
| | | | GSM5737122 | | | |
| Old | PRIMARY | Skin_Control_6540 | GEO accession | 22 | Control (PBS | — |
| | | | number: | months | used as vehicle) | |
| | | | GSM5737052 | | | |
| Old | PRIMARY | Skin_Control_6541 | GEO accession | 22 | Control (PBS | — |
| | | | number: | months | used as vehicle) | |
| | | | GSM5737052 | | | |
| Old | PRIMARY | Skin_Control_6542 | GEO accession | 22 | Control (PBS | — |
| | | | number: | months | used as vehicle) | |
| | | | GSM5737052 | | | |
| Old | PRIMARY | Skin_Control_6543 | GEO accession | 22 | Control (PBS | — |
| | | | number: | months | used as vehicle) | |
| | | | GSM5737052 | | | |
| Old | PRIMARY | Skin_Control_6544 | GEO accession | 22 | Control (PBS | — |
| | | | number: | months | used as vehicle) | |
| | | | GSM5737052 | | | |
| Old + | PRIMARY | Skin_4F_6545 | GEO accession | 22 | Cyclic induction | 15 |
| OSKM | | | number: | months | of OSKM via | months |
| | | | GSM5737047 | | doxycycline * | |
| Old + | PRIMARY | Skin_4F_6546 | GEO accession | 22 | Cyclic induction | 15 |
| OSKM | | | number: | months | of OSKM via | months |
| | | | GSM5737048 | | doxycycline * | |
| Old + | PRIMARY | Skin_4F_6547 | GEO accession | 22 | Cyclic induction | 15 |
| OSKM | | | number: | months | of OSKM via | months |
| | | | GSM5737049 | | doxycycline * | |
| Old + | PRIMARY | Skin_4F_6548 | GEO accession | 22 | Cyclic induction | 15 |
| OSKM | | | number: | months | of OSKM via | months |
| | | | GSM5737050 | | doxycycline* | |

* Mice treated with doxycycline (1 mg/ml) in drinking water for 2-days to induce 4F overexpression followed by 5 days of withdraw per week for 1 months.
** RNA-seq data published in: Browder, K. C. et al. In vivo partial reprogramming alters age-associated molecular changes during physiological aging in mice. Nature Aging 2022 2: 32, 243-253 (2022).

TABLE 3
(Supplementary Table S2a. Ranked genes by differential methylation
(Old + OSKM vs Old-2): rank comp., p-value comp., and q-value comp.)

| | RANK | p-value | q-value | p-value | p-value | p-value | p-value | p-value |
|---|---|---|---|---|---|---|---|---|
| Gene | comp. | comp. | comp. | comp. TR1 | comp. TR2 | comp. TR3 | comp. TR4 | comp. TR5 |
| Cntnap2 | 1 | 2.22E−16 | 1.64E−12 | 8.79E−05 | 4.88E−05 | 2.93E−05 | 1.95E−05 | 0.000244 |
| Def8 | 1 | 2.22E−16 | 1.64E−12 | 4.88E−05 | 6.84E−05 | 1.95E−05 | 8.79E−05 | 4.88E−05 |
| Hic1 | 3 | 2.22E−16 | 1.64E−12 | 0.128187 | 0 | 0.007141 | 0.061407 | 0.108268 |
| Gm13034 | 4 | 4.61E−16 | 2.55E−12 | 6.58E−05 | 8.34E−05 | 4.31E−05 | 7.35E−05 | 0.000113 |
| Galnt13 | 5 | 4.15E−15 | 1.84E−11 | 4.88E−05 | 1.95E−05 | 0.000234 | 6.84E−05 | 0.001416 |
| Rbfox1 | 6 | 1.96E−14 | 7.25E−11 | 1.72E−07 | 0.030459 | 0.027372 | 5.46E−07 | 0.001514 |
| Hoxa2 | 7 | 2.71E−14 | 8.58E−11 | 0.000166 | 4.88E−05 | 0.000537 | 0.000166 | 0.000234 |
| Sox11 | 8 | 7.53E−14 | 2.08E−10 | 0.000469 | 0.004425 | 5.86E−05 | 4.88E−05 | 8.79E−05 |
| Pcdh8 | 9 | 2.30E−13 | 5.67E−10 | 5.86E−05 | 0.000186 | 0.001895 | 0.000234 | 0.000371 |
| Gm9748 | 10 | 2.61E−12 | 5.79E−09 | 0.000215 | 0.000313 | 0.002149 | 0.000368 | 0.000498 |
| Hoxaas3 | 11 | 3.08E−12 | 6.20E−09 | 8.79E−05 | 0.000283 | 0.010062 | 8.79E−05 | 0.001446 |
| Hoxd1 | 12 | 4.49E−12 | 8.28E−09 | 0.00528 | 0.000504 | 0.000774 | 0.000126 | 0.000187 |
| Mup2 | 13 | 9.96E−12 | 1.64E−08 | 0.000712 | 0.000155 | 0.003777 | 0.000433 | 0.000654 |
| Mdga2 | 14 | 1.03E−11 | 1.64E−08 | 0.000469 | 0.000352 | 0.000352 | 0.001543 | 0.001377 |
| Gm10377 | 15 | 1.40E−11 | 2.06E−08 | 0.001038 | 0.000673 | 0.001203 | 0.000311 | 0.00066 |
| Hoxa3 | 16 | 1.53E−11 | 2.11E−08 | 0.000762 | 0.000186 | 0.001612 | 0.000528 | 0.001583 |
| Skint11 | 17 | 1.90E−11 | 2.47E−08 | 0.000108 | 0.001167 | 0.000789 | 0.000698 | 0.00349 |
| Mmp16 | 18 | 3.05E−11 | 3.75E−08 | 0.000205 | 0.002051 | 0.000918 | 0.00211 | 0.000508 |
| Tdpoz5 | 19 | 4.81E−11 | 5.60E−08 | 0.00041 | 0.00016 | 0.000316 | 0.002839 | 0.011766 |
| Senp8 | 20 | 5.07E−11 | 5.60E−08 | 0.001817 | 0.001006 | 0.001563 | 0.000381 | 0.000674 |
| Haglr | 21 | 5.31E−11 | 5.60E−08 | 0.003947 | 0.00169 | 0.001446 | 0.000342 | 0.000234 |
| Gm45351 | 22 | 7.59E−11 | 7.35E−08 | 0.00092 | 0.000917 | 0.001541 | 0.001047 | 0.000848 |
| Vmn2r47 | 23 | 7.64E−11 | 7.35E−08 | 0.00092 | 0.000917 | 0.001541 | 0.001047 | 0.000854 |
| Ajm1 | 24 | 9.16E−11 | 8.33E−08 | 0.000545 | 0.003046 | 0.002185 | 0.000761 | 0.000517 |
| Foxa1 | 25 | 9.41E−11 | 8.33E−08 | 0.000948 | 0.00105 | 0.0027 | 0.000783 | 0.0007 |
| Peg12 | 26 | 1.04E−10 | 8.85E−08 | 0.009084 | 0.000214 | 0.000172 | 0.000423 | 0.011618 |
| Defa23 | 27 | 1.32E−10 | 1.08E−07 | 0.001543 | 0.000123 | 0.003019 | 0.000976 | 0.003845 |
| Nfix | 28 | 2.18E−10 | 1.70E−07 | 0.000381 | 5.86E−05 | 0.004699 | 0.007307 | 0.004953 |
| Grid2 | 29 | 2.22E−10 | 1.70E−07 | 0.002511 | 0.000596 | 0.001797 | 0.000381 | 0.00379 |
| Pcdhga5 | 30 | 3.53E−10 | 2.53E−07 | 0.001954 | 0.004992 | 0.002091 | 8.79E−05 | 0.003663 |
| Cldn6 | 31 | 3.54E−10 | 2.53E−07 | 0.001771 | 0.00391 | 0.000646 | 0.001569 | 0.000938 |
| Hoxb3 | 32 | 3.78E−10 | 2.62E−07 | 0.004328 | 0.000283 | 8.79E−05 | 0.041908 | 0.001573 |
| Camk2b | 33 | 4.45E−10 | 2.99E−07 | 0.000849 | 0.006624 | 0.000352 | 0.0095 | 0.000455 |
| Ltbp3 | 34 | 6.97E−10 | 4.54E−07 | 0.000371 | 0.018776 | 0.000322 | 0.009675 | 0.000655 |
| Otx1 | 35 | 7.19E−10 | 4.55E−07 | 0.001036 | 0.015659 | 0.000361 | 0.003839 | 0.000655 |
| Nefm | 36 | 8.59E−10 | 5.28E−07 | 0.008431 | 0.000361 | 0.000293 | 0.001954 | 0.010345 |
| Nr4a2 | 37 | 9.07E−10 | 5.43E−07 | 0.004308 | 0.000742 | 0.002276 | 7.82E−05 | 0.033742 |
| Sgcz | 38 | 1.09E−09 | 6.37E−07 | 0.002071 | 0.000733 | 0.008391 | 0.000723 | 0.002579 |
| Lrp1b | 39 | 1.39E−09 | 7.88E−07 | 0.021872 | 0.001123 | 0.003028 | 0.000322 | 0.001299 |
| Atn1 | 40 | 1.45E−09 | 7.89E−07 | 0.000463 | 0.004245 | 0.006265 | 0.004964 | 0.000535 |
| Crtc1 | 41 | 1.46E−09 | 7.89E−07 | 0.001439 | 7.45E−05 | 0.025629 | 0.002186 | 0.005502 |
| Skint5 | 42 | 2.09E−09 | 1.10E−06 | 0.00243 | 0.003119 | 0.002376 | 0.00102 | 0.002713 |
| Csmd1 | 43 | 2.56E−09 | 1.32E−06 | 0.01944 | 0.001387 | 0.001895 | 0.003146 | 0.000391 |
| Dlgap1 | 44 | 4.48E−09 | 2.26E−06 | 0.000469 | 0.003546 | 0.009271 | 0.000391 | 0.019831 |
| Cdh2 | 45 | 5.22E−09 | 2.57E−06 | 0.001534 | 0.007317 | 0.001934 | 0.005783 | 0.001133 |
| Hspa1a | 46 | 6.48E−09 | 3.12E−06 | 0.001196 | 0.000168 | 0.000809 | 0.010754 | ND |
| Skint6 | 47 | 7.11E−09 | 3.35E−06 | 0.002144 | 0.010416 | 0.001592 | 0.001323 | 0.004321 |
| Ptprd | 48 | 7.34E−09 | 3.38E−06 | 0.010238 | 0.006477 | 7.22E−05 | 0.005949 | 0.007395 |
| Hs3st4 | 49 | 7.83E−09 | 3.49E−06 | 0.004425 | 0.000899 | 0.001192 | 0.002286 | 0.020935 |
| Pim1 | 50 | 7.87E−09 | 3.49E−06 | ND | 0.00023 | 0.000105 | ND | 0.001004 |
| Ebf1 | 51 | 9.37E−09 | 4.07E−06 | 0.001729 | 0.000381 | 0.050554 | 0.000508 | 0.016509 |
| Trank1 | 52 | 1.10E−08 | 4.69E−06 | 0.003771 | 0.002511 | 0.011517 | 0.00421 | 0.000733 |
| Serpina1b | 53 | 1.34E−08 | 5.61E−06 | 0.012836 | 0.008939 | 0.006877 | 0.000195 | 0.002745 |
| Skint4 | 54 | 1.52E−08 | 6.24E−06 | 0.00089 | 0.011679 | 0.00106 | 0.000381 | 0.116518 |
| Smad3 | 55 | 1.94E−08 | 7.82E−06 | 0.000967 | 0.00761 | 0.006184 | 0.017799 | 0.000801 |
| Rassf3 | 56 | 2.07E−08 | 8.20E−06 | 0.009505 | 0.000869 | 0.000596 | 0.004386 | 0.032374 |
| Hoxaas2 | 57 | 2.17E−08 | 8.45E−06 | 0.002032 | 0.006174 | 0.027216 | 0.002091 | 0.001036 |
| Msi1 | 58 | 2.46E−08 | 9.39E−06 | 0.005317 | 0.088786 | 0.001371 | 0.000502 | 0.002626 |
| Zfp503 | 59 | 2.56E−08 | 9.60E−06 | 0.000664 | 0.002384 | 0.007737 | 0.010443 | 0.006975 |
| 1700034I23Rik | 60 | 3.18E−08 | 1.18E−05 | 0.00258 | 0.005629 | 0.000512 | 0.001404 | 0.110316 |
| Csmd2 | 61 | 3.27E−08 | 1.19E−05 | 0.001192 | 0.025753 | 0.000655 | 0.013157 | 0.004484 |
| Nox4 | 62 | 3.64E−08 | 1.30E−05 | 0.030205 | 0.006614 | 0.005207 | 0.000703 | 0.001837 |
| Pcdhga6 | 63 | 4.00E−08 | 1.38E−05 | 0.021208 | 0.004601 | 0.004171 | 0.000948 | 0.003888 |
| Snord116l1 | 64 | 4.06E−08 | 1.38E−05 | 0.009173 | 0.000625 | 0.009915 | 0.005734 | 0.004679 |
| Lrfn1 | 65 | 4.09E−08 | 1.38E−05 | 0.013129 | 0.008118 | 0.00043 | 0.001221 | 0.027529 |
| Has1 | 66 | 4.10E−08 | 1.38E−05 | 0.011801 | 0.000528 | 0.004679 | 0.005558 | 0.009544 |
| Gvin2 | 67 | 4.36E−08 | 1.42E−05 | 0.001838 | 0.005407 | 0.006102 | 0.014893 | 0.001836 |
| Gvin-ps2 | 67 | 4.36E−08 | 1.42E−05 | 0.001838 | 0.005407 | 0.006102 | 0.014893 | 0.001836 |
| Lzts3 | 69 | 4.70E−08 | 1.51E−05 | 0.002931 | 0.001543 | 0.007219 | 0.005949 | 0.009329 |
| Nxph4 | 70 | 4.83E−08 | 1.53E−05 | 0.029453 | 0.000528 | 0.004884 | 0.00466 | 0.005285 |
| Irx3 | 71 | 5.11E−08 | 1.59E−05 | 0.001885 | 0.002081 | 0.002032 | 0.036164 | 0.006926 |
| Bhlhe40 | 72 | 5.31E−08 | 1.63E−05 | 0.052215 | 0.003155 | 0.00086 | 0.001543 | 0.009544 |
| Grik2 | 73 | 5.90E−08 | 1.79E−05 | 0.009945 | 0.002081 | 0.013168 | 0.000586 | 0.01478 |
| Kcnj3 | 74 | 6.18E−08 | 1.83E−05 | 0.01143 | 0.000899 | 0.003507 | 0.002091 | 0.033107 |
| Lingo2 | 75 | 6.21E−08 | 1.83E−05 | 0.001407 | 0.001543 | 0.004836 | 0.003859 | 0.061847 |
| Syngap1 | 76 | 6.46E−08 | 1.88E−05 | 0.187247 | 0.003107 | 0.000401 | 0.005196 | 0.002167 |
| Cdca7l | 77 | 6.57E−08 | 1.89E−05 | 0.011791 | 0.000381 | 0.00719 | 0.011176 | 0.007415 |
| St6gal2 | 78 | 6.78E−08 | 1.93E−05 | 0.051257 | 0.017574 | 0.000977 | 0.00043 | 0.007346 |
| Vmn2r-ps11 | 79 | 7.31E−08 | 2.05E−05 | 0.003113 | 0.001252 | 0.012277 | 0.005258 | 0.012047 |
| Tox3 | 80 | 7.68E−08 | 2.13E−05 | 0.002569 | 0.011841 | 0.017437 | 0.005392 | 0.001123 |
| Dnah7c | 81 | 8.05E−08 | 2.20E−05 | 0.116171 | 0.000703 | 0.000371 | 0.017818 | 0.006281 |
| Pcdhga8 | 82 | 8.53E−08 | 2.30E−05 | 0.002579 | 0.010433 | 0.008626 | 0.00336 | 0.00466 |
| Pcdhgb5 | 83 | 8.83E−08 | 2.36E−05 | 0.000899 | 0.037337 | 0.049196 | 0.00041 | 0.005588 |
| Nrg1 | 84 | 9.44E−08 | 2.49E−05 | 0.004699 | 0.004865 | 0.027529 | 0.001934 | 0.00336 |
| Col28a1 | 85 | 9.69E−08 | 2.52E−05 | 0.000301 | 0.006769 | 0.001008 | 0.063 | 0.032585 |
| Smarca5-ps | 86 | 1.10E−07 | 2.83E−05 | ND | 0.000944 | 0.004356 | 0.0028 | 0.003701 |
| Gm20110 | 87 | 1.21E−07 | 3.02E−05 | 0.012827 | 0.013462 | 0.000586 | 0.002911 | 0.018522 |
| Adcy2 | 88 | 1.34E−07 | 3.02E−05 | 0.014399 | 0.000772 | 0.00632 | 0.005158 | 0.016998 |
| Eif4a3l1 | 89 | 1.39E−07 | 3.02E−05 | 0.005946 | 0.00222 | 0.008391 | 0.007273 | 0.007967 |
| Wnt3a | 90 | 1.40E−07 | 3.02E−05 | ND | 0.000482 | 0.000364 | 0.135773 | 0.002361 |
| Vmn1r101 | 91 | 1.40E−07 | 3.02E−05 | ND | 0.000482 | 0.000364 | 0.135773 | 0.002361 |
| Vmn1r250 | 92 | 1.40E−07 | 3.02E−05 | ND | 0.000482 | 0.000364 | 0.135773 | 0.002361 |
| Gm10665 | 93 | 1.40E−07 | 3.02E−05 | ND | 0.000482 | 0.000364 | 0.135773 | 0.002361 |
| Hmbox1 | 94 | 1.40E−07 | 3.02E−05 | ND | 0.000482 | 0.000364 | 0.135773 | 0.002361 |
| Vmn1r100 | 95 | 1.40E−07 | 3.02E−05 | ND | 0.000482 | 0.000364 | 0.135773 | 0.002361 |
| Gm10668 | 96 | 1.40E−07 | 3.02E−05 | ND | 0.000482 | 0.000364 | 0.135773 | 0.002361 |
| Gm4513 | 97 | 1.40E−07 | 3.02E−05 | ND | 0.000482 | 0.000364 | 0.135773 | 0.002361 |
| Vmn1r142 | 98 | 1.40E−07 | 3.02E−05 | ND | 0.000482 | 0.000364 | 0.135773 | 0.002361 |
| Vmn1r143 | 99 | 1.40E−07 | 3.02E−05 | ND | 0.000482 | 0.000364 | 0.135773 | 0.002361 |
| Vmn1r251 | 100 | 1.40E−07 | 3.02E−05 | ND | 0.000482 | 0.000364 | 0.135773 | 0.002361 |
| Vmnlr254 | 101 | 1.40E−07 | 3.02E−05 | ND | 0.000482 | 0.000364 | 0.135773 | 0.002361 |
| Vmn1r152 | 102 | 1.40E−07 | 3.02E−05 | ND | 0.000482 | 0.000364 | 0.135773 | 0.002361 |
| Vmn1r255 | 103 | 1.40E−07 | 3.02E−05 | ND | 0.000482 | 0.000364 | 0.135773 | 0.002361 |
| Gal3st3 | 104 | 1.42E−07 | 3.02E−05 | 0.020182 | 0.002862 | 0.002335 | 0.002335 | 0.020925 |
| Cyp3a41a | 105 | 1.43E−07 | 3.02E−05 | 0.016696 | 0.009276 | 0.002876 | 0.001856 | 0.008069 |
| Pantr1 | 106 | 1.45E−07 | 3.02E−05 | 0.002862 | 0.023494 | 0.00125 | 0.006184 | 0.012973 |
| Gria4 | 107 | 1.47E−07 | 3.04E−05 | 0.000244 | 0.00297 | 0.013979 | 0.024588 | 0.027558 |
| Klrc1 | 108 | 1.50E−07 | 3.07E−05 | 0.006055 | 0.062333 | 0.003907 | 0.001246 | 0.003822 |
| Gm3002 | 109 | 1.54E−07 | 3.13E−05 | 0.011721 | 0.015203 | 0.000551 | 0.002999 | 0.024716 |
| Stx1b | 110 | 1.58E−07 | 3.17E−05 | 0.005158 | 0.002188 | 0.000694 | 0.01015 | 0.093957 |
| Hmx1 | 111 | 1.67E−07 | 3.33E−05 | 0.008186 | 0.001514 | 0.030313 | 0.00125 | 0.016988 |
| 5730596B20Rik | 112 | 1.80E−07 | 3.56E−05 | 0.005881 | 0.029177 | 0.004939 | 0.001949 | 0.005289 |
| Galnt10 | 113 | 1.83E−07 | 3.59E−05 | 0.000786 | 0.000469 | 0.345916 | 0.000261 | 0.268116 |
| Ephb4 | 114 | 1.87E−07 | 3.63E−05 | 0.063019 | 0.006281 | 0.005783 | 0.000469 | 0.008499 |
| Mapk8ip2 | 115 | 1.89E−07 | 3.64E−05 | 0.008382 | 0.006184 | 0.000596 | 0.020583 | 0.014526 |
| Zbtb7a | 116 | 1.93E−07 | 3.66E−05 | 0.064543 | 0.000322 | 0.00043 | 0.597444 | 0.001768 |
| Zfp697 | 117 | 1.93E−07 | 3.66E−05 | 0.004347 | 0.000254 | 0.029092 | 0.021404 | 0.013813 |
| Mrgpra2a | 118 | 2.07E−07 | 3.89E−05 | 0.034991 | 0.019222 | 0.001354 | 0.000999 | 0.011324 |
| Obox1 | 119 | 2.20E−07 | 4.08E−05 | 0.001734 | 0.004548 | 0.001708 | 0.00545 | 0.150198 |
| Kcnn4 | 120 | 2.22E−07 | 4.09E−05 | 0.045392 | 0.000508 | 0.001245 | 0.064019 | 0.006076 |
| Galnt2 | 121 | 2.35E−07 | 4.30E−05 | 0.004484 | 0.004953 | 0.007297 | 0.005519 | 0.013354 |
| Tmem181a | 122 | 2.44E−07 | 4.40E−05 | 0.000195 | 0.005651 | 0.000381 | 0.234398 | 0.126412 |
| Trappc5 | 123 | 2.44E−07 | 4.40E−05 | 0.009867 | 0.014653 | 0.006184 | 0.006721 | 0.002081 |
| Cilp2 | 124 | 2.68E−07 | 4.75E−05 | 0.003077 | 0.029023 | 0.002667 | 0.046041 | 0.00127 |
| Obox3 | 125 | 2.68E−07 | 4.75E−05 | 0.001931 | 0.008108 | 0.002797 | 0.005615 | 0.056801 |
| Hoxd3 | 126 | 2.73E−07 | 4.80E−05 | 0.544859 | 0.000147 | 0.147305 | 0.004279 | 0.000283 |
| Nrg3 | 127 | 2.82E−07 | 4.92E−05 | 0.007258 | 0.005734 | 0.001377 | 0.004093 | 0.063156 |
| Tmem181c-ps | 128 | 2.89E−07 | 5.01E−05 | 0.010404 | 0.000674 | 0.029043 | 0.004142 | 0.018111 |
| Gm2042 | 129 | 3.00E−07 | 5.15E−05 | 0.002869 | 0.001456 | 0.008088 | 0.040471 | 0.011656 |
| Olfr18 | 130 | 3.11E−07 | 5.28E−05 | 0.033932 | 0.015606 | 0.008078 | 0.000809 | 0.00481 |
| Vmn1r107 | 131 | 3.12E−07 | 5.28E−05 | ND | 0.000823 | 0.001054 | 0.051147 | 0.003159 |
| Xkr6 | 132 | 3.41E−07 | 5.72E−05 | 0.013598 | 0.00043 | 0.090841 | 0.002384 | 0.014673 |
| Pcdhgb4 | 133 | 3.60E−07 | 5.99E−05 | 0.004005 | 0.032491 | 0.017848 | 0.001436 | 0.00593 |
| Lgals6 | 134 | 3.66E−07 | 6.05E−05 | 0.003182 | 0.001006 | 0.01934 | 0.129538 | 0.002517 |
| Col1a1 | 135 | 4.14E−07 | 6.80E−05 | 0.00041 | 0.058291 | 0.071537 | 0.000977 | 0.013979 |
| Vwc2 | 136 | 4.22E−07 | 6.86E−05 | 0.015982 | 0.003995 | 0.007952 | 0.00253 | 0.018561 |
| Nkx2-1 | 137 | 4.28E−07 | 6.91E−05 | 0.017691 | 0.003878 | 0.060039 | 0.008391 | 0.000703 |
| Trhde | 138 | 4.31E−07 | 6.91E−05 | 0.039134 | 0.005803 | 0.036037 | 0.008069 | 0.000371 |
| Sntg1 | 139 | 4.34E−07 | 6.91E−05 | 0.011029 | 0.001192 | 0.021472 | 0.004171 | 0.020954 |
| Fbln1 | 140 | 4.71E−07 | 7.44E−05 | 0.00126 | 0.024149 | 0.006477 | 0.030752 | 0.004484 |
| Erbb4 | 141 | 4.85E−07 | 7.61E−05 | 0.024764 | 0.004171 | 0.008206 | 0.006487 | 0.005119 |
| Gm20752 | 142 | 5.11E−07 | 7.97E−05 | 0.00697 | 0.00085 | 0.016993 | 0.002525 | 0.117979 |
| Pcdhga3 | 143 | 5.60E−07 | 8.67E−05 | 0.008352 | 0.036819 | 0.022097 | 0.000371 | 0.013237 |
| Lin28b | 144 | 6.06E−07 | 9.32E−05 | 0.002315 | 0.001426 | 0.006193 | 0.002667 | 0.673261 |
| Hoxb5os | 145 | 6.13E−07 | 9.36E−05 | 0.000186 | 0.002677 | 0.008157 | 0.055438 | 0.165641 |
| 9030404E10Rik | 146 | 6.52E−07 | 9.89E−05 | 0.013969 | 0.004308 | 0.004005 | 0.040277 | 0.004122 |
| Krt5 | 147 | 6.61E−07 | 9.92E−05 | 0.02833 | 0.000244 | 0.003136 | 0.032423 | 0.057861 |
| Fat3 | 148 | 6.63E−07 | 9.92E−05 | 0.006604 | 0.010687 | 0.012094 | 0.004992 | 0.009583 |
| Hebp1 | 149 | 6.79E−07 | 0.000101 | 0.001895 | 0.002286 | 0.006946 | 0.107037 | 0.013051 |
| Usp29 | 150 | 7.25E−07 | 0.000106 | 0.000625 | 0.003546 | 0.119493 | 0.001534 | 0.111854 |
| Vmn1r131 | 151 | 7.26E−07 | 0.000106 | ND | 0.000304 | 0.001037 | 0.068984 | 0.016953 |
| Cpq | 152 | 7.98E−07 | 0.000116 | 0.012289 | 0.006125 | 0.012866 | 0.003292 | 0.015982 |
| Ankrd33b | 153 | 8.48E−07 | 0.000123 | 0.005246 | 0.010433 | 0.009114 | 0.014067 | 0.007805 |
| Uggt2 | 154 | 8.62E−07 | 0.000124 | 0.041772 | 0.004484 | 0.028476 | 0.007336 | 0.001426 |
| Csf2ra | 155 | 8.75E−07 | 0.000125 | 0.203622 | 0.085233 | 0.001612 | 0.000596 | 0.003409 |
| L3mbt14 | 156 | 9.34E−07 | 0.000133 | 0.004777 | 0.02027 | 0.001817 | 0.007356 | 0.047447 |
| Hoxb3os | 157 | 9.46E−07 | 0.000133 | 0.029688 | 0.00846 | 5.86E−05 | 0.125325 | 0.033791 |
| Pcdh15 | 158 | 9.55E−07 | 0.000134 | 0.015161 | 0.00719 | 0.003566 | 0.003888 | 0.041752 |
| Usp10 | 159 | 1.06E−06 | 0.000147 | 0.003947 | 0.021882 | 0.003204 | 0.007278 | 0.035373 |
| Ugt8a | 160 | 1.08E−06 | 0.000149 | 0.012748 | 0.012827 | 0.003947 | 0.011381 | 0.009935 |
| Rftn1 | 161 | 1.10E−06 | 0.000152 | 0.001583 | 0.018551 | 0.007131 | 0.013989 | 0.025634 |
| Mansc4 | 162 | 1.12E−06 | 0.000154 | 0.024149 | 0.029522 | 0.013774 | 0.000723 | 0.010795 |
| Prdm5 | 163 | 1.14E−06 | 0.000154 | 0.013657 | 0.000801 | 0.014507 | 0.009114 | 0.05366 |
| Evx1 | 164 | 1.15E−06 | 0.000155 | 0.050925 | 0.004757 | 0.007004 | 0.003556 | 0.013041 |
| 2410018L13Rik | 165 | 1.16E−06 | 0.000156 | 0.024365 | 0.03448 | 9.77E−05 | ND | 0.007757 |
| A330008L17Rik | 166 | 1.17E−06 | 0.000157 | 0.005383 | 0.011312 | 0.01478 | 0.004347 | 0.020622 |
| Gm19345 | 167 | 1.24E−06 | 0.000164 | 0.012025 | 0.407508 | 0.013481 | 5.86E−05 | 0.022244 |
| D830030K20Rik | 168 | 1.24E−06 | 0.000164 | 0.003985 | 0.005076 | 0.005781 | 0.010873 | 0.068011 |
| 4930486L24Rik | 169 | 1.27E−06 | 0.000167 | 0.03636 | 0.015298 | 0.006926 | 0.002452 | 0.009398 |
| Slc7a11 | 170 | 1.32E−06 | 0.000172 | 0.003614 | 0.039046 | 0.058574 | 0.00421 | 0.002667 |
| Osm | 171 | 1.34E−06 | 0.000173 | 0.006672 | 0.004611 | 0.002081 | 0.020915 | 0.070932 |
| Gucy1a2 | 172 | 1.35E−06 | 0.000173 | 0.033683 | 0.055526 | 0.000967 | 0.005871 | 0.008948 |
| Cd84 | 173 | 1.43E−06 | 0.000182 | 0.001964 | 0.005431 | 0.005041 | 0.004406 | 0.430104 |
| Rims2 | 174 | 1.44E−06 | 0.000184 | 0.032667 | 0.01224 | 0.013422 | 0.015786 | 0.001221 |
| Nkx6-2 | 175 | 1.46E−06 | 0.000185 | 0.007775 | 0.027392 | 0.021775 | 0.003363 | 0.006715 |
| Espn | 176 | 1.48E−06 | 0.000186 | 0.002091 | 0.005041 | 0.142303 | 0.005324 | 0.013354 |
| Esyt2 | 177 | 1.49E−06 | 0.000186 | 0.000801 | 0.009046 | 0.002696 | 0.074048 | 0.074341 |
| Psg25 | 178 | 1.49E−06 | 0.000186 | 0.042825 | 0.00243 | 0.018303 | 0.005009 | 0.011303 |
| Dync2i1 | 179 | 1.56E−06 | 0.000192 | 0.000414 | 0.00494 | 0.003538 | 0.104703 | 0.149229 |
| Mup18 | 180 | 1.64E−06 | 0.000202 | 0.003861 | 0.011968 | ND | 0.002695 | 0.00762 |
| Elavl4 | 181 | 1.68E−06 | 0.000205 | 0.005861 | 0.02154 | 0.00677 | 0.002257 | 0.064279 |
| Myo9a | 182 | 1.69E−06 | 0.000205 | 0.011596 | 0.012582 | 0.010609 | 0.006252 | 0.012885 |
| Gemin5 | 183 | 1.71E−06 | 0.000207 | 0.008518 | 0.031466 | 0.008626 | 0.000234 | 0.233769 |
| Chd9 | 184 | 1.78E−06 | 0.000214 | 0.000371 | 0.005129 | 0.018346 | 0.294453 | 0.012924 |
| Btn19 | 185 | 1.83E−06 | 0.000219 | 0.160405 | 0.004025 | 0.011381 | 0.000508 | 0.036858 |
| Cacna1a | 186 | 1.84E−06 | 0.000219 | 0.004716 | 0.00127 | 0.013706 | 0.027548 | 0.061163 |
| Elfn1 | 187 | 1.90E−06 | 0.000225 | 0.059981 | 0.006828 | 0.071791 | 0.000801 | 0.006096 |
| Tap2 | 188 | 1.99E−06 | 0.000234 | 0.0072 | 0.057714 | 0.006262 | 0.004425 | 0.013188 |
| Tspan5 | 189 | 2.01E−06 | 0.000235 | 0.011605 | 0.018336 | 0.00506 | 0.00805 | 0.017721 |
| Cdcp1 | 190 | 2.03E−06 | 0.000235 | 0.069574 | 0.002696 | 0.003966 | 0.005637 | 0.037161 |
| Mir6991 | 191 | 2.04E−06 | 0.000235 | 0.006175 | 0.007155 | 0.042574 | 0.020977 | 0.003963 |
| Gm11627 | 192 | 2.04E−06 | 0.000235 | 0.003672 | 0.097259 | 0.023616 | 0.005153 | 0.003607 |
| Barx1 | 193 | 2.05E−06 | 0.000235 | 0.020222 | 0.016939 | 0.006692 | 0.000459 | 0.149376 |
| Asap2 | 194 | 2.11E−06 | 0.000239 | 0.041509 | 0.090154 | 0.002921 | 0.000841 | 0.017701 |
| Gm3772 | 195 | 2.11E−06 | 0.000239 | 0.002858 | 0.000443 | 0.03062 | 0.014888 | 0.282779 |
| Gm14322 | 196 | 2.13E−06 | 0.000241 | 0.03128 | 0.001075 | 0.024305 | 0.016431 | 0.012289 |

TABLE 4
(Supplementary Table S2b. Ranked genes by differential methylation
(Old + OSKM vs Old-2): RANK prom., p-value prom., and q-value prom.)

| | RANK | p-value | q-value | p-value | p-value | p-value | p-value | p-value |
|---|---|---|---|---|---|---|---|---|
| Gene | prom. | prom. | prom. | prom. TR1 | prom. TR2 | prom. TR3 | prom. TR4 | prom. TR5 |
| Cntnap2 | 3 | 2.03E−14 | 1.45E−10 | 7.25E−05 | 0.000506 | 8.61E−05 | 9.95E−05 | 0.000394 |
| Def8 | 8739 | 0.413812 | 1 | 0.534373 | 0.725189 | 0.085919 | 0.739253 | 0.234555 |
| Hic1 | 657 | 0.001069 | 0.034801 | 0.155262 | 0.00162 | 0.085836 | 0.324889 | 0.058549 |
| Gm13034 | 2 | 4.61E−16 | 4.93E−12 | 6.58E−05 | 8.34E−05 | 4.31E−05 | 7.35E−05 | 0.000113 |
| Galnt13 | 1 | 4.36E−16 | 4.93E−12 | 8.66E−06 | 1.41E−05 | 0.000225 | 0.000113 | 0.000595 |
| Rbfox1 | 872 | 0.00271 | 0.066486 | ND | 0.329594 | 0.05155 | ND | 0.002602 |
| Hoxa2 | 3770 | 0.091396 | 0.518494 | 0.510326 | 0.119788 | 0.050888 | 0.645082 | 0.14398 |
| Sox11 | 5 | 1.31E−13 | 5.59E−10 | 0.001914 | 0.002525 | 4.52E−05 | 2.72E−05 | 0.000161 |
| Pcdh8 | 6 | 8.54E−13 | 3.04E−09 | 0.00026 | 0.000207 | 0.001656 | 0.000275 | 0.000312 |
| Gm9748 | 8 | 2.61E−12 | 6.99E−09 | 0.000215 | 0.000313 | 0.002149 | 0.000368 | 0.000498 |
| Hoxaas3 | 218 | 3.75E−05 | 0.003678 | 0.008345 | 0.033186 | ND | 0.014075 | 0.009643 |
| Hoxd1 | 10 | 4.49E−12 | 9.60E−09 | 0.00528 | 0.000504 | 0.000774 | 0.000126 | 0.000187 |
| Mup2 | NA | ND | ND | ND | ND | ND | ND | ND |
| Mdga2 | 4 | 4.46E−14 | 2.39E−10 | 8.44E−05 | 0.000206 | 8.35E−05 | 0.001073 | 0.000188 |
| Gm10377 | NA | ND | ND | ND | ND | ND | ND | ND |
| Hoxa3 | 3784 | 0.092152 | 0.520849 | 0.509826 | 0.120631 | 0.050969 | 0.645191 | 0.144903 |
| Skint11 | NA | ND | ND | ND | ND | ND | ND | ND |
| Mmp16 | 12 | 1.97E−11 | 3.51E−08 | 9.35E−05 | 0.003228 | 0.000725 | 0.003262 | 0.000356 |
| Tdpoz5 | NA | ND | ND | ND | ND | ND | ND | ND |
| Senp8 | 23 | 3.64E−09 | 3.19E−06 | 0.006361 | 0.00176 | 0.002492 | 0.001928 | 0.00175 |
| Haglr | 11 | 5.40E−12 | 1.05E−08 | 0.005282 | 0.000534 | 0.000809 | 0.000132 | 0.000198 |
| Gm45351 | NA | ND | ND | ND | ND | ND | ND | ND |
| Vmn2r47 | NA | ND | ND | ND | ND | ND | ND | ND |
| Ajm1 | 15 | 9.16E−11 | 1.31E−07 | 0.000545 | 0.003046 | 0.002185 | 0.000761 | 0.000517 |
| Foxa1 | NA | ND | ND | ND | ND | ND | ND | ND |
| Peg12 | 16 | 1.04E−10 | 1.39E−07 | 0.009084 | 0.000214 | 0.000172 | 0.000423 | 0.011618 |
| Defa23 | NA | ND | ND | ND | ND | ND | ND | ND |
| Nfix | 1040 | 0.004497 | 0.092465 | 0.011878 | 0.012646 | ND | 0.416124 | 0.237443 |
| Grid2 | 19 | 7.93E−10 | 8.92E−07 | 0.002672 | 0.001816 | 0.001513 | 0.001717 | 0.001307 |
| Pcdhga5 | 100 | 1.93E−06 | 0.000412 | 0.011899 | 0.021455 | 0.013041 | 0.001325 | 0.033157 |
| Cldn6 | 18 | 3.54E−10 | 4.21E−07 | 0.001771 | 0.00391 | 0.000646 | 0.001569 | 0.000938 |
| Hoxb3 | 6274 | 0.238388 | 0.812194 | 0.19899 | 0.034747 | 0.507314 | 0.739261 | 0.659228 |
| Camk2b | NA | ND | ND | ND | ND | ND | ND | ND |
| Ltbp3 | 244 | 5.38E−05 | 0.00471 | 0.00817 | 0.145901 | 0.008864 | ND | 0.005495 |
| Otx1 | 1285 | 0.00801 | 0.133357 | 0.028954 | 0.409113 | 0.020726 | 0.229006 | 0.117827 |
| Nefm | 37 | 2.91E−08 | 1.66E−05 | 0.002991 | 0.003749 | 0.000598 | 0.010374 | 0.014892 |
| Nr4a2 | 7173 | 0.300021 | 0.894346 | 0.323697 | 0.480574 | 0.575597 | 0.053017 | 0.582746 |
| Sgcz | 30 | 1.51E−08 | 1.08E−05 | 0.002088 | 0.001971 | 0.018157 | 0.003194 | 0.00203 |
| Lrp1b | 1374 | 0.00954 | 0.148539 | 0.926912 | 0.261891 | 0.090873 | 0.092072 | 0.004196 |
| Atn1 | NA | ND | ND | ND | ND | ND | ND | ND |
| Crtc1 | NA | ND | ND | ND | ND | ND | ND | ND |
| Skint5 | NA | ND | ND | ND | ND | ND | ND | ND |
| Csmd1 | 20 | 1.17E−09 | 1.25E−06 | 0.01963 | 0.002561 | 0.001537 | 0.003305 | 0.0001 |
| Dlgap1 | 27 | 6.72E−09 | 5.32E−06 | 0.000394 | 0.006106 | 0.012702 | 0.00032 | 0.019462 |
| Cdh2 | 9 | 3.21E−12 | 7.63E−09 | 0.000189 | 0.001999 | 0.000403 | 0.002695 | 8.11E−05 |
| Hspa1a | 26 | 6.48E−09 | 5.32E−06 | 0.001196 | 0.000168 | 0.000809 | 0.010754 | ND |
| Skint6 | NA | ND | ND | ND | ND | ND | ND | ND |
| Ptprd | 8432 | 0.389951 | 0.988565 | 0.317149 | 0.605588 | ND | 0.62476 | 0.121329 |
| Hs3st4 | 7 | 1.66E−12 | 5.07E−09 | 0.00087 | 0.000107 | 0.000186 | 0.000281 | 0.003289 |
| Pim1 | NA | ND | ND | ND | ND | ND | ND | ND |
| Ebf1 | 31 | 1.95E−08 | 1.30E−05 | 0.002862 | 0.001226 | 0.040098 | 0.000623 | 0.00743 |
| Trank1 | 13 | 5.26E−11 | 8.66E−08 | 0.000512 | 0.000858 | 0.004064 | 0.001831 | 0.000234 |
| Serpina1b | 5918 | 0.21494 | 0.776592 | 0.387201 | 0.615757 | 0.225185 | 0.051998 | 0.497482 |
| Skint4 | NA | ND | ND | ND | ND | ND | ND | ND |
| Smad3 | 3058 | 0.058054 | 0.405909 | 0.045507 | 0.359994 | 0.217366 | 0.749219 | 0.05058 |
| Rassf3 | 668 | 0.001136 | 0.03637 | 0.054096 | 0.035435 | 0.065385 | 0.02317 | 0.153374 |
| Hoxaas2 | 105 | 2.29E−06 | 0.000464 | 0.00536 | 0.003593 | 0.041678 | 0.007884 | 0.028513 |
| Msi1 | NA | ND | ND | ND | ND | ND | ND | ND |
| Zfp503 | 2277 | 0.030838 | 0.289534 | 0.030357 | 0.35733 | 0.114864 | 0.147209 | 0.268615 |
| 1700034I23Rik | 39 | 3.18E−08 | 1.71E−05 | 0.00258 | 0.005629 | 0.000512 | 0.001404 | 0.110316 |
| Csmd2 | 66 | 2.68E−07 | 8.70E−05 | 0.000873 | ND | 0.000267 | ND | 0.004933 |
| Nox4 | 35 | 2.50E−08 | 1.53E−05 | 0.021696 | 0.003883 | 0.018642 | 0.001356 | 0.000409 |
| Pcdhga6 | 512 | 0.000495 | 0.020678 | 0.354311 | 0.014106 | 0.027363 | 0.033441 | 0.03245 |
| Snord116l1 | 5232 | 0.172881 | 0.706619 | 0.715829 | 0.03416 | 0.956558 | 0.181631 | 0.214382 |
| Lrfn1 | 126 | 3.89E−06 | 0.00066 | 0.094011 | 0.120047 | 0.003163 | 0.001761 | 0.005434 |
| Has1 | 2504 | 0.037416 | 0.319496 | 0.180355 | 0.018719 | 0.219043 | 0.318069 | 0.283409 |
| Gvin2 | NA | ND | ND | ND | ND | ND | ND | ND |
| Gvin-ps2 | NA | ND | ND | ND | ND | ND | ND | ND |
| Lzts3 | 15924 | 0.854752 | 1 | 0.803428 | 0.329099 | 0.608818 | 0.841945 | 0.469743 |
| Nxph4 | 14877 | 0.801358 | 1 | 0.939649 | 0.249268 | 0.317595 | 0.68877 | 0.895503 |
| Irx3 | 145 | 8.68E−06 | 0.001281 | 0.023568 | 0.009968 | 0.003433 | 0.052979 | 0.021221 |
| Bhlhe40 | 5296 | 0.177546 | 0.716765 | 0.22265 | 0.68516 | 0.128307 | 0.162146 | 0.301873 |
| Grik2 | 260 | 6.31E−05 | 0.005188 | 0.033663 | 0.024422 | 0.101202 | 0.004102 | 0.031025 |
| Kcnj3 | 73 | 4.18E−07 | 0.000123 | 0.024991 | 0.002476 | 0.002402 | 0.006734 | 0.023622 |
| Lingo2 | 347 | 0.000156 | 0.009592 | 0.003719 | 0.031787 | 0.02418 | 0.047438 | 0.245126 |
| Syngap1 | 1373 | 0.009538 | 0.148539 | ND | 0.022218 | 0.055804 | ND | ND |
| Cdca7l | 49 | 1.07E−07 | 4.61E−05 | 0.004263 | 0.001317 | 0.013765 | 0.01054 | 0.005827 |
| St6gal2 | 91 | 1.15E−06 | 0.00027 | 0.052397 | 0.010287 | 0.003672 | 0.002858 | 0.013908 |
| Vmn2r-ps11 | NA | ND | ND | ND | ND | ND | ND | ND |
| Tox3 | 131 | 4.37E−06 | 0.000713 | 0.028983 | ND | 0.007933 | 0.006983 | 0.001843 |
| Dnah7c | 411 | 0.000263 | 0.013694 | 0.663198 | 0.002575 | 0.004829 | 0.3324 | 0.023806 |
| Pcdhga8 | 611 | 0.000842 | 0.029475 | 0.016601 | 0.03806 | 0.065017 | 0.261897 | 0.027782 |
| Pcdhgb5 | 315 | 0.000115 | 0.007805 | 0.001201 | 0.14339 | 0.612245 | 0.014473 | 0.014811 |
| Nrg1 | 17 | 3.18E−10 | 4.00E−07 | 0.001517 | 0.001665 | 0.008079 | 0.000581 | 0.000492 |
| Col28a1 | NA | ND | ND | ND | ND | ND | ND | ND |
| Smarca5-ps | 51 | 1.10E−07 | 4.61E−05 | ND | 0.000944 | 0.004356 | 0.0028 | 0.003701 |
| Gm20110 | 21 | 2.28E−09 | 2.32E−06 | 0.002116 | 0.004856 | 0.000427 | 0.002293 | 0.005454 |
| Adcy2 | 1893 | 0.020693 | 0.233852 | 0.4944 | 0.030916 | 0.2036 | 0.138705 | 0.06197 |
| Eif4a3l1 | 54 | 1.39E−07 | 5.49E−05 | 0.005946 | 0.00222 | 0.008391 | 0.007273 | 0.007967 |
| Wnt3a | NA | ND | ND | ND | ND | ND | ND | ND |
| Vmn1r101 | NA | ND | ND | ND | ND | ND | ND | ND |
| Vmn1r250 | NA | ND | ND | ND | ND | ND | ND | ND |
| Gm10665 | NA | ND | ND | ND | ND | ND | ND | ND |
| Hmbox1 | NA | ND | ND | ND | ND | ND | ND | ND |
| Vmn1r100 | NA | ND | ND | ND | ND | ND | ND | ND |
| Gm10668 | NA | ND | ND | ND | ND | ND | ND | ND |
| Gm4513 | NA | ND | ND | ND | ND | ND | ND | ND |
| Vmn1r142 | NA | ND | ND | ND | ND | ND | ND | ND |
| Vmn1r143 | NA | ND | ND | ND | ND | ND | ND | ND |
| Vmn1r251 | NA | ND | ND | ND | ND | ND | ND | ND |
| Vmn1r254 | NA | ND | ND | ND | ND | ND | ND | ND |
| Vmn1r152 | NA | ND | ND | ND | ND | ND | ND | ND |
| Vmn1r255 | NA | ND | ND | ND | ND | ND | ND | ND |
| Gal3st3 | 14 | 9.03E−11 | 1.31E−07 | 0.004304 | 0.000517 | 0.000354 | 0.000464 | 0.003841 |
| Cyp3a41a | NA | ND | ND | ND | ND | ND | ND | ND |
| Pantr1 | 278 | 7.43E−05 | 0.005702 | 0.01026 | 0.214065 | 0.004549 | 0.006461 | 0.201493 |
| Gria4 | 76 | 5.49E−07 | 0.000153 | 0.000272 | 0.001681 | 0.011993 | 0.206032 | 0.028888 |
| Klrc1 | NA | ND | ND | ND | ND | ND | ND | ND |
| Gm3002 | NA | ND | ND | ND | ND | ND | ND | ND |
| Stx1b | 139 | 5.40E−06 | 0.000831 | 0.010056 | 0.023195 | 0.001424 | 0.020417 | 0.074953 |
| Hmx1 | 50 | 1.08E−07 | 4.61E−05 | 0.00301 | 0.00327 | 0.005736 | 0.005313 | 0.015945 |
| 5730596B20Rik | 58 | 1.80E−07 | 6.64E−05 | 0.005881 | 0.029177 | 0.004939 | 0.001949 | 0.005289 |
| Galnt10 | 6679 | 0.265574 | 0.850226 | ND | 0.043714 | 0.959164 | ND | 0.522631 |
| Ephb4 | 222 | 4.07E−05 | 0.003922 | 0.449143 | 0.018787 | 0.022359 | 0.004137 | 0.007834 |
| Mapk8ip2 | 720 | 0.001481 | 0.043963 | 0.020886 | 0.243873 | 0.015222 | 0.057456 | 0.142784 |
| Zbtb7a | 4469 | 0.129062 | 0.617446 | 0.090537 | 0.190028 | 0.661684 | 0.579046 | 0.080491 |
| Zfp697 | 285 | 7.89E−05 | 0.005926 | 0.011703 | 0.003547 | 0.075089 | 0.081673 | 0.055167 |
| Mrgpra2a | NA | ND | ND | ND | ND | ND | ND | ND |
| Obox1 | NA | ND | ND | ND | ND | ND | ND | ND |
| Kcnn4 | 1502 | 0.011912 | 0.169645 | ND | 0.011114 | ND | ND | 0.144116 |
| Galnt2 | 60 | 1.90E−07 | 6.78E−05 | 0.003071 | 0.012336 | 0.006076 | 0.006542 | 0.006183 |
| Tmem181a | 75 | 4.96E−07 | 0.000141 | 5.88E−05 | ND | 0.000458 | ND | ND |
| Trappc5 | 569 | 0.000657 | 0.024714 | 0.210596 | 0.009274 | 0.043968 | 0.033743 | 0.074366 |
| Cilp2 | 3258 | 0.067178 | 0.440978 | ND | 0.729178 | 0.035027 | 0.6229 | 0.042241 |
| Obox3 | NA | ND | ND | ND | ND | ND | ND | ND |
| Hoxd3 | 4059 | 0.106557 | 0.560946 | 0.378379 | 0.196446 | 0.409541 | 0.139168 | 0.089053 |
| Nrg3 | 169 | 1.56E−05 | 0.001974 | 0.00823 | 0.038407 | 0.002094 | 0.037436 | 0.075094 |
| Tmem181c-ps | 5418 | 0.184266 | 0.727306 | 0.764541 | 0.022551 | 0.419878 | 0.208976 | 0.679911 |
| Gm2042 | NA | ND | ND | ND | ND | ND | ND | ND |
| Olfr18 | NA | ND | ND | ND | ND | ND | ND | ND |
| Vmn1r107 | NA | ND | ND | ND | ND | ND | ND | ND |
| Xkr6 | 67 | 3.20E−07 | 0.000102 | 0.009951 | 0.000577 | 0.127798 | 0.003232 | 0.007248 |
| Pcdhgb4 | 1241 | 0.00733 | 0.126276 | 0.04349 | 0.204293 | 0.179391 | 0.07631 | 0.047965 |
| Lgals6 | 70 | 3.66E−07 | 0.000112 | 0.003182 | 0.001006 | 0.01934 | 0.129538 | 0.002517 |
| Col1a1 | 282 | 7.64E−05 | 0.005797 | 0.022613 | 0.09112 | 0.024472 | 0.00334 | 0.080034 |
| Vwc2 | 99 | 1.69E−06 | 0.000364 | 0.021907 | 0.006722 | 0.014521 | 0.00356 | 0.016362 |
| Nkx2-1 | 214 | 3.73E−05 | 0.003678 | 0.007711 | 0.139014 | 0.040972 | 0.018938 | 0.006586 |
| Trhde | 40 | 3.20E−08 | 1.71E−05 | 0.011339 | 0.00905 | 0.015459 | 0.007748 | 9.42E−05 |
| Sntg1 | 591 | 0.000745 | 0.026952 | 0.078575 | 0.009246 | 0.120566 | 0.085171 | 0.034051 |
| Fbln1 | 539 | 0.00059 | 0.023411 | 0.037835 | 0.070309 | 0.030752 | 0.043553 | 0.052441 |
| Erbb4 | 8743 | 0.413934 | 1 | 0.718775 | 0.627808 | 0.332769 | 0.56668 | 0.067894 |
| Gm20752 | NA | ND | ND | ND | ND | ND | ND | ND |
| Pcdhga3 | 1602 | 0.013946 | 0.186189 | 0.122663 | 0.234164 | 0.363991 | 0.010243 | 0.13859 |
| Lin28b | 36 | 2.88E−08 | 1.66E−05 | 0.003458 | 0.000281 | 0.001017 | 0.001713 | 0.603504 |
| Hoxb5os | 172 | 1.70E−05 | 0.002109 | 0.001124 | 0.019169 | 0.009167 | 0.054467 | 0.191689 |
| 9030404E10Rik | 61 | 2.22E−07 | 7.78E−05 | 0.012863 | 0.002976 | 0.004267 | 0.011255 | 0.006075 |
| Krt5 | 1036 | 0.004412 | 0.09111 | 0.105383 | 0.001527 | 0.166061 | 0.162519 | 0.655233 |
| Fat3 | 44 | 4.72E−08 | 2.30E−05 | 0.004053 | 0.005322 | 0.003661 | 0.006554 | 0.003518 |
| Hebp1 | 1369 | 0.009494 | 0.148354 | 0.018081 | 0.02581 | 0.183537 | 0.326383 | 0.302701 |
| Usp29 | 219 | 3.77E−05 | 0.003679 | 0.00396 | 0.005136 | 0.143677 | 0.012029 | 0.157902 |
| Vmn1r131 | NA | ND | ND | ND | ND | ND | ND | ND |
| Cpq | 8153 | 0.369882 | 0.970073 | 0.703706 | 0.16872 | 0.785528 | 0.054914 | 0.863124 |
| Ankrd33b | 7684 | 0.334791 | 0.931441 | 0.38118 | 0.278781 | 0.107984 | 0.62217 | 0.49323 |
| Uggt2 | 83 | 8.76E−07 | 0.000226 | 0.030702 | 0.007286 | 0.014522 | 0.010855 | 0.001614 |
| Csf2ra | 116 | 3.56E−06 | 0.000654 | 0.105159 | 0.016578 | 0.004835 | 0.003551 | 0.010247 |
| L3mbtl4 | 223 | 4.09E−05 | 0.003926 | 0.006579 | 0.128542 | 0.004375 | 0.042521 | 0.039122 |
| Hoxb3os | 148 | 9.23E−06 | 0.001335 | 0.142675 | 0.005476 | 0.007546 | 0.028895 | 0.00574 |
| Pcdh15 | 191 | 2.84E−05 | 0.003132 | 0.012689 | 0.047771 | 0.006663 | 0.021151 | 0.04571 |
| Usp10 | 64 | 2.64E−07 | 8.70E−05 | 0.003086 | 0.012577 | 0.007394 | 0.005146 | 0.009299 |
| Ugt8a | 57 | 1.78E−07 | 6.64E−05 | 0.003445 | 0.033534 | 0.001745 | 0.01819 | 0.002356 |
| Rftn1 | 391 | 0.000229 | 0.012488 | 0.00717 | 0.092071 | 0.03423 | 0.013494 | 0.178582 |
| Mansc4 | 86 | 9.16E−07 | 0.000228 | 0.012556 | 0.02177 | 0.005305 | 0.001772 | 0.023347 |
| Prdm5 | 32 | 1.95E−08 | 1.30E−05 | 0.002087 | 0.000439 | 0.00535 | 0.006793 | 0.019559 |
| Evx1 | 5039 | 0.161729 | 0.686344 | 0.21735 | 0.369059 | 0.232357 | 0.078078 | 0.551942 |
| 2410018L13Rik | 376 | 0.000201 | 0.011451 | ND | ND | 0.000201 | ND | ND |
| A330008L17Rik | 92 | 1.28E−06 | 0.000297 | 0.010576 | 0.028032 | 0.006698 | 0.006119 | 0.007355 |
| Gm19345 | 1398 | 0.010064 | 0.153896 | 0.007935 | 0.668076 | 0.390698 | 0.005536 | 0.80322 |
| D830030K20Rik | NA | ND | ND | ND | ND | ND | ND | ND |
| 4930486L24Rik | 3527 | 0.078832 | 0.47794 | 0.787775 | 0.083332 | 0.085176 | 0.554232 | 0.072442 |
| Slc7a11 | 212 | 3.67E−05 | 0.003678 | 0.024494 | 0.123956 | 0.219294 | 0.002934 | 0.002753 |
| Osm | 78 | 6.46E−07 | 0.000177 | 0.00208 | 0.007924 | 0.011844 | 0.013318 | 0.015222 |
| Gucy1a2 | 1342 | 0.008913 | 0.142087 | 0.592861 | 0.47745 | 0.01201 | 0.109544 | 0.020745 |
| Cd84 | 165 | 1.52E−05 | 0.001971 | 0.002785 | 0.006449 | 0.014298 | 0.010512 | 0.669448 |
| Rims2 | 2284 | 0.031088 | 0.290974 | 0.898715 | 0.331215 | 0.100138 | 0.290145 | 0.005769 |
| Nkx6-2 | 94 | 1.46E−06 | 0.000332 | 0.007775 | 0.027392 | 0.021775 | 0.003363 | 0.006715 |
| Espn | 392 | 0.000229 | 0.012488 | 0.004585 | 0.192444 | 0.143763 | 0.037128 | 0.011576 |
| Esyt2 | 10646 | 0.541226 | 1 | 0.237221 | 0.368629 | 0.365108 | 0.793687 | 0.459918 |
| Psg25 | NA | ND | ND | ND | ND | ND | ND | ND |
| Dync2i1 | 13425 | 0.719104 | 1 | ND | ND | ND | 0.788791 | 0.445734 |
| Mup18 | 98 | 1.64E−06 | 0.000359 | 0.003861 | 0.011968 | ND | 0.002695 | 0.00762 |
| Elavl4 | 59 | 1.85E−07 | 6.72E−05 | 0.00158 | 0.018675 | 0.004345 | 0.001437 | 0.049005 |
| Myo9a | 25 | 3.73E−09 | 3.19E−06 | 0.006592 | 0.001777 | 0.002532 | 0.001853 | 0.001762 |
| Gemin5 | 254 | 5.96E−05 | 0.005013 | 0.007416 | ND | 0.011049 | 0.003493 | 0.229122 |
| Chd9 | 1064 | 0.004784 | 0.096193 | 0.01093 | 0.096229 | 0.051305 | 0.948429 | 0.062294 |
| Btnl9 | 341 | 0.000149 | 0.009319 | 0.337733 | 0.017796 | 0.014207 | 0.006263 | 0.058732 |
| Cacna1a | 9768 | 0.481433 | 1 | ND | ND | 0.117281 | 0.621549 | 0.876888 |
| Elfn1 | 642 | 0.000984 | 0.032782 | 0.068068 | 0.022198 | 0.408278 | 0.016087 | 0.037089 |
| Tap2 | 1562 | 0.013096 | 0.179363 | 0.035217 | 0.299693 | 0.07512 | 0.203577 | 0.08382 |
| Tspan5 | 41 | 3.54E−08 | 1.85E−05 | 0.012118 | 0.003429 | 0.002523 | 0.002504 | 0.00496 |
| Cdcp1 | 330 | 0.000135 | 0.008751 | 0.049648 | 0.009741 | 0.038973 | 0.014519 | 0.101326 |
| Mir6991 | 102 | 2.04E−06 | 0.000424 | 0.006175 | 0.007155 | 0.042574 | 0.020977 | 0.003963 |
| Gm11627 | 103 | 2.04E−06 | 0.000424 | 0.003672 | 0.097259 | 0.023616 | 0.005153 | 0.003607 |
| Barx1 | 377 | 0.000202 | 0.011451 | 0.037822 | 0.082978 | 0.037154 | 0.001619 | 0.245714 |
| Asap2 | 430 | 0.000303 | 0.015063 | ND | ND | 0.001308 | ND | 0.020043 |
| Gm3772 | 5767 | 0.20571 | 0.762697 | ND | ND | ND | ND | 0.20571 |
| Gm14322 | 968 | 0.003647 | 0.080593 | 0.357923 | 0.003913 | 0.214926 | 0.091526 | 0.079205 |

TABLE 5
(Supplementary Table S2c. Ranked genes by differential methylation
(Old + OSKM vs Old-2): Rank_body, p-value body, and q-value body)

| | Rank | p-value | q-value | p-value | p-value | p-value | p-value | p-value |
|---|---|---|---|---|---|---|---|---|
| Gene | body | body | body | body TR1 | body TR2 | body TR3 | body TR4 | body TR5 |
| Cntnap2 | 118 | 6.30E−07 | 9.49E−05 | 0.038285 | 0.002202 | 0.00675 | 0.003326 | 0.02029 |
| Def8 | 1 | 2.22E−16 | 1.99E−12 | 2.23E−06 | 2.71E−06 | 3.78E−06 | 3.62E−06 | 4.20E−06 |
| Hic1 | 253 | 1.59E−05 | 0.001123 | 0.17787 | 0.000105 | 0.008368 | 0.032268 | 0.378935 |
| Gm13034 | NA | ND | ND | ND | ND | ND | ND | ND |
| Galnt13 | 648 | 0.000517 | 0.01427 | 0.099328 | 0.021104 | 0.032379 | 0.016549 | 0.140066 |
| Rbfox1 | 5 | 3.87E−13 | 1.39E−09 | 1.72E−07 | 0.013185 | 0.072943 | 5.46E−07 | 0.035152 |
| Hoxa2 | 1 | 2.22E−16 | 1.99E−12 | 8.93E−06 | 6.81E−06 | 0.000569 | 7.10E−06 | 4.85E−05 |
| Sox11 | 451 | 0.00018 | 0.007164 | 0.011212 | 0.147655 | 0.040218 | 0.036375 | 0.016592 |
| Pcdh8 | 358 | 7.72E−05 | 0.003858 | 0.006601 | 0.023545 | 0.074911 | 0.026411 | 0.044392 |
| Gm9748 | NA | ND | ND | ND | ND | ND | ND | ND |
| Hoxaas3 | 16 | 8.25E−11 | 9.25E−08 | 0.000298 | 0.000297 | 0.010062 | 0.000157 | 0.009053 |
| Hoxd1 | NA | ND | ND | ND | ND | ND | ND | ND |
| Mup2 | 7 | 9.96E−12 | 2.51E−08 | 0.000712 | 0.000155 | 0.003777 | 0.000433 | 0.000654 |
| Mdga2 | 2985 | 0.04456 | 0.267382 | 0.26182 | 0.060409 | 0.148052 | 0.086803 | 0.432759 |
| Gm10377 | 9 | 1.40E−11 | 2.78E−08 | 0.001038 | 0.000673 | 0.001203 | 0.000311 | 0.00066 |
| Hoxa3 | 3 | 3.32E−14 | 1.98E−10 | 8.45E−05 | 4.56E−05 | 0.001977 | 4.13E−05 | 0.000673 |
| Skint11 | 11 | 1.90E−11 | 3.09E−08 | 0.000108 | 0.001167 | 0.000789 | 0.000698 | 0.00349 |
| Mmp16 | 908 | 0.001436 | 0.028319 | 0.064501 | 0.041542 | 0.073531 | 0.043624 | 0.071018 |
| Tdpoz5 | 12 | 4.81E−11 | 7.19E−08 | 0.00041 | 0.00016 | 0.000316 | 0.002839 | 0.011766 |
| Senp8 | 287 | 2.95E−05 | 0.001835 | 0.018284 | 0.03244 | 0.038196 | 0.008391 | 0.021559 |
| Haglr | 1460 | 0.005845 | 0.071735 | 0.05952 | 0.196736 | 0.108663 | 0.091882 | 0.03616 |
| Gm45351 | 14 | 7.59E−11 | 9.14E−08 | 0.00092 | 0.000917 | 0.001541 | 0.001047 | 0.000848 |
| Vmn2r47 | 15 | 7.64E−11 | 9.14E−08 | 0.00092 | 0.000917 | 0.001541 | 0.001047 | 0.000854 |
| Ajm1 | NA | ND | ND | ND | ND | ND | ND | ND |
| Foxa1 | 17 | 9.41E−11 | 9.63E−08 | 0.000948 | 0.00105 | 0.0027 | 0.000783 | 0.0007 |
| Peg12 | NA | ND | ND | ND | ND | ND | ND | ND |
| Defa23 | 19 | 1.32E−10 | 1.25E−07 | 0.001543 | 0.000123 | 0.003019 | 0.000976 | 0.003845 |
| Nfix | 21 | 1.59E−10 | 1.36E−07 | 0.00134 | 0.000126 | 0.004699 | 0.00182 | 0.001841 |
| Grid2 | 671 | 0.000567 | 0.015123 | 0.065827 | 0.016975 | 0.075617 | 0.009322 | 0.225297 |
| Pcdhga5 | 116 | 5.78E−07 | 8.87E−05 | 0.010733 | 0.020675 | 0.010665 | 0.001735 | 0.00845 |
| Cldn6 | NA | ND | ND | ND | ND | ND | ND | ND |
| Hoxb3 | 4 | 3.24E−13 | 1.39E−09 | 0.001809 | 0.000251 | 4.50E−06 | 0.008756 | 0.000146 |
| Camk2b | 24 | 4.45E−10 | 3.33E−07 | 0.000849 | 0.006624 | 0.000352 | 0.0095 | 0.000455 |
| Ltbp3 | 55 | 5.76E−08 | 1.84E−05 | 0.001728 | 0.016264 | 0.001342 | 0.009675 | 0.006288 |
| Otx1 | 20 | 1.43E−10 | 1.28E−07 | 0.002047 | 0.004694 | 0.000634 | 0.001314 | 0.000294 |
| Nefm | 382 | 9.14E−05 | 0.004283 | 0.295225 | 0.00347 | 0.017421 | 0.012298 | 0.07703 |
| Nr4a2 | 6 | 9.74E−13 | 2.91E−09 | 0.0011 | 8.67E−05 | 0.000265 | 4.14E−05 | 0.008461 |
| Sgcz | 521 | 0.000286 | 0.00978 | 0.064965 | 0.020529 | 0.048394 | 0.01257 | 0.089452 |
| Lrp1b | 22 | 2.44E−10 | 1.99E−07 | 0.003101 | 0.000247 | 0.002435 | 0.000129 | 0.017948 |
| Atn1 | 27 | 1.45E−09 | 9.04E−07 | 0.000463 | 0.004245 | 0.006265 | 0.004964 | 0.000535 |
| Crtc1 | 28 | 1.46E−09 | 9.04E−07 | 0.001439 | 7.45E−05 | 0.025629 | 0.002186 | 0.005502 |
| Skint5 | 30 | 2.09E−09 | 1.21E−06 | 0.00243 | 0.003119 | 0.002376 | 0.00102 | 0.002713 |
| Csmd1 | 1449 | 0.005723 | 0.070776 | 0.126433 | 0.032202 | 0.080324 | 0.07152 | 0.175452 |
| Dlgap1 | 1097 | 0.002644 | 0.043167 | 0.05674 | 0.043422 | 0.079235 | 0.054887 | 0.130619 |
| Cdh2 | 9217 | 0.503315 | 0.979448 | 0.491406 | 0.380245 | 0.315533 | 0.199553 | 0.810187 |
| Hspa1a | NA | ND | ND | ND | ND | ND | ND | ND |
| Skint6 | 37 | 7.11E−09 | 3.36E−06 | 0.002144 | 0.010416 | 0.001592 | 0.001323 | 0.004321 |
| Ptprd | 18 | 9.66E−11 | 9.63E−08 | 0.00357 | 0.001036 | 7.22E−05 | 0.000896 | 0.006334 |
| Hs3st4 | 12157 | 0.753529 | 1 | 0.42856 | 0.48785 | 0.368903 | 0.549696 | 0.827967 |
| Pim1 | 39 | 7.87E−09 | 3.53E−06 | ND | 0.00023 | 0.000105 | ND | 0.001004 |
| Ebf1 | 1027 | 0.002203 | 0.038419 | 0.037301 | 0.012739 | 0.203223 | 0.041065 | 0.275007 |
| Trank1 | 5685 | 0.203265 | 0.64098 | 0.570952 | 0.204985 | 0.322862 | 0.188702 | 0.174481 |
| Serpina1b | 13 | 5.60E−11 | 7.72E−08 | 0.003939 | 0.001563 | 0.003022 | 0.000112 | 0.000393 |
| Skint4 | 43 | 1.52E−08 | 6.21E−06 | 0.00089 | 0.011679 | 0.00106 | 0.000381 | 0.116518 |
| Smad3 | 26 | 9.28E−10 | 6.40E−07 | 0.001217 | 0.002189 | 0.002728 | 0.002967 | 0.000914 |
| Rassf3 | 64 | 1.38E−07 | 3.11E−05 | 0.019219 | 0.001418 | 0.000481 | 0.015936 | 0.030654 |
| Hoxaas2 | 357 | 7.63E−05 | 0.003824 | 0.024764 | 0.164103 | 0.089862 | 0.017814 | 0.002068 |
| Msi1 | 44 | 2.46E−08 | 9.81E−06 | 0.005317 | 0.088786 | 0.001371 | 0.000502 | 0.002626 |
| Zfp503 | 31 | 3.18E−09 | 1.78E−06 | 0.001224 | 0.000454 | 0.006993 | 0.007869 | 0.002626 |
| 1700034I23Rik | NA | ND | ND | ND | ND | ND | ND | ND |
| Csmd2 | 753 | 0.000787 | 0.018708 | 0.080024 | 0.025753 | 0.129471 | 0.013157 | 0.077889 |
| Nox4 | 1614 | 0.008331 | 0.092486 | 0.198145 | 0.166462 | 0.025196 | 0.029041 | 0.290387 |
| Pcdhga6 | 120 | 6.78E−07 | 0.0001 | 0.007809 | 0.028158 | 0.012392 | 0.001619 | 0.009502 |
| Snord116l1 | 23 | 4.38E−10 | 3.33E−07 | 0.001393 | 0.000962 | 0.001142 | 0.002898 | 0.001891 |
| Lrfn1 | 397 | 0.000106 | 0.004791 | 0.01669 | 0.007046 | 0.006234 | 0.040085 | 0.696126 |
| Has1 | 32 | 4.51E−09 | 2.45E−06 | 0.007524 | 0.001475 | 0.001853 | 0.001583 | 0.00369 |
| Gvin2 | 47 | 4.36E−08 | 1.58E−05 | 0.001838 | 0.005407 | 0.006102 | 0.014893 | 0.001836 |
| Gvin-ps2 | 47 | 4.36E−08 | 1.58E−05 | 0.001838 | 0.005407 | 0.006102 | 0.014893 | 0.001836 |
| Lzts3 | 8 | 1.12E−11 | 2.51E−08 | 0.000267 | 0.000288 | 0.001208 | 0.000667 | 0.002166 |
| Nxph4 | 10 | 1.62E−11 | 2.91E−08 | 0.004431 | 0.000108 | 0.001347 | 0.000587 | 0.000537 |
| Irx3 | 337 | 5.65E−05 | 0.002998 | 0.005198 | 0.013713 | 0.038738 | 0.102536 | 0.032546 |
| Bhlhe40 | 25 | 5.19E−10 | 3.72E−07 | 0.038223 | 0.000346 | 0.000386 | 0.000575 | 0.003468 |
| Grik2 | 227 | 1.11E−05 | 0.000874 | 0.032788 | 0.005628 | 0.015562 | 0.007321 | 0.058274 |
| Kcnj3 | 876 | 0.001269 | 0.025931 | 0.051961 | 0.021191 | 0.109878 | 0.020862 | 0.204805 |
| Lingo2 | 169 | 3.65E−06 | 0.000385 | 0.022303 | 0.002956 | 0.017495 | 0.006348 | 0.043163 |
| Syngap1 | 83 | 1.52E−07 | 3.25E−05 | 0.187247 | 0.010429 | 0.000326 | 0.005196 | 0.002167 |
| Cdca71 | 1389 | 0.005038 | 0.064995 | 0.317743 | 0.012879 | 0.05299 | 0.119706 | 0.132085 |
| St6gal2 | 687 | 0.000594 | 0.015466 | 0.158584 | 0.213312 | 0.015155 | 0.006689 | 0.054972 |
| Vmn2r-ps11 | 58 | 7.31E−08 | 2.22E−05 | 0.003113 | 0.001252 | 0.012277 | 0.005258 | 0.012047 |
| Tox3 | 474 | 0.000216 | 0.008123 | 0.006248 | 0.011841 | 0.273887 | 0.070428 | 0.035405 |
| Dnah7c | 165 | 3.40E−06 | 0.000368 | 0.036695 | 0.015092 | 0.003047 | 0.00669 | 0.025701 |
| Pcdhga8 | 132 | 9.93E−07 | 0.000134 | 0.010947 | 0.030319 | 0.014206 | 0.000964 | 0.014546 |
| Pcdhgb5 | 211 | 9.04E−06 | 0.000765 | 0.043679 | 0.039404 | 0.012813 | 0.00126 | 0.034293 |
| Nrg1 | 7332 | 0.33839 | 0.827598 | 0.268367 | 0.255819 | 0.468257 | 0.21918 | 0.511904 |
| Col28a1 | 61 | 9.69E−08 | 2.80E−05 | 0.000301 | 0.006769 | 0.001008 | 0.063 | 0.032585 |
| Smarca5-ps | NA | ND | ND | ND | ND | ND | ND | ND |
| Gm20110 | 4853 | 0.146767 | 0.542225 | 0.719777 | 0.331634 | 0.071431 | 0.092288 | 0.426229 |
| Adcy2 | 50 | 4.50E−08 | 1.58E−05 | 0.003558 | 0.001416 | 0.003033 | 0.003323 | 0.033917 |
| Eif4a311 | NA | ND | ND | ND | ND | ND | ND | ND |
| Wnt3a | 67 | 1.40E−07 | 3.11E−05 | ND | 0.000482 | 0.000364 | 0.135773 | 0.002361 |
| Vmn1r101 | 68 | 1.40E−07 | 3.11E−05 | ND | 0.000482 | 0.000364 | 0.135773 | 0.002361 |
| Vmn1r250 | 69 | 1.40E−07 | 3.11E−05 | ND | 0.000482 | 0.000364 | 0.135773 | 0.002361 |
| Gm10665 | 70 | 1.40E−07 | 3.11E−05 | ND | 0.000482 | 0.000364 | 0.135773 | 0.002361 |
| Hmbox1 | 71 | 1.40E−07 | 3.11E−05 | ND | 0.000482 | 0.000364 | 0.135773 | 0.002361 |
| Vmn1r100 | 72 | 1.40E−07 | 3.11E−05 | ND | 0.000482 | 0.000364 | 0.135773 | 0.002361 |
| Gm10668 | 73 | 1.40E−07 | 3.11E−05 | ND | 0.000482 | 0.000364 | 0.135773 | 0.002361 |
| Gm4513 | 74 | 1.40E−07 | 3.11E−05 | ND | 0.000482 | 0.000364 | 0.135773 | 0.002361 |
| Vmn1r142 | 75 | 1.40E−07 | 3.11E−05 | ND | 0.000482 | 0.000364 | 0.135773 | 0.002361 |
| Vmn1r143 | 76 | 1.40E−07 | 3.11E−05 | ND | 0.000482 | 0.000364 | 0.135773 | 0.002361 |
| Vmn1r251 | 77 | 1.40E−07 | 3.11E−05 | ND | 0.000482 | 0.000364 | 0.135773 | 0.002361 |
| Vmn1r254 | 78 | 1.40E−07 | 3.11E−05 | ND | 0.000482 | 0.000364 | 0.135773 | 0.002361 |
| Vmn1r152 | 79 | 1.40E−07 | 3.11E−05 | ND | 0.000482 | 0.000364 | 0.135773 | 0.002361 |
| Vmn1r255 | 80 | 1.40E−07 | 3.11E−05 | ND | 0.000482 | 0.000364 | 0.135773 | 0.002361 |
| Gal3st3 | 11510 | 0.699103 | 1 | 0.60662 | 0.401333 | 0.446792 | 0.341578 | 0.707781 |
| Cyp3a41a | 81 | 1.43E−07 | 3.13E−05 | 0.016696 | 0.009276 | 0.002876 | 0.001856 | 0.008069 |
| Pantr1 | 269 | 2.45E−05 | 0.001619 | 0.020168 | 0.014663 | 0.015697 | 0.091331 | 0.007686 |
| Gria4 | 1035 | 0.002247 | 0.038889 | 0.029217 | 0.129344 | 0.140746 | 0.016043 | 0.131297 |
| Klrc1 | 82 | 1.50E−07 | 3.23E−05 | 0.006055 | 0.062333 | 0.003907 | 0.001246 | 0.003822 |
| Gm3002 | 84 | 1.54E−07 | 3.26E−05 | 0.011721 | 0.015203 | 0.000551 | 0.002999 | 0.024716 |
| Stx1b | 585 | 0.000382 | 0.011665 | 0.045857 | 0.006378 | 0.026912 | 0.054714 | 0.245411 |
| Hmx1 | 1776 | 0.011005 | 0.111062 | 0.282847 | 0.027942 | 0.752497 | 0.013389 | 0.131696 |
| 5730596B20Rik | NA | ND | ND | ND | ND | ND | ND | ND |
| Galnt10 | 51 | 4.57E−08 | 1.58E−05 | 0.000786 | 0.000514 | 0.115129 | 0.000261 | 0.144529 |
| Ephb4 | 345 | 6.91E−05 | 0.003586 | 0.024194 | 0.032472 | 0.024037 | 0.005485 | 0.114738 |
| Mapk8ip2 | 144 | 1.65E−06 | 0.000204 | 0.041828 | 0.002426 | 0.002062 | 0.046706 | 0.012418 |
| Zbtb7a | 35 | 5.19E−09 | 2.54E−06 | 0.123873 | 6.27E−05 | 2.89E−05 | 0.453868 | 0.001389 |
| Zfp697 | 290 | 2.99E−05 | 0.001841 | 0.031118 | 0.002363 | 0.054706 | 0.03421 | 0.030203 |
| Mrgpra2a | 89 | 2.07E−07 | 4.13E−05 | 0.034991 | 0.019222 | 0.001354 | 0.000999 | 0.011324 |
| Obox1 | 90 | 2.20E−07 | 4.33E−05 | 0.001734 | 0.004548 | 0.001708 | 0.00545 | 0.150198 |
| Kcnn4 | 114 | 5.64E−07 | 8.80E−05 | 0.045392 | 0.002312 | 0.001245 | 0.064019 | 0.004026 |
| Galnt2 | 1789 | 0.011251 | 0.11272 | 0.124235 | 0.03556 | 0.123937 | 0.076663 | 0.258009 |
| Tmem181a | 896 | 0.001375 | 0.027467 | 0.098859 | 0.005651 | 0.03476 | 0.234398 | 0.126412 |
| Trappc5 | 185 | 5.17E−06 | 0.000499 | 0.00516 | 0.192377 | 0.013486 | 0.019504 | 0.001846 |
| Cilp2 | 52 | 4.84E−08 | 1.64E−05 | 0.003077 | 0.005616 | 0.005391 | 0.011631 | 0.001729 |
| Obox3 | 92 | 2.68E−07 | 5.17E−05 | 0.001931 | 0.008108 | 0.002797 | 0.005615 | 0.056801 |
| Hoxd3 | 40 | 8.06E−09 | 3.53E−06 | 0.595335 | 1.92E−05 | 0.08136 | 0.002523 | 0.0001 |
| Nrg3 | 501 | 0.000252 | 0.008991 | 0.090333 | 0.01373 | 0.039056 | 0.008777 | 0.145223 |
| Tmem181c-ps | 33 | 4.84E−09 | 2.54E−06 | 0.001504 | 0.001671 | 0.009761 | 0.001598 | 0.003328 |
| Gm2042 | 98 | 3.00E−07 | 5.44E−05 | 0.002869 | 0.001456 | 0.008088 | 0.040471 | 0.011656 |
| Olfr18 | 99 | 3.11E−07 | 5.55E−05 | 0.033932 | 0.015606 | 0.008078 | 0.000809 | 0.00481 |
| Vmn1r107 | 100 | 3.12E−07 | 5.55E−05 | ND | 0.000823 | 0.001054 | 0.051147 | 0.003159 |
| Xkr6 | 1724 | 0.010286 | 0.106941 | 0.16381 | 0.033982 | 0.137143 | 0.0505 | 0.246562 |
| Pcdhgb4 | 108 | 4.81E−07 | 7.92E−05 | 0.007368 | 0.023115 | 0.012452 | 0.001136 | 0.011576 |
| Lgals6 | NA | ND | ND | ND | ND | ND | ND | ND |
| Col1a1 | 374 | 8.77E−05 | 0.004195 | 0.000806 | 0.107284 | 0.523357 | 0.016743 | 0.021153 |
| Vwc2 | 1162 | 0.003078 | 0.047454 | 0.090096 | 0.047484 | 0.05695 | 0.049644 | 0.142681 |
| Nkx2-1 | 455 | 0.000189 | 0.007428 | 0.286667 | 0.002186 | 0.247758 | 0.046216 | 0.00595 |
| Trhde | 3581 | 0.069908 | 0.349894 | 0.527762 | 0.059641 | 0.348993 | 0.1087 | 0.15365 |
| Sntg1 | 207 | 8.40E−06 | 0.000725 | 0.015855 | 0.007428 | 0.023269 | 0.003973 | 0.080028 |
| Fbln1 | 238 | 1.19E−05 | 0.000893 | 0.001911 | 0.045992 | 0.020415 | 0.101026 | 0.007353 |
| Erbb4 | 29 | 2.04E−09 | 1.21E−06 | 0.004639 | 0.000541 | 0.002571 | 0.001111 | 0.006739 |
| Gm20752 | 109 | 5.11E−07 | 8.27E−05 | 0.00697 | 0.00085 | 0.016993 | 0.002525 | 0.117979 |
| Pcdhga3 | 107 | 3.86E−07 | 6.42E−05 | 0.007087 | 0.023738 | 0.008035 | 0.001391 | 0.011439 |
| Lin28b | 4466 | 0.121113 | 0.486187 | 0.045414 | 0.296849 | 0.586872 | 0.110285 | 0.542477 |
| Hoxb5os | 626 | 0.000467 | 0.013332 | 0.004352 | 0.009937 | 0.092774 | 0.16872 | 0.202966 |
| 9030404E10Rik | 2347 | 0.02493 | 0.190357 | 0.130774 | 0.120232 | 0.075794 | 0.549304 | 0.054234 |
| Krt5 | 143 | 1.63E−06 | 0.000203 | 0.037349 | 0.005309 | 0.001414 | 0.028959 | 0.014742 |
| Fat3 | 3920 | 0.089678 | 0.410019 | 0.159065 | 0.22454 | 0.385276 | 0.068021 | 0.298772 |
| Hebp1 | 127 | 8.36E−07 | 0.000117 | 0.006882 | 0.005992 | 0.003776 | 0.067037 | 0.005157 |
| Usp29 | 549 | 0.000324 | 0.010534 | 0.008313 | 0.05207 | 0.175468 | 0.007696 | 0.146351 |
| Vmn1r131 | 123 | 7.26E−07 | 0.000105 | ND | 0.000304 | 0.001037 | 0.068984 | 0.016953 |
| Cpq | 36 | 5.23E−09 | 2.54E−06 | 0.002043 | 0.003463 | 0.00195 | 0.00452 | 0.002287 |
| Ankrd33b | 38 | 7.45E−09 | 3.43E−06 | 0.00125 | 0.004137 | 0.009191 | 0.002747 | 0.001641 |
| Uggt2 | 1798 | 0.011464 | 0.114266 | 0.209693 | 0.052682 | 0.274381 | 0.070265 | 0.052262 |
| Csf2ra | 1223 | 0.003592 | 0.052623 | 0.488108 | 0.9727 | 0.020469 | 0.008782 | 0.025042 |
| L3mbtl4 | 609 | 0.000421 | 0.012363 | 0.063283 | 0.020437 | 0.026801 | 0.018041 | 0.192088 |
| Hoxb3os | 986 | 0.001935 | 0.035143 | 0.029479 | 0.16181 | 0.000239 | 0.929239 | 0.862087 |
| Pcdh15 | 679 | 0.000583 | 0.015361 | 0.146067 | 0.01527 | 0.040553 | 0.014459 | 0.140672 |
| Usp10 | 2736 | 0.035989 | 0.235733 | 0.101731 | 0.229001 | 0.032904 | 0.145404 | 0.562613 |
| Ugt8a | 3225 | 0.053765 | 0.298712 | 0.438887 | 0.045435 | 0.180263 | 0.071058 | 0.466127 |
| Rftn1 | 387 | 9.49E−05 | 0.00438 | 0.013593 | 0.02533 | 0.020928 | 0.125732 | 0.019552 |
| Mansc4 | 1990 | 0.015969 | 0.14373 | 0.257541 | 0.191939 | 0.312489 | 0.022646 | 0.05186 |
| Prdm5 | 6162 | 0.241776 | 0.703572 | 0.786488 | 0.102751 | 0.330882 | 0.146231 | 0.449916 |
| Evx1 | 45 | 3.43E−08 | 1.32E−05 | 0.037949 | 0.001118 | 0.003046 | 0.003435 | 0.002822 |
| 2410018L13Rik | 369 | 8.39E−05 | 0.004069 | 0.024365 | 0.03448 | 0.015214 | ND | 0.007757 |
| A330008L17Rik | 1838 | 0.012334 | 0.120281 | 0.046312 | 0.0456 | 0.270128 | 0.059493 | 0.365028 |
| Gm19345 | 148 | 2.06E−06 | 0.000248 | 0.176262 | 0.211357 | 0.004134 | 0.000281 | 0.003666 |
| D830030K20Rik | 136 | 1.24E−06 | 0.000163 | 0.003985 | 0.005076 | 0.005781 | 0.010873 | 0.068011 |
| 4930486L24Rik | 62 | 1.20E−07 | 3.11E−05 | 0.006935 | 0.022477 | 0.008114 | 0.000303 | 0.014173 |
| Slc7a11 | 694 | 0.000611 | 0.015762 | 0.011246 | 0.04815 | 0.044812 | 0.117797 | 0.068522 |
| Osm | 2267 | 0.022932 | 0.181271 | 0.313894 | 0.050399 | 0.011647 | 0.204106 | 0.831092 |
| Gucy1a2 | 149 | 2.19E−06 | 0.000262 | 0.008298 | 0.019279 | 0.004585 | 0.005011 | 0.046491 |
| Cd84 | 985 | 0.001933 | 0.035138 | 0.046038 | 0.077248 | 0.031709 | 0.035205 | 0.229792 |
| Rims2 | 110 | 5.12E−07 | 8.27E−05 | 0.00528 | 0.004322 | 0.016035 | 0.00669 | 0.012266 |
| Nkx6-2 | NA | ND | ND | ND | ND | ND | ND | ND |
| Espn | 429 | 0.000138 | 0.005776 | 0.03047 | 0.002358 | 0.221414 | 0.013058 | 0.13782 |
| Esyt2 | 34 | 5.01E−09 | 2.54E−06 | 0.000196 | 0.002668 | 0.000524 | 0.016879 | 0.029281 |
| Psg25 | 140 | 1.49E−06 | 0.00019 | 0.042825 | 0.00243 | 0.018303 | 0.005009 | 0.011303 |
| Dync2i1 | 95 | 2.82E−07 | 5.25E−05 | 0.000414 | 0.00494 | 0.003538 | 0.026893 | 0.076061 |
| Mup18 | NA | ND | ND | ND | ND | ND | ND | ND |
| Elavl4 | 3621 | 0.072132 | 0.356981 | 0.345485 | 0.151027 | 0.153852 | 0.105906 | 0.227396 |
| Myo9a | 11102 | 0.665257 | 1 | 0.200678 | 0.831681 | 0.467876 | 0.324984 | 0.869869 |
| Gemin5 | 688 | 0.000596 | 0.015491 | 0.122285 | 0.031466 | 0.083576 | 0.002163 | 0.272045 |
| Chd9 | 198 | 6.58E−06 | 0.000588 | 0.00135 | 0.004757 | 0.044759 | 0.091091 | 0.024703 |
| Btn19 | 519 | 0.000281 | 0.009675 | 0.11036 | 0.018243 | 0.090956 | 0.004098 | 0.094659 |
| Cacna1a | 63 | 1.33E−07 | 3.11E−05 | 0.004716 | 0.00127 | 0.014071 | 0.006095 | 0.011877 |
| Elfn1 | 311 | 4.29E−05 | 0.002467 | 0.148941 | 0.030219 | 0.031486 | 0.00294 | 0.015671 |
| Tap2 | 152 | 2.39E−06 | 0.00028 | 0.020844 | 0.032148 | 0.008083 | 0.001853 | 0.018839 |
| Tspan5 | 6320 | 0.255945 | 0.72623 | 0.109229 | 0.669227 | 0.180333 | 0.335724 | 0.446803 |
| Cdcp1 | 532 | 0.000304 | 0.010217 | 0.249132 | 0.01976 | 0.008172 | 0.035344 | 0.055414 |
| Mir6991 | NA | ND | ND | ND | ND | ND | ND | ND |
| Gm11627 | NA | ND | ND | ND | ND | ND | ND | ND |
| Barx1 | 482 | 0.000228 | 0.008443 | 0.069239 | 0.02522 | 0.017615 | 0.012783 | 0.138135 |
| Asap2 | 489 | 0.000236 | 0.008616 | 0.041509 | 0.090154 | 0.163135 | 0.000841 | 0.110377 |
| Gm3772 | 157 | 2.79E−06 | 0.000317 | 0.002858 | 0.000443 | 0.03062 | 0.014888 | 0.395967 |
| Gm14322 | 226 | 1.10E−05 | 0.000872 | 0.012576 | 0.015864 | 0.015164 | 0.022165 | 0.018133 |

TABLE 6
(Supplementary Table S3a. Ranked genes by differential methylation
(Y vs Old-2): RANK comp., p-value comp., q-value comp.)

| | RANK | p-value | q-value | p-value | p-value | p-value | p-value |
|---|---|---|---|---|---|---|---|
| Gene | comp. | comp. | comp. | comp. TR1 | comp. TR2 | comp. TR3 | comp. TR4 |
| Lncppara | 1 | 2.22E−16 | 8.19E−14 | 0 | 0 | 0 | 0 |
| B3glct | 1 | 2.22E−16 | 8.19E−14 | 0 | 0 | 0 | 0 |
| Egr3 | 3 | 2.22E−16 | 8.19E−14 | 0.000176 | 0.000137 | 0 | 0 |
| Lncpint | 4 | 2.22E−16 | 8.19E−14 | 0.003195 | 0.000118 | 0 | 0 |
| Grm7 | 5 | 2.22E−16 | 8.19E−14 | 0.006528 | 0 | 0 | 3.92E−05 |
| Sox11 | 6 | 2.22E−16 | 8.19E−14 | 0.015781 | 0 | 0 | 0 |
| Bhlhe40 | 7 | 2.22E−16 | 8.19E−14 | 0.001686 | 0 | 0.000137 | 5.88E−05 |
| Mmp16 | 8 | 2.22E−16 | 8.19E−14 | 0.00347 | 0 | 0.000216 | 0 |
| 5730507C01Rik | 9 | 2.22E−16 | 8.19E−14 | 0.007626 | 0 | 0.000137 | 0 |
| Foxg1 | 10 | 2.22E−16 | 8.19E−14 | 0.011958 | 0 | 0 | 0.000294 |
| Gm4425 | 11 | 2.22E−16 | 8.19E−14 | 0.015164 | 0 | 0.000359 | 0 |
| Hoxa9 | 12 | 2.22E−16 | 8.19E−14 | 0 | 0.011331 | 0.000176 | 0.000255 |
| Tmem267 | 13 | 2.22E−16 | 8.19E−14 | 0 | 0.000569 | 0.000353 | 0.001725 |
| 9030624G23Rik | 14 | 2.22E−16 | 8.19E−14 | 0.024559 | 0 | 0.000216 | 0 |
| Cilp2 | 15 | 2.22E−16 | 8.19E−14 | 0.015075 | 0 | 0 | 0.001725 |
| Nrg3 | 16 | 2.22E−16 | 8.19E−14 | 0.004587 | 0 | 0.000235 | 0.00051 |
| Otx1 | 17 | 2.22E−16 | 8.19E−14 | 0.002294 | 0 | 0 | 0.018957 |
| Lingo2 | 18 | 2.22E−16 | 8.19E−14 | 0.161299 | 0 | 0 | 0.000235 |
| Mdga2 | 19 | 2.22E−16 | 8.19E−14 | 0.062261 | 0 | 0.000176 | 0.000176 |
| Nr4a2 | 20 | 2.22E−16 | 8.19E−14 | 0.033503 | 0.000451 | 0 | 0.000529 |
| Nkain3 | 21 | 2.22E−16 | 8.19E−14 | 0.004293 | 0 | 0.000216 | 0.005705 |
| Peg10 | 22 | 2.22E−16 | 8.19E−14 | 0.01782 | 0 | 0.000137 | 0.004646 |
| Haglr | 23 | 2.22E−16 | 8.19E−14 | 0.001745 | 0 | 0.001509 | 0.009057 |
| Zfta | 24 | 2.22E−16 | 8.19E−14 | 0 | 0.000353 | 0.01535 | 0.004352 |
| Sgcz | 25 | 2.22E−16 | 8.19E−14 | 0.154516 | 0 | 0 | 0.003627 |
| 3110039M20Rik | 26 | 2.22E−16 | 8.19E−14 | 0.077983 | 0 | 0 | 0.009233 |
| Pcdhga9 | 27 | 2.22E−16 | 8.19E−14 | 0.009253 | 0 | 7.84E−05 | 0.030268 |
| Pcdhga10 | 28 | 2.22E−16 | 8.19E−14 | 0.004646 | 0 | 0.000235 | 0.019466 |
| Pcdh8 | 29 | 2.22E−16 | 8.19E−14 | 0.148027 | 0 | 0.000137 | 0.001274 |
| Hoxaas3 | 30 | 2.22E−16 | 8.19E−14 | 0.01335 | 0 | 0.007685 | 0.000983 |
| Csmd1 | 31 | 2.22E−16 | 8.19E−14 | 0.035875 | 0 | 0.000176 | 0.007057 |
| Tcfl5 | 32 | 2.22E−16 | 8.19E−14 | 0.003509 | 0.000176 | 0.246228 | 0 |
| Xkr4 | 33 | 2.22E−16 | 8.19E−14 | 0.067907 | 0.002882 | 0 | 0.002078 |
| Pcdh10 | 34 | 2.22E−16 | 8.19E−14 | 0.073612 | 0 | 0 | 0.051479 |
| Cntnap2 | 35 | 2.22E−16 | 8.19E−14 | 0.05046 | 0 | 0.009214 | 0.000392 |
| Prdm13 | 36 | 2.22E−16 | 8.19E−14 | 0.016447 | 0 | 0.004274 | 0.003685 |
| Galnt13 | 37 | 2.22E−16 | 8.19E−14 | 0.302856 | 0 | 5.88E−05 | 0.005371 |
| Pcdhgb2 | 38 | 2.22E−16 | 8.19E−14 | 0.002215 | 0 | 0.000588 | 0.176923 |
| Pcdhga6 | 39 | 2.22E−16 | 8.19E−14 | 0.01384 | 0 | 0.000706 | 0.020976 |
| Csf2ra | 40 | 2.22E−16 | 8.19E−14 | 0.595244 | 0 | 0.000216 | 0.000823 |
| Rims2 | 41 | 2.22E−16 | 8.19E−14 | 0.171884 | 0.002333 | 0 | 0.003235 |
| Nat8f3 | 42 | 2.22E−16 | 8.19E−14 | 0.000843 | 0 | 0.208577 | 0.006822 |
| Pcdhga12 | 43 | 2.22E−16 | 8.19E−14 | 0.008567 | 0 | 0.000137 | 0.353257 |
| Pcdhgb6 | 44 | 2.22E−16 | 8.19E−14 | 0.015056 | 0 | 0.000176 | 0.126149 |
| Pcdhga7 | 45 | 2.22E−16 | 8.19E−14 | 0.052832 | 0 | 0.002431 | 0.015585 |
| Nefm | 46 | 2.22E−16 | 8.19E−14 | 0.009174 | 0 | 0.051656 | 0.011919 |
| Pcdhga4 | 47 | 2.22E−16 | 8.19E−14 | 0.061046 | 0 | 0.000431 | 0.109643 |
| 2410018L13Rik | 48 | 2.22E−16 | 8.19E−14 | 0.317273 | 0 | 0.000823 | 0.012448 |
| A330008L17Rik | 49 | 2.22E−16 | 8.19E−14 | 0.100194 | 0 | 0.000216 | 0.136304 |
| Pcdhgb5 | 50 | 2.22E−16 | 8.19E−14 | 0.052793 | 0 | 0.001039 | 0.113936 |
| Pcdhgb4 | 51 | 2.22E−16 | 8.19E−14 | 0.102723 | 0 | 0.003764 | 0.026249 |
| Pcdhgb7 | 52 | 2.22E−16 | 8.19E−14 | 0.119464 | 0 | 0.000588 | 0.117151 |
| Grid2 | 53 | 2.22E−16 | 8.19E−14 | 0.89959 | 0 | 0.001764 | 0.012115 |
| Pcdhga11 | 54 | 2.22E−16 | 8.19E−14 | 0.074396 | 0 | 0.002666 | 0.168336 |
| Kcnh7 | 55 | 2.22E−16 | 8.19E−14 | 0.538884 | 0.000353 | 0 | 0.251399 |
| Pcdhga8 | 56 | 2.22E−16 | 8.19E−14 | 0.086942 | 0 | 0.003431 | 0.251279 |
| Pcdhga5 | 57 | 2.22E−16 | 8.19E−14 | 0.143165 | 0 | 0.00496 | 0.128051 |
| Dlgap1 | 58 | 2.22E−16 | 8.19E−14 | 0.15134 | 0 | 0.073298 | 0.037698 |
| Nlrp5-ps | 59 | 2.22E−16 | 8.19E−14 | 0.084629 | 0.317966 | 0 | 0.107522 |
| Cdh2 | 60 | 2.22E−16 | 8.19E−14 | 0.832487 | 0 | 0.080747 | 0.047127 |
| Gm10377 | 61 | 2.22E−13 | 8.04E−11 | 8.65E−05 | 5.38E−05 | 6.54E−05 | 7.15E−05 |
| Gm3002 | 62 | 3.98E−13 | 1.42E−10 | 0.000464 | 5.26E−05 | 8.32E−06 | 0.000202 |
| C2cd4b | 63 | 3.51E−12 | 1.24E−09 | 0.000252 | 0.000378 | 3.66E−05 | 0.000126 |
| M5C1000I18Rik | 64 | 7.39E−12 | 2.56E−09 | 0.000312 | 0.000474 | 4.52E−05 | 0.000148 |
| Npr1 | 65 | 1.44E−11 | 4.92E−09 | 0.000216 | 0.000137 | 0.000588 | 0.000118 |
| Gm45351 | 66 | 4.60E−11 | 1.54E−08 | 0.003892 | 0.00017 | 0.000155 | 7.08E−05 |
| Vmn2r47 | 67 | 5.03E−11 | 1.66E−08 | 0.003892 | 0.000181 | 0.000161 | 7.08E−05 |
| 3110070M22Rik | 68 | 7.03E−11 | 2.29E−08 | 8.01E−05 | 0.000257 | 0.000235 | 0.002402 |
| Hoxd1 | 69 | 1.31E−10 | 4.22E−08 | 0.001609 | 1.02E−05 | 0.000328 | 0.004304 |
| Septin9 | 70 | 2.88E−10 | 9.11E−08 | 0.000294 | 0.000608 | 0.000627 | 0.00049 |
| Ptprd | 71 | 3.05E−10 | 9.50E−08 | 0.052773 | 0.000137 | 0.000137 | 5.88E−05 |
| Fam205a1 | 72 | 4.81E−10 | 1.48E−07 | 0.001764 | 0.001764 | 0.000176 | 0.000176 |
| Mir6991 | 73 | 6.19E−10 | 1.88E−07 | 1.59E−05 | 0.000149 | 0.03263 | 0.001659 |
| Gm9748 | 74 | 8.32E−10 | 2.49E−07 | 0.06956 | 4.28E−06 | 0.000263 | 0.002272 |
| Cep85 | 75 | 9.20E−10 | 2.72E−07 | 0.000353 | 0.002294 | 0.000176 | 0.001392 |
| 4930558F17Rik | 76 | 1.52E−09 | 4.43E−07 | 0.003134 | 0.00081 | 0.000214 | 0.00064 |
| Hsf4 | 77 | 1.95E−09 | 5.62E−07 | 0.005626 | 0.000549 | 0.000843 | 0.000176 |
| Skint6 | 78 | 2.70E−09 | 7.65E−07 | 0.000976 | 0.001661 | 0.000874 | 0.000464 |
| Mup2 | 79 | 3.13E−09 | 8.77E−07 | 0.019506 | 9.35E−05 | 0.000176 | 0.002411 |
| Skint5 | 80 | 4.57E−09 | 1.26E−06 | 0.002162 | 0.002723 | 0.00117 | 0.000172 |
| Hic1 | 81 | 5.15E−09 | 1.41E−06 | 0.000137 | 0.008077 | 0.031189 | 3.92E−05 |
| St6gal2 | 82 | 5.27E−09 | 1.42E−06 | 0.011252 | 0.000176 | 0.001784 | 0.000392 |
| Rsph6a | 83 | 6.46E−09 | 1.72E−06 | 0.002411 | 0.00047 | 0.000451 | 0.003411 |
| Cacna1b | 84 | 7.43E−09 | 1.96E−06 | 0.005391 | 0.000176 | 0.001764 | 0.001215 |
| Defa23 | 85 | 9.55E−09 | 2.49E−06 | 0.000407 | 0.000138 | 0.001688 | 0.028594 |
| Hlx | 86 | 1.05E−08 | 2.71E−06 | 0.001059 | 0.001568 | 0.003195 | 0.000569 |
| Shmt2 | 87 | 1.15E−08 | 2.92E−06 | 0.055753 | 0.000216 | 5.88E−05 | 0.004705 |
| Kcnj3 | 88 | 1.85E−08 | 4.67E−06 | 0.011801 | 0.000118 | 0.000961 | 0.004274 |
| Adamts12 | 89 | 2.22E−08 | 5.51E−06 | 0.000176 | 0.007273 | 0.000137 | 0.03958 |
| Gm16513 | 90 | 2.24E−08 | 5.51E−06 | 0.032133 | 0.000712 | 0.000114 | 0.002703 |
| Tbx1 | 91 | 2.36E−08 | 5.75E−06 | 0.000196 | 0.001509 | 0.003666 | 0.0069 |
| Sp9 | 92 | 2.55E−08 | 6.14E−06 | 0.011311 | 0.010429 | 0.000392 | 0.000176 |
| Cntnap5c | 93 | 2.88E−08 | 6.85E−06 | 0.038952 | 0.001157 | 0.000882 | 0.000235 |
| Gm53 | 94 | 3.07E−08 | 7.24E−06 | 0.004901 | 0.009567 | 0.000843 | 0.000255 |
| Hspa1a | 95 | 3.26E−08 | 7.59E−06 | 3.05E−05 | 3.76E−05 | 0.099301 | ND |
| Grid1 | 96 | 3.56E−08 | 8.21E−06 | 0.043285 | 0.000118 | 0.000216 | 0.010821 |
| Hmx1 | 97 | 3.62E−08 | 8.26E−06 | 0.056008 | 0.000137 | 0.000176 | 0.008939 |
| Nkx6-2 | 98 | 3.66E−08 | 8.26E−06 | 0.005062 | 0.00016 | 0.007016 | 0.00215 |
| 4930442J19Rik | 99 | 3.81E−08 | 8.45E−06 | 0.092902 | 0.000137 | 0.004274 | 0.000235 |
| Hcn2 | 100 | 3.82E−08 | 8.45E−06 | 0.050989 | 0.000588 | 0.002431 | 0.000176 |
| Pax1 | 101 | 4.15E−08 | 9.10E−06 | 0.000588 | 0.000706 | 0.015232 | 0.002235 |
| Egfem1 | 102 | 4.24E−08 | 9.20E−06 | 0.006567 | 0.000137 | 0.001725 | 0.009312 |
| Vmn1r252 | 103 | 4.72E−08 | 1.01E−05 | 0.001776 | 0.000408 | 0.001853 | 0.012165 |
| Gm6623 | 104 | 5.00E−08 | 1.06E−05 | 0.000888 | 0.000745 | 0.008922 | 0.002951 |
| Ugt8a | 105 | 5.25E−08 | 1.11E−05 | 0.332713 | 0.000176 | 0.000372 | 0.000843 |
| Gldc | 106 | 5.31E−08 | 1.11E−05 | 0.001725 | 0.000804 | 0.000823 | 0.016369 |
| Lrp1b | 107 | 5.91E−08 | 1.22E−05 | 0.230303 | 0.000118 | 0.00049 | 0.001588 |
| Ccdc194 | 108 | 7.26E−08 | 1.49E−05 | 0.002372 | 0.002882 | 0.005842 | 0.000667 |
| Hoxa3 | 109 | 7.99E−08 | 1.62E−05 | 0.006293 | 0.000176 | 0.011272 | 0.002372 |
| Prpsap1 | 110 | 9.38E−08 | 1.88E−05 | 0.00641 | 0.000431 | 0.003039 | 0.004234 |
| Gna14 | 111 | 9.43E−08 | 1.88E−05 | 0.000412 | 0.015134 | 0.002764 | 0.002078 |
| Hoxa2 | 112 | 9.69E−08 | 1.92E−05 | 0.006293 | 0.000137 | 0.009959 | 0.004293 |
| Ccdc61 | 113 | 9.93E−08 | 1.95E−05 | 0.007273 | 0.008488 | 0.000392 | 0.001568 |
| Hspa12b | 114 | 1.02E−07 | 1.98E−05 | 0.000235 | 0.006273 | 0.024112 | 0.001098 |
| Ebf1 | 115 | 1.04E−07 | 2.00E−05 | 0.009508 | 0.000176 | 0.004391 | 0.005411 |
| Zcchc3 | 116 | 1.51E−07 | 2.88E−05 | 0.062805 | 0.00013 | 0.000467 | 0.016013 |
| Zfp951 | 117 | 1.56E−07 | 2.96E−05 | 0.015173 | 0.000137 | 0.018329 | 0.001666 |
| Hoxd3 | 118 | 1.78E−07 | 3.35E−05 | 0.0198 | 0.003019 | 0.000137 | 0.009018 |
| Lgals6 | 119 | 2.02E−07 | 3.62E−05 | 0.000901 | 0.003867 | 0.006505 | 0.003749 |
| Ryr2 | 120 | 2.08E−07 | 3.62E−05 | 0.152869 | 0.000216 | 0.00051 | 0.005254 |
| Btc | 121 | 2.14E−07 | 3.62E−05 | 0.017663 | 0.00198 | 0.000863 | 0.003019 |
| Vmn1r-ps79 | 122 | 2.21E−07 | 3.62E−05 | 0.003592 | 0.000683 | 0.004769 | 0.008051 |
| Vmn1r101 | 122 | 2.21E−07 | 3.62E−05 | 0.003592 | 0.000683 | 0.004769 | 0.008051 |
| Vmn1r250 | 122 | 2.21E−07 | 3.62E−05 | 0.003592 | 0.000683 | 0.004769 | 0.008051 |
| Gm10665 | 122 | 2.21E−07 | 3.62E−05 | 0.003592 | 0.000683 | 0.004769 | 0.008051 |
| Vmn1r256 | 122 | 2.21E−07 | 3.62E−05 | 0.003592 | 0.000683 | 0.004769 | 0.008051 |
| Vmn1r100 | 122 | 2.21E−07 | 3.62E−05 | 0.003592 | 0.000683 | 0.004769 | 0.008051 |
| Gm10668 | 122 | 2.21E−07 | 3.62E−05 | 0.003592 | 0.000683 | 0.004769 | 0.008051 |
| Gm4513 | 122 | 2.21E−07 | 3.62E−05 | 0.003592 | 0.000683 | 0.004769 | 0.008051 |
| Vmn1r142 | 122 | 2.21E−07 | 3.62E−05 | 0.003592 | 0.000683 | 0.004769 | 0.008051 |
| Vmn1r143 | 122 | 2.21E−07 | 3.62E−05 | 0.003592 | 0.000683 | 0.004769 | 0.008051 |
| Vmn1r251 | 122 | 2.21E−07 | 3.62E−05 | 0.003592 | 0.000683 | 0.004769 | 0.008051 |
| Vmn1r254 | 122 | 2.21E−07 | 3.62E−05 | 0.003592 | 0.000683 | 0.004769 | 0.008051 |
| Vmn1r152 | 122 | 2.21E−07 | 3.62E−05 | 0.003592 | 0.000683 | 0.004769 | 0.008051 |
| Vmn1r255 | 122 | 2.21E−07 | 3.62E−05 | 0.003592 | 0.000683 | 0.004769 | 0.008051 |
| Snx33 | 136 | 2.24E−07 | 3.64E−05 | 0.014664 | 0.002646 | 0.000882 | 0.002803 |
| Cacna1a | 137 | 2.25E−07 | 3.64E−05 | 0.01729 | 0.001588 | 0.000255 | 0.013781 |
| Cbln1 | 138 | 2.46E−07 | 3.95E−05 | 0.004921 | 0.000216 | 0.019466 | 0.005175 |
| Speg | 139 | 2.50E−07 | 3.99E−05 | 0.001843 | 0.000176 | 0.005528 | 0.060556 |
| Pxn | 140 | 2.70E−07 | 4.27E−05 | 0.009312 | 0.007822 | 0.003195 | 0.00051 |
| Grik2 | 141 | 2.80E−07 | 4.40E−05 | 0.640568 | 0.000118 | 0.000294 | 0.005587 |
| Mir497 | 142 | 2.93E−07 | 4.53E−05 | 0.024305 | 0.001849 | 0.005753 | 0.000503 |
| Sgce | 143 | 2.93E−07 | 4.53E−05 | 0.020486 | 0.000235 | 0.003725 | 0.007273 |
| Cdkn2b | 144 | 2.95E−07 | 4.53E−05 | 0.00051 | 0.003195 | 0.009018 | 0.008939 |
| Skint11 | 145 | 3.22E−07 | 4.92E−05 | 0.003401 | 0.000267 | 0.044747 | 0.003576 |
| Gm13034 | 146 | 3.32E−07 | 5.00E−05 | 0.012615 | 1.30E−05 | 0.028795 | 0.031864 |
| Barh11 | 147 | 3.34E−07 | 5.00E−05 | 0.056988 | 0.000137 | 0.001862 | 0.01041 |
| Irx3 | 148 | 3.34E−07 | 5.00E−05 | 0.002274 | 0.001784 | 0.000137 | 0.272353 |
| Nkain2 | 149 | 3.39E−07 | 5.03E−05 | 0.009351 | 0.000608 | 0.002078 | 0.013056 |
| Vmn1r107 | 150 | 3.41E−07 | 5.03E−05 | 0.009379 | 0.001227 | 0.003721 | 0.003621 |
| Mir497b | 151 | 3.47E−07 | 5.09E−05 | 0.024479 | 0.001872 | 0.005988 | 0.000577 |
| 5031439G07Rik | 152 | 3.62E−07 | 5.28E−05 | 0.000431 | 0.00249 | 0.002058 | 0.075219 |
| Hs6st3 | 153 | 3.65E−07 | 5.28E−05 | 0.042755 | 0.000235 | 0.001764 | 0.009449 |
| Cdo1 | 154 | 3.68E−07 | 5.30E−05 | 0.016447 | 0.001098 | 0.004391 | 0.002137 |
| Smarca5-ps | 155 | 4.02E−07 | 5.75E−05 | 0.066132 | 0.000419 | 0.000801 | 0.008449 |
| Prkd2 | 156 | 4.07E−07 | 5.77E−05 | 0.058105 | 0.000392 | 0.007449 | 0.001117 |
| Epha6 | 157 | 4.31E−07 | 6.08E−05 | 0.524201 | 0.000137 | 0.000137 | 0.020545 |
| Gm12371 | 158 | 4.40E−07 | 6.16E−05 | 0.166317 | 0.000176 | 0.003372 | 0.002098 |
| Vwc2 | 159 | 4.56E−07 | 6.34E−05 | 0.079434 | 0.000137 | 0.009723 | 0.002039 |
| Adcy2 | 160 | 4.75E−07 | 6.58E−05 | 0.002921 | 0.000255 | 0.022211 | 0.013723 |
| Mab21l2 | 161 | 5.01E−07 | 6.89E−05 | 0.45086 | 0.000653 | 0.000674 | 0.001214 |
| Gas2l2 | 162 | 5.44E−07 | 7.44E−05 | 0.004332 | 0.002039 | 0.001588 | 0.018898 |
| Gm7102 | 163 | 5.64E−07 | 7.64E−05 | 0.000968 | 0.00083 | 0.007492 | 0.045941 |
| Grb10 | 164 | 5.66E−07 | 7.64E−05 | 0.621435 | 0.000137 | 0.001823 | 0.001784 |
| Hoxb3 | 165 | 6.09E−07 | 8.13E−05 | 0.022466 | 0.001431 | 0.001666 | 0.005626 |
| Slc38a1 | 166 | 6.09E−07 | 8.13E−05 | 0.024681 | 0.000882 | 0.001098 | 0.012625 |
| Erbb4 | 167 | 6.19E−07 | 8.20E−05 | 0.019721 | 0.002764 | 0.002039 | 0.002764 |
| Obox3 | 168 | 6.70E−07 | 8.83E−05 | 0.024411 | 0.001184 | 0.00198 | 0.00588 |
| Brinp1 | 169 | 6.87E−07 | 9.01E−05 | 0.280959 | 0.000176 | 0.000686 | 0.010194 |
| Gm8579 | 170 | 7.24E−07 | 9.43E−05 | 0.01041 | 0.000588 | 0.007391 | 0.008136 |
| Jak3 | 171 | 7.38E−07 | 9.51E−05 | 0.017526 | 0.000176 | 0.002137 | 0.056988 |
| Dnajb1 | 172 | 7.39E−07 | 9.51E−05 | 0.033973 | 0.000451 | 0.010547 | 0.002333 |
| Foxd2os | 173 | 7.99E−07 | 0.000102 | 0.306581 | 0.002274 | 0.000392 | 0.001509 |
| Negr1 | 174 | 8.48E−07 | 0.000108 | 0.386701 | 0.000176 | 0.000706 | 0.009174 |
| Gm10710 | 175 | 8.96E−07 | 0.000113 | 0.150477 | 0.004842 | 0.001098 | 0.000588 |
| Kcnk12 | 176 | 9.07E−07 | 0.000114 | 0.077826 | 0.000627 | 0.001725 | 0.005665 |
| Bcat1 | 177 | 9.16E−07 | 0.000115 | 0.084786 | 0.001764 | 0.000588 | 0.005489 |
| Ina | 178 | 1.03E−06 | 0.000128 | 0.02374 | 0.000961 | 0.003333 | 0.007293 |
| Srrm4 | 179 | 1.05E−06 | 0.00013 | 0.065202 | 0.000176 | 0.044618 | 0.001098 |
| Guca2a | 180 | 1.07E−06 | 0.000132 | 0.02706 | 0.006975 | 0.001835 | 0.001667 |
| Hoxa7 | 181 | 1.09E−06 | 0.000134 | 0.007449 | 0.064241 | 0.003705 | 0.000333 |
| Evx2 | 182 | 1.12E−06 | 0.000136 | 0.043285 | 0.000176 | 0.021584 | 0.003685 |
| Rpp25l | 183 | 1.12E−06 | 0.000136 | 0.042231 | 0.018087 | 0.000484 | 0.001653 |
| Celf3 | 184 | 1.13E−06 | 0.000136 | 0.221854 | 0.000137 | 0.010213 | 0.00198 |
| Kcnj11 | 185 | 1.14E−06 | 0.000137 | 0.003489 | 0.003352 | 0.004921 | 0.010802 |
| Prr36 | 186 | 1.16E−06 | 0.000138 | 0.175668 | 0.000235 | 0.002078 | 0.007391 |
| Pitx2 | 187 | 1.19E−06 | 0.000141 | 0.002411 | 0.005175 | 0.002039 | 0.02574 |
| Mir762 | 188 | 1.20E−06 | 0.000141 | 0.029717 | 0.002775 | 0.002347 | 0.003398 |
| Tmprss11d | 189 | 1.22E−06 | 0.000142 | 0.003951 | 0.019915 | 0.003121 | 0.002724 |
| Raet1c | 190 | 1.23E−06 | 0.000143 | 0.006097 | 0.007633 | 0.0032 | 0.004546 |
| Exoc3l2 | 191 | 1.27E−06 | 0.000147 | 0.002686 | 0.000588 | 0.03264 | 0.013605 |
| Sstr2 | 192 | 1.27E−06 | 0.000147 | 0.008469 | 0.00198 | 0.003313 | 0.012723 |
| Plekhm2 | 193 | 1.29E−06 | 0.000148 | 0.114838 | 0.036012 | 0.000294 | 0.000588 |
| Elavl4 | 194 | 1.30E−06 | 0.000148 | 0.075984 | 0.000216 | 0.000588 | 0.074749 |
| Kbtbd7 | 195 | 1.30E−06 | 0.000148 | 0.00764 | 0.254264 | 0.001605 | 0.000232 |
| Usp13 | 196 | 1.33E−06 | 0.00015 | 0.143087 | 0.006293 | 0.000176 | 0.004666 |
| Lrp3 | 197 | 1.38E−06 | 0.000155 | 0.001215 | 0.002137 | 0.008449 | 0.035188 |
| Foxd3 | 198 | 1.41E−06 | 0.000158 | 0.025086 | 4.91E−05 | 0.012616 | 0.051384 |
| Fut9 | 199 | 1.42E−06 | 0.000158 | 0.052224 | 0.000235 | 0.000863 | 0.075631 |
| Nr4a3 | 200 | 1.60E−06 | 0.000177 | 0.066103 | 0.011919 | 0.001215 | 0.000961 |
| Ccdc124 | 201 | 1.61E−06 | 0.000177 | 0.154927 | 0.003764 | 0.000627 | 0.002529 |
| Otud7a | 202 | 1.62E−06 | 0.000177 | 0.0257 | 0.000627 | 0.000216 | 0.267472 |
| Kctd8 | 203 | 1.66E−06 | 0.000181 | 0.061301 | 0.000255 | 0.001588 | 0.038698 |
| Clmp | 204 | 1.81E−06 | 0.000197 | 0.027151 | 0.006704 | 0.001365 | 0.004274 |
| Obox1 | 205 | 1.82E−06 | 0.000197 | 0.055097 | 0.000939 | 0.002582 | 0.008005 |
| Ano2 | 206 | 1.85E−06 | 0.000199 | 0.01484 | 0.010645 | 0.010645 | 0.000647 |
| Nrbp1 | 207 | 1.91E−06 | 0.000205 | 0.005273 | 0.013193 | 0.004999 | 0.003254 |
| Dcdc2c | 208 | 2.06E−06 | 0.000219 | 0.013879 | 0.002901 | 0.00347 | 0.008802 |
| Rasip1 | 209 | 2.10E−06 | 0.000222 | 0.045402 | 0.049538 | 0.001098 | 0.00051 |
| Syt14 | 210 | 2.25E−06 | 0.000237 | 0.072396 | 0.050401 | 0.000235 | 0.001588 |
| Mrgpra2b | 211 | 2.30E−06 | 0.000241 | 0.153717 | 0.054899 | 7.56E−05 | 0.002195 |
| Dtx1 | 212 | 2.30E−06 | 0.000241 | 0.006508 | 0.003078 | 0.002529 | 0.02772 |
| Klf4 | 213 | 2.39E−06 | 0.000247 | 0.033346 | 0.025034 | 0.001627 | 0.001078 |
| Klf14 | 214 | 2.39E−06 | 0.000247 | 0.064776 | 0.017013 | 0.001019 | 0.001306 |
| Trim52 | 215 | 2.40E−06 | 0.000247 | 0.003999 | 0.004097 | 0.01482 | 0.006058 |
| Yjefn3 | 216 | 2.50E−06 | 0.000256 | 0.011076 | 0.006371 | 0.003646 | 0.005999 |
| Mir1192 | 217 | 2.51E−06 | 0.000257 | 0.00382 | 0.010834 | 0.001853 | 0.020267 |
| Ptpro | 218 | 2.60E−06 | 0.000264 | 0.048244 | 0.000549 | 0.012919 | 0.004724 |
| Sync | 219 | 2.70E−06 | 0.000273 | 0.006646 | 0.000677 | 0.025796 | 0.014564 |
| Gm38666 | 220 | 2.79E−06 | 0.000281 | 0.014703 | 0.000294 | 0.020153 | 0.020153 |
| Nt5c2 | 221 | 2.85E−06 | 0.000285 | 0.060791 | 0.002235 | 0.001725 | 0.007665 |
| Fbxl7 | 222 | 2.87E−06 | 0.000285 | 0.175472 | 0.002078 | 0.000118 | 0.042324 |
| Scn8a | 223 | 2.88E−06 | 0.000285 | 0.010468 | 0.007097 | 0.003705 | 0.006626 |
| 6820408C15Rik | 224 | 2.89E−06 | 0.000285 | 0.05983 | 0.000529 | 0.000843 | 0.068417 |
| Trpc6 | 225 | 2.89E−06 | 0.000285 | 0.064045 | 0.000569 | 0.017447 | 0.002882 |
| Gm3867 | 226 | 2.92E−06 | 0.000286 | 0.002689 | 0.007547 | 0.001774 | 0.051439 |
| Cyp26c1 | 227 | 3.00E−06 | 0.000292 | 0.043285 | 0.000137 | 0.001411 | 0.227304 |
| Cdkn2a | 228 | 3.04E−06 | 0.000294 | 0.064065 | 0.000353 | 0.006312 | 0.013566 |
| Il11ra1 | 229 | 3.04E−06 | 0.000294 | 0.015369 | 0.073886 | 0.003489 | 0.00049 |
| Ak5 | 230 | 3.14E−06 | 0.000302 | 0.020643 | 0.001313 | 0.007214 | 0.010292 |
| Adamts4 | 231 | 3.18E−06 | 0.000304 | 0.015585 | 0.000431 | 0.00645 | 0.047088 |
| Unc5c | 232 | 3.18E−06 | 0.000304 | 0.308463 | 0.000588 | 0.000549 | 0.020525 |
| Gse1 | 233 | 3.21E−06 | 0.000305 | 0.036012 | 0.009194 | 0.010625 | 0.000588 |
| A830018L16Rik | 234 | 3.22E−06 | 0.000305 | 0.324832 | 0.000431 | 0.000137 | 0.107977 |
| Irgc1 | 235 | 3.37E−06 | 0.000318 | 0.014742 | 0.033855 | 0.022348 | 0.000196 |
| Pcdhga2 | 236 | 3.42E−06 | 0.000321 | 0.118837 | 0.000137 | 0.003117 | 0.043794 |
| Galnt2 | 237 | 3.48E−06 | 0.000324 | 0.378428 | 0.000216 | 0.001411 | 0.019702 |
| Riiad1 | 238 | 3.48E−06 | 0.000324 | 0.126953 | 0.00049 | 0.004646 | 0.007861 |
| Cxxc5 | 239 | 3.51E−06 | 0.000325 | 0.027582 | 0.013076 | 0.016193 | 0.000392 |
| Gpr139 | 240 | 3.55E−06 | 0.000328 | 0.149693 | 0.001117 | 0.004352 | 0.003195 |
| Wdtc1 | 241 | 3.67E−06 | 0.000337 | 0.016291 | 0.02176 | 0.001059 | 0.00643 |
| Espn | 242 | 3.95E−06 | 0.000361 | 0.041324 | 0.001823 | 0.011488 | 0.003039 |
| Fignl2 | 243 | 3.99E−06 | 0.000364 | 0.315285 | 0.023818 | 0.000118 | 0.003019 |
| Nr2f2 | 244 | 4.05E−06 | 0.000367 | 0.00741 | 0.011958 | 0.000882 | 0.03464 |
| Nox4 | 245 | 4.10E−06 | 0.000371 | 0.069024 | 0.000235 | 0.001137 | 0.148889 |
| Actl10 | 246 | 4.15E−06 | 0.000372 | 0.004555 | 0.155601 | 0.000736 | 0.005349 |
| Pcdhgb8 | 247 | 4.15E−06 | 0.000372 | 0.062457 | 0.000176 | 0.000569 | 0.44561 |
| Krt19 | 248 | 4.23E−06 | 0.000378 | 0.008998 | 0.001353 | 0.01235 | 0.018976 |
| Gp1bb | 249 | 4.41E−06 | 0.000392 | 0.005492 | 0.076875 | 0.000561 | 0.012623 |
| Cdh7 | 250 | 4.44E−06 | 0.000394 | 0.083453 | 0.000451 | 0.017271 | 0.004646 |
| Lrrc4c | 251 | 4.50E−06 | 0.000397 | 0.0999 | 0.001509 | 0.000196 | 0.103566 |
| Hoxc5 | 252 | 4.54E−06 | 0.000397 | 0.091902 | 0.01037 | 0.000627 | 0.005175 |
| Tafa5 | 253 | 4.54E−06 | 0.000397 | 0.019466 | 0.000137 | 0.003489 | 0.332242 |
| Runx1t1 | 254 | 4.58E−06 | 0.0004 | 0.135579 | 0.00051 | 0.002411 | 0.0188 |
| BC021767 | 255 | 4.97E−06 | 0.000431 | 0.037364 | 0.001294 | 0.004372 | 0.016271 |
| Tmem181c-ps | 256 | 5.00E−06 | 0.000432 | 0.006293 | 0.006528 | 0.001098 | 0.076866 |
| Hdac3 | 257 | 5.11E−06 | 0.00044 | 0.007979 | 0.004038 | 0.008939 | 0.01235 |
| Mamstr | 258 | 5.13E−06 | 0.00044 | 0.066633 | 0.000706 | 0.006097 | 0.012468 |
| Khdrbs2 | 259 | 5.51E−06 | 0.000471 | 0.204897 | 0.000294 | 0.007979 | 0.008077 |
| Zfp423 | 260 | 5.53E−06 | 0.000471 | 0.023368 | 0.004489 | 0.003882 | 0.009586 |
| Tmem88 | 261 | 5.63E−06 | 0.000478 | 0.005438 | 0.005374 | 0.005695 | 0.023931 |
| Arhgef1 | 262 | 5.80E−06 | 0.00049 | 0.043089 | 0.00447 | 0.003195 | 0.006704 |
| Evpl | 263 | 5.82E−06 | 0.00049 | 0.002137 | 0.003019 | 0.026935 | 0.023838 |
| Depp1 | 264 | 5.86E−06 | 0.000492 | 0.011228 | 0.004389 | 0.008791 | 0.009642 |
| Ptf1a | 265 | 6.07E−06 | 0.000504 | 0.067009 | 0.007063 | 0.002929 | 0.00314 |
| Pcdh15 | 266 | 6.09E−06 | 0.000504 | 0.128012 | 0.000255 | 0.000843 | 0.158868 |
| Fign | 267 | 6.12E−06 | 0.000504 | 0.269903 | 0.002333 | 0.003489 | 0.002 |
| Gm7030 | 268 | 6.12E−06 | 0.000504 | 0.014017 | 0.001 | 0.013174 | 0.023818 |
| Zfp575 | 269 | 6.13E−06 | 0.000504 | 0.252311 | 0.016126 | 0.001445 | 0.000748 |
| Clec2d | 270 | 6.19E−06 | 0.000506 | 0.026053 | 0.009567 | 0.000882 | 0.020251 |
| Klh11 | 271 | 6.20E−06 | 0.000506 | 0.050283 | 0.001137 | 0.002431 | 0.032111 |
| Gria4 | 272 | 6.21E−06 | 0.000506 | 0.669346 | 0.000706 | 5.88E−05 | 0.160946 |
| Sirt4 | 273 | 6.34E−06 | 0.000514 | 0.00496 | 0.006959 | 0.005411 | 0.024505 |
| Arhgap28 | 274 | 6.42E−06 | 0.000518 | 0.108232 | 0.003195 | 0.008057 | 0.001666 |
| I116 | 275 | 6.52E−06 | 0.000525 | 0.004587 | 0.034365 | 0.012448 | 0.002411 |
| Colla1 | 276 | 6.54E−06 | 0.000525 | 0.015291 | 0.001098 | 0.007057 | 0.040109 |
| Car10 | 277 | 6.76E−06 | 0.00054 | 0.830115 | 0.000137 | 0.003744 | 0.011566 |
| St3gal2 | 278 | 7.14E−06 | 0.000568 | 0.03856 | 0.012017 | 0.001627 | 0.006979 |
| Ctnnd2 | 279 | 7.50E−06 | 0.000593 | 0.257356 | 0.000843 | 0.002862 | 0.008978 |
| C1rb | 280 | 7.55E−06 | 0.000593 | 0.022348 | 0.00198 | 0.091294 | 0.001392 |
| Slc22a18 | 281 | 7.56E−06 | 0.000593 | 0.017486 | 0.006724 | 0.004607 | 0.01039 |
| Nlgn1 | 282 | 7.56E−06 | 0.000593 | 0.135794 | 0.000235 | 0.004901 | 0.035953 |
| Pcbp1 | 283 | 7.70E−06 | 0.000603 | 0.005262 | 0.009591 | 0.007491 | 0.015224 |
| Usp10 | 284 | 7.74E−06 | 0.000603 | 0.627159 | 0.000137 | 0.00792 | 0.008488 |
| Mir3074-2 | 285 | 8.30E−06 | 0.000645 | 0.005309 | 0.01625 | 0.005391 | 0.013518 |
| Fh13 | 286 | 8.34E−06 | 0.000646 | 0.010076 | 0.004372 | 0.033738 | 0.004254 |
| Igsf23 | 287 | 9.00E−06 | 0.000695 | 0.008743 | 0.004666 | 0.004979 | 0.034051 |
| 1700123012Rik | 288 | 9.38E−06 | 0.000721 | 0.094431 | 0.001215 | 0.009273 | 0.006822 |
| Gpr158 | 289 | 9.53E−06 | 0.00073 | 0.179255 | 0.002588 | 0.000255 | 0.062555 |
| Creld1 | 290 | 9.60E−06 | 0.000732 | 0.01731 | 0.000706 | 0.008449 | 0.07222 |
| Mir24-2 | 291 | 9.63E−06 | 0.000732 | 0.005189 | 0.017043 | 0.005135 | 0.016475 |
| Cyp26b1 | 292 | 9.65E−06 | 0.000732 | 0.003548 | 0.030131 | 0.023093 | 0.003039 |
| D830030K20Rik | 293 | 9.78E−06 | 0.000739 | 0.048204 | 0.012386 | 0.000334 | 0.038199 |
| Sox17 | 294 | 1.00E−05 | 0.000753 | 0.222168 | 0.000137 | 0.001117 | 0.229872 |
| Kdelr2 | 295 | 1.00E−05 | 0.000753 | 0.009684 | 0.045657 | 0.004901 | 0.003627 |
| Ropn1l | 296 | 1.02E−05 | 0.00076 | 0.408931 | 0.022309 | 0.000137 | 0.006371 |
| Mir23a | 297 | 1.04E−05 | 0.000773 | 0.007117 | 0.014371 | 0.003869 | 0.020644 |
| Ajap1 | 298 | 1.04E−05 | 0.000776 | 0.476878 | 0.002137 | 0.000843 | 0.009586 |
| Tecr | 299 | 1.05E−05 | 0.000781 | 0.046833 | 0.002764 | 0.010076 | 0.006391 |
| Vasp | 300 | 1.06E−05 | 0.000781 | 0.042344 | 0.002078 | 0.004921 | 0.019349 |
| Vmn2r55 | 301 | 1.06E−05 | 0.000781 | 0.011077 | 0.012568 | 0.013633 | 0.004422 |
| Cbx8 | 302 | 1.07E−05 | 0.000786 | 0.006861 | 0.033209 | 0.002882 | 0.012938 |
| Shisa6 | 303 | 1.08E−05 | 0.00079 | 0.314912 | 0.00051 | 0.003823 | 0.013977 |
| Pou3f3 | 304 | 1.09E−05 | 0.00079 | 0.10188 | 0.000588 | 0.253024 | 0.000569 |
| Irgm2 | 305 | 1.11E−05 | 0.000805 | 0.004411 | 0.031346 | 0.120719 | 0.000529 |
| Igsf9b | 306 | 1.11E−05 | 0.000807 | 0.083805 | 0.000569 | 0.029131 | 0.00641 |
| Limd1 | 307 | 1.12E−05 | 0.000807 | 0.014683 | 0.006626 | 0.016545 | 0.005567 |
| Gent2 | 308 | 1.12E−05 | 0.000807 | 0.06328 | 0.00545 | 0.001725 | 0.015075 |
| Prss21 | 309 | 1.13E−05 | 0.00081 | 0.356315 | 0.004587 | 0.00147 | 0.003764 |
| Rara | 310 | 1.14E−05 | 0.000814 | 0.036149 | 0.032326 | 0.011076 | 0.000706 |
| Capn11 | 311 | 1.14E−05 | 0.000814 | 0.021211 | 0.190096 | 0.005273 | 0.000431 |
| Nfix | 312 | 1.16E−05 | 0.000821 | 0.048088 | 0.012723 | 0.002215 | 0.006861 |
| 5830428M24Rik | 313 | 1.17E−05 | 0.000828 | 0.032111 | 0.009821 | 0.006861 | 0.004352 |
| Gucy1a2 | 314 | 1.19E−05 | 0.000839 | 0.255592 | 0.005371 | 0.000196 | 0.035718 |
| Tdrd6 | 315 | 1.20E−05 | 0.000846 | 0.064143 | 0.002646 | 0.004254 | 0.013487 |
| Gm10406 | 316 | 1.24E−05 | 0.000866 | 0.029018 | 0.074121 | 0.004411 | 0.001059 |
| Cdc14b | 317 | 1.25E−05 | 0.000867 | 0.009057 | 0.006508 | 0.011076 | 0.015585 |
| C1qtnf1 | 318 | 1.25E−05 | 0.000867 | 0.00939 | 0.039835 | 0.005822 | 0.004685 |
| Sntg1 | 319 | 1.25E−05 | 0.000867 | 0.508988 | 0.000137 | 0.00396 | 0.036894 |
| Apba2 | 320 | 1.26E−05 | 0.000867 | 0.311188 | 0.002058 | 0.000235 | 0.067907 |
| Dchs2 | 321 | 1.26E−05 | 0.000867 | 0.087648 | 0.004489 | 0.005273 | 0.00494 |
| Foxf2 | 322 | 1.26E−05 | 0.000867 | 0.015408 | 0.005097 | 0.02917 | 0.004489 |
| Prkcb | 323 | 1.26E−05 | 0.000867 | 0.018427 | 0.001627 | 0.240713 | 0.001431 |
| Skint4 | 324 | 1.27E−05 | 0.000871 | 0.167323 | 0.00173 | 0.010875 | 0.00331 |
| Stra8 | 325 | 1.28E−05 | 0.000873 | 0.000882 | 0.024505 | 0.007214 | 0.067436 |
| A730013G03Rik | 326 | 1.28E−05 | 0.000873 | 0.004195 | 0.017153 | 0.010664 | 0.013708 |

TABLE 7
(Supplementary Table S3b. Ranked genes by differential methylation
(Y vs Old-2): RANK prom., p-value prom., q-value prom.)

| | RANK | p-value | q-value | p-value | p-value | p-value | p-value |
|---|---|---|---|---|---|---|---|
| Gene | prom. | prom. | prom. | prom. TR1 | prom. TR2 | prom. TR3 | prom. TR4 |
| Lncppara | 2107 | 0.016948 | 0.171503 | 0.066093 | 0.13437 | 0.103204 | 0.098092 |
| B3glct | 1 | 2.22E−16 | 4.74E−12 | 8.57E−06 | 5.56E−06 | 6.41E−06 | 7.28E−06 |
| Egr3 | 3979 | 0.07243 | 0.388135 | 0.276478 | 0.313542 | 0.10506 | 0.082882 |
| Lncpint | 269 | 4.37E−05 | 0.003461 | 0.030716 | 0.058061 | 0.002236 | 0.011318 |
| Grm7 | 17 | 1.50E−10 | 1.78E−07 | 0.004197 | 0.000305 | 0.000348 | 6.02E−05 |
| Sox11 | 10 | 3.17E−11 | 6.77E−08 | 0.548704 | 8.08E−07 | 2.20E−05 | 0.000496 |
| Bhlhe40 | 2800 | 0.033349 | 0.253929 | 0.34194 | 0.066577 | 0.094303 | 0.109919 |
| Mmp16 | 29 | 5.89E−09 | 4.33E−06 | 0.001339 | 0.0002 | 0.06548 | 8.96E−05 |
| 5730507C01Rik | 2 | 1.31E−13 | 1.13E−09 | 0.001185 | 1.70E−05 | 0.000106 | 5.78E−06 |
| Foxg1 | 26 | 1.75E−09 | 1.44E−06 | 0.019953 | 7.90E−05 | 6.43E−05 | 0.004013 |
| Gm4425 | 98 | 9.37E−07 | 0.000203 | ND | 0.000151 | ND | 0.000349 |
| Hoxa9 | 896 | 0.001782 | 0.042411 | 0.001764 | 0.475851 | 0.010243 | 0.516278 |
| Tmem267 | 22 | 9.32E−10 | 9.03E−07 | 0.000136 | 0.00055 | 0.000419 | 0.006434 |
| 9030624G23Rik | 9 | 3.08E−11 | 6.77E−08 | ND | 1.36E−05 | 0.000137 | 3.34E−05 |
| Cilp2 | 27 | 2.35E−09 | 1.86E−06 | 0.004828 | 0.001227 | 0.000197 | 0.000484 |
| Nrg3 | 14 | 1.20E−10 | 1.75E−07 | 0.000871 | 3.04E−05 | 0.001165 | 0.000675 |
| Otx1 | 451 | 0.000236 | 0.011138 | 0.024504 | 0.006613 | 0.027991 | 0.077226 |
| Lingo2 | 44 | 6.28E−08 | 3.05E−05 | 0.074167 | 0.000149 | 0.000959 | 0.002126 |
| Mdga2 | 4 | 1.64E−12 | 8.73E−09 | 0.011411 | 1.88E−06 | 0.00031 | 2.87E−05 |
| Nr4a2 | 1287 | 0.004786 | 0.079312 | 0.157532 | 0.169013 | 0.025827 | 0.023441 |
| Nkain3 | 35 | 2.99E−08 | 1.82E−05 | 0.003097 | 0.000387 | 0.000944 | 0.008638 |
| Peg10 | 15 | 1.29E−10 | 1.75E−07 | 0.00309 | 3.95E−05 | 0.000241 | 0.00077 |
| Haglr | 18 | 1.50E−10 | 1.78E−07 | 0.001654 | 1.04E−05 | 0.000343 | 0.004551 |
| Zfta | 2323 | 0.021815 | 0.200243 | 0.059767 | 0.225463 | 0.048935 | 0.19454 |
| Sgcz | 63 | 2.69E−07 | 9.06E−05 | 0.331335 | 3.24E−05 | 0.000837 | 0.013114 |
| 3110039M20Rik | 11 | 5.13E−11 | 9.22E−08 | 0.029056 | 2.58E−05 | 8.68E−06 | 0.001262 |
| Pcdhga9 | 402 | 0.000163 | 0.008625 | 0.061685 | 0.011921 | 0.005937 | 0.05079 |
| Pcdhga10 | 205 | 1.62E−05 | 0.001687 | 0.009369 | 0.003993 | 0.024877 | 0.014894 |
| Pcdh8 | 30 | 6.72E−09 | 4.78E−06 | 0.10986 | 1.04E−05 | 0.000597 | 0.00267 |
| Hoxaas3 | 1301 | 0.004967 | 0.081421 | 0.326401 | 0.001243 | 0.229401 | ND |
| Csmd1 | 71 | 3.36E−07 | 0.0001 | 0.03318 | 4.49E−05 | 0.004758 | 0.021522 |
| Tcfl5 | 8 | 2.51E−11 | 6.70E−08 | 0.000684 | 1.93E−05 | ND | 3.81E−06 |
| Xkr4 | 217 | 1.95E−05 | 0.001913 | 0.163921 | 0.066007 | 0.000226 | 0.007044 |
| Pcdh10 | 1469 | 0.006933 | 0.100661 | 0.420358 | 0.011554 | 0.012088 | 0.450215 |
| Cntnap2 | 226 | 2.33E−05 | 0.00219 | 0.126316 | 7.74E−05 | 0.785194 | 0.00277 |
| Prdm13 | 7768 | 0.272976 | 0.749201 | 0.757332 | 0.040201 | 0.325484 | 0.719116 |
| Galnt13 | 24 | 1.18E−09 | 1.05E−06 | 0.19193 | 5.97E−07 | 0.0003 | 0.007624 |
| Pcdhgb2 | 1162 | 0.003625 | 0.066543 | 0.005119 | 0.044721 | 0.052858 | 0.923425 |
| Pcdhga6 | 625 | 0.000604 | 0.020589 | 0.052515 | 0.003981 | 0.087952 | 0.061247 |
| Csf2ra | 423 | 0.000195 | 0.009823 | 0.902966 | 0.000449 | 0.029289 | 0.023289 |
| Rims2 | 251 | 3.24E−05 | 0.002751 | 0.081055 | 0.054913 | 0.000755 | 0.009387 |
| Nat8f3 | 288 | 5.19E−05 | 0.003843 | 0.009773 | 0.006923 | 0.208577 | 0.003935 |
| Pcdhga12 | 66 | 2.86E−07 | 9.06E−05 | 0.018001 | 0.000163 | 0.000157 | 0.274152 |
| Pcdhgb6 | 692 | 0.000791 | 0.024364 | 0.096034 | 0.003074 | 0.016653 | 0.321004 |
| Pcdhga7 | 2919 | 0.03619 | 0.264338 | 0.287763 | 0.073843 | 0.330683 | 0.03784 |
| Nefm | 53 | 1.82E−07 | 7.24E−05 | 0.001659 | 7.20E−05 | 0.02917 | 0.021761 |
| Pcdhga4 | 3636 | 0.059203 | 0.347104 | 0.605856 | 0.053892 | 0.04349 | 0.390072 |
| 2410018L13Rik | 339 | 9.67E−05 | 0.006081 | ND | 9.71E−05 | 0.071182 | 0.124342 |
| A330008L17Rik | 210 | 1.84E−05 | 0.001871 | 0.027841 | 0.000126 | 0.005644 | 0.814586 |
| Pcdhgb5 | 3089 | 0.041558 | 0.286825 | 0.647191 | 0.004708 | 0.297711 | 0.359216 |
| Pcdhgb4 | 4441 | 0.091896 | 0.441231 | 0.902148 | 0.031072 | 0.732732 | 0.053383 |
| Pcdhgb7 | 1517 | 0.007559 | 0.106259 | 0.724343 | 0.009701 | 0.047047 | 0.089829 |
| Grid2 | 366 | 0.000123 | 0.007136 | 0.808525 | 0.000493 | 0.011197 | 0.035207 |
| Pcdhga11 | 1443 | 0.006567 | 0.097065 | 0.469532 | 0.002558 | 0.197167 | 0.103792 |
| Kcnh7 | 315 | 7.77E−05 | 0.005258 | 0.5867 | 0.005036 | 0.000226 | ND |
| Pcdhga8 | 13557 | 0.666549 | 0.999999 | 0.995473 | 0.063686 | 0.878962 | 0.973947 |
| Pcdhga5 | 4980 | 0.114266 | 0.489202 | 0.960797 | 0.004197 | 0.545298 | 0.707998 |
| Dlgap1 | 642 | 0.000655 | 0.021746 | 0.201468 | 7.90E−05 | 0.79973 | 0.097829 |
| Nlrp5-ps | 220 | 2.02E−05 | 0.001957 | 0.272301 | ND | 5.12E−06 | ND |
| Cdh2 | 171 | 8.25E−06 | 0.001029 | 0.586315 | 1.73E−05 | 0.063406 | 0.009713 |
| Gm10377 | NA | ND | ND | ND | ND | ND | ND |
| Gm3002 | NA | ND | ND | ND | ND | ND | ND |
| C2cd4b | 6 | 3.51E−12 | 1.25E−08 | 0.000252 | 0.000378 | 3.66E−05 | 0.000126 |
| M5C1000I18Rik | 7 | 7.39E−12 | 2.25E−08 | 0.000312 | 0.000474 | 4.52E−05 | 0.000148 |
| Npr1 | 3 | 1.59E−13 | 1.13E−09 | 0.000276 | 1.27E−05 | 0.000185 | 2.34E−05 |
| Gm45351 | NA | ND | ND | ND | ND | ND | ND |
| Vmn2r47 | NA | ND | ND | ND | ND | ND | ND |
| 3110070M22Rik | 13 | 7.03E−11 | 1.15E−07 | 8.01E−05 | 0.000257 | 0.000235 | 0.002402 |
| Hoxd1 | 16 | 1.31E−10 | 1.75E−07 | 0.001609 | 1.02E−05 | 0.000328 | 0.004304 |
| Septin9 | 97 | 8.66E−07 | 0.00019 | 0.000906 | 0.008756 | 0.006578 | 0.008671 |
| Ptprd | 401 | 0.000162 | 0.008624 | 0.252088 | 0.056411 | 0.0364 | 0.000427 |
| Fam205a1 | 551 | 0.000396 | 0.015325 | 0.022993 | 0.032526 | 0.052054 | 0.017061 |
| Mir6991 | 20 | 6.19E−10 | 6.60E−07 | 1.59E−05 | 0.000149 | 0.03263 | 0.001659 |
| Gm9748 | 21 | 8.32E−10 | 8.45E−07 | 0.06956 | 4.28E−06 | 0.000263 | 0.002272 |
| Cep85 | 1327 | 0.005224 | 0.083919 | 0.123609 | 0.051324 | 0.048181 | 0.059238 |
| 4930558F17Rik | NA | ND | ND | ND | ND | ND | ND |
| Hsf4 | 5 | 3.10E−12 | 1.25E−08 | 0.000747 | 6.22E−05 | 7.94E−05 | 0.000103 |
| Skint6 | NA | ND | ND | ND | ND | ND | ND |
| Mup2 | 7791 | 0.274987 | 0.752452 | 0.745136 | ND | 0.071381 | 0.43661 |
| Skint5 | NA | ND | ND | ND | ND | ND | ND |
| Hic1 | 698 | 0.000823 | 0.02512 | 0.008049 | 0.384124 | 0.249908 | 0.002149 |
| St6gal2 | 31 | 1.05E−08 | 7.24E−06 | 0.018687 | 0.000222 | 0.00193 | 0.000377 |
| Rsph6a | 50 | 1.72E−07 | 7.24E−05 | 0.001941 | 0.004288 | 0.004207 | 0.002032 |
| Cacna1b | 12 | 5.19E−11 | 9.22E−08 | 0.000787 | 4.56E−05 | 0.000763 | 0.000304 |
| Defa23 | NA | ND | ND | ND | ND | ND | ND |
| Hlx | 239 | 2.71E−05 | 0.002409 | 0.014556 | 0.010635 | 0.020131 | 0.008186 |
| Shmt2 | 568 | 0.000432 | 0.016211 | 0.195623 | 0.006957 | 0.010798 | 0.050331 |
| Kcnj3 | 28 | 5.40E−09 | 4.11E−06 | 0.007562 | 0.000107 | 0.00047 | 0.003755 |
| Adamts12 | 25 | 1.75E−09 | 1.44E−06 | 2.31E−05 | 0.007388 | 0.00018 | 0.013206 |
| Gm16513 | NA | ND | ND | ND | ND | ND | ND |
| Tbx1 | 830 | 0.001415 | 0.036288 | 0.127884 | 0.047322 | 0.019822 | 0.027555 |
| Sp9 | 646 | 0.000671 | 0.022157 | 0.347982 | 0.212112 | 0.013033 | 0.001335 |
| Cntnap5c | 971 | 0.002212 | 0.048538 | 0.11823 | 0.12513 | 0.128488 | 0.003087 |
| Gm53 | 61 | 2.21E−07 | 7.64E−05 | 0.010994 | 0.00149 | 0.004681 | 0.00123 |
| Hspa1a | 37 | 3.26E−08 | 1.88E−05 | 3.05E−05 | 3.76E−05 | 0.099301 | ND |
| Grid1 | 47 | 9.61E−08 | 4.36E−05 | 0.014784 | 0.000479 | 0.000664 | 0.007774 |
| Hmx1 | 109 | 1.24E−06 | 0.000242 | 0.12815 | 0.000257 | 0.005758 | 0.003601 |
| Nkx6-2 | 38 | 3.66E−08 | 2.02E−05 | 0.005062 | 0.00016 | 0.007016 | 0.00215 |
| 4930442J19Rik | 509 | 0.000336 | 0.01407 | 0.541187 | 0.001247 | 0.034825 | 0.023031 |
| Hcn2 | 3298 | 0.048068 | 0.310692 | 0.075857 | 0.127246 | 0.588173 | 0.071262 |
| Pax1 | 733 | 0.000956 | 0.027817 | 0.028454 | 0.01386 | 0.077818 | 0.065373 |
| Egfem1 | 36 | 3.18E−08 | 1.88E−05 | 0.000913 | 0.000823 | 0.00348 | 0.004005 |
| Vmn1r252 | NA | ND | ND | ND | ND | ND | ND |
| Gm6623 | 41 | 5.00E−08 | 2.60E−05 | 0.000888 | 0.000745 | 0.008922 | 0.002951 |
| Ugt8a | 62 | 2.22E−07 | 7.64E−05 | 0.575561 | 0.000363 | 0.000358 | 0.00127 |
| Gldc | 34 | 2.88E−08 | 1.80E−05 | 0.000513 | 0.002268 | 0.000573 | 0.014027 |
| Lrp1b | 1159 | 0.003606 | 0.066339 | 0.818979 | 0.008599 | 0.129946 | 0.012126 |
| Ccdc194 | 2391 | 0.02324 | 0.207301 | 0.220058 | 0.066288 | 0.112254 | 0.08571 |
| Hoxa3 | 7105 | 0.233793 | 0.701538 | 0.050748 | 0.610486 | 0.991684 | 0.17368 |
| Prpsap1 | 114 | 1.49E−06 | 0.000279 | 0.010186 | 0.003706 | 0.004892 | 0.004585 |
| Gna14 | 23 | 1.10E−09 | 1.02E−06 | 0.000139 | 0.007882 | 0.000491 | 0.000449 |
| Hoxa2 | 7096 | 0.233629 | 0.701538 | 0.050735 | 0.609231 | 0.991533 | 0.173882 |
| Ccdc61 | 2706 | 0.03109 | 0.244959 | 0.099141 | 0.121021 | 0.039216 | 0.452968 |
| Hspa12b | 769 | 0.001129 | 0.031317 | 0.014205 | 0.032983 | 0.159388 | 0.033184 |
| Ebf1 | 83 | 5.41E−07 | 0.000139 | 0.0085 | 0.000412 | 0.014479 | 0.005196 |
| Zcchc3 | 49 | 1.51E−07 | 6.56E−05 | 0.062805 | 0.00013 | 0.000467 | 0.016013 |
| Zfp951 | 306 | 7.22E−05 | 0.005033 | 0.178857 | 0.000837 | 0.257665 | 0.002144 |
| Hoxd3 | 1054 | 0.002879 | 0.058267 | 0.032174 | 0.31454 | 0.004189 | 0.195005 |
| Lgals6 | 57 | 2.02E−07 | 7.54E−05 | 0.000901 | 0.003867 | 0.006505 | 0.003749 |
| Ryr2 | 93 | 7.60E−07 | 0.000174 | 0.039722 | 0.00065 | 0.002642 | 0.005702 |
| Btc | 39 | 3.69E−08 | 2.02E−05 | 0.005743 | 0.001546 | 0.0005 | 0.002789 |
| Vmn1r-ps79 | NA | ND | ND | ND | ND | ND | ND |
| Vmn1r101 | NA | ND | ND | ND | ND | ND | ND |
| Vmn1r250 | NA | ND | ND | ND | ND | ND | ND |
| Gm10665 | NA | ND | ND | ND | ND | ND | ND |
| Vmn1r256 | NA | ND | ND | ND | ND | ND | ND |
| Vmn1r100 | NA | ND | ND | ND | ND | ND | ND |
| Gm10668 | NA | ND | ND | ND | ND | ND | ND |
| Gm4513 | NA | ND | ND | ND | ND | ND | ND |
| Vmn1r142 | NA | ND | ND | ND | ND | ND | ND |
| Vmn1r143 | NA | ND | ND | ND | ND | ND | ND |
| Vmn1r251 | NA | ND | ND | ND | ND | ND | ND |
| Vmn1r254 | NA | ND | ND | ND | ND | ND | ND |
| Vmn1r152 | NA | ND | ND | ND | ND | ND | ND |
| Vmn1r255 | NA | ND | ND | ND | ND | ND | ND |
| Snx33 | 446 | 0.000226 | 0.01079 | 0.043169 | 0.025045 | 0.008051 | 0.038079 |
| Cacna1a | 3171 | 0.043822 | 0.294651 | 0.302239 | 0.070498 | 0.038017 | 0.435184 |
| Cbln1 | 298 | 6.31E−05 | 0.004514 | 0.067701 | 0.000548 | 0.036264 | 0.052204 |
| Speg | 574 | 0.000457 | 0.016932 | 0.008011 | 0.007463 | 0.057881 | 0.229237 |
| Pxn | 832 | 0.001416 | 0.036288 | 0.068263 | 0.060749 | 0.025423 | 0.031355 |
| Grik2 | 521 | 0.000353 | 0.014398 | 0.51969 | 0.004164 | 0.012614 | 0.021107 |
| Mir497 | 68 | 2.93E−07 | 9.06E−05 | 0.024305 | 0.001849 | 0.005753 | 0.000503 |
| Sgce | 19 | 4.73E−10 | 5.31E−07 | 0.003357 | 6.39E−05 | 0.00048 | 0.000923 |
| Cdkn2b | 1461 | 0.00683 | 0.099678 | 0.019525 | 0.049541 | 0.318057 | 0.084205 |
| Skint11 | NA | ND | ND | ND | ND | ND | ND |
| Gm13034 | 70 | 3.32E−07 | 0.0001 | 0.012615 | 1.30E−05 | 0.028795 | 0.031864 |
| Barhl1 | 546 | 0.000389 | 0.015209 | 0.391628 | 0.006378 | 0.005844 | 0.044563 |
| Irx3 | 2517 | 0.026622 | 0.225582 | 0.104281 | 0.014733 | 0.177019 | 0.626599 |
| Nkain2 | 293 | 5.63E−05 | 0.004101 | 0.002533 | 0.015022 | 0.051783 | 0.031122 |
| Vmn1r107 | NA | ND | ND | ND | ND | ND | ND |
| Mir497b | 73 | 3.47E−07 | 0.000101 | 0.024479 | 0.001872 | 0.005988 | 0.000577 |
| 5031439G07Rik | 295 | 6.08E−05 | 0.004394 | 0.000843 | 0.022866 | 0.011174 | 0.312016 |
| Hs6st3 | 46 | 7.41E−08 | 3.44E−05 | 0.013228 | 0.000188 | 0.002871 | 0.003825 |
| Cdo1 | 59 | 2.14E−07 | 7.64E−05 | 0.024052 | 0.000409 | 0.001261 | 0.007357 |
| Smarca5-ps | 75 | 4.02E−07 | 0.000114 | 0.066132 | 0.000419 | 0.000801 | 0.008449 |
| Prkd2 | 4053 | 0.075918 | 0.399402 | 0.283986 | 0.526055 | 0.219914 | 0.024717 |
| Epha6 | 504 | 0.000323 | 0.013654 | 0.748771 | 0.002364 | 0.003442 | 0.084588 |
| Gm12371 | 32 | 2.00E−08 | 1.33E−05 | 0.05175 | 0.000127 | 0.00112 | 0.000844 |
| Vwc2 | 150 | 5.19E−06 | 0.000737 | 0.077118 | 0.000452 | 0.026133 | 0.003972 |
| Adcy2 | 585 | 0.000476 | 0.017343 | 0.002015 | 0.007734 | 0.523534 | 0.102528 |
| Mab21l2 | 79 | 5.01E−07 | 0.000135 | 0.45086 | 0.000653 | 0.000674 | 0.001214 |
| Gas2l2 | 976 | 0.002237 | 0.048876 | 0.044253 | 0.052674 | 0.042347 | 0.060312 |
| Gm7102 | 84 | 5.64E−07 | 0.000143 | 0.000968 | 0.00083 | 0.007492 | 0.045941 |
| Grb10 | 92 | 7.37E−07 | 0.000171 | 0.383542 | 9.37E−05 | 0.002195 | 0.004761 |
| Hoxb3 | 5680 | 0.148326 | 0.556573 | 0.663387 | 0.313877 | 0.129655 | 0.088879 |
| Slc38a1 | 81 | 5.21E−07 | 0.000136 | 0.010263 | 0.000824 | 0.002003 | 0.014881 |
| Erbb4 | 10880 | 0.484786 | 0.94979 | 0.676535 | 0.557071 | 0.377187 | 0.166256 |
| Obox3 | NA | ND | ND | ND | ND | ND | ND |
| Brinp1 | 60 | 2.16E−07 | 7.64E−05 | 0.090094 | 0.000126 | 0.001682 | 0.004809 |
| Gm8579 | 117 | 1.61E−06 | 0.000293 | 0.004936 | 0.001741 | 0.016803 | 0.006408 |
| Jak3 | 7004 | 0.22779 | 0.693449 | 0.157765 | 0.324143 | 0.197566 | 0.503607 |
| Dnajb1 | 2850 | 0.034423 | 0.257605 | 0.206911 | 0.107071 | 0.123805 | 0.090104 |
| Foxd2os | 1913 | 0.01353 | 0.150851 | 0.869502 | 0.044301 | 0.011036 | 0.154625 |
| Negr1 | 432 | 0.000204 | 0.010064 | 0.744499 | 0.00312 | 0.015323 | 0.008221 |
| Gm10710 | 178 | 9.36E−06 | 0.001121 | 0.035497 | 0.001669 | 0.015251 | 0.008007 |
| Kcnk12 | 836 | 0.001426 | 0.03637 | 0.092389 | 0.011258 | 0.056512 | 0.056748 |
| Bcat1 | 8819 | 0.342977 | 0.829018 | 0.630775 | 0.469381 | 0.068653 | 0.54874 |
| Ina | 42 | 5.37E−08 | 2.73E−05 | 0.017477 | 0.000294 | 0.000692 | 0.005311 |
| Srrm4 | 245 | 3.15E−05 | 0.002728 | 0.140096 | 0.000416 | 0.098081 | 0.00533 |
| Guca2a | 102 | 1.07E−06 | 0.000224 | 0.02706 | 0.006975 | 0.001835 | 0.001667 |
| Hoxa7 | 51 | 1.80E−07 | 7.24E−05 | 0.003059 | 0.023717 | 0.006926 | 0.000148 |
| Evx2 | 639 | 0.000641 | 0.021397 | 0.044342 | 0.005122 | 0.106843 | 0.049955 |
| Rpp25l | 104 | 1.12E−06 | 0.000229 | 0.042231 | 0.018087 | 0.000484 | 0.001653 |
| Celf3 | 249 | 3.21E−05 | 0.002751 | 0.207535 | 0.002893 | 0.016679 | 0.003121 |
| Kcnj11 | 2339 | 0.022144 | 0.201868 | 0.111572 | 0.150601 | 0.019434 | 0.401282 |
| Prr36 | 143 | 3.94E−06 | 0.000588 | 0.133242 | 0.000623 | 0.002985 | 0.010585 |
| Pitx2 | 180 | 9.61E−06 | 0.001134 | 0.090422 | 0.002365 | 0.000463 | 0.075448 |
| Mir762 | 107 | 1.20E−06 | 0.000239 | 0.029717 | 0.002775 | 0.002347 | 0.003398 |
| Tmprss11d | NA | ND | ND | ND | ND | ND | ND |
| Raet1c | 3331 | 0.049346 | 0.315862 | 0.049346 | ND | ND | ND |
| Exoc3l2 | 3554 | 0.055894 | 0.335333 | 0.052441 | 0.025729 | 0.542416 | 0.693589 |
| Sstr2 | 1059 | 0.002903 | 0.05839 | 0.080453 | 0.023053 | 0.084895 | 0.053064 |
| Plekhm2 | 1168 | 0.003684 | 0.067264 | 0.16994 | 0.069419 | 0.058023 | 0.01667 |
| Elavl4 | 103 | 1.09E−06 | 0.000226 | 0.024133 | 0.000302 | 0.001112 | 0.072769 |
| Kbtbd7 | 111 | 1.30E−06 | 0.00025 | 0.00764 | 0.254264 | 0.001605 | 0.000232 |
| Usp13 | 138 | 3.34E−06 | 0.000517 | 0.039661 | 0.015305 | 0.001053 | 0.003388 |
| Lrp3 | 91 | 7.28E−07 | 0.000171 | 0.001136 | 0.000956 | 0.003192 | ND |
| Foxd3 | 113 | 1.41E−06 | 0.000267 | 0.025086 | 4.91E−05 | 0.012616 | 0.051384 |
| Fut9 | 466 | 0.000266 | 0.012151 | 0.073076 | 0.000996 | 0.03711 | 0.150029 |
| Nr4a3 | 10042 | 0.425338 | 0.902845 | 0.349291 | 0.87026 | 0.469475 | 0.123083 |
| Ccdc124 | 2645 | 0.02934 | 0.23658 | 0.207696 | 0.195663 | 0.024991 | 0.19299 |
| Otud7a | 174 | 8.73E−06 | 0.00107 | 0.007533 | 0.002993 | 0.00073 | 0.405455 |
| Kctd8 | 153 | 5.64E−06 | 0.000781 | 0.020882 | 0.000531 | 0.008104 | 0.044449 |
| Clmp | 8173 | 0.299316 | 0.780522 | 0.563104 | 0.210589 | ND | 0.225976 |
| Obox1 | NA | ND | ND | ND | ND | ND | ND |
| Ano2 | 707 | 0.000859 | 0.025884 | 0.051482 | 0.053862 | 0.095133 | 0.00664 |
| Nrbp1 | 85 | 5.77E−07 | 0.000144 | 0.003697 | 0.004802 | 0.005679 | 0.002814 |
| Dcdc2c | 196 | 1.34E−05 | 0.00146 | 0.004501 | 0.008174 | 0.01467 | 0.020515 |
| Rasip1 | 457 | 0.000245 | 0.011375 | 0.027241 | 0.163281 | 0.03095 | 0.002658 |
| Syt14 | 184 | 1.03E−05 | 0.001193 | 0.0842 | 0.062386 | 0.000396 | 0.003887 |
| Mrgpra2b | NA | ND | ND | ND | ND | ND | ND |
| Dtx1 | 134 | 2.87E−06 | 0.000457 | 0.015886 | 0.003932 | 0.002927 | 0.009918 |
| Klf4 | 1805 | 0.011851 | 0.140038 | 0.53489 | 0.295233 | 0.039532 | 0.008772 |
| Klf14 | 127 | 2.39E−06 | 0.000402 | 0.064776 | 0.017013 | 0.001019 | 0.001306 |
| Trim52 | 149 | 4.72E−06 | 0.000676 | 0.001237 | 0.004581 | 0.158095 | 0.003618 |
| Yjefn3 | 3797 | 0.064781 | 0.363781 | 0.046723 | 0.138522 | 0.228895 | 0.429008 |
| Mir1192 | 129 | 2.51E−06 | 0.000416 | 0.00382 | 0.010834 | 0.001853 | 0.020267 |
| Ptpro | 928 | 0.001964 | 0.045135 | 0.398748 | 0.003411 | 0.033905 | 0.109142 |
| Sync | NA | ND | ND | ND | ND | ND | ND |
| Gm38666 | 1772 | 0.011281 | 0.135628 | 0.017926 | 0.062129 | 0.404664 | 0.113545 |
| Nt5c2 | 45 | 6.65E−08 | 3.15E−05 | 0.026407 | 0.000279 | 0.000754 | 0.00434 |
| Fbxl7 | 87 | 6.35E−07 | 0.000156 | 0.05042 | 0.005211 | 7.89E−05 | 0.015283 |
| Scn8a | 140 | 3.63E−06 | 0.00055 | 0.002869 | 0.00557 | 0.006853 | 0.021796 |
| 6820408C15Rik | 54 | 1.83E−07 | 7.24E−05 | 0.050032 | 0.000185 | 0.000484 | 0.017055 |
| Trpc6 | 289 | 5.25E−05 | 0.003877 | 0.086198 | 0.003341 | 0.030872 | 0.006338 |
| Gm3867 | 136 | 2.92E−06 | 0.000458 | 0.002689 | 0.007547 | 0.001774 | 0.051439 |
| Cyp26c1 | 346 | 0.000105 | 0.006454 | 0.06753 | 0.004694 | 0.002035 | 0.20121 |
| Cdkn2a | 1716 | 0.010326 | 0.12834 | 0.069625 | 0.076132 | 0.046098 | 0.185548 |
| Il11ra1 | 225 | 2.25E−05 | 0.00213 | 0.007103 | 0.211158 | 0.005881 | 0.002314 |
| Ak5 | 137 | 3.01E−06 | 0.000469 | 0.010019 | 0.001665 | 0.010207 | 0.011276 |
| Adamts4 | 40 | 4.68E−08 | 2.49E−05 | 0.00245 | 0.000119 | 0.002892 | 0.019119 |
| Unc5c | 110 | 1.29E−06 | 0.00025 | 0.200944 | 0.001107 | 0.00017 | 0.018907 |
| Gse1 | 1595 | 0.00885 | 0.118127 | 0.374142 | 0.025269 | 0.184454 | 0.021076 |
| A830018L16Rik | 917 | 0.001909 | 0.044385 | 0.298132 | 0.014999 | 0.005093 | 0.213035 |
| Irgc1 | 4181 | 0.081008 | 0.413137 | 0.127703 | 0.153706 | 0.420848 | 0.108789 |
| Pcdhga2 | 6207 | 0.179126 | 0.615301 | 0.901374 | 0.22706 | 0.202361 | 0.080069 |
| Galnt2 | 500 | 0.000319 | 0.01357 | 0.258098 | 0.000721 | 0.016589 | 0.164765 |
| Riiad1 | 90 | 7.08E−07 | 0.000168 | 0.066342 | 0.001382 | 0.004106 | 0.000954 |
| Cxxc5 | 4514 | 0.094505 | 0.446423 | 0.746372 | 0.070392 | 0.132447 | 0.164748 |
| Gpr139 | 95 | 7.98E−07 | 0.000179 | 0.051846 | 0.001459 | 0.003277 | 0.001661 |
| Wdtc1 | 497 | 0.000318 | 0.01357 | 0.056297 | 0.265842 | 0.004742 | 0.007124 |
| Espn | 1199 | 0.003974 | 0.070646 | 0.116371 | 0.002631 | 0.112066 | 0.367508 |
| Fignl2 | 43 | 5.60E−08 | 2.78E−05 | 0.160334 | 0.011805 | 3.18E−05 | 0.00033 |
| Nr2f2 | 2193 | 0.018611 | 0.180996 | 0.502537 | 0.053406 | 0.114249 | 0.033435 |
| Nox4 | 133 | 2.86E−06 | 0.000457 | 0.04433 | 0.000643 | 0.000971 | 0.06524 |
| Actl10 | 146 | 4.15E−06 | 0.000607 | 0.004555 | 0.155601 | 0.000736 | 0.005349 |
| Pcdhgb8 | 1981 | 0.014791 | 0.159241 | 0.437351 | 0.012533 | 0.033847 | 0.400854 |
| Krt19 | 6154 | 0.175466 | 0.607918 | 0.109382 | 0.134943 | 0.7233 | 0.299663 |
| Gp1bb | 148 | 4.41E−06 | 0.000635 | 0.005492 | 0.076875 | 0.000561 | 0.012623 |
| Cdh7 | 67 | 2.90E−07 | 9.06E−05 | 0.019187 | 0.001137 | 0.00922 | 0.000641 |
| Lrrc4c | 2988 | 0.038456 | 0.274329 | 0.251827 | 0.112195 | 0.015962 | 0.644502 |
| Hoxc5 | 355 | 0.000109 | 0.006565 | 0.308667 | 0.017318 | 0.007208 | 0.003545 |
| Tafa5 | 121 | 1.69E−06 | 0.000299 | 0.003396 | 0.000129 | 0.005124 | 0.437889 |
| Runx1t1 | 277 | 4.79E−05 | 0.003689 | 0.187425 | 0.000908 | 0.014936 | 0.019852 |
| BC021767 | 4406 | 0.090497 | 0.437957 | 0.441421 | 0.16032 | 0.07383 | 0.204809 |
| Tmem181c-ps | 4500 | 0.09403 | 0.445522 | 0.138406 | 0.43354 | 0.042415 | 0.446836 |
| Hdac3 | 262 | 4.07E−05 | 0.003303 | 0.085369 | 0.002859 | 0.007877 | 0.021567 |
| Mamstr | 1062 | 0.002911 | 0.058391 | 0.376315 | 0.019291 | 0.046187 | 0.025004 |
| Khdrbs2 | 263 | 4.09E−05 | 0.003303 | 0.122548 | 0.002052 | 0.058816 | 0.002823 |
| Zfp423 | 3871 | 0.068004 | 0.374505 | 0.082286 | 0.134576 | 0.322701 | 0.191629 |
| Tmem88 | 152 | 5.63E−06 | 0.000781 | 0.005438 | 0.005374 | 0.005695 | 0.023931 |
| Arhgef1 | 11683 | 0.54181 | 0.988683 | 0.923887 | 0.186433 | 0.973221 | 0.18451 |
| Evpl | 522 | 0.000353 | 0.014398 | 0.006079 | 0.035928 | 0.235331 | 0.011217 |
| Depp1 | 157 | 5.86E−06 | 0.000796 | 0.011228 | 0.004389 | 0.008791 | 0.009642 |
| Ptf1a | 159 | 6.07E−06 | 0.000815 | 0.067009 | 0.007063 | 0.002929 | 0.00314 |
| Pcdh15 | 856 | 0.001522 | 0.037915 | 0.116367 | 0.004116 | 0.02287 | 0.331048 |
| Fign | 112 | 1.39E−06 | 0.000265 | 0.106472 | 0.003337 | 0.004195 | 0.000524 |
| Gm7030 | 141 | 3.64E−06 | 0.00055 | 0.00825 | 0.000295 | 0.025469 | 0.038607 |
| Zfp575 | 160 | 6.13E−06 | 0.000817 | 0.252311 | 0.016126 | 0.001445 | 0.000748 |
| Clec2d | 223 | 2.20E−05 | 0.002105 | 0.011487 | 0.018643 | 0.006762 | 0.013754 |
| Klhl1 | 563 | 0.000422 | 0.015955 | 0.021151 | 0.01027 | 0.045638 | 0.072441 |
| Gria4 | 590 | 0.000495 | 0.017858 | 0.849695 | 0.004943 | 0.000968 | 0.215585 |
| Sirt4 | 1038 | 0.002734 | 0.056173 | 0.087428 | 0.0214 | 0.025666 | 0.160929 |
| Arhgap28 | 228 | 2.35E−05 | 0.002195 | 0.067397 | 0.009854 | 0.017226 | 0.001881 |
| Il16 | 69 | 2.93E−07 | 9.06E−05 | 0.00119 | 0.029222 | 0.003996 | 0.000938 |
| Col1a1 | 1609 | 0.009015 | 0.119403 | 0.394798 | 0.021551 | 0.046501 | 0.095254 |
| Car10 | 294 | 6.05E−05 | 0.004385 | 0.646847 | 0.000467 | 0.030776 | 0.007182 |
| St3gal2 | 3449 | 0.05247 | 0.324282 | 0.148897 | 0.142014 | 0.122674 | 0.177881 |
| Ctnnd2 | 1946 | 0.014269 | 0.156316 | 0.086804 | 0.024692 | 0.139436 | 0.236738 |
| C1rb | 353 | 0.000108 | 0.006531 | 0.018753 | 0.011 | 0.205569 | 0.003179 |
| Slc22a18 | 33 | 2.30E−08 | 1.49E−05 | 0.002805 | 0.001423 | 0.001026 | 0.001775 |
| Nlgn1 | 204 | 1.56E−05 | 0.001634 | 0.030444 | 0.00162 | 0.016953 | 0.015869 |
| Pcbp1 | 168 | 7.70E−06 | 0.000978 | 0.005262 | 0.009591 | 0.007491 | 0.015224 |
| Usp10 | 1120 | 0.003331 | 0.063368 | 0.447234 | 0.00144 | 0.046429 | 0.334474 |
| Mir3074-2 | 172 | 8.30E−06 | 0.00103 | 0.005309 | 0.01625 | 0.005391 | 0.013518 |
| Fhl3 | 579 | 0.000461 | 0.016999 | 0.003177 | 0.23935 | 0.007885 | 0.13404 |
| Igsf23 | 1102 | 0.003225 | 0.062382 | 0.193407 | 0.014746 | 0.015505 | 0.216773 |
| 1700123O12Rik | 7812 | 0.27661 | 0.754898 | 0.773527 | 0.304876 | 0.175423 | 0.176647 |
| Gpr158 | 664 | 0.000717 | 0.023001 | 0.534503 | 0.002864 | 0.003383 | 0.269497 |
| Creld1 | 74 | 3.80E−07 | 0.00011 | 0.018419 | 0.000258 | 0.002016 | 0.018333 |
| Mir24-2 | 181 | 9.63E−06 | 0.001134 | 0.005189 | 0.017043 | 0.005135 | 0.016475 |
| Cyp26b1 | 3322 | 0.048893 | 0.313808 | 0.190165 | 0.2901 | 0.261081 | 0.028814 |
| D830030K20Rik | NA | ND | ND | ND | ND | ND | ND |
| Sox17 | 1047 | 0.002831 | 0.057666 | 0.123181 | 0.007843 | 0.034165 | 0.244941 |
| Kdelr2 | 163 | 6.62E−06 | 0.000867 | 0.00934 | 0.020547 | 0.008953 | 0.002805 |
| Ropn1l | 923 | 0.001934 | 0.044688 | 0.253963 | 0.026101 | 0.019115 | 0.03894 |
| Mir23a | 185 | 1.04E−05 | 0.001196 | 0.007117 | 0.014371 | 0.003869 | 0.020644 |
| Ajap1 | 78 | 4.65E−07 | 0.000127 | 0.199003 | 0.000737 | 0.000276 | 0.005464 |
| Tecr | 5673 | 0.147794 | 0.555444 | 0.293078 | 0.187153 | 0.177601 | 0.244822 |
| Vasp | 827 | 0.001396 | 0.035992 | 0.158041 | 0.005794 | 0.058231 | 0.060878 |
| Vmn2r55 | NA | ND | ND | ND | ND | ND | ND |
| Cbx8 | 4288 | 0.085078 | 0.423067 | 0.314398 | 0.2245 | 0.280891 | 0.048957 |
| Shisa6 | 265 | 4.10E−05 | 0.003303 | 0.180336 | 0.000931 | 0.007523 | 0.033165 |
| Pou3f3 | 177 | 9.28E−06 | 0.001118 | 0.096468 | 0.00028 | 0.141276 | 0.00188 |
| Irgm2 | 776 | 0.00116 | 0.031875 | 0.153644 | 0.031654 | 0.061449 | 0.008578 |
| Igsf9b | 167 | 7.25E−06 | 0.000926 | 0.16774 | 0.000547 | 0.025626 | 0.002283 |
| Limd1 | 198 | 1.39E−05 | 0.001495 | 0.008544 | 0.011794 | 0.007802 | 0.014655 |
| Gent2 | 165 | 7.02E−06 | 0.000907 | 0.069789 | 0.003239 | 0.001831 | 0.012472 |
| Prss21 | 469 | 0.000268 | 0.012199 | 0.549499 | 0.010326 | 0.003424 | 0.021114 |
| Rara | 317 | 7.85E−05 | 0.00528 | 0.029885 | 0.021376 | 0.017585 | 0.008142 |
| Capn11 | 1196 | 0.003937 | 0.070208 | 0.10139 | 0.339042 | 0.016304 | 0.02222 |
| Nfix | 7980 | 0.286548 | 0.765467 | 0.132236 | 0.406196 | 0.188169 | 0.773648 |
| 5830428M24Rik | 853 | 0.001511 | 0.03778 | 0.061375 | 0.043057 | 0.028759 | 0.04728 |
| Gucy1a2 | 4713 | 0.103292 | 0.467332 | 0.294118 | 0.617344 | 0.020331 | 0.358072 |
| Tdrd6 | 944 | 0.002037 | 0.046005 | 0.093128 | 0.024619 | 0.047943 | 0.047984 |
| Gm10406 | 440 | 0.000214 | 0.010392 | ND | 0.039618 | 0.009783 | 0.005581 |
| Cdc14b | 873 | 0.001644 | 0.040148 | 0.01948 | 0.018743 | 0.045372 | 0.241683 |
| C1qtnf1 | 7275 | 0.243583 | 0.71387 | 0.403229 | 0.457349 | 0.349312 | 0.089336 |
| Sntg1 | 3528 | 0.054935 | 0.33194 | 0.975653 | 0.003556 | 0.187548 | 0.759908 |
| Apba2 | 76 | 4.25E−07 | 0.000119 | 0.096114 | 0.000807 | 0.000113 | 0.022758 |
| Dchs2 | 106 | 1.19E−06 | 0.000239 | 0.057139 | 0.002064 | 0.003154 | 0.001755 |
| Foxf2 | 617 | 0.000581 | 0.020052 | 0.017413 | 0.028932 | 0.314045 | 0.006767 |
| Prkcb | 131 | 2.77E−06 | 0.00045 | 0.002682 | 0.001578 | 0.341781 | 0.001201 |
| Skint4 | NA | ND | ND | ND | ND | ND | ND |
| Stra8 | 58 | 2.08E−07 | 7.64E−05 | 0.00043 | 0.004106 | 0.001115 | 0.044661 |
| A730013G03Rik | 194 | 1.28E−05 | 0.001412 | 0.004195 | 0.017153 | 0.010664 | 0.013708 |

TABLE 8
(Supplementary Table S3c. Ranked genes by differential methylation
(Y vs Old-2): Rank_body, p-value body, q-value body)

| | Rank_{—} | p-value | q-value | p-value body | p-value body | p-value body | p-value body |
|---|---|---|---|---|---|---|---|
| Gene | body | body | body | TR1 | TR2 | TR3 | TR4 |
| Lncppara | 1 | 2.22E−16 | 3.98E−12 | 1.09E−05 | 2.20E−06 | 9.89E−07 | 4.21E−06 |
| B3glct | 3268 | 0.057603 | 0.315785 | 0.189293 | 0.283613 | 0.087507 | 0.113096 |
| Egr3 | 2 | 1.79E−15 | 1.60E−11 | 3.13E−05 | 2.58E−05 | 7.50E−06 | 1.99E−05 |
| Lncpint | 6 | 6.20E−12 | 1.85E−08 | 0.009263 | 5.50E−05 | 2.23E−05 | 7.15E−05 |
| Grm7 | 264 | 3.97E−05 | 0.002685 | 0.164979 | 0.002111 | 0.003871 | 0.029914 |
| Sox11 | 14 | 1.15E−09 | 1.45E−06 | 0.0038 | 7.07E−05 | 0.000372 | 0.002557 |
| Bhlhe40 | 3 | 1.47E−14 | 8.82E−11 | 0.000403 | 3.36E−06 | 4.38E−05 | 1.97E−05 |
| Mmp16 | 91 | 7.85E−07 | 0.000153 | 0.229966 | 0.000852 | 0.000277 | 0.007445 |
| 5730507C01Rik | 1156 | 0.004089 | 0.063409 | 0.699819 | 0.006052 | 0.070381 | 0.04391 |
| Foxg1 | 45 | 5.22E−08 | 2.00E−05 | 0.074592 | 0.000262 | 0.000129 | 0.007257 |
| Gm4425 | 27 | 1.05E−08 | 6.93E−06 | 0.015164 | 0.001168 | 0.000359 | 0.000475 |
| Hoxa9 | 10 | 7.61E−11 | 1.37E−07 | 7.31E−05 | 0.002892 | 0.001108 | 5.41E−05 |
| Tmem267 | 535 | 0.000402 | 0.013468 | 0.004244 | 0.094924 | 0.076404 | 0.022013 |
| 9030624G23Rik | 482 | 0.000298 | 0.011051 | 0.024559 | 0.005877 | 0.133392 | 0.024225 |
| Cilp2 | 460 | 0.000259 | 0.010063 | 0.407547 | 0.000435 | 0.007684 | 0.287931 |
| Nrg3 | 936 | 0.002157 | 0.041309 | 0.481917 | 0.008786 | 0.018539 | 0.072375 |
| Otx1 | 7 | 4.35E−11 | 1.00E−07 | 0.008045 | 9.21E−06 | 2.72E−06 | 0.03399 |
| Lingo2 | 112 | 1.82E−06 | 0.000287 | 0.508475 | 0.00039 | 0.000512 | 0.010529 |
| Mdga2 | 2742 | 0.038041 | 0.248477 | 0.963858 | 0.019511 | 0.041719 | 0.364617 |
| Nr4a2 | 20 | 4.22E−09 | 3.61E−06 | 0.033016 | 0.000266 | 5.84E−05 | 0.002115 |
| Nkain3 | 503 | 0.000333 | 0.011866 | 0.125378 | 0.003209 | 0.019177 | 0.069527 |
| Peg10 | 2869 | 0.042011 | 0.262284 | 0.791016 | 0.03438 | 0.021892 | 0.556213 |
| Haglr | 3057 | 0.048453 | 0.283816 | 0.085859 | 0.059415 | 0.356 | 0.225452 |
| Zfta | 13 | 6.61E−10 | 9.12E−07 | 1.12E−05 | 0.000146 | 0.04135 | 0.002038 |
| Sgcz | 157 | 7.38E−06 | 0.000838 | 0.107796 | 0.001268 | 0.001627 | 0.024601 |
| 3110039M20Rik | 2418 | 0.027537 | 0.203914 | 0.504455 | 0.007127 | 0.059348 | 0.838348 |
| Pcdhga9 | 39 | 4.16E−08 | 1.80E−05 | 0.017247 | 2.07E−05 | 0.000438 | 0.090716 |
| Pcdhga10 | 98 | 9.96E−07 | 0.000179 | 0.045603 | 6.79E−05 | 0.000945 | 0.181548 |
| Pcdh8 | 319 | 8.49E−05 | 0.004752 | 0.305042 | 0.000876 | 0.009278 | 0.040576 |
| Hoxaas3 | 26 | 1.02E−08 | 6.93E−06 | 0.005202 | 0.000156 | 0.003649 | 0.000983 |
| Csmd1 | 236 | 3.10E−05 | 0.002338 | 0.170341 | 0.002734 | 0.001836 | 0.035083 |
| Tcfl5 | 7769 | 0.348865 | 0.804839 | 0.457522 | 0.517737 | 0.246228 | 0.197978 |
| Xkr4 | 241 | 3.17E−05 | 0.002351 | 0.075232 | 0.003719 | 0.004463 | 0.02465 |
| Pcdh10 | 24 | 9.51E−09 | 6.85E−06 | 0.032392 | 0.000101 | 4.20E−05 | 0.019482 |
| Cntnap2 | 109 | 1.42E−06 | 0.000232 | 0.06786 | 0.000688 | 0.001343 | 0.012832 |
| Prdm13 | 12 | 1.94E−10 | 2.90E−07 | 0.002915 | 2.24E−05 | 0.001184 | 0.00046 |
| Galnt13 | 559 | 0.000449 | 0.014381 | 0.467884 | 0.002915 | 0.00784 | 0.072592 |
| Pcdhgb2 | 50 | 6.34E−08 | 2.23E−05 | 0.036507 | 1.29E−05 | 0.001046 | 0.046458 |
| Pcdhga6 | 49 | 6.06E−08 | 2.18E−05 | 0.033992 | 1.76E−05 | 0.000764 | 0.047522 |
| Csf2ra | 59 | 1.75E−07 | 5.14E−05 | 0.290096 | 0.000139 | 0.000575 | 0.003112 |
| Rims2 | 330 | 9.28E−05 | 0.005032 | 0.507891 | 0.003673 | 0.001967 | 0.030544 |
| Nat8f3 | 196 | 1.59E−05 | 0.001452 | 0.007524 | 7.93E−05 | ND | 0.18373 |
| Pcdhga12 | 941 | 0.002196 | 0.041828 | 0.052912 | 0.010326 | 0.025812 | 0.412234 |
| Pcdhgb6 | 51 | 8.32E−08 | 2.87E−05 | 0.020486 | 3.09E−05 | 0.000581 | 0.08456 |
| Pcdhga7 | 44 | 4.78E−08 | 1.91E−05 | 0.031582 | 1.50E−05 | 0.000642 | 0.05443 |
| Nefm | 2684 | 0.036293 | 0.242109 | 0.627669 | 0.020673 | 0.302141 | 0.068105 |
| Pcdhga4 | 38 | 3.85E−08 | 1.77E−05 | 0.017686 | 1.34E−05 | 0.000947 | 0.0579 |
| 2410018L13Rik | 142 | 4.81E−06 | 0.000603 | 0.317273 | 0.000813 | 0.001015 | 0.012638 |
| A330008L17Rik | 612 | 0.000576 | 0.016829 | 0.719642 | 0.013167 | 0.003035 | 0.036836 |
| Pcdhgb5 | 29 | 1.58E−08 | 9.48E−06 | 0.014023 | 1.76E−05 | 0.000293 | 0.066172 |
| Pcdhgb4 | 36 | 3.42E−08 | 1.66E−05 | 0.022957 | 1.45E−05 | 0.000469 | 0.072628 |
| Pcdhgb7 | 114 | 1.98E−06 | 0.000309 | 0.034898 | 0.000104 | 0.00118 | 0.274576 |
| Grid2 | 644 | 0.000682 | 0.018957 | 0.75614 | 0.003153 | 0.012819 | 0.042889 |
| Pcdhga11 | 145 | 5.72E−06 | 0.000698 | 0.029418 | 0.000306 | 0.001167 | 0.38577 |
| Kcnh7 | 653 | 0.000715 | 0.019621 | 0.378229 | 0.006363 | 0.002299 | 0.251399 |
| Pcdhga8 | 30 | 1.85E−08 | 1.07E−05 | 0.016844 | 1.36E−05 | 0.000349 | 0.070791 |
| Pcdhga5 | 48 | 5.64E−08 | 2.06E−05 | 0.033343 | 1.70E−05 | 0.000901 | 0.039145 |
| Dlgap1 | 690 | 0.000877 | 0.02275 | 0.172367 | 0.010023 | 0.016937 | 0.061489 |
| Nlrp5-ps | 1935 | 0.015548 | 0.143874 | 0.059873 | 0.317966 | 0.038943 | 0.107522 |
| Cdh2 | 8404 | 0.399576 | 0.852204 | 0.85653 | 0.091741 | 0.241366 | 0.808605 |
| Gm10377 | 4 | 2.22E−13 | 9.94E−10 | 8.65E−05 | 5.38E−05 | 6.54E−05 | 7.15E−05 |
| Gm3002 | 5 | 3.98E−13 | 1.43E−09 | 0.000464 | 5.26E−05 | 8.32E−06 | 0.000202 |
| C2cd4b | NA | ND | ND | ND | ND | ND | ND |
| M5C1000I18Rik | NA | ND | ND | ND | ND | ND | ND |
| Npr1 | 3732 | 0.076965 | 0.369543 | 0.067278 | 0.337296 | 0.311516 | 0.117351 |
| Gm45351 | 8 | 4.60E−11 | 1.00E−07 | 0.003892 | 0.00017 | 0.000155 | 7.08E−05 |
| Vmn2r47 | 9 | 5.03E−11 | 1.00E−07 | 0.003892 | 0.000181 | 0.000161 | 7.08E−05 |
| 3110070M22Rik | NA | ND | ND | ND | ND | ND | ND |
| Hoxd1 | NA | ND | ND | ND | ND | ND | ND |
| Septin9 | 190 | 1.39E−05 | 0.001308 | 0.032254 | 0.007043 | 0.009509 | 0.005365 |
| Ptprd | 23 | 6.63E−09 | 5.17E−06 | 0.035903 | 0.000109 | 9.49E−05 | 0.00485 |
| Fam205a1 | 31 | 1.95E−08 | 1.09E−05 | 0.006374 | 0.004482 | 0.000261 | 0.000811 |
| Mir6991 | NA | ND | ND | ND | ND | ND | ND |
| Gm9748 | NA | ND | ND | ND | ND | ND | ND |
| Cep85 | 18 | 2.24E−09 | 2.23E−06 | 0.000258 | 0.003838 | 0.000272 | 0.001986 |
| 4930558F17Rik | 17 | 1.52E−09 | 1.60E−06 | 0.003134 | 0.00081 | 0.000214 | 0.00064 |
| Hsf4 | 11943 | 0.682492 | 1 | 0.790473 | 0.831724 | 0.970397 | 0.091358 |
| Skint6 | 19 | 2.70E−09 | 2.55E−06 | 0.000976 | 0.001661 | 0.000874 | 0.000464 |
| Mup2 | 11 | 1.39E−10 | 2.27E−07 | 0.00364 | 9.35E−05 | 0.000152 | 0.000475 |
| Skint5 | 22 | 4.57E−09 | 3.72E−06 | 0.002162 | 0.002723 | 0.00117 | 0.000172 |
| Hic1 | 46 | 5.26E−08 | 2.00E−05 | 0.000477 | 0.002323 | 0.019149 | 0.000871 |
| St6gal2 | 1309 | 0.005825 | 0.079781 | 0.072698 | 0.039577 | 0.076656 | 0.094906 |
| Rsph6a | 693 | 0.000884 | 0.022854 | 0.108134 | 0.010587 | 0.010637 | 0.149194 |
| Cacna1b | 8127 | 0.377376 | 0.832259 | 0.70491 | 0.295812 | 0.189107 | 0.34452 |
| Defa23 | 25 | 9.55E−09 | 6.85E−06 | 0.000407 | 0.000138 | 0.001688 | 0.028594 |
| Hlx | 165 | 8.74E−06 | 0.000944 | 0.006088 | 0.011756 | 0.014068 | 0.006629 |
| Shmt2 | 86 | 4.97E−07 | 0.000102 | 0.049054 | 0.002588 | 0.00021 | 0.008959 |
| Kcnj3 | 2006 | 0.017317 | 0.15455 | 0.193868 | 0.025704 | 0.181529 | 0.102437 |
| Adamts12 | 3642 | 0.073779 | 0.362832 | 0.396302 | 0.106025 | 0.03779 | 0.489189 |
| Gm16513 | 33 | 2.24E−08 | 1.18E−05 | 0.032133 | 0.000712 | 0.000114 | 0.002703 |
| Tbx1 | 77 | 3.07E−07 | 7.07E−05 | 0.000121 | 0.002575 | 0.016608 | 0.026576 |
| Sp9 | 94 | 8.75E−07 | 0.000164 | 0.003935 | 0.005865 | 0.002817 | 0.007044 |
| Cntnap5c | 82 | 4.76E−07 | 0.000102 | 0.053291 | 0.000813 | 0.000639 | 0.008212 |
| Gm53 | 1142 | 0.00396 | 0.062158 | 0.04289 | 0.751143 | 0.016778 | 0.023216 |
| Hspa1a | NA | ND | ND | ND | ND | ND | ND |
| Grid1 | 1229 | 0.004748 | 0.069261 | 0.485996 | 0.007079 | 0.027954 | 0.165841 |
| Hmx1 | 522 | 0.000364 | 0.012483 | 0.075491 | 0.017612 | 0.001602 | 0.280784 |
| Nkx6-2 | NA | ND | ND | ND | ND | ND | ND |
| 4930442J19Rik | 134 | 3.76E−06 | 0.000499 | 0.033555 | 0.006084 | 0.011116 | 0.001096 |
| Hcn2 | 28 | 1.08E−08 | 6.93E−06 | 0.114043 | 0.000435 | 0.000366 | 0.000171 |
| Pax1 | 106 | 1.37E−06 | 0.000229 | 0.002047 | 0.005032 | 0.025808 | 0.002891 |
| Egfem1 | 1920 | 0.015214 | 0.14182 | 0.761888 | 0.00935 | 0.040294 | 0.269445 |
| Vmn1r252 | 43 | 4.72E−08 | 1.91E−05 | 0.001776 | 0.000408 | 0.001853 | 0.012165 |
| Gm6623 | NA | ND | ND | ND | ND | ND | ND |
| Ugt8a | 1456 | 0.00771 | 0.094751 | 0.179563 | 0.030356 | 0.092531 | 0.060461 |
| Gldc | 2386 | 0.026634 | 0.199793 | 0.272702 | 0.03156 | 0.126404 | 0.156749 |
| Lrp1b | 62 | 2.20E−07 | 5.14E−05 | 0.074662 | 0.000332 | 0.000358 | 0.010571 |
| Ccdc194 | 41 | 4.22E−08 | 1.80E−05 | 0.000926 | 0.003724 | 0.005484 | 0.000762 |
| Hoxa3 | 16 | 1.36E−09 | 1.53E−06 | 0.012927 | 1.47E−05 | 0.001372 | 0.001178 |
| Prpsap1 | 828 | 0.001439 | 0.031153 | 0.066084 | 0.011319 | 0.054332 | 0.083091 |
| Gna14 | 8039 | 0.370574 | 0.826195 | 0.274573 | 0.252578 | 0.483817 | 0.389778 |
| Hoxa2 | 15 | 1.21E−09 | 1.45E−06 | 0.012926 | 7.86E−06 | 0.001183 | 0.002242 |
| Ccdc61 | 42 | 4.71E−08 | 1.91E−05 | 0.007908 | 0.007772 | 0.00096 | 0.000276 |
| Hspa12b | 125 | 3.08E−06 | 0.000434 | 0.001609 | 0.019832 | 0.021945 | 0.002806 |
| Ebf1 | 1086 | 0.003515 | 0.057963 | 0.130056 | 0.027874 | 0.027682 | 0.106898 |
| Zcchc3 | NA | ND | ND | ND | ND | ND | ND |
| Zfp951 | 259 | 3.79E−05 | 0.002612 | 0.011201 | 0.0055 | 0.009854 | 0.062748 |
| Hoxd3 | 105 | 1.29E−06 | 0.000218 | 0.085614 | 0.000822 | 0.001942 | 0.005237 |
| Lgals6 | NA | ND | ND | ND | ND | ND | ND |
| Ryr2 | 1648 | 0.010339 | 0.112245 | 0.886818 | 0.029604 | 0.018501 | 0.093508 |
| Btc | 3440 | 0.065177 | 0.339274 | 0.421592 | 0.102925 | 0.157457 | 0.093894 |
| Vmn1r-ps79 | 63 | 2.21E−07 | 5.14E−05 | 0.003592 | 0.000683 | 0.004769 | 0.008051 |
| Vmn1r101 | 63 | 2.21E−07 | 5.14E−05 | 0.003592 | 0.000683 | 0.004769 | 0.008051 |
| Vmn1r250 | 63 | 2.21E−07 | 5.14E−05 | 0.003592 | 0.000683 | 0.004769 | 0.008051 |
| Gm10665 | 63 | 2.21E−07 | 5.14E−05 | 0.003592 | 0.000683 | 0.004769 | 0.008051 |
| Vmn1r256 | 63 | 2.21E−07 | 5.14E−05 | 0.003592 | 0.000683 | 0.004769 | 0.008051 |
| Vmn1r100 | 63 | 2.21E−07 | 5.14E−05 | 0.003592 | 0.000683 | 0.004769 | 0.008051 |
| Gm10668 | 63 | 2.21E−07 | 5.14E−05 | 0.003592 | 0.000683 | 0.004769 | 0.008051 |
| Gm4513 | 63 | 2.21E−07 | 5.14E−05 | 0.003592 | 0.000683 | 0.004769 | 0.008051 |
| Vmn1r142 | 63 | 2.21E−07 | 5.14E−05 | 0.003592 | 0.000683 | 0.004769 | 0.008051 |
| Vmn1r143 | 63 | 2.21E−07 | 5.14E−05 | 0.003592 | 0.000683 | 0.004769 | 0.008051 |
| Vmn1r251 | 63 | 2.21E−07 | 5.14E−05 | 0.003592 | 0.000683 | 0.004769 | 0.008051 |
| Vmn1r254 | 63 | 2.21E−07 | 5.14E−05 | 0.003592 | 0.000683 | 0.004769 | 0.008051 |
| Vmn1r152 | 63 | 2.21E−07 | 5.14E−05 | 0.003592 | 0.000683 | 0.004769 | 0.008051 |
| Vmn1r255 | 63 | 2.21E−07 | 5.14E−05 | 0.003592 | 0.000683 | 0.004769 | 0.008051 |
| Snx33 | 228 | 2.68E−05 | 0.002099 | 0.043814 | 0.009104 | 0.009998 | 0.006309 |
| Cacna1a | 55 | 1.11E−07 | 3.54E−05 | 0.00773 | 0.001844 | 0.000739 | 0.004071 |
| Cbln1 | 365 | 0.000121 | 0.005932 | 0.007022 | 0.02981 | 0.074657 | 0.009896 |
| Speg | 192 | 1.42E−05 | 0.001323 | 0.018953 | 0.001353 | 0.010018 | 0.046225 |
| Pxn | 143 | 5.17E−06 | 0.000644 | 0.015807 | 0.014008 | 0.010844 | 0.001502 |
| Grik2 | 231 | 2.89E−05 | 0.002236 | 0.572605 | 0.000748 | 0.002312 | 0.02783 |
| Mir497 | NA | ND | ND | ND | ND | ND | ND |
| Sgce | 15283 | 0.913796 | 1 | 0.848574 | 0.377857 | 0.702047 | 0.851089 |
| Cdkn2b | 100 | 1.01E−06 | 0.00018 | 0.002461 | 0.005669 | 0.003221 | 0.012047 |
| Skint11 | 78 | 3.22E−07 | 7.32E−05 | 0.003401 | 0.000267 | 0.044747 | 0.003576 |
| Gm13034 | NA | ND | ND | ND | ND | ND | ND |
| Barhl1 | 187 | 1.37E−05 | 0.001299 | 0.025162 | 0.000622 | 0.026068 | 0.02775 |
| Irx3 | 57 | 1.54E−07 | 4.78E−05 | 0.001858 | 0.010206 | 2.69E−05 | 0.122978 |
| Nkain2 | 439 | 0.000216 | 0.008789 | 0.428545 | 0.004108 | 0.003333 | 0.053398 |
| Vmn1r107 | 80 | 3.41E−07 | 7.55E−05 | 0.009379 | 0.001227 | 0.003721 | 0.003621 |
| Mir497b | NA | ND | ND | ND | ND | ND | ND |
| 5031439G07Rik | 437 | 0.000213 | 0.008707 | 0.046822 | 0.009569 | 0.015261 | 0.045044 |
| Hs6st3 | 3905 | 0.085346 | 0.391539 | 0.532997 | 0.128608 | 0.049515 | 0.287386 |
| Cdo1 | 2364 | 0.026061 | 0.197389 | 0.092078 | 0.228981 | 0.318648 | 0.024603 |
| Smarca5-ps | NA | ND | ND | ND | ND | ND | ND |
| Prkd2 | 52 | 9.64E−08 | 3.25E−05 | 0.035475 | 6.86E−05 | 0.003705 | 0.004075 |
| Epha6 | 245 | 3.24E−05 | 0.002363 | 0.283042 | 0.002375 | 0.001391 | 0.033769 |
| Gm12371 | 5806 | 0.199841 | 0.616686 | 0.761863 | 0.094853 | 0.264985 | 0.209934 |
| Vwc2 | 805 | 0.001328 | 0.029566 | 0.19497 | 0.008421 | 0.044047 | 0.042136 |
| Adcy2 | 269 | 4.52E−05 | 0.003004 | 0.125317 | 0.003674 | 0.005954 | 0.017162 |
| Mab21l2 | NA | ND | ND | ND | ND | ND | ND |
| Gas2l2 | 144 | 5.23E−06 | 0.000647 | 0.008859 | 0.003204 | 0.002974 | 0.043281 |
| Gm7102 | NA | ND | ND | ND | ND | ND | ND |
| Grb10 | 1909 | 0.014969 | 0.140406 | 0.734513 | 0.047727 | 0.069047 | 0.031242 |
| Hoxb3 | 37 | 3.79E−08 | 1.77E−05 | 0.004759 | 0.000382 | 0.001055 | 0.006657 |
| Slc38a1 | 2390 | 0.026688 | 0.199942 | 0.351447 | 0.097267 | 0.046608 | 0.107342 |
| Erbb4 | 21 | 4.23E−09 | 3.61E−06 | 0.004053 | 0.000424 | 0.000443 | 0.001426 |
| Obox3 | 89 | 6.70E−07 | 0.000133 | 0.024411 | 0.001184 | 0.00198 | 0.00588 |
| Brinp1 | 3889 | 0.084828 | 0.390874 | 0.893813 | 0.109338 | 0.039385 | 0.251006 |
| Gm8579 | 1784 | 0.012407 | 0.124374 | 0.251759 | 0.034298 | 0.047928 | 0.140928 |
| Jak3 | 35 | 2.98E−08 | 1.48E−05 | 0.015199 | 3.56E−05 | 0.000918 | 0.019564 |
| Dnajb1 | 87 | 5.00E−07 | 0.000102 | 0.025737 | 0.000414 | 0.010115 | 0.002229 |
| Foxd2os | 115 | 2.04E−06 | 0.000313 | 0.104991 | 0.004388 | 0.003312 | 0.000799 |
| Negr1 | 430 | 0.000197 | 0.008153 | 0.174116 | 0.002873 | 0.004396 | 0.12725 |
| Gm10710 | 1137 | 0.003924 | 0.061868 | 0.970231 | 0.277807 | 0.006198 | 0.007422 |
| Kcnk12 | 224 | 2.51E−05 | 0.002005 | 0.158152 | 0.005585 | 0.002508 | 0.010536 |
| Bcat1 | 32 | 2.20E−08 | 1.18E−05 | 0.025894 | 0.000303 | 0.000847 | 0.001043 |
| Ina | 5627 | 0.185913 | 0.5921 | 0.195715 | 0.286338 | 0.424664 | 0.1487 |
| Srrm4 | 825 | 0.001424 | 0.03094 | 0.083377 | 0.030523 | 0.075733 | 0.017287 |
| Guca2a | NA | ND | ND | ND | ND | ND | ND |
| Hoxa7 | 4248 | 0.102446 | 0.432209 | 0.266925 | 0.482817 | 0.04812 | 0.210344 |
| Evx2 | 300 | 6.57E−05 | 0.003916 | 0.162242 | 0.002438 | 0.028168 | 0.006621 |
| Rpp25l | NA | ND | ND | ND | ND | ND | ND |
| Celf3 | 679 | 0.00085 | 0.022426 | 0.280059 | 0.001653 | 0.072878 | 0.051256 |
| Kcnj11 | 93 | 8.25E−07 | 0.000157 | 0.002776 | 0.001965 | 0.024436 | 0.00321 |
| Prr36 | 1725 | 0.011565 | 0.119852 | 0.318781 | 0.037729 | 0.057769 | 0.076207 |
| Pitx2 | 1060 | 0.003129 | 0.052922 | 0.0023 | 0.221641 | 0.358694 | 0.050386 |
| Mir762 | NA | ND | ND | ND | ND | ND | ND |
| Tmprss11d | 104 | 1.22E−06 | 0.000208 | 0.003951 | 0.019915 | 0.003121 | 0.002724 |
| Raet1c | 119 | 2.36E−06 | 0.000353 | 0.013033 | 0.007633 | 0.0032 | 0.004546 |
| Exoc3l2 | 84 | 4.85E−07 | 0.000102 | 0.004394 | 0.002252 | 0.009337 | 0.002513 |
| Sstr2 | 171 | 1.05E−05 | 0.001099 | 0.011659 | 0.006812 | 0.00344 | 0.030451 |
| Plekhm2 | 209 | 2.17E−05 | 0.001845 | 0.14118 | 0.081889 | 0.000513 | 0.003296 |
| Elavl4 | 2978 | 0.045354 | 0.272806 | 0.587429 | 0.065254 | 0.050609 | 0.191235 |
| Kbtbd7 | NA | ND | ND | ND | ND | ND | ND |
| Usp13 | 1581 | 0.009372 | 0.106093 | 0.807448 | 0.043146 | 0.008889 | 0.128286 |
| Lrp3 | 2428 | 0.027696 | 0.204256 | 0.092536 | 0.189848 | 0.291799 | 0.035188 |
| Foxd3 | NA | ND | ND | ND | ND | ND | ND |
| Fut9 | 524 | 0.000373 | 0.012732 | 0.122155 | 0.025317 | 0.00212 | 0.094109 |
| Nr4a3 | 34 | 2.80E−08 | 1.44E−05 | 0.033878 | 0.001703 | 0.000226 | 0.000697 |
| Ccdc124 | 111 | 1.53E−06 | 0.000246 | 0.17253 | 0.001756 | 0.002502 | 0.001155 |
| Otud7a | 1678 | 0.010862 | 0.115813 | 0.50212 | 0.021101 | 0.024747 | 0.18532 |
| Kctd8 | 1819 | 0.013013 | 0.128085 | 0.517082 | 0.05351 | 0.016065 | 0.140129 |
| Clmp | 56 | 1.41E−07 | 4.44E−05 | 0.007181 | 0.003389 | 0.001365 | 0.001701 |
| Obox1 | 113 | 1.82E−06 | 0.000287 | 0.055097 | 0.000939 | 0.002582 | 0.008005 |
| Ano2 | 334 | 9.41E−05 | 0.00504 | 0.037435 | 0.023582 | 0.013334 | 0.009679 |
| Nrbp1 | 3157 | 0.052134 | 0.295817 | 0.145025 | 0.348746 | 0.088051 | 0.102615 |
| Dcdc2c | 1141 | 0.003955 | 0.062136 | 0.399209 | 0.030796 | 0.020988 | 0.048552 |
| Rasip1 | 557 | 0.000445 | 0.014313 | 0.277786 | 0.051481 | 0.002999 | 0.017924 |
| Syt14 | 1615 | 0.009846 | 0.109115 | 0.158655 | 0.137163 | 0.058617 | 0.033309 |
| Mrgpra2b | 118 | 2.30E−06 | 0.000347 | 0.153717 | 0.054899 | 7.56E−05 | 0.002195 |
| Dtx1 | 2010 | 0.017384 | 0.154874 | 0.043337 | 0.067777 | 0.075754 | 0.418693 |
| Klf4 | 146 | 5.72E−06 | 0.000698 | 0.009674 | 0.012376 | 0.00334 | 0.010144 |
| Klf14 | NA | ND | ND | ND | ND | ND | ND |
| Trim52 | 1687 | 0.010956 | 0.116256 | 0.288426 | 0.079836 | 0.012151 | 0.175719 |
| Yjefn3 | 88 | 5.89E−07 | 0.000119 | 0.028536 | 0.004826 | 0.001435 | 0.001469 |
| Mir1192 | NA | ND | ND | ND | ND | ND | ND |
| Ptpro | 289 | 5.65E−05 | 0.003493 | 0.020425 | 0.015059 | 0.048245 | 0.004142 |
| Sync | 122 | 2.70E−06 | 0.000394 | 0.006646 | 0.000677 | 0.025796 | 0.014564 |
| Gm38666 | 174 | 1.09E−05 | 0.00111 | 0.106753 | 0.00047 | 0.006948 | 0.024738 |
| Nt5c2 | 7425 | 0.321568 | 0.776253 | 0.403029 | 0.677403 | 0.186996 | 0.191937 |
| Fbxl7 | 3295 | 0.05854 | 0.318299 | 0.84089 | 0.033853 | 0.041928 | 0.456197 |
| Scn8a | 2018 | 0.017522 | 0.15527 | 0.435286 | 0.138369 | 0.048629 | 0.032163 |
| 6820408C15Rik | 6555 | 0.253992 | 0.694482 | 0.209733 | 0.267127 | 0.152224 | 0.729405 |
| Trpc6 | 960 | 0.002341 | 0.043706 | 0.132341 | 0.016177 | 0.07666 | 0.038478 |
| Gm3867 | NA | ND | ND | ND | ND | ND | ND |
| Cyp26c1 | 734 | 0.001016 | 0.024801 | 0.106444 | 0.001171 | 0.058196 | 0.298799 |
| Cdkn2a | 180 | 1.18E−05 | 0.001169 | 0.163929 | 0.00043 | 0.014393 | 0.009369 |
| Il11ra1 | 1287 | 0.005554 | 0.077366 | 0.285935 | 0.064861 | 0.052496 | 0.020175 |
| Ak5 | 2343 | 0.025582 | 0.195425 | 0.286422 | 0.06767 | 0.076255 | 0.108907 |
| Adamts4 | 9750 | 0.508736 | 0.935367 | 0.839048 | 0.325425 | 0.23632 | 0.410698 |
| Unc5c | 4039 | 0.091885 | 0.407687 | 0.458283 | 0.052817 | 0.300021 | 0.150956 |
| Gse1 | 188 | 1.37E−05 | 0.001299 | 0.015176 | 0.041522 | 0.006819 | 0.002637 |
| A830018L16Rik | 354 | 0.00011 | 0.005556 | 0.334378 | 0.00284 | 0.0014 | 0.103797 |
| Irgc1 | 99 | 9.97E−07 | 0.000179 | 0.015005 | 0.034458 | 0.007455 | 0.000138 |
| Pcdhga2 | 60 | 1.76E−07 | 5.14E−05 | 0.027854 | 2.15E−05 | 0.001335 | 0.09128 |
| Galnt2 | 657 | 0.000735 | 0.020052 | 0.487051 | 0.02508 | 0.007102 | 0.016605 |
| Riiad1 | 4249 | 0.102498 | 0.432329 | 0.413073 | 0.033837 | 0.103748 | 0.900305 |
| Cxxc5 | 95 | 8.77E−07 | 0.000164 | 0.00552 | 0.023635 | 0.016265 | 0.000216 |
| Gpr139 | 3857 | 0.083408 | 0.387326 | 0.657591 | 0.069117 | 0.120567 | 0.171686 |
| Wdtc1 | 562 | 0.000456 | 0.014538 | 0.038668 | 0.011422 | 0.018739 | 0.095708 |
| Espn | 240 | 3.14E−05 | 0.002338 | 0.058367 | 0.057391 | 0.012596 | 0.000721 |
| Fignl2 | 8163 | 0.379547 | 0.833336 | 0.594177 | 0.291718 | 0.100245 | 0.791336 |
| Nr2f2 | 154 | 7.02E−06 | 0.000811 | 0.001617 | 0.027866 | 0.000701 | 0.163486 |
| Nox4 | 3291 | 0.058398 | 0.317846 | 0.283471 | 0.035375 | 0.104354 | 0.518416 |
| Actl10 | NA | ND | ND | ND | ND | ND | ND |
| Pcdhgb8 | 189 | 1.39E−05 | 0.001308 | 0.025218 | 0.000709 | 0.001599 | 0.403044 |
| Krt19 | 79 | 3.32E−07 | 7.44E−05 | 0.00932 | 0.000857 | 0.002137 | 0.008794 |
| Gp1bb | NA | ND | ND | ND | ND | ND | ND |
| Cdh7 | 6330 | 0.237258 | 0.671788 | 0.831472 | 0.039245 | 0.252783 | 0.664625 |
| Lrrc4c | 137 | 4.09E−06 | 0.000532 | 0.079296 | 0.001102 | 0.000968 | 0.032398 |
| Hoxc5 | 919 | 0.002063 | 0.040211 | 0.05815 | 0.071107 | 0.008813 | 0.14712 |
| Tafa5 | 3123 | 0.050981 | 0.292388 | 0.79725 | 0.039137 | 0.060172 | 0.235354 |
| Runx1t1 | 1212 | 0.00462 | 0.068334 | 0.159323 | 0.052229 | 0.0141 | 0.131093 |
| BC021767 | 97 | 9.93E−07 | 0.000179 | 0.013442 | 0.000691 | 0.005377 | 0.010604 |
| Tmem181c-ps | 96 | 9.81E−07 | 0.000179 | 0.004755 | 0.001598 | 0.00213 | 0.032255 |
| Hdac3 | 1124 | 0.003814 | 0.060825 | 0.010314 | 0.125628 | 0.128724 | 0.071602 |
| Mamstr | 318 | 8.42E−05 | 0.004739 | 0.031813 | 0.00358 | 0.013886 | 0.063017 |
| Khdrbs2 | 1424 | 0.007351 | 0.092319 | 0.425201 | 0.014145 | 0.014997 | 0.317042 |
| Zfp423 | 108 | 1.42E−06 | 0.000232 | 0.040842 | 0.003067 | 0.001088 | 0.005857 |
| Tmem88 | NA | ND | ND | ND | ND | ND | ND |
| Arhgef1 | 47 | 5.34E−08 | 2.00E−05 | 0.007739 | 0.002203 | 0.000284 | 0.003881 |
| Evpl | 622 | 0.000596 | 0.017161 | 0.029492 | 0.00716 | 0.017035 | 0.307476 |
| Depp1 | NA | ND | ND | ND | ND | ND | ND |
| Ptf1a | NA | ND | ND | ND | ND | ND | ND |
| Pcdh15 | 512 | 0.000346 | 0.012115 | 0.238058 | 0.006499 | 0.003255 | 0.111717 |
| Fign | 3917 | 0.085951 | 0.393113 | 0.715454 | 0.060318 | 0.07377 | 0.309832 |
| Gm7030 | 2995 | 0.045902 | 0.27454 | 0.220283 | 0.293148 | 0.065546 | 0.089231 |
| Zfp575 | NA | ND | ND | ND | ND | ND | ND |
| Clec2d | 1689 | 0.010989 | 0.11636 | 0.333718 | 0.059754 | 0.012048 | 0.20548 |
| Klhl1 | 638 | 0.000664 | 0.018637 | 0.403147 | 0.009864 | 0.004642 | 0.068601 |
| Gria4 | 743 | 0.001046 | 0.025195 | 0.379547 | 0.013929 | 0.002436 | 0.174513 |
| Sirt4 | 315 | 7.99E−05 | 0.004538 | 0.005606 | 0.034585 | 0.021706 | 0.022212 |
| Arhgap28 | 1519 | 0.008388 | 0.098824 | 0.329521 | 0.027972 | 0.051517 | 0.071972 |
| Il16 | 6389 | 0.241407 | 0.677217 | 0.352439 | 0.185727 | 0.391259 | 0.22101 |
| Col1a1 | 227 | 2.64E−05 | 0.002078 | 0.005107 | 0.004312 | 0.016356 | 0.068698 |
| Car10 | 1322 | 0.00599 | 0.081243 | 0.770815 | 0.012947 | 0.010994 | 0.198064 |
| St3gal2 | 127 | 3.10E−06 | 0.000434 | 0.041561 | 0.010518 | 0.001084 | 0.004182 |
| Ctnnd2 | 207 | 2.06E−05 | 0.00177 | 0.82043 | 0.002982 | 0.001746 | 0.004297 |
| C1rb | 1013 | 0.002706 | 0.047877 | 0.167315 | 0.014288 | 0.086586 | 0.036822 |
| Slc22a18 | 14009 | 0.839096 | 1 | 0.850434 | 0.504541 | 0.411188 | 0.69574 |
| Nlgn1 | 2272 | 0.023619 | 0.186123 | 0.982694 | 0.014721 | 0.027803 | 0.357091 |
| Pcbp1 | NA | ND | ND | ND | ND | ND | ND |
| Usp10 | 359 | 0.000114 | 0.005661 | 0.640285 | 0.004213 | 0.018878 | 0.002812 |
| Mir3074-2 | NA | ND | ND | ND | ND | ND | ND |
| Fhl3 | 629 | 0.000631 | 0.017973 | 0.376455 | 0.001662 | 0.66679 | 0.002851 |
| Igsf23 | 338 | 9.71E−05 | 0.005139 | 0.005084 | 0.029545 | 0.031908 | 0.02469 |
| 1700123O12Rik | 81 | 4.41E−07 | 9.66E−05 | 0.023999 | 0.000345 | 0.006132 | 0.0041 |
| Gpr158 | 718 | 0.000973 | 0.024233 | 0.081515 | 0.078821 | 0.00778 | 0.041048 |
| Creld1 | 7745 | 0.347126 | 0.803365 | 0.126941 | 0.266915 | 0.464691 | 0.725957 |
| Mir24-2 | NA | ND | ND | ND | ND | ND | ND |
| Cyp26b1 | 140 | 4.49E−06 | 0.000572 | 0.001669 | 0.015811 | 0.012581 | 0.009214 |
| D830030K20Rik | 167 | 9.78E−06 | 0.001044 | 0.048204 | 0.012386 | 0.000334 | 0.038199 |
| Sox17 | 413 | 0.000182 | 0.007884 | 0.472914 | 0.000748 | 0.002888 | 0.248975 |
| Kdelr2 | 2707 | 0.036899 | 0.244035 | 0.12121 | 0.370802 | 0.052755 | 0.115372 |
| Ropn1l | 485 | 0.000302 | 0.011133 | 0.556333 | 0.1201 | 0.000414 | 0.017175 |
| Mir23a | NA | ND | ND | ND | ND | ND | ND |
| Ajap1 | 7906 | 0.359626 | 0.815222 | 0.9077 | 0.244022 | 0.27016 | 0.205379 |
| Tecr | 103 | 1.15E−06 | 0.000198 | 0.026682 | 0.001274 | 0.006728 | 0.002744 |
| Vasp | 489 | 0.000306 | 0.01118 | 0.044177 | 0.030409 | 0.008148 | 0.044166 |
| Vmn2r55 | 172 | 1.06E−05 | 0.0011 | 0.011077 | 0.012568 | 0.013633 | 0.004422 |
| Cbx8 | 120 | 2.53E−06 | 0.000375 | 0.002323 | 0.023008 | 0.000876 | 0.033444 |
| Shisa6 | 1849 | 0.013714 | 0.1328 | 0.527109 | 0.050539 | 0.046094 | 0.054543 |
| Pou3f3 | 3390 | 0.062907 | 0.332444 | 0.212358 | 0.20367 | 0.492887 | 0.028502 |
| Irgm2 | 597 | 0.000537 | 0.016103 | 0.00264 | 0.152083 | 0.416457 | 0.005807 |
| Igsf9b | 3165 | 0.052375 | 0.296443 | 0.095682 | 0.095212 | 0.170993 | 0.295402 |
| Limd1 | 2307 | 0.024559 | 0.190596 | 0.222818 | 0.059868 | 0.286038 | 0.039794 |
| Gent2 | 2894 | 0.042706 | 0.26399 | 0.161356 | 0.174358 | 0.076427 | 0.157795 |
| Prss21 | 893 | 0.001912 | 0.038376 | 0.208322 | 0.040599 | 0.035388 | 0.016245 |
| Rara | 1430 | 0.007392 | 0.092388 | 0.191821 | 0.234405 | 0.075409 | 0.008498 |
| Capn11 | 409 | 0.000179 | 0.007827 | 0.029147 | 0.13906 | 0.032886 | 0.00187 |
| Nfix | 83 | 4.82E−07 | 0.000102 | 0.061421 | 0.003999 | 0.000998 | 0.00094 |
| 5830428M24Rik | 480 | 0.000295 | 0.010994 | 0.081087 | 0.026882 | 0.025387 | 0.008342 |
| Gucy1a2 | 133 | 3.74E−06 | 0.000499 | 0.240034 | 0.000895 | 0.000732 | 0.015726 |
| Tdrd6 | 441 | 0.000222 | 0.009008 | 0.122765 | 0.009281 | 0.007968 | 0.035768 |
| Gm10406 | 972 | 0.00241 | 0.044441 | 0.029018 | 0.347049 | 0.041149 | 0.015825 |
| Cdc14b | 491 | 0.000307 | 0.01118 | 0.052681 | 0.036666 | 0.029451 | 0.008503 |
| C1qtnf1 | 90 | 6.74E−07 | 0.000133 | 0.00271 | 0.014163 | 0.001753 | 0.005035 |
| Sntg1 | 150 | 6.31E−06 | 0.00075 | 0.206582 | 0.001522 | 0.001885 | 0.007682 |
| Apba2 | 9567 | 0.494809 | 0.927063 | 0.971413 | 0.211777 | 0.222635 | 0.541502 |
| Dchs2 | 5261 | 0.162421 | 0.553315 | 0.2965 | 0.200686 | 0.170573 | 0.275888 |
| Foxf2 | 684 | 0.000859 | 0.022497 | 0.116971 | 0.017546 | 0.01402 | 0.060884 |
| Prkcb | 4446 | 0.112266 | 0.452572 | 0.951021 | 0.08381 | 0.189973 | 0.099893 |
| Skint4 | 183 | 1.27E−05 | 0.001243 | 0.167323 | 0.00173 | 0.010875 | 0.00331 |
| Stra8 | 10223 | 0.545113 | 0.955881 | 0.189069 | 0.870387 | 0.699549 | 0.272797 |
| A730013G03Rik | NA | ND | ND | ND | ND | ND | ND |

TABLE 9
(Supplementary Table S4. Ranked genes by differential methylation (O + OSKM vs O))

| | baseMean | log2FoldChange | lfcSE | stat | pvalue | padj | symbol | entrez | name |
|---|---|---|---|---|---|---|---|---|---|
| ENSMUSG00000091957.3 | 8484.629 | 3.36297 | 0.270973 | 12.41073 | 2.29E−35 | 2.59E−31 | Rps2-ps10 | 667279 | ribosomal protein S2, |
| | | | | | | | | | pseudogene 10 |
| ENSMUSG00000047347.7 | 107.04 | 5.826176 | 0.593311 | 9.819765 | 9.26E−23 | 3.49E−19 | Tdg-ps | 545124 | thymine DNA |
| | | | | | | | | | glycosylase, |
| | | | | | | | | | pseudogene |
| ENSMUSG00000075605.3 | 300.4284 | −4.31857 | 0.462137 | −9.34478 | 9.21E−21 | 2.08E−17 | Slurp2 | 69462 | secreted Ly6/Plaur |
| | | | | | | | | | domain containing 2 |
| ENSMUSG00000020609.14 | 131.1765 | −7.48876 | 0.878663 | −8.5229 | 1.56E−17 | 2.20E−14 | Apob | 238055 | apolipoprotein B |
| ENSMUSG00000026984.4 | 103.4309 | −4.67507 | 0.550052 | −8.49932 | 1.91E−17 | 2.54E−14 | Il36a | 54448 | interleukin 36A |
| ENSMUSG00000091697.2 | 40.83207 | 6.322325 | 0.752237 | 8.404699 | 4.29E−17 | 5.39E−14 | Eif3s6-ps2 | 16343 | eukaryotic translation |
| | | | | | | | | | initiation factor 3, |
| | | | | | | | | | subunit 6, pseudogene |
| | | | | | | | | | 2 |
| ENSMUSG00000022596.3 | 626.242 | −2.40704 | 0.292806 | −8.2206 | 2.02E−16 | 2.18E−13 | Slurp1 | 57277 | secreted Ly6/Plaur |
| | | | | | | | | | domain containing 1 |
| ENSMUSG00000109564.1 | 38.38174 | −23.4258 | 3.027083 | −7.73873 | 1.00E−14 | 9.88E−12 | Muc16 | 73732 | mucin 16 |
| ENSMUSG00000028001.16 | 104.3911 | −5.86896 | 0.770424 | −7.61784 | 2.58E−14 | 2.25E−11 | Fga | 14161 | fibrinogen alpha chain |
| ENSMUSG00000054905.2 | 689.3208 | −6.52622 | 0.856244 | −7.62192 | 2.50E−14 | 2.25E−11 | Stfa3 | 20863 | stefin A3 |
| ENSMUSG00000035540.12 | 196.0184 | −5.69239 | 0.749819 | −7.59169 | 3.16E−14 | 2.65E−11 | Gc | 14473 | vitamin D binding |
| | | | | | | | | | protein |
| ENSMUSG00000025745.12 | 6885.176 | 0.760374 | 0.10106 | 7.524014 | 5.31E−14 | 4.01E−11 | Hadha | 97212 | hydroxyacyl-CoA |
| | | | | | | | | | dehydrogenase |
| | | | | | | | | | trifunctional |
| | | | | | | | | | multienzyme complex |
| | | | | | | | | | subunit alpha |
| ENSMUSG00000116545.1 | 28.38316 | 7.127079 | 0.954143 | 7.469617 | 8.04E−14 | 5.87E−11 | Eif3s6-ps3 | 16344 | eukaryotic translation |
| | | | | | | | | | initiation factor 3, |
| | | | | | | | | | subunit 6, pseudogene |
| | | | | | | | | | 3 |
| ENSMUSG00000071561.2 | 1269.927 | −7.26435 | 0.978948 | −7.42057 | 1.17E−13 | 8.25E−11 | Cstdc5 | 1E+08 | cystatin domain |
| | | | | | | | | | containing 5 |
| ENSMUSG00000033860.13 | 122.3737 | −6.22605 | 0.841966 | −7.39466 | 1.42E−13 | 9.72E−11 | Fgg | 99571 | fibrinogen gamma |
| | | | | | | | | | chain |
| ENSMUSG00000066867.8 | 61.55208 | −2.81779 | 0.385911 | −7.30167 | 2.84E−13 | 1.89E−10 | Oas1e | 231699 | 2′-5′ oligoadenylate |
| | | | | | | | | | synthetase 1E |
| ENSMUSG00000033831.5 | 139.2055 | −6.35306 | 0.887882 | −7.1553 | 8.35E−13 | 5.11E−10 | Fgb | 110135 | fibrinogen beta chain |
| ENSMUSG00000005681.12 | 129.1236 | −4.56024 | 0.643875 | −7.08248 | 1.42E−12 | 8.43E−10 | Apoa2 | 11807 | apolipoprotein A-II |
| ENSMUSG00000071562.6 | 117.1814 | −6.80528 | 0.980614 | −6.93981 | 3.93E−12 | 2.22E−09 | Stfa1 | 20861 | stefin A1 |
| ENSMUSG00000054422.7 | 50.683 | −6.62375 | 0.967351 | −6.84731 | 7.53E−12 | 4.15E−09 | Fabp1 | 14080 | fatty acid binding |
| | | | | | | | | | protein 1, liver |
| ENSMUSG00000021210.16 | 17.70866 | −7.57034 | 1.116839 | −6.77836 | 1.22E−11 | 6.55E−09 | Akr1c6 | 83702 | aldo-keto reductase |
| | | | | | | | | | family 1, member C6 |
| ENSMUSG00000050315.14 | 7375.587 | 2.113535 | 0.316118 | 6.685897 | 2.30E−11 | 1.21E−08 | Synpo2 | 118449 | synaptopodin 2 |
| ENSMUSG00000023070.6 | 41.79324 | −3.36248 | 0.513039 | −6.55404 | 5.60E−11 | 2.88E−08 | Rgn | 19733 | regucalcin |
| ENSMUSG00000056035.8 | 58.24533 | −8.1277 | 1.250445 | −6.49984 | 8.04E−11 | 4.01E−08 | Cyp3a11 | 13112 | cytochrome P450, |
| | | | | | | | | | family 3, subfamily a, |
| | | | | | | | | | polypeptide 11 |
| ENSMUSG00000059447.13 | 9008.17 | 1.06205 | 0.166654 | 6.372783 | 1.86E−10 | 8.94E−08 | Hadhb | 231086 | hydroxyacyl-CoA |
| | | | | | | | | | dehydrogenase |
| | | | | | | | | | trifunctional |
| | | | | | | | | | multienzyme complex |
| | | | | | | | | | subunit beta |
| ENSMUSG00000051855.15 | 2095.768 | 4.704029 | 0.739621 | 6.360053 | 2.02E−10 | 9.51E−08 | Mest | 17294 | mesoderm specific |
| | | | | | | | | | transcript |
| ENSMUSG00000057400.14 | 55.26357 | −6.27004 | 0.990682 | −6.32901 | 2.47E−10 | 1.14E−07 | Ces1c | 13884 | carboxylesterase 1C |
| ENSMUSG00000003053.17 | 23.44274 | −7.39316 | 1.184769 | −6.24017 | 4.37E−10 | 1.90E−07 | Cyp2c29 | 13095 | cytochrome P450, |
| | | | | | | | | | family 2, subfamily c, |
| | | | | | | | | | polypeptide 29 |
| ENSMUSG00000059908.9 | 63.75604 | −5.47479 | 0.896683 | −6.10561 | 1.02E−09 | 4.21E−07 | Mug1 | 17836 | murinoglobulin 1 |
| ENSMUSG00000022868.6 | 172.3335 | −4.45418 | 0.732109 | −6.08404 | 1.17E−09 | 4.74E−07 | Ahsg | 11625 | alpha-2-HS- |
| | | | | | | | | | glycoprotein |
| ENSMUSG00000029368.10 | 2767.973 | −4.62255 | 0.760633 | −6.07724 | 1.22E−09 | 4.81E−07 | Alb | 11657 | albumin |
| ENSMUSG00000049154.12 | 18.08901 | 6.99494 | 1.151228 | 6.076066 | 1.23E−09 | 4.81E−07 | Fam183b | 75429 | family with sequence |
| | | | | | | | | | similarity 183, member |
| | | | | | | | | | B |
| ENSMUSG00000026985.1 | 280.6877 | −2.85287 | 0.474748 | 6.00922 | 1.86E−09 | 7.15E−07 | Il36b | 69677 | interleukin 36B |
| ENSMUSG00000027809.14 | 2675.366 | 0.853999 | 0.142254 | 6.003341 | 1.93E−09 | 7.29E−07 | Etfdh | 66841 | electron transferring |
| | | | | | | | | | flavoprotein, |
| | | | | | | | | | dehydrogenase |
| ENSMUSG00000059481.5 | 43.10701 | −3.8174 | 0.638606 | −5.9777 | 2.26E−09 | 8.40E−07 | Plg | 18815 | plasminogen |
| ENSMUSG00000037798.7 | 47.0596 | −6.31101 | 1.072908 | −5.88216 | 4.05E−09 | 1.43E−06 | Mat1a | 11720 | methionine |
| | | | | | | | | | adenosyltransferase I, |
| | | | | | | | | | alpha |
| ENSMUSG00000073602.10 | 290.3222 | −2.47194 | 0.422615 | −5.84914 | 4.94E−09 | 1.72E−06 | Serpinb3b | 383548 | serine (or cysteine) |
| | | | | | | | | | peptidase inhibitor, |
| | | | | | | | | | clade B (ovalbumin), |
| | | | | | | | | | member 3B |
| ENSMUSG00000006764.8 | 426.0014 | 6.937554 | 1.191405 | 5.823001 | 5.78E−09 | 1.95E−06 | Tph2 | 216343 | tryptophan |
| | | | | | | | | | hydroxylase 2 |
| ENSMUSG00000019478.16 | 190.0791 | 1.150468 | 0.19794 | 5.812205 | 6.17E−09 | 2.05E−06 | Rab4a | 19341 | RAB4A, member RAS |
| | | | | | | | | | oncogene family |
| ENSMUSG00000096255.2 | 2457.265 | 1.971772 | 0.339787 | 5.802966 | 6.52E−09 | 2.14E−06 | Dynlt1b | 21648 | dynein light chain |
| | | | | | | | | | Tctex-type 1B |
| ENSMUSG00000116207.1 | 1188.756 | 2.764778 | 0.476972 | 5.796519 | 6.77E−09 | 2.16E−06 | Nnt | 18115 | nicotinamide |
| | | | | | | | | | nucleotide |
| | | | | | | | | | transhydrogenase |
| ENSMUSG00000028393.10 | 1313.8 | 1.127648 | 0.195297 | 5.774002 | 7.74E−09 | 2.43E−06 | Alad | 17025 | aminolevulinate, delta-, |
| | | | | | | | | | dehydratase |
| ENSMUSG00000061808.4 | 158.5861 | −3.72296 | 0.645508 | −5.76749 | 8.05E−09 | 2.49E−06 | Ttr | 22139 | transthyretin |
| ENSMUSG00000028996.9 | 310.4782 | 2.776667 | 0.482324 | 5.756854 | 8.57E−09 | 2.59E−06 | Rbp7 | 63954 | retinol binding protein |
| | | | | | | | | | 7, cellular |
| ENSMUSG00000079012.11 | 199.7333 | 3.534202 | 0.613907 | 5.756899 | 8.57E−09 | 2.59E−06 | Serpina3m | 20717 | serine (or cysteine) |
| | | | | | | | | | peptidase inhibitor, |
| | | | | | | | | | clade A, member 3M |
| ENSMUSG00000055341.10 | 33.07624 | 2.124177 | 0.369891 | 5.742709 | 9.32E−09 | 2.77E−06 | Zfp457 | 431706 | zinc finger protein 457 |
| ENSMUSG00000047631.4 | 21.33813 | −4.59568 | 0.803971 | −5.71623 | 1.09E−08 | 3.20E−06 | Apof | 103161 | apolipoprotein F |
| ENSMUSG00000074768.6 | 55.69271 | −4.42996 | 0.777806 | −5.69545 | 1.23E−08 | 3.48E−06 | Bhmt | 12116 | betaine-homocysteine |
| | | | | | | | | | methyltransferase |
| ENSMUSG00000107585.1 | 189.5852 | 2.175846 | 0.381904 | 5.69736 | 1.22E−08 | 3.48E−06 | 3300002P13Rik | 70230 | RIKEN cDNA |
| | | | | | | | | | 3300002P13 gene |
| ENSMUSG00000035356.17 | 1141.593 | −1.33349 | 0.234897 | −5.67692 | 1.37E−08 | 3.78E−06 | Nfkbiz | 80859 | nuclear factor of kappa |
| | | | | | | | | | light polypeptide gene |
| | | | | | | | | | enhancer in B cells |
| | | | | | | | | | inhibitor, zeta |
| ENSMUSG00000032125.21 | 389.3269 | 2.157073 | 0.381663 | 5.651772 | 1.59E−08 | 4.28E−06 | Robo4 | 74144 | roundabout guidance |
| | | | | | | | | | receptor 4 |
| ENSMUSG00000030359.14 | 142.2192 | −5.68249 | 1.010037 | −5.62602 | 1.84E−08 | 4.91E−06 | Pzp | 11287 | PZP, alpha-2- |
| | | | | | | | | | macroglobulin like |
| ENSMUSG00000051236.13 | 2177.999 | 1.210938 | 0.215474 | 5.619885 | 1.91E−08 | 5.03E−06 | Msrb3 | 320183 | methionine sulfoxide |
| | | | | | | | | | reductase B3 |
| ENSMUSG00000037440.8 | 206.9009 | 3.004412 | 0.540264 | 5.561006 | 2.68E−08 | 6.90E−06 | Vnn1 | 22361 | vanin 1 |
| ENSMUSG00000042677.7 | 654.1933 | 1.18862 | 0.214309 | −5.54628 | 2.92E−08 | 7.42E−06 | Zc3h12a | 230738 | zinc finger CCCH type |
| | | | | | | | | | containing 12A |
| ENSMUSG00000028356.4 | 44.71722 | 5.34134 | 0.964039 | −5.54059 | 3.01E−08 | 7.58E−06 | Ambp | 11699 | alpha 1 |
| | | | | | | | | | microglobulin/bikunin |
| | | | | | | | | | precursor |
| ENSMUSG00000001687.15 | 2906.829 | 0.56219 | 0.102018 | −5.51067 | 3.57E−08 | 8.79E−06 | Ubl3 | 24109 | ubiquitin-like 3 |
| ENSMUSG00000096001.2 | 142.1899 | 3.98583 | 0.723603 | −5.50831 | 3.62E−08 | 8.82E−06 | 2610528A11Rik | 70045 | RIKEN cDNA |
| | | | | | | | | | 2610528A11 gene |
| ENSMUSG00000032083.8 | 236.8163 | −4.85029 | 0.886626 | −5.47051 | 4.49E−08 | 1.07E−05 | Apoa1 | 11806 | apolipoprotein A-I |
| ENSMUSG00000046203.11 | 10.7793 | −5.67008 | 1.037194 | −5.46675 | 4.58E−08 | 1.08E−05 | Sprr2g | 20761 | small proline-rich |
| | | | | | | | | | protein 2G |
| ENSMUSG00000044533.15 | 26488.12 | −0.718 | 0.131728 | −5.45061 | 5.02E−08 | 1.17E−05 | Rps2 | 16898 | ribosomal protein S2 |
| ENSMUSG00000094806.2 | 11.45422 | −6.35223 | 1.171244 | −5.42349 | 5.84E−08 | 1.32E−05 | Cyp2d10 | 13101 | cytochrome P450, |
| | | | | | | | | | family 2, subfamily d, |
| | | | | | | | | | polypeptide 10 |
| ENSMUSG00000025396.7 | 10.807 | −6.85299 | 1.278363 | −5.36075 | 8.29E−08 | 1.84E−05 | Hsd17b6 | 27400 | hydroxysteroid (17- |
| | | | | | | | | | beta) dehydrogenase 6 |
| ENSMUSG00000057123.14 | 140.6849 | 1.846619 | 0.346349 | 5.331672 | 9.73E−08 | 2.14E−05 | Gja5 | 14613 | gap junction protein, |
| | | | | | | | | | alpha 5 |
| ENSMUSG00000069922.12 | 11.86693 | −6.39205 | 1.19989 | −5.3272 | 9.97E−08 | 2.17E−05 | Ces3a | 382053 | carboxylesterase 3A |
| ENSMUSG00000001670.13 | 33.67591 | −5.14393 | 0.972985 | −5.28675 | 1.25E−07 | 2.59E−05 | Tat | 234724 | tyrosine |
| | | | | | | | | | aminotransferase |
| ENSMUSG00000059956.14 | 758.3917 | −3.68209 | 0.696746 | −5.2847 | 1.26E−07 | 2.59E−05 | Serpinb12 | 71869 | serine (or cysteine) |
| | | | | | | | | | peptidase inhibitor, |
| | | | | | | | | | clade B (ovalbumin), |
| | | | | | | | | | member 12 |
| ENSMUSG00000026405.14 | 22.40627 | −3.596 | 0.682775 | −5.26674 | 1.39E−07 | 2.83E−05 | C4bp | 12269 | complement |
| | | | | | | | | | component 4 binding |
| | | | | | | | | | protein |
| ENSMUSG00000032094.8 | 157.3629 | −2.38585 | 0.456074 | −5.23127 | 1.68E−07 | 3.40E−05 | Cd3d | 12500 | CD3 antigen, delta |
| | | | | | | | | | polypeptide |
| ENSMUSG00000025270.13 | 964.1356 | 4.422443 | 0.849267 | 5.207364 | 1.92E−07 | 3.80E−05 | Alas2 | 11656 | aminolevulinic acid |
| | | | | | | | | | synthase 2, erythroid |
| ENSMUSG00000078503.9 | 25.77485 | 4.3931 | 0.845308 | 5.19704 | 2.02E−07 | 3.95E−05 | Zfp990 | 1.01E+08 | zinc finger protein 990 |
| ENSMUSG00000054827.12 | 15.39969 | −6.18004 | 1.189647 | −5.19485 | 2.05E−07 | 3.96E−05 | Cyp2c50 | 107141 | cytochrome P450, |
| | | | | | | | | | family 2, subfamily c, |
| | | | | | | | | | polypeptide 50 |
| ENSMUSG00000074064.6 | 1083.032 | 1.186962 | 0.228624 | 5.191755 | 2.08E−07 | 4.00E−05 | Mlycd | 56690 | malonyl-CoA |
| | | | | | | | | | decarboxylase |
| ENSMUSG00000003545.3 | 734.2323 | −4.66918 | 0.89992 | −5.18844 | 2.12E−07 | 4.00E−05 | Fosb | 14282 | FBJ osteosarcoma |
| | | | | | | | | | oncogene B |
| ENSMUSG00000021226.7 | 604.8366 | 1.188391 | 0.229421 | 5.179961 | 2.22E−07 | 4.15E−05 | Acot2 | 171210 | acyl-CoA thioesterase |
| | | | | | | | | | 2 |
| ENSMUSG00000080885.1 | 102.8912 | −2.0201 | 0.390945 | −5.16723 | 2.38E−07 | 4.41E−05 | Rpl10-ps6 | 1E+08 | ribosomal protein L10, |
| | | | | | | | | | pseudogene 6 |
| ENSMUSG00000011305.11 | 217.7828 | 1.981851 | 0.383872 | 5.162784 | 2.43E−07 | 4.48E−05 | Plin5 | 66968 | perilipin 5 |
| ENSMUSG00000046834.7 | 42701.85 | −3.23537 | 0.628832 | −5.14505 | 2.67E−07 | 4.88E−05 | Krt1 | 16678 | keratin 1 |
| ENSMUSG00000029630.15 | 9.517885 | −6.14208 | 1.198829 | −5.1234 | 3.00E−07 | 5.43E−05 | Cyp3a25 | 56388 | cytochrome P450, |
| | | | | | | | | | family 3, subfamily a, |
| | | | | | | | | | polypeptide 25 |
| ENSMUSG00000102439.4 | 16593.2 | −4.97591 | 0.975639 | −5.10015 | 3.39E−07 | 6.06E−05 | Flg | 14246 | filaggrin |
| ENSMUSG00000048489.12 | 1032.927 | 2.594529 | 0.5129 | 5.058545 | 4.22E−07 | 7.35E−05 | Depp1 | 213393 | DEPP1 autophagy |
| | | | | | | | | | regulator |
| ENSMUSG00000105547.1 | 720.9665 | −7.25347 | 1.437381 | −5.04631 | 4.50E−07 | 7.78E−05 | Iglc3 | 110787 | immunoglobulin |
| | | | | | | | | | lambda constant 3 |
| ENSMUSG00000074882.3 | 10.10792 | −6.16519 | 1.223207 | −5.04019 | 4.65E−07 | 7.97E−05 | Cyp2c68 | 433247 | cytochrome P450, |
| | | | | | | | | | family 2, subfamily c, |
| | | | | | | | | | polypeptide 68 |
| ENSMUSG00000079015.2 | 133.4013 | −5.22337 | 1.037184 | −5.0361 | 4.75E−07 | 8.08E−05 | Serpina1c | 20702 | serine (or cysteine) |
| | | | | | | | | | peptidase inhibitor, |
| | | | | | | | | | clade A, member 1C |
| ENSMUSG00000024863.6 | 12.1771 | −6.43893 | 1.279272 | −5.03328 | 4.82E−07 | 8.14E−05 | Mbl2 | 17195 | mannose-binding |
| | | | | | | | | | lectin (protein C) 2 |
| ENSMUSG00000100164.1 | 149.0193 | 0.695967 | 0.138353 | 5.030356 | 4.90E−07 | 8.21E−05 | 2610306M01Rik | 67170 | RIKEN cDNA |
| | | | | | | | | | 2610306M01 gene |
| ENSMUSG00000072571.3 | 13.84941 | 2.575018 | 0.512317 | 5.026224 | 5.00E−07 | 8.32E−05 | Tmem253 | 619301 | transmembrane protein |
| | | | | | | | | | 253 |
| ENSMUSG00000070713.5 | 271.8278 | 2.253721 | 0.449096 | 5.018355 | 5.21E−07 | 8.61E−05 | Hmgn2-ps | 1E+08 | high mobility group |
| | | | | | | | | | nucleosomal binding |
| | | | | | | | | | domain 2, pseudogene |
| ENSMUSG00000039178.9 | 520.5199 | 0.935511 | 0.186955 | 5.003941 | 5.62E−07 | 9.21E−05 | Tbc1d19 | 67249 | TBC1 domain family, |
| | | | | | | | | | member 19 |
| ENSMUSG00000037953.7 | 25.13957 | −4.03562 | 0.810683 | −4.97804 | 6.42E−07 | 0.000103 | A4gnt | 333424 | alpha-1,4-N- |
| | | | | | | | | | acetylglucosaminyltransferase |
| ENSMUSG00000073940.3 | 13262.17 | 4.174591 | 0.840861 | 4.964665 | 6.88E−07 | 0.00011 | Hbb-bt | 1.01E+08 | hemoglobin, beta adult |
| | | | | | | | | | t chain |
| ENSMUSG00000052305.6 | 29836.34 | 4.148887 | 0.841211 | 4.932038 | 8.14E−07 | 0.000127 | Hbb-b1 | 15129 | hemoglobin, beta adult |
| | | | | | | | | | major chain |
| ENSMUSG00000052560.15 | 1833.058 | −1.17228 | 0.239031 | 4.90432 | 9.38E−07 | 0.000144 | Cpne8 | 66871 | copine VIII |
| ENSMUSG00000022445.7 | 19.76177 | −4.55448 | 0.930158 | −4.89646 | 9.76E−07 | 0.000149 | Cyp2d26 | 76279 | cytochrome P450, |
| | | | | | | | | | family 2, subfamily d, |
| | | | | | | | | | polypeptide 26 |
| ENSMUSG00000033533.14 | 52.03375 | −3.47282 | 0.710732 | −4.88626 | 1.03E−06 | 0.000156 | Acsm1 | 117147 | acyl-CoA synthetase |
| | | | | | | | | | medium-chain family |
| | | | | | | | | | member 1 |
| ENSMUSG00000022347.8 | 38.15796 | −8.68048 | 1.784187 | −4.86523 | 1.14E−06 | 0.000172 | A1bg | 117586 | alpha-1-B glycoprotein |
| ENSMUSG00000042909.4 | 68.22643 | 1.00704 | 0.207258 | 4.858871 | 1.18E−06 | 0.000176 | Or52h1 | 258746 | olfactory receptor |
| | | | | | | | | | family 52 subfamily H |
| | | | | | | | | | member 1 |
| ENSMUSG00000031748.16 | 222.1579 | 1.27056 | 0.262227 | 4.845269 | 1.26E−06 | 0.000185 | Gnao1 | 14681 | guanine nucleotide |
| | | | | | | | | | binding protein, alpha |
| | | | | | | | | | O |
| ENSMUSG00000038641.12 | 38.54283 | −6.1889 | 1.277218 | −4.84561 | 1.26E−06 | 0.000185 | Akr1d1 | 208665 | aldo-keto reductase |
| | | | | | | | | | family 1, member D1 |
| ENSMUSG00000032079.12 | 27.72977 | −4.95595 | 1.023802 | −4.84073 | 1.29E−06 | 0.000188 | Apoa5 | 66113 | apolipoprotein A-V |
| ENSMUSG00000020839.16 | 94.07033 | −1.49099 | 0.308249 | −4.83698 | 1.32E−06 | 0.00019 | Tmigd1 | 66601 | transmembrane and |
| | | | | | | | | | immunoglobulin |
| | | | | | | | | | domain containing 1 |
| ENSMUSG00000030403.9 | 1616.639 | −0.77229 | 0.159948 | −4.82837 | 1.38E−06 | 0.000196 | Vasp | 22323 | vasodilator-stimulated |
| | | | | | | | | | phosphoprotein |
| ENSMUSG00000028690.4 | 601.1387 | 0.698435 | 0.144773 | 4.82433 | 1.40E−06 | 0.000199 | Mmachc | 67096 | methylmalonic |
| | | | | | | | | | aciduria cblC type, |
| | | | | | | | | | with homocystinuria |
| ENSMUSG00000086848.3 | 589.511 | −1.90584 | 0.395553 | −4.81817 | 1.45E−06 | 0.000204 | Lce6a | 78382 | late cornified envelope |
| | | | | | | | | | 6A |
| ENSMUSG00000024912.6 | 554.4776 | −5.41261 | 1.123718 | −4.8167 | 1.46E−06 | 0.000204 | Fosl1 | 14283 | fos-like antigen 1 |
| ENSMUSG00000025610.7 | 79.41586 | 1.489431 | 0.309479 | 4.812696 | 1.49E−06 | 0.000205 | Map3k7cl | 224419 | Map3k7 C-terminal |
| | | | | | | | | | like |
| ENSMUSG00000116953.1 | 443.2866 | 1.153206 | 0.239593 | 4.813196 | 1.49E−06 | 0.000205 | 9030025P20Rik | 1E+08 | RIKEN cDNA |
| | | | | | | | | | 9030025P20 gene |
| ENSMUSG00000032602.6 | 1703.272 | 0.918761 | 0.191046 | 4.809111 | 1.52E−06 | 0.000208 | Slc25a20 | 57279 | solute carrier family 25 |
| | | | | | | | | | (mitochondrial |
| | | | | | | | | | carnitine/acylcarnitine |
| | | | | | | | | | translocase), member |
| | | | | | | | | | 20 |
| ENSMUSG00000022875.18 | 73.47134 | −4.3149 | 0.899543 | −4.79677 | 1.61E−06 | 0.000219 | Kng1 | 16644 | kininogen 1 |
| ENSMUSG00000038086.4 | 574.3411 | 1.411866 | 0.294806 | 4.789141 | 1.67E−06 | 0.000226 | Hspb2 | 69253 | heat shock protein 2 |
| ENSMUSG00000046727.13 | 1339.982 | 1.195236 | 0.24999 | 4.781132 | 1.74E−06 | 0.000233 | Cystm1 | 66060 | cysteine-rich |
| | | | | | | | | | transmembrane |
| | | | | | | | | | module containing 1 |
| ENSMUSG00000028773.8 | 3377.596 | 2.162308 | 0.452561 | 4.777937 | 1.77E−06 | 0.000234 | Fabp3 | 14077 | fatty acid binding |
| | | | | | | | | | protein 3, muscle and |
| | | | | | | | | | heart |
| ENSMUSG00000040134.8 | 15.30535 | −6.18191 | 1.293847 | −4.77793 | 1.77E−06 | 0.000234 | Rdh7 | 54150 | retinol dehydrogenase |
| | | | | | | | | | 7 |
| ENSMUSG00000079451.9 | 47.14031 | −2.07243 | 0.434115 | −4.77393 | 1.81E−06 | 0.000238 | Tmprss11g | 320454 | transmembrane |
| | | | | | | | | | protease, serine 11g |
| ENSMUSG00000009378.4 | 348.9521 | 2.837929 | 0.594715 | 4.771916 | 1.82E−06 | 0.000239 | Slc16a12 | 240638 | solute carrier family 16 |
| | | | | | | | | | (monocarboxylic acid |
| | | | | | | | | | transporters), member |
| | | | | | | | | | 12 |
| ENSMUSG00000044968.16 | 566.379 | 1.231736 | 0.259185 | 4.752352 | 2.01E−06 | 0.000261 | Napepld | 242864 | N-acyl |
| | | | | | | | | | phosphatidylethanolamine |
| | | | | | | | | | phospholipase D |
| ENSMUSG00000029449.11 | 160.5043 | −2.58052 | 0.543565 | −4.7474 | 2.06E−06 | 0.000265 | Rhof | 23912 | ras homolog family |
| | | | | | | | | | member F (in |
| | | | | | | | | | filopodia) |
| ENSMUSG00000078798.4 | 9.146973 | −5.42514 | 1.146269 | −4.73287 | 2.21E−06 | 0.000281 | Sult2a1 | 20859 | sulfotransferase family |
| | | | | | | | | | 2A, |
| | | | | | | | | | dehydroepiandrosterone |
| | | | | | | | | | (DHEA)-preferring, |
| | | | | | | | | | member 1 |
| ENSMUSG00000069917.7 | 11229.11 | 4.151639 | 0.886185 | 4.684846 | 2.80E−06 | 0.000349 | Hba-a2 | 110257 | hemoglobin alpha, |
| | | | | | | | | | adult chain 2 |
| ENSMUSG00000032487.8 | 155.3287 | −2.48393 | 0.530453 | −4.68265 | 2.83E−06 | 0.00035 | Ptgs2 | 19225 | prostaglandin- |
| | | | | | | | | | endoperoxide synthase |
| | | | | | | | | | 2 |
| ENSMUSG00000041991.18 | 12387.19 | −2.66062 | 0.570173 | −4.66633 | 3.07E−06 | 0.000373 | Hrnr | 68723 | hornerin |
| ENSMUSG00000050578.10 | 82.19997 | −3.96386 | 0.849376 | −4.66679 | 3.06E−06 | 0.000373 | Mmp13 | 17386 | matrix |
| | | | | | | | | | metallopeptidase 13 |
| ENSMUSG00000089728.3 | 27.79424 | −2.57646 | 0.55214 | −4.66632 | 3.07E−06 | 0.000373 | Clec2f | 435921 | C-type lectin domain |
| | | | | | | | | | family 2, member f |
| ENSMUSG00000041798.15 | 278.2727 | 2.096999 | 0.451799 | 4.641441 | 3.46E−06 | 0.000419 | Gck | 103988 | glucokinase |
| ENSMUSG00000033634.7 | 18.52753 | −3.9292 | 0.847002 | −4.63895 | 3.50E−06 | 0.000422 | Nat8f2 | 93673 | N-acetyltransferase 8 |
| | | | | | | | | | (GCN5-related) family |
| | | | | | | | | | member 2 |
| ENSMUSG00000030131.8 | 32.29082 | −4.9936 | 1.077157 | −4.63591 | 3.55E−06 | 0.000426 | Mug2 | 17837 | murinoglobulin 2 |
| ENSMUSG00000026698.8 | 877.4635 | 0.38851 | 0.08391 | 4.630068 | 3.66E−06 | 0.000435 | Pigc | 67292 | phosphatidylinositol |
| | | | | | | | | | glycan anchor |
| | | | | | | | | | biosynthesis, class C |
| ENSMUSG00000069919.7 | 24864.15 | 3.954896 | 0.854365 | 4.629048 | 3.67E−06 | 0.000435 | Hba-a1 | 15122 | hemoglobin alpha, |
| | | | | | | | | | adult chain 1 |
| ENSMUSG00000033526.16 | 2044.366 | 0.387932 | 0.084012 | 4.617566 | 3.88E−06 | 0.000453 | Ppip5k1 | 327655 | diphosphoinositol |
| | | | | | | | | | pentakisphosphate |
| | | | | | | | | | kinase 1 |
| ENSMUSG00000044594.14 | 30.3588 | −6.68387 | 1.449673 | −4.61061 | 4.01E−06 | 0.000461 | Serpinb3a | 20248 | serine (or cysteine) |
| | | | | | | | | | peptidase inhibitor, |
| | | | | | | | | | clade B (ovalbumin), |
| | | | | | | | | | member 3A |
| ENSMUSG00000030895.9 | 146.055 | −2.84903 | 0.618401 | −4.60709 | 4.08E−06 | 0.000467 | Hpx | 15458 | hemopexin |
| ENSMUSG00000023930.14 | 36.89063 | 3.060059 | 0.666073 | 4.594177 | 4.34E−06 | 0.000489 | Crisp2 | 22024 | cysteine-rich secretory |
| | | | | | | | | | protein 2 |
| ENSMUSG00000026688.5 | 5274.593 | 1.193371 | 0.259977 | 4.590287 | 4.43E−06 | 0.000496 | Mgst3 | 66447 | microsomal |
| | | | | | | | | | glutathione S- |
| | | | | | | | | | transferase 3 |
| ENSMUSG00000026003.5 | 4409.253 | 1.18304 | 0.258051 | 4.584518 | 4.55E−06 | 0.000505 | Acadl | 11363 | acyl-Coenzyme A |
| | | | | | | | | | dehydrogenase, long- |
| | | | | | | | | | chain |
| ENSMUSG00000026542.6 | 4.270712 | −5.51383 | 1.203702 | −4.58073 | 4.63E−06 | 0.000512 | Apcs | 20219 | amyloid P component, |
| | | | | | | | | | serum |
| ENSMUSG00000061947.10 | 20.0411 | −4.40334 | 0.96161 | −4.57914 | 4.67E−06 | 0.000513 | Serpina10 | 217847 | serine (or cysteine) |
| | | | | | | | | | peptidase inhibitor, |
| | | | | | | | | | clade A (alpha-1 |
| | | | | | | | | | antiproteinase, |
| | | | | | | | | | antitrypsin), member |
| | | | | | | | | | 10 |
| ENSMUSG00000074415.14 | 865.842 | −0.92698 | 0.202472 | −4.57831 | 4.69E−06 | 0.000513 | Mir100hg | 73144 | Mir100 Mirlet7a-2 |
| | | | | | | | | | Mir125b-1 cluster host |
| | | | | | | | | | gene |
| ENSMUSG00000037996.17 | 27.72702 | −2.09971 | 0.460227 | −4.56234 | 5.06E−06 | 0.00055 | Slc24a2 | 76376 | solute carrier family 24 |
| | | | | | | | | | (sodium/potassium/calcium |
| | | | | | | | | | exchanger), |
| | | | | | | | | | member 2 |
| ENSMUSG00000027859.10 | 66.99487 | −2.73053 | 0.600241 | −4.54905 | 5.39E−06 | 0.000578 | Ngf | 18049 | nerve growth factor |
| ENSMUSG00000032878.16 | 183.2595 | 1.709555 | 0.375808 | 4.549014 | 5.39E−06 | 0.000578 | Ccdc85a | 216613 | coiled-coil domain |
| | | | | | | | | | containing 85A |
| ENSMUSG00000037031.10 | 336.4508 | 1.083088 | 0.238134 | 4.548225 | 5.41E−06 | 0.000578 | Tspan15 | 70423 | tetraspanin 15 |
| ENSMUSG00000060807.7 | 8.913275 | −6.57869 | 1.445818 | −4.55015 | 5.36E−06 | 0.000578 | Serpina6 | 12401 | serine (or cysteine) |
| | | | | | | | | | peptidase inhibitor, |
| | | | | | | | | | clade A, member 6 |
| ENSMUSG00000032289.15 | 248.7968 | −1.37863 | 0.303407 | −4.54382 | 5.52E−06 | 0.000587 | Thsd4 | 207596 | thrombospondin, type |
| | | | | | | | | | I, domain containing 4 |
| ENSMUSG00000029375.6 | 38.50115 | −8.69362 | 1.914668 | −4.54053 | 5.61E−06 | 0.000593 | Cxcl15 | 20309 | chemokine (C-X-C |
| | | | | | | | | | motif) ligand 15 |
| ENSMUSG00000083282.3 | 1605.607 | 0.764555 | 0.168683 | 4.532503 | 5.83E−06 | 0.000611 | Ctsf | 56464 | cathepsin F |
| ENSMUSG00000022877.9 | 14.03555 | −5.0828 | 1.123982 | −4.52214 | 6.12E−06 | 0.000636 | Hrg | 94175 | histidine-rich |
| | | | | | | | | | glycoprotein |
| ENSMUSG00000054630.7 | 10.53802 | −5.62975 | 1.247839 | −4.5116 | 6.43E−06 | 0.000662 | Ugt2b5 | 22238 | UDP |
| | | | | | | | | | glucuronosyltransferase |
| | | | | | | | | | 2 family, polypeptide |
| | | | | | | | | | B5 |
| ENSMUSG00000050730.17 | 1006.848 | −0.50004 | 0.110923 | −4.50793 | 6.55E−06 | 0.000667 | Arhgap42 | 71544 | Rho GTPase activating |
| | | | | | | | | | protein 42 |
| ENSMUSG00000096965.7 | 112.4569 | −2.2161 | 0.492155 | −4.50285 | 6.70E−06 | 0.000677 | 3300005D01Rik | 78512 | RIKEN cDNA |
| | | | | | | | | | 3300005D01 gene |
| ENSMUSG00000030382.15 | 19.06514 | −2.92606 | 0.650631 | −4.49726 | 6.88E−06 | 0.000692 | Slc27a5 | 26459 | solute carrier family 27 |
| | | | | | | | | | (fatty acid transporter), |
| | | | | | | | | | member 5 |
| ENSMUSG00000006191.17 | 579.2359 | 0.490235 | 0.109223 | 4.488383 | 7.18E−06 | 0.00071 | Cdkal1 | 68916 | CDK5 regulatory |
| | | | | | | | | | subunit associated |
| | | | | | | | | | protein 1-like 1 |
| ENSMUSG00000015357.10 | 2251.116 | 0.466037 | 0.10385 | 4.487576 | 7.20E−06 | 0.00071 | Clpx | 270166 | caseinolytic |
| | | | | | | | | | mitochondrial matrix |
| | | | | | | | | | peptidase chaperone |
| | | | | | | | | | subunit |
| ENSMUSG00000030236.10 | 10.36013 | −5.60786 | 1.249477 | −4.48816 | 7.18E−06 | 0.00071 | Slco1b2 | 28253 | solute carrier organic |
| | | | | | | | | | anion transporter |
| | | | | | | | | | family, member 1b2 |
| ENSMUSG00000076613.4 | 1063.353 | −7.59621 | 1.692857 | −4.48721 | 7.22E−06 | 0.00071 | Ighg2b | 16016 | immunoglobulin heavy |
| | | | | | | | | | constant gamma 2B |
| ENSMUSG00000031173.13 | 8.373913 | −5.90391 | 1.316882 | −4.48325 | 7.35E−06 | 0.00072 | Otc | 18416 | ornithine |
| | | | | | | | | | transcarbamylase |
| ENSMUSG00000076937.3 | 2538.645 | −7.36606 | 1.645961 | −4.47523 | 7.63E−06 | 0.000745 | Iglc2 | 110786 | immunoglobulin |
| | | | | | | | | | lambda constant 2 |
| ENSMUSG00000095730.3 | 22.75219 | −3.62944 | 0.812075 | −4.46934 | 7.85E−06 | 0.000762 | Vmn2r29 | 76229 | vomeronasal 2, |
| | | | | | | | | | receptor 29 |
| ENSMUSG00000079710.10 | 66.48105 | 1.170651 | 0.262501 | 4.45961 | 8.21E−06 | 0.000794 | Dynlt2a2 | 1E+08 | dynein light chain |
| | | | | | | | | | Tctex-type 2A2 |
| ENSMUSG00000035776.14 | 2436.34 | 0.778594 | 0.174855 | 4.452792 | 8.48E−06 | 0.000816 | Cd9912 | 171486 | CD99 antigen-like 2 |
| ENSMUSG00000022551.8 | 4511.225 | 0.595674 | 0.133858 | 4.450045 | 8.59E−06 | 0.000823 | Cyc1 | 66445 | cytochrome c-1 |
| ENSMUSG00000037161.14 | 11.4116 | −2.80719 | 0.631653 | −4.4442 | 8.82E−06 | 0.000842 | Mgarp | 67749 | mitochondria localized |
| | | | | | | | | | glutamic acid rich |
| | | | | | | | | | protein |
| ENSMUSG00000079013.2 | 557.047 | 2.10856 | 0.475637 | −4.43312 | 9.29E−06 | 0.000879 | Serpina3j | 238395 | serine (or cysteine) |
| | | | | | | | | | peptidase inhibitor, |
| | | | | | | | | | clade A (alpha-1 |
| | | | | | | | | | antiproteinase, |
| | | | | | | | | | antitrypsin), member |
| | | | | | | | | | 3J |
| ENSMUSG00000027999.15 | 1049.027 | 0.849483 | 0.191769 | 4.42972 | 9.44E−06 | 0.00089 | Pla2g12a | 66350 | phospholipase A2, |
| | | | | | | | | | group XIIA |
| ENSMUSG00000026715.12 | 102.7362 | −2.73664 | 0.618195 | −4.42682 | 9.56E−06 | 0.000898 | Serpine1 | 11905 | serine (or cysteine) |
| | | | | | | | | | peptidase inhibitor, |
| | | | | | | | | | clade C (antithrombin), |
| | | | | | | | | | member 1 |
| ENSMUSG00000059136.4 | 80.35131 | −0.98879 | 0.223428 | −4.42553 | 9.62E−06 | 0.0009 | Or13a25 | 258963 | olfactory receptor |
| | | | | | | | | | family 13 subfamily A |
| | | | | | | | | | member 25 |
| ENSMUSG00000022755.4 | 8.571598 | −3.92995 | 0.889292 | −4.41918 | 9.91E−06 | 0.000923 | Adgrg7 | 239853 | adhesion G protein- |
| | | | | | | | | | coupled receptor G7 |
| ENSMUSG00000051627.3 | 108.9353 | 1.144678 | 0.259777 | 4.406391 | 1.05E−05 | 0.000973 | H1f4 | 50709 | H1.4 linker histone, |
| | | | | | | | | | cluster member |
| ENSMUSG00000067149.6 | 2387.547 | −5.84491 | 1.326577 | −4.40601 | 1.05E−05 | 0.000973 | Jchain | 16069 | immunoglobulin |
| | | | | | | | | | joining chain |
| ENSMUSG00000076609.2 | 17941.06 | −6.14733 | 1.397907 | −4.39752 | 1.09E−05 | 0.001007 | Igkc | 16071 | immunoglobulin kappa |
| | | | | | | | | | constant |
| ENSMUSG00000022131.3 | 775.2714 | 0.631517 | 0.143776 | 4.392382 | 1.12E−05 | 0.001027 | Gpr180 | 58245 | G protein-coupled |
| | | | | | | | | | receptor 180 |
| ENSMUSG00000039232.12 | 187.3504 | −1.39681 | 0.318633 | −4.38376 | 1.17E−05 | 0.001065 | Stx11 | 74732 | syntaxin 11 |
| ENSMUSG00000049608.8 | 73.41602 | −4.17245 | 0.952226 | −4.38178 | 1.18E−05 | 0.00107 | Gpr55 | 227326 | G protein-coupled |
| | | | | | | | | | receptor 55 |
| ENSMUSG00000062580.9 | 2269.773 | 0.820055 | 0.187359 | 4.376927 | 1.20E−05 | 0.00109 | Timm17a | 21854 | translocase of inner |
| | | | | | | | | | mitochondrial |
| | | | | | | | | | membrane 17a |
| ENSMUSG00000023826.16 | 272.8425 | 1.412214 | 0.323016 | 4.371958 | 1.23E−05 | 0.00111 | Prkn | 50873 | parkin RBR E3 |
| | | | | | | | | | ubiquitin protein ligase |
| ENSMUSG00000021492.15 | 10.13385 | −2.91068 | 0.666628 | −4.36628 | 1.26E−05 | 0.001135 | F12 | 58992 | coagulation factor XII |
| | | | | | | | | | (Hageman factor) |
| ENSMUSG00000086429.9 | 374.7544 | −0.96663 | 0.221696 | −4.36017 | 1.30E−05 | 0.001163 | Gt(ROSA)26Sor | 14910 | gene trap ROSA 26, |
| | | | | | | | | | Philippe Soriano |
| ENSMUSG00000004317.14 | 594.4578 | −0.80315 | 0.184478 | −4.35363 | 1.34E−05 | 0.001193 | Clcn5 | 12728 | chloride channel, |
| | | | | | | | | | voltage-sensitive 5 |
| ENSMUSG00000050440.8 | 28.86994 | −5.17114 | 1.188855 | −4.34968 | 1.36E−05 | 0.001205 | Hamp | 84506 | hepcidin antimicrobial |
| | | | | | | | | | peptide |
| ENSMUSG00000053175.17 | 330.3099 | −2.07983 | 0.478145 | −4.34979 | 1.36E−05 | 0.001205 | Bcl3 | 12051 | B cell |
| | | | | | | | | | leukemia/lymphoma 3 |
| ENSMUSG00000099759.1 | 62.06928 | −2.6107 | 0.600578 | −4.34697 | 1.38E−05 | 0.001216 | 1700030C10Rik | 69513 | RIKEN cDNA |
| | | | | | | | | | 1700030C10 gene |
| ENSMUSG00000029530.16 | 57.76568 | 1.80421 | 0.415227 | −4.34511 | 1.39E−05 | 0.001221 | Ccr9 | 12769 | chemokine (C-C |
| | | | | | | | | | motif) receptor 9 |
| ENSMUSG00000064201.8 | 239.3317 | −2.59279 | 0.598042 | −4.33547 | 1.45E−05 | 0.001261 | Krt2 | 16681 | keratin 2 |
| ENSMUSG00000095079.6 | 292.3077 | −4.11127 | 0.950534 | −4.32522 | 1.52E−05 | 0.001311 | Igha | 238447 | immunoglobulin heavy |
| | | | | | | | | | constant alpha |
| ENSMUSG00000052056.14 | 1195.375 | −0.73125 | 0.169227 | −4.32115 | 1.55E−05 | 0.001331 | Zfp217 | 228913 | zinc finger protein 217 |
| ENSMUSG00000024534.15 | 117.9552 | 1.311868 | 0.303763 | 4.318716 | 1.57E−05 | 0.001335 | Sncaip | 67847 | synuclein, alpha |
| | | | | | | | | | interacting protein |
| | | | | | | | | | (synphilin) |
| ENSMUSG00000007682.6 | 378.1208 | −2.94958 | 0.683466 | −4.31562 | 1.59E−05 | 0.001349 | Dio2 | 13371 | deiodinase, |
| | | | | | | | | | iodothyronine, type II |
| ENSMUSG00000032279.11 | 5152.257 | 1.076321 | 0.249485 | 4.314163 | 1.60E−05 | 0.001353 | Idh3a | 67834 | isocitrate |
| | | | | | | | | | dehydrogenase 3 |
| | | | | | | | | | (NAD+) alpha |
| ENSMUSG00000000838.17 | 3536.982 | 1.682355 | 0.390489 | 4.308327 | 1.64E−05 | 0.001374 | Fmr1 | 14265 | fragile X messenger |
| | | | | | | | | | ribonucleoprotein 1 |
| ENSMUSG00000025348.9 | 2124.221 | 1.775525 | 0.412057 | 4.308935 | 1.64E−05 | 0.001374 | Itga7 | 16404 | integrin alpha 7 |
| ENSMUSG00000021417.15 | 2574.694 | 1.024124 | 0.237765 | 4.3073 | 1.65E−05 | 0.001375 | Eci2 | 23986 | enoyl-Coenzyme A |
| | | | | | | | | | delta isomerase 2 |
| ENSMUSG00000004945.15 | 1804.889 | 0.864953 | 0.201129 | 4.300496 | 1.70E−05 | 0.001411 | Tmem242 | 70544 | transmembrane protein |
| | | | | | | | | | 242 |
| ENSMUSG00000047511.6 | 8.526824 | 2.827259 | 0.657499 | 4.300021 | 1.71E−05 | 0.001411 | Or2v2 | 258334 | olfactory receptor |
| | | | | | | | | | family 2 subfamily V |
| | | | | | | | | | member 2 |
| ENSMUSG00000035105.5 | 1768.172 | −1.35221 | 0.315194 | −4.29011 | 1.79E−05 | 0.001464 | Egln3 | 112407 | egl-9 family hypoxia- |
| | | | | | | | | | inducible factor 3 |
| ENSMUSG00000037095.8 | 2199.168 | 2.741333 | 0.638898 | 4.290722 | 1.78E−05 | 0.001464 | Lrg1 | 76905 | leucine-rich alpha-2- |
| | | | | | | | | | glycoprotein 1 |
| ENSMUSG00000038740.9 | 591.9758 | 0.582458 | 0.135848 | 4.287566 | 1.81E−05 | 0.001476 | Mvb12b | 72543 | multivesicular body |
| | | | | | | | | | subunit 12B |
| ENSMUSG00000026645.11 | 17.46397 | −4.76264 | 1.111783 | −4.28378 | 1.84E−05 | 0.00149 | Olah | 99035 | oleoyl-ACP hydrolase |
| ENSMUSG00000059810.18 | 1037.055 | 1.140822 | 0.266273 | 4.284414 | 1.83E−05 | 0.00149 | Rgs3 | 50780 | regulator of G-protein |
| | | | | | | | | | signaling 3 |
| ENSMUSG00000017677.11 | 2251.372 | −0.73125 | 0.17084 | −4.2803 | 1.87E−05 | 0.001502 | Wsb1 | 78889 | WD repeat and SOCS |
| | | | | | | | | | box-containing 1 |
| ENSMUSG00000032411.15 | 1123.009 | 0.643566 | 0.150379 | 4.279638 | 1.87E−05 | 0.001502 | Tfdp2 | 211586 | transcription factor Dp |
| | | | | | | | | | 2 |
| ENSMUSG00000034829.8 | 61.70978 | 3.880822 | 0.90663 | 4.28049 | 1.86E−05 | 0.001502 | Nxnl1 | 234404 | nucleoredoxin-like 1 |
| ENSMUSG00000015314.10 | 202.6794 | −4.99551 | 1.167964 | −4.27711 | 1.89E−05 | 0.001511 | Slamf6 | 30925 | SLAM family member |
| | | | | | | | | | 6 |
| ENSMUSG00000027961.7 | 864.211 | 1.459355 | 0.341228 | 4.276768 | 1.90E−05 | 0.001511 | Lrrc39 | 109245 | leucine rich repeat |
| | | | | | | | | | containing 39 |
| ENSMUSG00000020884.15 | 11.12443 | −3.86341 | 0.904125 | −4.27309 | 1.93E−05 | 0.00152 | Asgr1 | 11889 | asialoglycoprotein |
| | | | | | | | | | receptor 1 |
| ENSMUSG00000068663.14 | 1469.528 | −0.80779 | 0.189031 | −4.27334 | 1.93E−05 | 0.00152 | Clec16a | 74374 | C-type lectin domain |
| | | | | | | | | | family 16, member A |
| ENSMUSG00000022602.14 | 741.0212 | −4.36118 | 1.022737 | −4.26423 | 2.01E−05 | 0.001571 | Arc | 11838 | activity regulated |
| | | | | | | | | | cytoskeletal-associated |
| | | | | | | | | | protein |
| ENSMUSG00000024168.8 | 397.3237 | 1.707075 | 0.400704 | 4.260191 | 2.04E−05 | 0.001589 | Tmem204 | 407831 | transmembrane protein |
| | | | | | | | | | 204 |
| ENSMUSG00000027249.15 | 33.73425 | −3.24384 | 0.761314 | −4.26084 | 2.04E−05 | 0.001589 | F2 | 14061 | coagulation factor II |
| ENSMUSG00000026672.11 | 1066.502 | 1.543699 | 0.362625 | 4.257009 | 2.07E−05 | 0.001606 | Optn | 71648 | optineurin |
| ENSMUSG00000025902.13 | 140.3466 | 2.033897 | 0.479587 | 4.240935 | 2.23E−05 | 0.001713 | Sox17 | 20671 | SRY (sex determining |
| | | | | | | | | | region Y)-box 17 |
| ENSMUSG00000034520.14 | 237.7075 | 1.793045 | 0.423431 | 4.234566 | 2.29E−05 | 0.001751 | Gjc1 | 14615 | gap junction protein, |
| | | | | | | | | | gamma 1 |
| ENSMUSG00000020474.11 | 663.6466 | −0.94888 | 0.22477 | −4.22154 | 2.43E−05 | 0.001843 | Polm | 54125 | polymerase (DNA |
| | | | | | | | | | directed), mu |
| ENSMUSG00000042248.4 | 9.973313 | −6.74124 | 1.600086 | −4.21305 | 2.52E−05 | 0.001907 | Cyp2c37 | 13096 | cytochrome P450, |
| | | | | | | | | | family 2. subfamily c, |
| | | | | | | | | | polypeptide 37 |
| ENSMUSG00000030470.15 | 5916.006 | 1.485765 | 0.352734 | 4.212143 | 2.53E−05 | 0.001908 | Csrp3 | 13009 | cysteine and glycine- |
| | | | | | | | | | rich protein 3 |
| ENSMUSG00000027919.5 | 781.2611 | −2.54929 | 0.605872 | −4.20764 | 2.58E−05 | 0.001934 | Lce1g | 66195 | late cornified envelope |
| | | | | | | | | | 1G |
| ENSMUSG00000071252.6 | 132.8243 | 1.178099 | 0.280107 | 4.205896 | 2.60E−05 | 0.001942 | 2210408I21Rik | 72371 | RIKEN cDNA |
| | | | | | | | | | 2210408I21 gene |
| ENSMUSG00000022419.16 | 2843.303 | 1.082504 | 0.257427 | 4.205092 | 2.61E−05 | 0.001943 | Deptor | 97998 | DEP domain |
| | | | | | | | | | containing MTOR- |
| | | | | | | | | | interacting protein |
| ENSMUSG00000043687.15 | 121.0758 | 1.603241 | 0.381447 | 4.20305 | 2.63E−05 | 0.001954 | 1190005I06Rik | 68918 | RIKEN cDNA |
| | | | | | | | | | 1190005I06 gene |
| ENSMUSG00000043342.9 | 229.5197 | 1.224387 | 0.29149 | 4.200439 | 2.66E−05 | 0.00197 | Hoxd9 | 15438 | homeobox D9 |
| ENSMUSG00000031613.9 | 1007.559 | 1.554474 | 0.370317 | 4.19768 | 2.70E−05 | 0.001981 | Hpgd | 15446 | hydroxyprostaglandin |
| | | | | | | | | | dehydrogenase 15 |
| | | | | | | | | | (NAD) |
| ENSMUSG00000058883.16 | 75.95968 | −0.98769 | 0.235398 | −4.19582 | 2.72E−05 | 0.001991 | Zfp708 | 432769 | zinc finger protein 708 |
| ENSMUSG00000029260.15 | 8.018132 | −4.66948 | 1.113176 | −4.19474 | 2.73E−05 | 0.001994 | Ugt2b34 | 100727 | UDP |
| | | | | | | | | | glucuronosyltransferase |
| | | | | | | | | | 2 family, polypeptide |
| | | | | | | | | | B34 |
| ENSMUSG00000027488.12 | 1772.873 | 1.231928 | 0.294103 | 4.188757 | 2.80E−05 | 0.002041 | Snta1 | 20648 | syntrophin, acidic 1 |
| ENSMUSG00000058997.8 | 1629.364 | 0.869931 | 0.208166 | 4.17902 | 2.93E−05 | 0.00211 | Vwa8 | 219189 | von Willebrand factor |
| | | | | | | | | | A domain containing 8 |
| ENSMUSG00000014030.15 | 304.8922 | −5.50999 | 1.320303 | −4.17327 | 3.00E−05 | 0.002157 | Pax5 | 18507 | paired box 5 |
| ENSMUSG00000032314.14 | 5286.175 | 0.914326 | 0.219417 | 4.167062 | 3.09E−05 | 0.002196 | Etfa | 110842 | electron transferring |
| | | | | | | | | | flavoprotein, alpha |
| | | | | | | | | | polypeptide |
| ENSMUSG00000096770.2 | 11.62428 | 5.823823 | 1.397587 | 4.167054 | 3.09E−05 | 0.002196 | Amy2a4 | 1E+08 | amylase 2a4 |
| ENSMUSG00000050368.4 | 43.33037 | 1.997451 | 0.480978 | 4.152897 | 3.28E−05 | 0.002329 | Hoxd10 | 15430 | homeobox D10 |
| ENSMUSG00000037053.6 | 22.57047 | −2.65841 | 0.640257 | −4.15209 | 3.29E−05 | 0.00233 | Azgp1 | 12007 | alpha-2-glycoprotein |
| | | | | | | | | | 1, zinc |
| ENSMUSG00000025588.4 | 22.63533 | 1.844918 | 0.444517 | 4.150383 | 3.32E−05 | 0.00234 | Nat1 | 17960 | N-acetyl transferase 1 |
| ENSMUSG00000021904.6 | 422.5526 | 1.371621 | 0.330652 | 4.14823 | 3.35E−05 | 0.002355 | Sema3g | 218877 | sema domain, |
| | | | | | | | | | immunoglobulin |
| | | | | | | | | | domain (Ig), short |
| | | | | | | | | | basic domain, secreted, |
| | | | | | | | | | (semaphorin) 3G |
| ENSMUSG00000047910.6 | 73.37666 | −0.80636 | 0.195116 | −4.13272 | 3.58E−05 | 0.002512 | Pcdhb16 | 93887 | protocadherin beta 16 |
| ENSMUSG00000028463.14 | 83.81452 | −1.71916 | 0.416555 | −4.12708 | 3.67E−05 | 0.002558 | Car9 | 230099 | carbonic anhydrase 9 |
| ENSMUSG00000046215.3 | 40.68927 | 3.577225 | 0.86664 | 4.127694 | 3.66E−05 | 0.002558 | Rprml | 104582 | reprimo-like |
| ENSMUSG00000027048.15 | 20.24782 | −3.39069 | 0.824163 | −4.11411 | 3.89E−05 | 0.002674 | Abcb11 | 27413 | ATP-binding cassette, |
| | | | | | | | | | sub-family B |
| | | | | | | | | | (MDR/TAP), member |
| | | | | | | | | | 11 |
| ENSMUSG00000049265.7 | 266.1307 | 3.607474 | 0.876726 | 4.114711 | 3.88E−05 | 0.002674 | Kcnk3 | 16527 | potassium channel, |
| | | | | | | | | | subfamily K, member |
| | | | | | | | | | 3 |
| ENSMUSG00000055555.1 | 67.68582 | 4.25749 | 1.036099 | 4.109154 | 3.97E−05 | 0.002723 | Ct55 | 75013 | cancer/testis antigen |
| | | | | | | | | | 55 |
| ENSMUSG00000020953.17 | 448.4261 | 2.032042 | 0.494733 | 4.107348 | 4.00E−05 | 0.002737 | Coch | 12810 | cochlin |
| ENSMUSG00000021263.11 | 57.50927 | −4.15997 | 1.013743 | −4.10357 | 4.07E−05 | 0.002773 | Degs2 | 70059 | delta(4)-desaturase, |
| | | | | | | | | | sphingolipid 2 |
| ENSMUSG00000056999.15 | 16163.47 | −1.30191 | 0.318018 | −4.09383 | 4.24E−05 | 0.002875 | Ide | 15925 | insulin degrading |
| | | | | | | | | | enzyme |
| ENSMUSG00000027984.8 | 5776.512 | 0.865301 | 0.21156 | 4.090103 | 4.31E−05 | 0.002904 | Hadh | 15107 | hydroxyacyl- |
| | | | | | | | | | Coenzyme A |
| | | | | | | | | | dehydrogenase |
| ENSMUSG00000026648.18 | 416.3658 | −0.87505 | 0.214235 | −4.08452 | 4.42E−05 | 0.002957 | Dclre1c | 227525 | DNA cross-link repair |
| | | | | | | | | | 1C |
| ENSMUSG00000014453.4 | 243.5502 | −4.42734 | 1.084915 | −4.08082 | 4.49E−05 | 0.002996 | Blk | 12143 | B lymphoid kinase |
| ENSMUSG00000032060.10 | 10563.18 | 1.413661 | 0.346652 | 4.078047 | 4.54E−05 | 0.00302 | Cryab | 12955 | crystallin, alpha B |
| ENSMUSG00000047632.11 | 140.1766 | −1.20885 | 0.296474 | −4.07744 | 4.55E−05 | 0.00302 | Fgfbp3 | 72514 | fibroblast growth |
| | | | | | | | | | factor binding protein |
| | | | | | | | | | 3 |
| ENSMUSG00000091945.2 | 106.0613 | −1.06627 | 0.261537 | −4.07696 | 4.56E−05 | 0.00302 | Vmn2r114 | 666002 | vomeronasal 2, |
| | | | | | | | | | receptor 114 |
| ENSMUSG00000026180.8 | 82.6854 | −1.66364 | 0.408747 | −4.07009 | 4.70E−05 | 0.003092 | Cxcr2 | 12765 | chemokine (C-X-C |
| | | | | | | | | | motif) receptor 2 |
| ENSMUSG00000004383.18 | 694.7656 | 0.684987 | 0.168434 | 4.066795 | 4.77E−05 | 0.003118 | Large1 | 16795 | LARGE xylosyl- and |
| | | | | | | | | | glucuronyltransferase |
| | | | | | | | | | 1 |
| ENSMUSG00000021771.14 | 5211.565 | 0.501452 | 0.123327 | 4.066033 | 4.78E−05 | 0.003118 | Vdac2 | 22334 | voltage-dependent |
| | | | | | | | | | anion channel 2 |
| ENSMUSG00000101397.6 | 7.402836 | −6.31011 | 1.552035 | −4.0657 | 4.79E−05 | 0.003118 | Mug-ps1 | 17835 | murinoglobulin, |
| | | | | | | | | | pseudogene 1 |
| ENSMUSG00000000214.11 | 78.67115 | 2.225005 | 0.548303 | 4.057984 | 4.95E−05 | 0.00321 | Th | 21823 | tyrosine hydroxylase |
| ENSMUSG00000027875.12 | 1053.949 | 2.467793 | 0.609066 | 4.051768 | 5.08E−05 | 0.003278 | Hmgcs2 | 15360 | 3-hydroxy-3- |
| | | | | | | | | | methylglutaryl- |
| | | | | | | | | | Coenzyme A synthase |
| | | | | | | | | | 2 |
| ENSMUSG00000018822.7 | 1185.411 | 2.607853 | 0.644079 | 4.048964 | 5.14E−05 | 0.003297 | Sfrp5 | 54612 | secreted frizzled- |
| | | | | | | | | | related sequence |
| | | | | | | | | | protein 5 |
| ENSMUSG00000021091.8 | 5784.521 | 2.903818 | 0.717287 | 4.048333 | 5.16E−05 | 0.003297 | Serpina3n | 20716 | serine (or cysteine) |
| | | | | | | | | | peptidase inhibitor, |
| | | | | | | | | | clade A, member 3N |
| ENSMUSG00000028223.8 | 3294.346 | 1.337878 | 0.33043 | 4.048902 | 5.15E−05 | 0.003297 | Decr1 | 67460 | 2,4-dienoy1 CoA |
| | | | | | | | | | reductase 1, |
| | | | | | | | | | mitochondrial |
| ENSMUSG00000026888.14 | 1014.823 | 1.844821 | 0.455969 | 4.045935 | 5.21E−05 | 0.003306 | Grb14 | 50915 | growth factor receptor |
| | | | | | | | | | bound protein 14 |
| ENSMUSG00000048728.15 | 44.95171 | 1.220575 | 0.301689 | 4.0458 | 5.21E−05 | 0.003306 | Zfp454 | 237758 | zinc finger protein 454 |
| ENSMUSG00000067924.4 | 1165.14 | 1.161739 | 0.287245 | 4.044415 | 5.25E−05 | 0.003316 | Rt18b | 553127 | retrotransposon Gag |
| | | | | | | | | | like 8B |
| ENSMUSG00000024524.17 | 220.2149 | 1.952522 | 0.483127 | 4.041421 | 5.31E−05 | 0.003349 | Gnal | 14680 | guanine nucleotide |
| | | | | | | | | | binding protein, alpha |
| | | | | | | | | | stimulating, olfactory |
| | | | | | | | | | type |
| ENSMUSG00000092008.2 | 9.511626 | −6.67651 | 1.65471 | −4.03485 | 5.46E−05 | 0.003425 | Cyp2c69 | 1E+08 | cytochrome P450, |
| | | | | | | | | | family 2, subfamily c, |
| | | | | | | | | | polypeptide 69 |
| ENSMUSG00000038845.11 | 2773.784 | 0.583617 | 0.144988 | 4.025267 | 5.69E−05 | 0.003558 | Phb | 18673 | prohibitin |
| ENSMUSG00000006522.17 | 41.71242 | −3.38975 | 0.843921 | −4.01667 | 5.90E−05 | 0.00368 | Itih3 | 16426 | inter-alpha trypsin |
| | | | | | | | | | inhibitor, heavy chain |
| | | | | | | | | | 3 |
| ENSMUSG00000001829.17 | 1097.483 | 0.707603 | 0.176284 | 4.013992 | 5.97E−05 | 0.003692 | Clpb | 20480 | ClpB caseinolytic |
| | | | | | | | | | peptidase B |
| ENSMUSG00000052698.15 | 2372.191 | 1.142657 | 0.284641 | 4.014372 | 5.96E−05 | 0.003692 | Tln2 | 70549 | talin 2 |
| ENSMUSG00000024669.8 | 80.06015 | −3.21749 | 0.801982 | −4.01193 | 6.02E−05 | 0.003709 | Cd5 | 12507 | CD5 antigen |
| ENSMUSG00000039209.12 | 319.5934 | 2.094571 | 0.522132 | 4.011571 | 6.03E−05 | 0.003709 | Rp1391 | 68172 | ribosomal protein L39- |
| | | | | | | | | | like |
| ENSMUSG00000045620.7 | 60.26336 | 2.980059 | 0.743277 | 4.009352 | 6.09E−05 | 0.003734 | Odf311 | 382075 | outer dense fiber of |
| | | | | | | | | | sperm tails 3-like 1 |
| ENSMUSG00000026535.9 | 583.7627 | −3.00942 | 0.751305 | −4.00559 | 6.19E−05 | 0.003784 | Ifi202b | 26388 | interferon activated |
| | | | | | | | | | gene 202B |
| ENSMUSG00000026921.20 | 1009.237 | 1.334368 | 0.333292 | 4.003597 | 6.24E−05 | 0.003806 | Egfl7 | 353156 | EGF-like domain 7 |
| ENSMUSG00000032251.12 | 161.8101 | 1.24979 | 0.312276 | 4.002193 | 6.28E−05 | 0.003818 | Irak1bp1 | 65099 | interleukin-1 receptor- |
| | | | | | | | | | associated kinase 1 |
| | | | | | | | | | binding protein 1 |
| ENSMUSG00000020911.14 | 355.0885 | −3.1381 | 0.78446 | −4.00033 | 6.33E−05 | 0.003838 | Krt19 | 16669 | keratin 19 |
| ENSMUSG00000042212.3 | 22.59342 | −4.29195 | 1.073305 | −3.99882 | 6.37E−05 | 0.003842 | Sprr2d | 20758 | small proline-rich |
| | | | | | | | | | protein 2D |
| ENSMUSG00000091694.9 | 41.2218 | 4.566384 | 1.142192 | 3.997912 | 6.39E−05 | 0.003846 | Apol11b | 328563 | apolipoprotein L 11b |
| ENSMUSG00000024173.11 | 105.8972 | 2.705699 | 0.677257 | 3.995086 | 6.47E−05 | 0.003872 | Tpsab1 | 1.01E+08 | tryptase alpha/beta 1 |
| ENSMUSG00000032047.5 | 5925.243 | 0.877208 | 0.219723 | 3.992341 | 6.54E−05 | 0.003897 | Acat1 | 110446 | acetyl-Coenzyme A |
| | | | | | | | | | acetyltransferase 1 |
| ENSMUSG00000052566.8 | 1082.04 | −1.1184 | 0.280293 | −3.9901 | 6.60E−05 | 0.003923 | Hook2 | 170833 | hook microtubule |
| | | | | | | | | | tethering protein 2 |
| ENSMUSG00000048416.15 | 2017.314 | 1.194512 | 0.299991 | 3.981831 | 6.84E−05 | 0.004041 | Mlf1 | 17349 | myeloid leukemia |
| | | | | | | | | | factor 1 |
| ENSMUSG00000023267.10 | 54.74162 | 2.298675 | 0.577613 | 3.979613 | 6.90E−05 | 0.004051 | Gabrr2 | 14409 | gamma-aminobutyric |
| | | | | | | | | | acid (GABA) C |
| | | | | | | | | | receptor, subunit rho 2 |
| ENSMUSG00000037216.5 | 160.5989 | 0.60129 | 0.151141 | 3.978341 | 6.94E−05 | 0.004051 | Lipt1 | 623661 | lipoyltransferase 1 |
| ENSMUSG00000042401.8 | 583.9501 | 4.689373 | 1.178649 | 3.9786 | 6.93E−05 | 0.004051 | Crtac1 | 72832 | cartilage acidic protein |
| | | | | | | | | | 1 |
| ENSMUSG00000068009.11 | 15.88365 | 3.642337 | 0.915304 | 3.979372 | 6.91E−05 | 0.004051 | Bpifb6 | 228796 | BPI fold containing |
| | | | | | | | | | family B, member 6 |
| ENSMUSG00000003484.4 | 240.6224 | −3.31303 | 0.834742 | −3.96892 | 7.22E−05 | 0.004126 | Cyp4f18 | 72054 | cytochrome P450, |
| | | | | | | | | | family 4, subfamily f, |
| | | | | | | | | | polypeptide 18 |
| ENSMUSG00000021187.14 | 40.13151 | 1.442283 | 0.363359 | 3.969307 | 7.21E−05 | 0.004126 | Tc2n | 74413 | tandem C2 domains, |
| | | | | | | | | | nuclear |
| ENSMUSG00000090619.2 | 85.93774 | −0.72508 | 0.18266 | −3.96958 | 7.20E−05 | 0.004126 | Vmn2r60 | 637898 | vomeronasal 2, |
| | | | | | | | | | receptor 60 |
| ENSMUSG00000094335.2 | 737.1481 | −9.57603 | 2.411222 | −3.97144 | 7.14E−05 | 0.004126 | Igkv1-117 | 16098 | immunoglobulin kappa |
| | | | | | | | | | variable 1-117 |
| ENSMUSG00000046908.5 | 318.7829 | −1.40792 | 0.35536 | −3.96195 | 7.43E−05 | 0.004238 | Ltb4r1 | 16995 | leukotriene B4 |
| | | | | | | | | | receptor 1 |
| ENSMUSG00000026220.6 | 310.5389 | −2.69062 | 0.680324 | 3.95491 | 7.66E−05 | 0.004306 | Slc16a14 | 71781 | solute carrier family 16 |
| | | | | | | | | | (monocarboxylic acid |
| | | | | | | | | | transporters), member |
| | | | | | | | | | 14 |
| ENSMUSG00000026420.16 | 16.23627 | −7.44958 | 1.8836 | −3.95497 | 7.65E−05 | 0.004306 | Il24 | 93672 | interleukin 24 |
| ENSMUSG00000043931.10 | 109.4328 | −5.44567 | 1.376034 | −3.95751 | 7.57E−05 | 0.004306 | Gimap7 | 231932 | GTPase, IMAP family |
| | | | | | | | | | member 7 |
| ENSMUSG00000047371.7 | 487.0659 | 0.37017 | 0.093603 | 3.95467 | 7.66E−05 | 0.004306 | Zfp768 | 233890 | zinc finger protein 768 |
| ENSMUSG00000066820.7 | 7.387942 | 2.60775 | 0.659426 | 3.954575 | 7.67E−05 | 0.004306 | Vmn2r28 | 665255 | vomeronasal 2, |
| | | | | | | | | | receptor 28 |
| ENSMUSG00000031906.9 | 2537.45 | −1.66036 | 0.419986 | −3.95338 | 7.71E−05 | 0.004317 | Smpd3 | 58994 | sphingomyelin |
| | | | | | | | | | phosphodiesterase 3, |
| | | | | | | | | | neutral |
| ENSMUSG00000034842.16 | 3657.346 | 1.661015 | 0.420276 | 3.952199 | 7.74E−05 | 0.004327 | Art3 | 109979 | ADP- |
| | | | | | | | | | ribosyltransferase 3 |
| ENSMUSG00000038028.9 | 1117.169 | 1.343565 | 0.340084 | 3.95069 | 7.79E−05 | 0.004344 | Tigar | 319801 | Trp53 induced |
| | | | | | | | | | glycolysis regulatory |
| | | | | | | | | | phosphatase |
| ENSMUSG00000030378.15 | 4.64634 | −5.56664 | 1.409876 | −3.94832 | 7.87E−05 | 0.004376 | Sult2a8 | 76971 | sulfotransferase family |
| | | | | | | | | | 2A, |
| | | | | | | | | | dehydroepiandrosterone |
| | | | | | | | | | (DHEA)-preferring, |
| | | | | | | | | | member 8 |
| ENSMUSG00000025172.3 | 553.7139 | 2.017211 | 0.511388 | 3.94458 | 7.99E−05 | 0.004423 | Ankrd2 | 56642 | ankyrin repeat domain |
| | | | | | | | | | 2 (stretch responsive |
| | | | | | | | | | muscle) |
| ENSMUSG00000033192.5 | 754.5619 | −1.0773 | 0.273409 | −3.94025 | 8.14E−05 | 0.004493 | Lpcat2 | 270084 | lysophosphatidylcholine |
| | | | | | | | | | acyltransferase 2 |
| ENSMUSG00000028607.16 | 1871.357 | 1.037914 | 0.263635 | 3.936941 | 8.25E−05 | 0.004544 | Cpt2 | 12896 | carnitine |
| | | | | | | | | | palmitoyltransferase 2 |
| ENSMUSG00000094747.3 | 185.3734 | −0.81049 | 0.205933 | −3.93568 | 8.30E−05 | 0.004557 | Or4f14b | 257956 | olfactory receptor |
| | | | | | | | | | family 4 subfamily F |
| | | | | | | | | | member 14B |
| ENSMUSG00000035226.5 | 51.49343 | 2.647175 | 0.67328 | 3.931756 | 8.43E−05 | 0.004621 | Rims4 | 241770 | regulating synaptic |
| | | | | | | | | | membrane exocytosis |
| | | | | | | | | | 4 |
| ENSMUSG00000050762.5 | 99.82324 | −1.29593 | 0.329806 | −3.92937 | 8.52E−05 | 0.004637 | Prss27 | 213171 | protease, serine 27 |
| ENSMUSG00000096594.2 | 18.97184 | −7.67069 | 1.95222 | −3.92921 | 8.52E−05 | 0.004637 | Igkv8-19 | 232065 | immunoglobulin kappa |
| | | | | | | | | | variable 8-19 |
| ENSMUSG00000046312.4 | 982.3523 | 1.471232 | 0.374502 | 3.928505 | 8.55E−05 | 0.004639 | Myorg | 329828 | myogenesis regulating |
| | | | | | | | | | glycosidase (putative) |
| ENSMUSG00000029199.11 | 1198.568 | 0.513167 | 0.130666 | 3.927336 | 8.59E−05 | 0.004651 | Lias | 79464 | lipoic acid synthetase |
| ENSMUSG00000112980.1 | 57.74233 | −1.72976 | 0.440509 | −3.92674 | 8.61E−05 | 0.004651 | D43002OJ02Rik | 319545 | RIKEN cDNA |
| | | | | | | | | | D430020J02 gene |
| ENSMUSG00000025597.13 | 62.33521 | 1.776373 | 0.452615 | 3.924688 | 8.68E−05 | 0.00468 | Klh14 | 237010 | kelch-like 4 |
| ENSMUSG00000022871.13 | 43.37834 | −1.7732 | 0.45259 | −3.91791 | 8.93E−05 | 0.00479 | Fetub | 59083 | fetuin beta |
| ENSMUSG00000046180.11 | 5.185013 | 5.230295 | 1.334852 | 3.918257 | 8.92E−05 | 0.00479 | Magea13 | 75352 | MAGE family member |
| | | | | | | | | | A13 |
| ENSMUSG00000018574.14 | 5385 | 0.65001 | 0.165959 | 3.916695 | 8.98E−05 | 0.004792 | Acadvl | 11370 | acyl-Coenzyme A |
| | | | | | | | | | dehydrogenase, very |
| | | | | | | | | | long chain |
| ENSMUSG00000024768.5 | 62.69229 | 9.428837 | 2.407264 | 3.916828 | 8.97E−05 | 0.004792 | Lipf | 67717 | lipase, gastric |
| ENSMUSG00000079588.3 | 4216.446 | 1.422891 | 0.363685 | 3.912432 | 9.14E−05 | 0.004864 | Tmem182 | 381339 | transmembrane protein |
| | | | | | | | | | 182 |
| ENSMUSG00000087579.7 | 147.3906 | 1.974127 | 0.504639 | 3.911955 | 9.16E−05 | 0.004864 | Hectd20S | 668215 | Hectd2, opposite |
| | | | | | | | | | strand |
| ENSMUSG00000028965.13 | 28.74478 | −2.5262 | 0.646237 | −3.90909 | 9.26E−05 | 0.004876 | Tnfrsf9 | 21942 | tumor necrosis factor |
| | | | | | | | | | receptor superfamily, |
| | | | | | | | | | member 9 |
| ENSMUSG00000053279.8 | 5620.023 | 1.450661 | 0.37102 | 3.909926 | 9.23E−05 | 0.004876 | Aldh1a1 | 11668 | aldehyde |
| | | | | | | | | | dehydrogenase family |
| | | | | | | | | | 1, subfamily A1 |
| ENSMUSG00000058153.15 | 62.72452 | 2.88647 | 0.738361 | 3.909292 | 9.26E−05 | 0.004876 | Sez61 | 56747 | seizure related 6 |
| | | | | | | | | | homolog like |
| ENSMUSG00000060560.8 | 125.6054 | −2.7813 | 0.711207 | −3.91067 | 9.20E−05 | 0.004876 | Ces4a | 234677 | carboxylesterase 4A |
| ENSMUSG00000066583.4 | 5.926573 | −5.3818 | 1.37745 | −3.90708 | 9.34E−05 | 0.004905 | Scgb1b27 | 11354 | secretoglobin, family |
| | | | | | | | | | 1B, member 27 |
| ENSMUSG00000049565.16 | 258.8286 | −1.50257 | 0.384643 | −3.9064 | 9.37E−05 | 0.004908 | Aknad1 | 329738 | AKNA domain |
| | | | | | | | | | containing 1 |
| ENSMUSG00000006731.10 | 934.5416 | −1.21442 | 0.311343 | −3.90059 | 9.60E−05 | 0.005004 | B4galnt1 | 14421 | beta-1,4-N-acetyl- |
| | | | | | | | | | galactosaminy1 |
| | | | | | | | | | transferase 1 |
| ENSMUSG00000028064.17 | 1907.029 | −0.76322 | 0.195739 | −3.89916 | 9.65E−05 | 0.005011 | Sema4a | 20351 | sema domain, |
| | | | | | | | | | immunoglobulin |
| | | | | | | | | | domain (Ig), |
| | | | | | | | | | transmembrane |
| | | | | | | | | | domain (TM) and short |
| | | | | | | | | | cytoplasmic domain, |
| | | | | | | | | | (semaphorin) 4A |
| ENSMUSG00000067925.4 | 988.961 | 1.12842 | 0.28939 | 3.899319 | 9.65E−05 | 0.005011 | Rtl8a | 66158 | retrotransposon Gag |
| | | | | | | | | | like 8A |
| ENSMUSG00000047562.3 | 20.03081 | −4.08007 | 1.046715 | −3.89797 | 9.70E−05 | 0.005012 | Mmp10 | 17384 | matrix |
| | | | | | | | | | metallopeptidase 10 |
| ENSMUSG00000020151.16 | 52.00642 | 1.700996 | 0.436968 | 3.892721 | 9.91E−05 | 0.00511 | Ptprr | 19279 | protein tyrosine |
| | | | | | | | | | phosphatase, receptor |
| | | | | | | | | | type, R |

TABLE 10
(Supplementary Table S5. Ranked genes by differential methylation (Y vs O))

| | Base Mean | log2FoldChange | lfcSE | stat | pvalue | padj | symbol | entrez | name |
|---|---|---|---|---|---|---|---|---|---|
| ENSMUSG00000026173.15 | 599.8638 | 3.28655 | 0.208369 | 15.77277 | 4.79E−56 | 1.12E−51 | Plcd4 | 18802 | phospholipase C, delta 4 |
| ENSMUSG00000026100.6 | 5371.714 | 3.691306 | 0.282776 | 13.05383 | 6.04E−39 | 7.05E−35 | Mstn | 17700 | myostatin |
| ENSMUSG00000005716.16 | 102726.5 | 3.457844 | 0.276322 | 12.51383 | 6.27E−36 | 4.25E−32 | Pvalb | 19293 | parvalbumin |
| ENSMUSG00000045667.14 | 2151.245 | 2.698353 | 0.215836 | 12.50184 | 7.29E−36 | 4.25E−32 | Smtnl2 | 276829 | smoothelin-like 2 |
| ENSMUSG00000068303.6 | 74.94969 | 3.793654 | 0.31036 | 12.22341 | 2.33E−34 | 1.09E−30 | NA | NA | NA |
| ENSMUSG00000100801.1 | 4030.249 | 11.46523 | 0.945014 | 12.13233 | 7.12E−34 | 2.77E−30 | Gm15459 | 727711 | heat shock protein 8 pseudogene |
| ENSMUSG00000028584.3 | 235.7366 | 2.986694 | 0.247023 | 12.09075 | 1.18E−33 | 3.94E−30 | Lrrc38 | 242735 | leucine rich repeat containing 38 |
| ENSMUSG00000110275.1 | 1258.324 | 7.947867 | 0.663862 | 11.97217 | 4.97E−33 | 1.45E−29 | Gm5905 | 546015 | ribosomal protein S9 pseudogene |
| ENSMUSG00000097666.2 | 94.49376 | 2.833654 | 0.239293 | 11.84177 | 2.37E−32 | 6.15E−29 | NA | NA | NA |
| ENSMUSG00000099891.1 | 196.5134 | 8.992906 | 0.76099 | 11.81737 | 3.17E−32 | 7.41E−29 | Gm5575 | 434047 | heat shock protein 8 pseudogene |
| ENSMUSG00000032114.9 | 1459.152 | 2.111351 | 0.17957 | 11.75783 | 6.44E−32 | 1.36E−28 | Slc37a4 | 14385 | solute carrier family 37 (glucose-6- |
| | | | | | | | | | phosphate transporter), member 4 |
| ENSMUSG00000048416.15 | 5761.131 | 3.468822 | 0.305621 | 11.35007 | 7.41E−30 | 1.44E−26 | Mlf1 | 17349 | myeloid leukemia factor 1 |
| ENSMUSG00000109925.1 | 111.3739 | 10.64451 | 0.939037 | 11.33556 | 8.75E−30 | 1.57E−26 | NA | NA | NA |
| ENSMUSG00000036352.16 | 3349.112 | 1.607966 | 0.146057 | 11.00918 | 3.45E−28 | 5.75E−25 | Ubac1 | 98766 | ubiquitin associated domain containing 1 |
| ENSMUSG00000113032.1 | 644.7551 | −1.8361 | 0.167612 | −10.9544 | 6.33E−28 | 9.84E−25 | NA | NA | NA |
| ENSMUSG00000085348.1 | 344.3381 | 2.890556 | 0.272793 | 10.59614 | 3.11E−26 | 4.53E−23 | Myhas | 1.03E+08 | myosin heavy chain gene antisense RNA |
| ENSMUSG00000033788.15 | 928.113 | 2.945687 | 0.281654 | 10.45855 | 1.34E−25 | 1.84E−22 | Dysf | 26903 | dysferlin |
| ENSMUSG00000022987.12 | 414.4811 | 2.622735 | 0.252955 | 10.36837 | 3.45E−25 | 4.48E−22 | Zfp641 | 239652 | zinc finger protein 641 |
| ENSMUSG00000070385.12 | 7822.019 | 2.570204 | 0.248817 | 10.3297 | 5.17E−25 | 6.35E−22 | Ampd1 | 229665 | adenosine monophosphate deaminase 1 |
| ENSMUSG00000112926.1 | 67.99211 | 7.204042 | 0.711975 | 10.1184 | 4.58E−24 | 5.34E−21 | NA | NA | NA |
| ENSMUSG00000023336.6 | 826.9946 | 2.682936 | 0.265991 | 10.08656 | 6.33E−24 | 7.04E−21 | Wfdc1 | 67866 | WAP four-disulfide core domain 1 |
| ENSMUSG00000096403.2 | 52.6855 | 9.561552 | 0.948475 | 10.08097 | 6.71E−24 | 7.11E−21 | NA | NA | NA |
| ENSMUSG00000097974.1 | 201.3756 | 2.844444 | 0.283602 | 10.02971 | 1.13E−23 | 1.14E−20 | NA | NA | NA |
| ENSMUSG00000104011.1 | 74.51911 | 3.534683 | 0.353705 | 9.993313 | 1.63E−23 | 1.58E−20 | Gm32391 | 1.03E+08 | predicted gene, 32391 |
| ENSMUSG00000002228.7 | 387.9039 | 2.615776 | 0.261867 | 9.988943 | 1.70E−23 | 1.59E−20 | Ppm1j | 71887 | protein phosphatase 1J |
| ENSMUSG00000107585.1 | 392.7446 | 3.777416 | 0.378463 | 9.980943 | 1.85E−23 | 1.66E−20 | 3300002P13Rik | 70230 | RIKEN cDNA 3300002P13 gene |
| ENSMUSG00000096606.2 | 797.8148 | 2.654551 | 0.266904 | 9.945705 | 2.63E−23 | 2.27E−20 | Tpbgl | 1.01E+08 | trophoblast glycoprotein-like |
| ENSMUSG00000087410.7 | 408.0513 | 2.954433 | 0.297611 | 9.927158 | 3.17E−23 | 2.64E−20 | 2310065F04Rik | 74184 | RIKEN cDNA 2310065F04 gene |
| ENSMUSG00000006457.4 | 41929.65 | 2.808263 | 0.285474 | 9.837185 | 7.79E−23 | 6.26E−20 | Actn3 | 11474 | actinin alpha 3 |
| ENSMUSG00000038777.19 | 778.6387 | 2.569288 | 0.261894 | 9.8104 | 1.02E−22 | 7.90E−20 | Sema6c | 20360 | sema domain, transmembrane domain (TM), |
| | | | | | | | | | and cytoplasmic domain, (semaphorin) 6C |
| ENSMUSG00000038403.10 | 4034.128 | 2.881697 | 0.294269 | 9.792719 | 1.21E−22 | 9.10E−20 | Hjv | 69585 | hemojuvelin BMP co-receptor |
| ENSMUSG00000028023.16 | 296.7934 | 3.191722 | 0.327184 | 9.755123 | 1.75E−22 | 1.28E−19 | Pitx2 | 18741 | paired-like homeodomain transcription |
| | | | | | | | | | factor 2 |
| ENSMUSG00000033032.15 | 1100.844 | 1.722678 | 0.177094 | 9.727462 | 2.30E−22 | 1.63E−19 | Afap1l1 | 106877 | actin filament associated protein 1-like 1 |
| ENSMUSG00000062694.7 | 1352.918 | 3.056377 | 0.317954 | 9.61265 | 7.07E−22 | 4.85E−19 | Cav3 | 12391 | caveolin 3 |
| ENSMUSG00000091957.3 | 8322.593 | 3.708821 | 0.388637 | 9.543143 | 1.39E−21 | 9.23E−19 | Rps2-ps10 | 667279 | ribosomal protein S2, pseudogene 10 |
| ENSMUSG00000059824.12 | 2852.104 | 2.843098 | 0.298622 | 9.520729 | 1.72E−21 | 1.11E−18 | Dbp | 13170 | D site albumin promoter binding protein |
| ENSMUSG00000073198.5 | 53.34934 | 9.583094 | 1.008308 | 9.504135 | 2.02E−21 | 1.27E−18 | Bnip31-ps | 1E+08 | BCL2/adenovirus E1B interacting protein 3-like, |
| | | | | | | | | | pseudogene |
| ENSMUSG00000051373.5 | 1133.001 | 3.161914 | 0.335531 | 9.423612 | 4.36E−21 | 2.68E−18 | Plpp7 | 227721 | phospholipid phosphatase 7 (inactive) |
| ENSMUSG00000023826.16 | 282.6236 | 1.938828 | 0.206032 | 9.410347 | 4.95E−21 | 2.96E−18 | Prkn | 50873 | parkin RBR E3 ubiquitin protein ligase |
| ENSMUSG00000034353.14 | 2460.686 | 2.98463 | 0.318954 | 9.357555 | 8.16E−21 | 4.76E−18 | Ramp1 | 51801 | receptor (calcitonin) activity modifying protein 1 |
| ENSMUSG00000019194.15 | 4371.632 | 2.656719 | 0.284692 | 9.33192 | 1.04E−20 | 5.92E−18 | Scn1b | 20266 | sodium channel, voltage-gated, type I, beta |
| ENSMUSG00000117813.1 | 45.1431 | 7.216697 | 0.775163 | 9.309913 | 1.28E−20 | 7.11E−18 | NA | NA | NA |
| ENSMUSG00000029156.11 | 1819.222 | 1.731866 | 0.186164 | 9.302903 | 1.37E−20 | 7.41E−18 | Sgcb | 24051 | sarcoglycan, beta (dystrophin-associated |
| | | | | | | | | | glycoprotein) |
| ENSMUSG00000028396.5 | 1533.789 | 2.938648 | 0.316514 | 9.284425 | 1.63E−20 | 8.62E−18 | 2310002L09Rik | 71886 | RIKEN cDNA 2310002L09 gene |
| ENSMUSG00000100622.1 | 47.53594 | 8.838097 | 0.954073 | 9.263545 | 1.98E−20 | 1.03E−17 | Gm20379 | 1.15E+08 | predicted gene, 20379 |
| ENSMUSG00000080242.5 | 46.239 | 9.376426 | 1.01284 | 9.257557 | 2.09E−20 | 1.06E−17 | NA | NA | NA |
| ENSMUSG00000083720.1 | 50.45856 | 9.493977 | 1.02634 | 9.250326 | 2.24E−20 | 1.11E−17 | Gm12901 | 194197 | ribosomal protein L11 pseudogene |
| ENSMUSG00000044951.17 | 10357.66 | 4.131841 | 0.447629 | 9.230497 | 2.69E−20 | 1.31E−17 | Mylk4 | 238564 | myosin light chain kinase family, member 4 |
| ENSMUSG00000022610.10 | 2743.411 | 2.434674 | 0.264531 | 9.203727 | 3.46E−20 | 1.65E−17 | Mapk12 | 29857 | mitogen-activated protein kinase 12 |
| ENSMUSG00000028949.13 | 1873.634 | 2.611893 | 0.284809 | 9.170692 | 4.70E−20 | 2.19E−17 | Smarcd3 | 66993 | SWI/SNF related, matrix associated, actin dependent |
| | | | | | | | | | regulator of chromatin, subfamily d, member 3 |
| ENSMUSG00000020216.13 | 3902.821 | 3.096358 | 0.338764 | 9.140153 | 6.24E−20 | 2.85E−17 | Jsrp1 | 71912 | junctional sarcoplasmic reticulum protein 1 |
| ENSMUSG00000047591.5 | 221.3382 | 1.931983 | 0.211594 | 9.130612 | 6.81E−20 | 3.06E−17 | Mafa | 378435 | v-maf musculoaponeurotic fibrosarcoma oncogene |
| | | | | | | | | | family, protein A (avian) |
| ENSMUSG00000026407.17 | 7415.636 | 2.666676 | 0.293081 | 9.098769 | 9.14E−20 | 4.02E−17 | Cacna1s | 12292 | calcium channel, voltage-dependent, L type, alpha |
| | | | | | | | | | 1S subunit |
| ENSMUSG00000027868.11 | 3948.174 | 2.550576 | 0.282642 | 9.024062 | 1.81E−19 | 7.83E−17 | Tbx15 | 21384 | T-box 15 |
| ENSMUSG00000026888.14 | 1252.284 | 2.724966 | 0.302912 | 8.9959 | 2.34E−19 | 9.94E−17 | Grb14 | 50915 | growth factor receptor bound protein 14 |
| ENSMUSG00000110632.1 | 146.2448 | 9.35907 | 1.04438 | 8.961363 | 3.21E−19 | 1.34E−16 | NA | NA | NA |
| ENSMUSG00000069014.4 | 503.2798 | −11.9774 | 1.342812 | −8.91962 | 4.68E−19 | 1.91E−16 | Gm5641 | 434807 | heterogeneous nuclear ribonucleoprotein A3 pseudogene |
| ENSMUSG00000027077.7 | 2873.542 | 3.309245 | 0.371393 | 8.910361 | 5.09E−19 | 2.05E−16 | Smtnl1 | 68678 | smoothelin-like 1 |
| ENSMUSG00000007877.2 | 41803.88 | 3.579558 | 0.403179 | 8.878328 | 6.79E−19 | 2.68E−16 | Tcap | 21393 | titin-cap |
| ENSMUSG00000017817.11 | 4550.439 | 3.034301 | 0.343845 | 8.824619 | 1.10E−18 | 4.27E−16 | Jph2 | 59091 | junctophilin 2 |
| ENSMUSG00000040652.16 | 3837.985 | 1.54715 | 0.175584 | 8.811468 | 1.24E−18 | 4.72E−16 | Oaz2 | 18247 | ornithine decarboxylase antizyme 2 |
| ENSMUSG00000038195.6 | 484.5639 | 2.664989 | 0.30309 | 8.792731 | 1.46E−18 | 5.49E−16 | Rilp | 280408 | Rab interacting lysosomal protein |
| ENSMUSG00000024937.15 | 4737.199 | 1.807186 | 0.205851 | 8.779107 | 1.65E−18 | 6.10E−16 | Ehbp1l1 | 114601 | EH domain binding protein 1-like 1 |
| ENSMUSG00000041889.7 | 2180.316 | 2.99359 | 0.34232 | 8.745017 | 2.23E−18 | 8.13E−16 | Shisa4 | 77552 | shisa family member 4 |
| ENSMUSG00000027257.13 | 4465.424 | 2.223835 | 0.254398 | 8.741566 | 2.30E−18 | 8.25E−16 | Pacsin3 | 80708 | protein kinase C and casein kinase substarte in |
| | | | | | | | | | neurons 3 |
| ENSMUSG00000094344.1 | 113.9113 | 6.728507 | 0.77189 | 8.716925 | 2.86E−18 | 1.01E−15 | NA | NA | NA |
| ENSMUSG00000021506.7 | 57.25944 | 3.784615 | 0.434593 | 8.708414 | 3.08E−18 | 1.07E−15 | Pitx1 | 18740 | paired-like homeodomain transcription factor 1 |
| ENSMUSG00000044938.8 | 4121.769 | 2.605326 | 0.300187 | 8.679001 | 3.99E−18 | 1.37E−15 | Klhl31 | 244923 | kelch-like 31 |
| ENSMUSG00000030554.16 | 10311.87 | 3.055729 | 0.352524 | 8.668157 | 4.39E−18 | 1.48E−15 | Synm | 233335 | synemin, intermediate filament protein |
| ENSMUSG00000032643.12 | 2150.91 | 2.367873 | 0.273826 | 8.647375 | 5.27E−18 | 1.76E−15 | Fhl3 | 14201 | four and a half LIM domains 3 |
| ENSMUSG00000063296.5 | 254.1561 | 2.843944 | 0.329676 | 8.626474 | 6.33E−18 | 2.08E−15 | Tmem117 | 320709 | transmembrane protein 117 |
| ENSMUSG00000041329.13 | 1494.886 | 3.045233 | 0.35378 | 8.607708 | 7.45E−18 | 2.41E−15 | Atp1b2 | 11932 | ATPase, Na+/K+ transporting, beta 2 polypeptide |
| ENSMUSG00000079434.8 | 7182.438 | 3.800815 | 0.442461 | 8.590165 | 8.68E−18 | 2.76E−15 | Neu2 | 23956 | neuraminidase 2 |
| ENSMUSG00000117300.1 | 28.9274 | 8.688273 | 1.011514 | 8.589373 | 8.74E−18 | 2.76E−15 | Gm18736 | 1E+08 | mitochondrial ribosomal protein S10 pseudogene |
| ENSMUSG00000081824.5 | 442.6146 | 3.896326 | 0.454341 | 8.575767 | 9.84E−18 | 3.06E−15 | NA | NA | NA |
| ENSMUSG00000099397.1 | 35.12624 | 8.984922 | 1.048197 | 8.57179 | 1.02E−17 | 3.13E−15 | NA | NA | NA |
| ENSMUSG00000018845.14 | 3042.477 | 2.517978 | 0.294144 | 8.560369 | 1.13E−17 | 3.41E−15 | Unc45b | 217012 | unc-45 myosin chaperone B |
| ENSMUSG00000026077.15 | 116.3547 | −2.60096 | 0.304434 | −8.5436 | 1.30E−17 | 3.87E−15 | Npas2 | 18143 | neuronal PAS domain protein 2 |
| ENSMUSG00000111375.4 | 306.523 | 1.768294 | 0.206992 | 8.542807 | 1.31E−17 | 3.87E−15 | Btbd8 | 1.01E+08 | BTB (POZ) domain containing 8 |
| ENSMUSG00000048096.7 | 1044.629 | 3.146359 | 0.368747 | 8.532575 | 1.43E−17 | 4.17E−15 | Lmod1 | 93689 | leiomodin 1 (smooth muscle) |
| ENSMUSG00000019927.6 | 1721.052 | 1.551646 | 0.182285 | 8.512176 | 1.71E−17 | 4.92E−15 | Ube2d1 | 216080 | ubiquitin-conjugating enzyme E2D 1 |
| ENSMUSG00000026571.12 | 2772.846 | 1.595547 | 0.187538 | 8.507842 | 1.77E−17 | 5.00E−15 | Dcaf6 | 74106 | DDB1 and CUL4 associated factor 6 |
| ENSMUSG00000032648.14 | 44530.81 | 3.076518 | 0.361633 | 8.507283 | 1.78E−17 | 5.00E−15 | Pygm | 19309 | muscle glycogen phosphorylase |
| ENSMUSG00000007122.11 | 27703.98 | 3.009006 | 0.35428 | 8.493297 | 2.01E−17 | 5.58E−15 | Casq1 | 12372 | calsequestrin 1 |
| ENSMUSG00000091014.3 | 122.2382 | 9.032462 | 1.069401 | 8.446278 | 3.01E−17 | 8.25E−15 | NA | NA | NA |
| ENSMUSG00000036879.15 | 5457.957 | 1.949229 | 0.231394 | 8.423837 | 3.64E−17 | 9.88E−15 | Phkb | 102093 | phosphorylase kinase beta |
| ENSMUSG00000005628.12 | 7699.994 | 2.87633 | 0.34363 | 8.370426 | 5.74E−17 | 1.52E−14 | Tmod4 | 50874 | tropomodulin 4 |
| ENSMUSG00000068697.7 | 28017.99 | 3.228039 | 0.385604 | 8.371391 | 5.69E−17 | 1.52E−14 | Myoz1 | 59011 | myozenin 1 |
| ENSMUSG00000039376.13 | 1474.823 | 2.903678 | 0.347846 | 8.347592 | 6.97E−17 | 1.83E−14 | Synpo2l | 68760 | synaptopodin 2-like |
| ENSMUSG00000025791.18 | 8873.681 | 2.501468 | 0.300224 | 8.332 | 7.95E−17 | 2.06E−14 | Pgm1 | 72157 | phosphoglucomutase 1 |
| ENSMUSG00000042359.18 | 501.5635 | 3.232627 | 0.388319 | 8.324664 | 8.46E−17 | 2.17E−14 | Osbpl6 | 99031 | oxysterol binding protein-like 6 |
| ENSMUSG00000117545.1 | 319.3723 | 2.706455 | 0.325963 | 8.30294 | 1.02E−16 | 2.58E−14 | Gm30794 | 1.03E+08 | predicted gene, 30794 |
| ENSMUSG00000110569.1 | 38.45667 | 7.946899 | 0.959778 | 8.279932 | 1.23E−16 | 3.09E−14 | NA | NA | NA |
| ENSMUSG00000096255.2 | 2645.745 | 2.508491 | 0.303028 | 8.278076 | 1.25E−16 | 3.11E−14 | Dynlt1b | 21648 | dynein light chain Tctex-type 1B |
| ENSMUSG00000042686.5 | 2218.752 | 2.608161 | 0.315165 | 8.275543 | 1.28E−16 | 3.14E−14 | Jph1 | 57339 | junctophilin 1 |
| ENSMUSG00000025141.3 | 1394.826 | 2.908306 | 0.351601 | 8.271619 | 1.32E−16 | 3.21E−14 | Myadml2 | 68515 | myeloid-associated differentiation marker-like 2 |
| ENSMUSG00000033044.12 | 2367.603 | 2.920413 | 0.35342 | 8.263297 | 1.42E−16 | 3.37E−14 | Dhrs7c | 68460 | dehydrogenase/reductase (SDR family) member 7C |
| ENSMUSG00000113036.1 | 22.36155 | 8.335243 | 1.008714 | 8.263238 | 1.42E−16 | 3.37E−14 | NA | NA | NA |
| ENSMUSG00000006542.13 | 506.3835 | 2.570277 | 0.311451 | 8.252595 | 1.55E−16 | 3.65E−14 | Prkag3 | 241113 | protein kinase, AMP-activated, gamma 3 non-catalytic |
| | | | | | | | | | subunit |
| ENSMUSG00000020882.17 | 2164.722 | 2.84052 | 0.344629 | 8.242257 | 1.69E−16 | 3.94E−14 | Cacnb1 | 12295 | calcium channel, voltage-dependent, beta 1 subunit |
| ENSMUSG00000038349.10 | 272.7533 | 2.210918 | 0.268458 | 8.23562 | 1.79E−16 | 4.13E−14 | Plcl1 | 227120 | phospholipase C-like 1 |
| ENSMUSG00000032366.15 | 136455.4 | 2.895654 | 0.351656 | 8.234346 | 1.81E−16 | 4.13E−14 | Tpm1 | 22003 | tropomyosin 1, alpha |
| ENSMUSG00000061723.18 | 245487.6 | 3.007785 | 0.365449 | 8.230376 | 1.87E−16 | 4.23E−14 | Tnnt3 | 21957 | troponin T3, skeletal, fast |
| ENSMUSG00000002012.13 | 184.2325 | 1.968433 | 0.239333 | 8.224649 | 1.96E−16 | 4.39E−14 | Pnck | 93843 | pregnancy upregulated non-ubiquitously expressed |
| | | | | | | | | | CaM kinase |
| ENSMUSG00000026544.6 | 456.1902 | 1.935848 | 0.235559 | 8.218085 | 2.07E−16 | 4.59E−14 | Dusp23 | 68440 | dual specificity phosphatase 23 |
| ENSMUSG00000025610.7 | 135.6202 | 2.877289 | 0.350443 | 8.210433 | 2.20E−16 | 4.85E−14 | Map3k7cl | 224419 | Map3k7 C-terminal like |
| ENSMUSG00000063229.15 | 62030.42 | 2.253756 | 0.274949 | 8.196987 | 2.46E−16 | 5.37E−14 | Ldha | 16828 | lactate dehydrogenase A |
| ENSMUSG00000038170.15 | 25598.85 | 2.605515 | 0.318171 | 8.189041 | 2.63E−16 | 5.69E−14 | Pde4dip | 83679 | phosphodiesterase 4D interacting protein (myomegalin) |
| ENSMUSG00000004558.15 | 29994.19 | 2.415663 | 0.295374 | 8.178321 | 2.88E−16 | 6.16E−14 | Ndrg2 | 29811 | N-myc downstream regulated gene 2 |
| ENSMUSG00000084329.1 | 39.66445 | 7.995572 | 0.978306 | 8.172874 | 3.01E−16 | 6.39E−14 | Gm6733 | 627119 | signal recognition particle receptor (‘docking protein’) |
| | | | | | | | | | pseudogene |
| ENSMUSG00000100599.2 | 535.5955 | 2.882017 | 0.353091 | 8.16224 | 3.29E−16 | 6.91E−14 | 1700120C14Rik | 73600 | RIKEN cDNA 1700120C14 gene |
| ENSMUSG00000017300.9 | 142594 | 3.296231 | 0.404438 | 8.150161 | 3.63E−16 | 7.57E−14 | Tnnc2 | 21925 | troponin C2, fast |
| ENSMUSG00000031097.15 | 151208.8 | 3.196187 | 0.392248 | 8.14838 | 3.69E−16 | 7.61E−14 | Tnni2 | 21953 | troponin I, skeletal, fast 2 |
| ENSMUSG00000078815.8 | 1367.677 | 2.637158 | 0.324662 | 8.122772 | 4.56E−16 | 9.32E−14 | Cacng6 | 54378 | calcium channel, voltage-dependent, gamma subunit 6 |
| ENSMUSG00000023456.16 | 40176.96 | 2.14419 | 0.264077 | 8.119574 | 4.68E−16 | 9.49E−14 | Tpi1 | 21991 | triosephosphate isomerase 1 |
| ENSMUSG00000090066.2 | 1975.161 | 3.140752 | 0.38691 | 8.117535 | 4.76E−16 | 9.57E−14 | 1110002E22Rik | 1.03E+08 | RIKEN cDNA 1110002E22 gene |
| ENSMUSG00000036333.11 | 2470.385 | 1.002811 | 0.123736 | 8.104468 | 5.30E−16 | 1.06E−13 | Kidins220 | 77480 | kinase D-interacting substrate 220 |
| ENSMUSG00000028207.18 | 4917.333 | 2.252677 | 0.278649 | 8.084288 | 6.25E−16 | 1.24E−13 | Asph | 65973 | aspartate-beta-hydroxylase |
| ENSMUSG00000016349.10 | 12841.63 | 3.302604 | 0.408693 | 8.080898 | 6.43E−16 | 1.25E−13 | Eef1a2 | 13628 | eukaryotic translation elongation factor 1 alpha 2 |
| ENSMUSG00000046345.5 | 671.2506 | 2.706757 | 0.334917 | 8.081871 | 6.38E−16 | 1.25E−13 | Smco1 | 69576 | single-pass membrane protein with coiled-coil domains 1 |
| ENSMUSG00000076591.3 | 202.3018 | −25.0122 | 3.099838 | −8.06888 | 7.09E−16 | 1.37E−13 | Igkv8-16 | 640340 | immunoglobulin kappa variable 8-16 |
| ENSMUSG00000105247.1 | 304.3436 | −2.5806 | 0.319973 | −8.06506 | 7.32E−16 | 1.40E−13 | NA | NA | NA |
| ENSMUSG00000079022.10 | 93.35085 | 2.920823 | 0.362323 | 8.061383 | 7.54E−16 | 1.43E−13 | Col22a1 | 69700 | collagen, type XXII, alpha 1 |
| ENSMUSG00000046727.13 | 2671.751 | 2.921878 | 0.362624 | 8.0576 | 7.78E−16 | 1.46E−13 | Cystm1 | 66060 | cysteine-rich transmembrane module containing 1 |
| ENSMUSG00000019933.7 | 1021.524 | 3.45374 | 0.42912 | 8.048418 | 8.39E−16 | 1.55E−13 | Mrln | 69563 | myoregulin |
| ENSMUSG00000062077.14 | 2260.387 | 3.123089 | 0.388031 | 8.048545 | 8.38E−16 | 1.55E−13 | Trim54 | 58522 | tripartite motif-containing 54 |
| ENSMUSG00000062563.15 | 311.6884 | 1.852534 | 0.230388 | 8.04092 | 8.92E−16 | 1.64E−13 | Cys1 | 12879 | cystin 1 |
| ENSMUSG00000096074.2 | 373.3953 | −25.844 | 3.226008 | −8.01114 | 1.14E−15 | 2.07E−13 | NA | NA | NA |
| ENSMUSG00000038086.4 | 994.2209 | 2.731385 | 0.341409 | 8.000337 | 1.24E−15 | 2.24E−13 | Hspb2 | 69253 | heat shock protein 2 |
| ENSMUSG00000074807.3 | 151.8494 | 1.717942 | 0.214784 | 7.998474 | 1.26E−15 | 2.26E−13 | NA | NA | NA |
| ENSMUSG00000022758.14 | 339.3897 | 2.817254 | 0.352886 | 7.983457 | 1.42E−15 | 2.53E−13 | P2rx6 | 18440 | purinergic receptor P2X, ligand-gated ion channel, 6 |
| ENSMUSG00000055865.8 | 90.37836 | 3.070737 | 0.384699 | 7.98219 | 1.44E−15 | 2.54E−13 | Tafa3 | 329731 | TAFA chemokine like family member 3 |
| ENSMUSG00000109783.1 | 73.72546 | 2.556881 | 0.320626 | 7.974648 | 1.53E−15 | 2.68E−13 | NA | NA | NA |
| ENSMUSG00000043126.5 | 139.2956 | 3.075946 | 0.385839 | 7.972104 | 1.56E−15 | 2.72E−13 | D830039M14Rik | 320949 | RIKEN cDNA D830039M14 gene |
| ENSMUSG00000025537.12 | 3256.316 | 2.825497 | 0.354702 | 7.965828 | 1.64E−15 | 2.84E−13 | Phkg1 | 18682 | phosphorylase kinase gamma 1 |
| ENSMUSG00000085779.1 | 1428.148 | 2.708425 | 0.340517 | 7.953869 | 1.81E−15 | 3.10E−13 | NA | NA | NA |
| ENSMUSG00000096780.7 | 454.5901 | 1.377368 | 0.174218 | 7.906001 | 2.66E−15 | 4.53E−13 | NA | NA | NA |
| ENSMUSG00000096146.2 | 1023.098 | 2.835614 | 0.358958 | 7.899574 | 2.80E−15 | 4.73E−13 | Kcnj11 | 16514 | potassium inwardly rectifying channel, subfamily J, |
| | | | | | | | | | member 11 |
| ENSMUSG00000035642.16 | 1824.893 | 2.340715 | 0.296614 | 7.891446 | 2.99E−15 | 5.01E−13 | Aamdc | 66273 | adipogenesis associated Mth938 domain containing |
| ENSMUSG00000095589.2 | 232.5715 | −25.2043 | 3.194972 | −7.88875 | 3.05E−15 | 5.09E−13 | Ighv1-55 | 780932 | immunoglobulin heavy variable 1-55 |
| ENSMUSG00000097413.2 | 139.0966 | 2.301402 | 0.291806 | 7.886741 | 3.10E−15 | 5.13E−13 | NA | NA | NA |
| ENSMUSG00000030730.12 | 198190.8 | 3.02115 | 0.383692 | 7.87389 | 3.44E−15 | 5.65E−13 | Atp2a1 | 11937 | ATPase, Ca++ transporting, cardiac muscle, fast |
| | | | | | | | | | twitch 1 |
| ENSMUSG00000116056.1 | 296.6072 | 3.016439 | 0.383234 | 7.87101 | 3.52E−15 | 5.74E−13 | Gm4544 | 1E+08 | predicted gene 4544 |
| ENSMUSG00000010492.10 | 571.1403 | 3.187608 | 0.405059 | 7.869486 | 3.56E−15 | 5.77E−13 | Uckl1os | 1E+08 | uridine-cytidine kinase 1-like 1, opposite strand |
| ENSMUSG00000020475.3 | 16301.41 | 2.950392 | 0.375003 | 7.867652 | 3.61E−15 | 5.81E−13 | Pgam2 | 56012 | phosphoglycerate mutase 2 |
| ENSMUSG00000105606.1 | 258.4734 | −25.3483 | 3.226044 | −7.8574 | 3.92E−15 | 6.27E−13 | Igkv2-109 | 628268 | immunoglobulin kappa variable 2-109 |
| ENSMUSG00000044086.8 | 3696.691 | 3.252878 | 0.414848 | 7.841133 | 4.46E−15 | 7.09E−13 | Lmod3 | 320502 | leiomodin 3 (fetal) |
| ENSMUSG00000096459.1 | 249.5382 | −25.2817 | 3.226048 | −7.83673 | 4.62E−15 | 7.29E−13 | Ighv9-3 | 780825 | immunoglobulin heavy variable V9-3 |
| ENSMUSG00000042961.13 | 466.5738 | 2.402681 | 0.307085 | 7.824165 | 5.11E−15 | 8.00E−13 | Egflam | 268780 | EGF-like, fibronectin type III and laminin G domains |
| ENSMUSG00000042066.16 | 468.1565 | 1.705895 | 0.218189 | 7.818434 | 5.35E−15 | 8.26E−13 | Tmcc2 | 68875 | transmembrane and coiled-coil domains 2 |
| ENSMUSG00000061816.15 | 91247.55 | 2.685045 | 0.343426 | 7.818414 | 5.35E−15 | 8.26E−13 | Myl1 | 17901 | myosin, light polypeptide 1 |
| ENSMUSG00000036570.14 | 6502.08 | 2.787885 | 0.357564 | 7.796881 | 6.35E−15 | 9.74E−13 | Fxyd1 | 56188 | FXYD domain-containing ion transport regulator 1 |
| ENSMUSG00000040666.19 | 3923 | 2.526834 | 0.324854 | 7.778373 | 7.35E−15 | 1.12E−12 | Sh3bgr | 50795 | SH3-binding domain glutamic acid-rich protein |
| ENSMUSG00000064317.5 | 38.53471 | 9.107113 | 1.172223 | 7.769099 | 7.90E−15 | 1.20E−12 | NA | NA | NA |
| ENSMUSG00000079278.2 | 2096.458 | 3.14561 | 0.405312 | 7.760966 | 8.43E−15 | 1.27E−12 | Tmem233 | 545798 | transmembrane protein 233 |
| ENSMUSG00000069008.3 | 142.0059 | 24.77524 | 3.193822 | 7.757241 | 8.68E−15 | 1.30E−12 | NA | NA | NA |
| ENSMUSG00000028328.13 | 4496.22 | 2.705305 | 0.348831 | 7.755345 | 8.81E−15 | 1.30E−12 | Tmod1 | 21916 | tropomodulin 1 |
| ENSMUSG00000062093.5 | 107.2972 | 2.891952 | 0.372893 | 7.755453 | 8.80E−15 | 1.30E−12 | Gm5088 | 328451 | poly(A)-binding protein, cytoplasmic pseudogene |
| ENSMUSG00000094694.6 | 323.8377 | −24.9345 | 3.22602 | −7.72917 | 1.08E−14 | 1.59E−12 | NA | NA | NA |
| ENSMUSG00000094502.5 | 188.4279 | −24.9168 | 3.226088 | −7.72354 | 1.13E−14 | 1.65E−12 | Ighv1-69 | 619833 | immunoglobulin heavy variable 1-69 |
| ENSMUSG00000044716.12 | 196.752 | 2.499339 | 0.324108 | 7.711444 | 1.24E−14 | 1.80E−12 | Dok7 | 231134 | docking protein 7 |
| ENSMUSG00000104117.5 | 79.61288 | 3.21169 | 0.416584 | 7.709593 | 1.26E−14 | 1.82E−12 | Gm20743 | 433374 | predicted gene, 20743 |
| ENSMUSG00000036278.7 | 2135.795 | 2.47634 | 0.321251 | 7.708429 | 1.27E−14 | 1.82E−12 | Macrod1 | 107227 | mono-ADP ribosylhydrolase 1 |
| ENSMUSG00000079110.12 | 2358.602 | 3.398717 | 0.441809 | 7.692734 | 1.44E−14 | 2.05E−12 | Capn3 | 12335 | calpain 3 |
| ENSMUSG00000020402.11 | 23786.84 | 1.770102 | 0.230163 | 7.690639 | 1.46E−14 | 2.07E−12 | Vdac1 | 22333 | voltage-dependent anion channel 1 |
| ENSMUSG00000087579.7 | 240.2467 | 3.284155 | 0.428 | 7.673254 | 1.68E−14 | 2.36E−12 | Hectd2os | 668215 | Hectd2, opposite strand |
| ENSMUSG00000056032.12 | 59.53974 | 7.692829 | 1.004091 | 7.661486 | 1.84E−14 | 2.55E−12 | NA | NA | NA |
| ENSMUSG00000061062.5 | 20.54264 | 8.212773 | 1.071966 | 7.661408 | 1.84E−14 | 2.55E−12 | NA | NA | NA |
| ENSMUSG00000026603.13 | 3027.456 | 2.065172 | 0.270003 | 7.648697 | 2.03E−14 | 2.80E−12 | Smyd2 | 226830 | SET and MYND domain containing 2 |
| ENSMUSG00000042903.8 | 2389.038 | 1.323926 | 0.173747 | 7.619836 | 2.54E−14 | 3.49E−12 | Foxo4 | 54601 | forkhead box O4 |
| ENSMUSG00000027887.11 | 3352.583 | 2.780937 | 0.365053 | 7.617896 | 2.58E−14 | 3.52E−12 | Sypl2 | 17306 | synaptophysin-like 2 |
| ENSMUSG00000030652.11 | 966.5078 | 1.519502 | 0.199596 | 7.612877 | 2.68E−14 | 3.61E−12 | Coq7 | 12850 | demethyl-Q 7 |
| ENSMUSG00000060923.5 | 1797.396 | 3.028504 | 0.397804 | 7.613065 | 2.68E−14 | 3.61E−12 | Acyp2 | 75572 | acylphosphatase 2, muscle type |
| ENSMUSG00000011148.14 | 11851.71 | 3.14734 | 0.413881 | 7.604462 | 2.86E−14 | 3.81E−12 | Adssl1 | 11565 | adenylosuccinate synthetase like 1 |
| ENSMUSG00000101655.1 | 1433.545 | 3.24576 | 0.426799 | 7.604885 | 2.85E−14 | 3.81E−12 | 2310040G24Rik | 381792 | RIKEN cDNA 2310040G24 gene |
| ENSMUSG00000108322.2 | 614.331 | 2.592343 | 0.341088 | 7.600214 | 2.96E−14 | 3.92E−12 | 5430431A17Rik | 71368 | RIKEN cDNA 5430431A17 gene |
| ENSMUSG00000026576.12 | 6902.908 | 2.5833 | 0.340212 | 7.59321 | 3.12E−14 | 4.11E−12 | Atp1b1 | 11931 | ATPase, Na+/K+ transporting, beta 1 polypeptide |
| ENSMUSG00000002500.15 | 5941.951 | 3.102897 | 0.409132 | 7.584104 | 3.35E−14 | 4.36E−12 | Rpl3l | 66211 | ribosomal protein L3-like |
| ENSMUSG00000090799.2 | 1908.159 | 2.887786 | 0.380742 | 7.584631 | 3.33E−14 | 4.36E−12 | Klhl33 | 546611 | kelch-like 33 |
| ENSMUSG00000116875.1 | 86.62862 | 24.21794 | 3.194027 | 7.582258 | 3.40E−14 | 4.40E−12 | NA | NA | NA |
| ENSMUSG00000060147.15 | 30710.35 | 2.256004 | 0.297819 | 7.575088 | 3.59E−14 | 4.63E−12 | Serpinb6a | 20719 | serine (or cysteine) peptidase inhibitor, clade B, |
| | | | | | | | | | member 6a |
| ENSMUSG00000095335.2 | 132.3774 | −24.4336 | 3.226158 | −7.57359 | 3.63E−14 | 4.65E−12 | Igkv3-5 | 667940 | immunoglobulin kappa chain variable 3-5 |
| ENSMUSG00000112022.2 | 82.11003 | 24.14451 | 3.194055 | 7.559202 | 4.06E−14 | 5.17E−12 | Gm2436 | 1E+08 | predicted gene 2436 |
| ENSMUSG00000026817.14 | 14634.2 | 2.543368 | 0.336604 | 7.555968 | 4.16E−14 | 5.27E−12 | Ak1 | 11636 | adenylate kinase 1 |
| ENSMUSG00000030996.8 | 4410.347 | 3.102844 | 0.410992 | 7.549639 | 4.36E−14 | 5.50E−12 | Art1 | 11870 | ADP-ribosyltransferase 1 |
| ENSMUSG00000038248.8 | 190.7285 | 2.313508 | 0.306613 | 7.545364 | 4.51E−14 | 5.66E−12 | Sobp | 109205 | sine oculis binding protein |
| ENSMUSG00000022525.13 | 917.7398 | 2.987761 | 0.396289 | 7.539345 | 4.72E−14 | 5.87E−12 | Plaat1 | 27281 | phospholipase A and acyltransferase 1 |
| ENSMUSG00000102070.1 | 240359 | 1.641472 | 0.217726 | 7.53915 | 4.73E−14 | 5.87E−12 | NA | NA | NA |
| ENSMUSG00000027022.14 | 20489.67 | 2.99848 | 0.397911 | 7.535549 | 4.86E−14 | 6.00E−12 | Xirp2 | 241431 | xin actin-binding repeat containing 2 |
| ENSMUSG00000049233.7 | 81.8801 | 24.06293 | 3.194057 | 7.533659 | 4.93E−14 | 6.06E−12 | Apoo-ps | 621156 | apolipoprotein O, pseudogene |
| ENSMUSG00000002409.18 | 867.8799 | 1.394355 | 0.185111 | 7.532518 | 4.98E−14 | 6.08E−12 | Dyrk1b | 13549 | dual-specificity tyrosine-(Y)-phosphorylation |
| | | | | | | | | | regulated kinase 1b |
| ENSMUSG00000030672.12 | 166639.8 | 3.357463 | 0.446335 | 7.522298 | 5.38E−14 | 6.54E−12 | Mylpf | 17907 | myosin light chain, phosphorylatable, fast skeletal |
| | | | | | | | | | muscle |
| ENSMUSG00000084929.1 | 274.0147 | 3.001665 | 0.399154 | 7.52006 | 5.48E−14 | 6.62E−12 | Foxo6os | 402730 | forkhead box O6, opposite strand |
| ENSMUSG00000039345.16 | 1585.916 | 2.556522 | 0.340409 | 7.510143 | 5.91E−14 | 7.10E−12 | Mettl22 | 239706 | methyltransferase like 22 |
| ENSMUSG00000097487.7 | 497.8809 | 1.688807 | 0.225531 | 7.488156 | 6.98E−14 | 8.36E−12 | Ptges3l | 73635 | prostaglandin E synthase 3 like |
| ENSMUSG00000000253.13 | 2228.001 | 3.063814 | 0.40976 | 7.477089 | 7.60E−14 | 9.04E−12 | Gmpr | 66355 | guanosine monophosphate reductase |
| ENSMUSG00000051456.4 | 439.0465 | 2.979167 | 0.398887 | 7.468704 | 8.10E−14 | 9.59E−12 | Hspb3 | 56534 | heat shock protein 3 |
| ENSMUSG00000080859.1 | 104.2244 | −24.0557 | 3.22622 | −7.4563 | 8.90E−14 | 1.05E−11 | Rpl10-ps1 | 1E+08 | ribosomal protein L10, pseudogene 1 |
| ENSMUSG00000069996.3 | 16.43405 | 7.883697 | 1.057541 | 7.454745 | 9.00E−14 | 1.06E−11 | Gm41341 | 1.05E+08 | predicted gene, 41341 |
| ENSMUSG00000022215.6 | 2923.702 | 3.278256 | 0.43986 | 7.452949 | 9.13E−14 | 1.06E−11 | Fitm1 | 68680 | fat storage-inducing transmembrane protein 1 |
| ENSMUSG00000021493.15 | 5351.76 | 3.045152 | 0.408674 | 7.451303 | 9.24E−14 | 1.07E−11 | Pdlim7 | 67399 | PDZ and LIM domain 7 |
| ENSMUSG00000030401.16 | 11586.62 | 3.110768 | 0.417482 | 7.451256 | 9.25E−14 | 1.07E−11 | Rtn2 | 20167 | reticulon 2 (Z-band associated protein) |
| ENSMUSG00000022594.14 | 2560.963 | 1.932156 | 0.259373 | 7.449335 | 9.38E−14 | 1.08E−11 | Lynx1 | 23936 | Ly6/neurotoxin 1 |
| ENSMUSG00000043683.4 | 5501.825 | 1.785495 | 0.240136 | 7.435336 | 1.04E−13 | 1.19E−11 | Fem1a | 14154 | fem 1 homolog a |
| ENSMUSG00000020061.18 | 29069.43 | 2.709566 | 0.364592 | 7.431778 | 1.07E−13 | 1.22E−11 | Mybpc1 | 109272 | myosin binding protein C, slow-type |
| ENSMUSG00000054426.11 | 417.0789 | 2.69105 | 0.362293 | 7.427833 | 1.10E−13 | 1.25E−11 | A930005H10Rik | 68161 | RIKEN cDNA A930005H10 gene |
| ENSMUSG00000036052.14 | 2415.101 | 2.048086 | 0.275912 | 7.422971 | 1.15E−13 | 1.29E−11 | Dnajb5 | 56323 | DnaJ heat shock protein family (Hsp40) member B5 |
| ENSMUSG00000076596.3 | 91.86008 | −23.9295 | 3.22626 | −7.41711 | 1.20E−13 | 1.34E−11 | Igkv3-10 | 667924 | immunoglobulin kappa variable 3-10 |
| ENSMUSG00000006435.16 | 1558.82 | 2.553204 | 0.344555 | 7.410161 | 1.26E−13 | 1.40E−11 | Neurl1a | 18011 | neuralized E3 ubiquitin protein ligase 1A |
| ENSMUSG00000087090.7 | 1760.883 | 2.988544 | 0.403312 | 7.410008 | 1.26E−13 | 1.40E−11 | Nctc1 | 330677 | non-coding transcript 1 |
| ENSMUSG00000027777.15 | 2089.237 | 2.536745 | 0.342417 | 7.408339 | 1.28E−13 | 1.41E−11 | Schip1 | 30953 | schwannomin interacting protein 1 |
| ENSMUSG00000114827.1 | 14.99121 | 7.745197 | 1.046274 | 7.402649 | 1.33E−13 | 1.47E−11 | Gm46432 | 1.08E+08 | predicted gene, 46432 |
| ENSMUSG00000021768.15 | 1271.372 | 2.635457 | 0.356305 | 7.396622 | 1.40E−13 | 1.53E−11 | Dusp13 | 27389 | dual specificity phosphatase 13 |
| ENSMUSG00000022389.15 | 7571.367 | 2.01838 | 0.273089 | 7.390929 | 1.46E−13 | 1.59E−11 | Tef | 21685 | thyrotroph embryonic factor |
| ENSMUSG00000040287.9 | 2433.85 | 2.858653 | 0.386814 | 7.390257 | 1.47E−13 | 1.59E−11 | Stac3 | 237611 | SH3 and cysteine rich domain 3 |
| ENSMUSG00000038028.9 | 1799.326 | 2.573576 | 0.348274 | 7.389509 | 1.47E−13 | 1.59E−11 | Tigar | 319801 | Trp53 induced glycolysis regulatory phosphatase |
| ENSMUSG00000028700.14 | 1483.954 | 0.966362 | 0.130951 | 7.3796 | 1.59E−13 | 1.71E−11 | Pomgnt1 | 68273 | protein O-linked mannose beta 1,2-N- |
| | | | | | | | | | acetylglucosaminyltransferase |
| ENSMUSG00000076547.2 | 81.92634 | −23.7842 | 3.226301 | −7.37197 | 1.68E−13 | 1.79E−11 | NA | NA | NA |
| ENSMUSG00000092341.3 | 16870 | −1.74014 | 0.236046 | −7.37204 | 1.68E−13 | 1.79E−11 | Malat1 | 72289 | metastasis associated lung adenocarcinoma transcript 1 |
| | | | | | | | | | (non-coding RNA) |
| ENSMUSG00000029312.12 | 745.1806 | 1.114218 | 0.15127 | 7.365752 | 1.76E−13 | 1.87E−11 | Klhl8 | 246293 | kelch-like 8 |
| ENSMUSG00000021420.13 | 662.1543 | 1.136321 | 0.154302 | 7.364286 | 1.78E−13 | 1.88E−11 | Fars2 | 69955 | phenylalanine-tRNA synthetase 2 (mitochondrial) |
| ENSMUSG00000085457.3 | 602.8297 | 3.019124 | 0.410243 | 7.359361 | 1.85E−13 | 1.94E−11 | 1110046J04Rik | 68808 | RIKEN cDNA 1110046J04 gene |
| ENSMUSG00000050315.14 | 8803.38 | 2.821934 | 0.383715 | 7.35424 | 1.92E−13 | 2.01E−11 | Synpo2 | 118449 | synaptopodin 2 |
| ENSMUSG00000007033.4 | 617.5704 | 3.218039 | 0.437719 | 7.351837 | 1.96E−13 | 2.04E−11 | Hspa1l | 15482 | heat shock protein 1-like |
| ENSMUSG00000094124.5 | 77.35089 | −23.7008 | 3.226323 | −7.34608 | 2.04E−13 | 2.12E−11 | Ighv1-74 | 1.01E+08 | immunoglobulin heavy variable V1-74 |
| ENSMUSG00000034055.16 | 3270.37 | 2.344468 | 0.319206 | 7.344697 | 2.06E−13 | 2.12E−11 | Phka1 | 18679 | phosphorylase kinase alpha 1 |
| ENSMUSG00000072680.11 | 95.35552 | 23.45956 | 3.193979 | 7.344933 | 2.06E−13 | 2.12E−11 | NA | NA | NA |
| ENSMUSG00000029472.13 | 13243.15 | 1.390084 | 0.189461 | 7.337061 | 2.18E−13 | 2.23E−11 | Anapc5 | 59008 | anaphase-promoting complex subunit 5 |
| ENSMUSG00000030319.8 | 1324.461 | 2.392709 | 0.327869 | 7.297764 | 2.93E−13 | 2.98E−11 | Cand2 | 67088 | cullin-associated and neddylation-dissociated 2 |
| | | | | | | | | | (putative) |
| ENSMUSG00000018599.5 | 1422.139 | 2.112166 | 0.289634 | 7.292524 | 3.04E−13 | 3.09E−11 | Mief2 | 237781 | mitochondrial elongation factor 2 |
| ENSMUSG00000063275.15 | 780.7201 | 2.284411 | 0.313312 | 7.291162 | 3.07E−13 | 3.10E−11 | Hacd1 | 30963 | 3-hydroxyacyl-CoA dehydratase 1 |
| ENSMUSG00000020913.3 | 95.01534 | −2.35951 | 0.32403 | −7.28177 | 3.29E−13 | 3.31E−11 | Krt24 | 75706 | keratin 24 |
| ENSMUSG00000026564.9 | 597.8614 | 2.836479 | 0.390007 | 7.272887 | 3.52E−13 | 3.52E−11 | Dusp27 | 240892 | dual specificity phosphatase 27 (putative) |
| ENSMUSG00000030515.9 | 852.8663 | 2.203888 | 0.303056 | 7.272219 | 3.54E−13 | 3.53E−11 | Tarsl2 | 272396 | threonyl-tRNA synthetase-like 2 |
| ENSMUSG00000032285.15 | 2628.169 | 1.374181 | 0.189032 | 7.269569 | 3.61E−13 | 3.58E−11 | Dnaja4 | 58233 | DnaJ heat shock protein family (Hsp40) member A4 |
| ENSMUSG00000102888.1 | 64.05638 | −23.4492 | 3.226405 | −7.26789 | 3.65E−13 | 3.61E−11 | NA | NA | NA |
| ENSMUSG00000095204.6 | 109.8911 | −23.4014 | 3.226205 | −7.25352 | 4.06E−13 | 4.00E−11 | NA | NA | NA |
| ENSMUSG00000072720.9 | 2765.283 | 2.894 | 0.399234 | 7.248883 | 4.20E−13 | 4.12E−11 | Myo18b | 74376 | myosin XVIIIb |
| ENSMUSG00000063428.8 | 384.5075 | 2.379518 | 0.32829 | 7.248222 | 4.22E−13 | 4.12E−11 | Ddo | 70503 | D-aspartate oxidase |
| ENSMUSG00000083844.8 | 1258.565 | 2.561022 | 0.353466 | 7.245461 | 4.31E−13 | 4.19E−11 | Ube2d-ps | 76508 | ubiquitin-conjugating enzyme E2D, pseudogene |
| ENSMUSG00000100860.1 | 94.90087 | 4.168161 | 0.575805 | 7.238838 | 4.53E−13 | 4.38E−11 | 2010009K17Rik | 72340 | RIKEN cDNA 2010009K17 gene |
| ENSMUSG00000042258.13 | 376.694 | 3.05932 | 0.42276 | 7.236535 | 4.60E−13 | 4.44E−11 | Isl1 | 16392 | ISL1 transcription factor LIM/homeodomain |
| ENSMUSG00000043795.10 | 1542.61 | 3.183189 | 0.440215 | 7.230981 | 4.80E−13 | 4.60E−11 | Prr33 | 677289 | proline rich 33 |
| ENSMUSG00000023990.18 | 1022.217 | 1.550492 | 0.214497 | 7.22849 | 4.88E−13 | 4.67E−11 | Tfeb | 21425 | transcription factor EB |
| ENSMUSG00000071342.5 | 433.0897 | 2.467733 | 0.341524 | 7.225648 | 4.99E−13 | 4.73E−11 | Lsmem1 | 380755 | leucine-rich single-pass membrane protein 1 |
| ENSMUSG00000099411.1 | 303.8024 | 2.769959 | 0.383323 | 7.226173 | 4.97E−13 | 4.73E−11 | 2310015D24Rik | 70100 | RIKEN cDNA 2310015D24 gene |
| ENSMUSG00000043342.9 | 272.4563 | 1.878502 | 0.260193 | 7.219635 | 5.21E−13 | 4.92E−11 | Hoxd9 | 15438 | homeobox D9 |
| ENSMUSG00000010064.15 | 1985.972 | 1.940053 | 0.269236 | 7.205763 | 5.77E−13 | 5.43E−11 | Slc38a3 | 76257 | solute carrier family 38, member 3 |
| ENSMUSG00000028756.12 | 10402.43 | 1.832441 | 0.254422 | 7.202368 | 5.92E−13 | 5.54E−11 | Pink1 | 68943 | PTEN induced putative kinase 1 |
| ENSMUSG00000025216.9 | 212.0302 | 2.991811 | 0.415742 | 7.196314 | 6.19E−13 | 5.77E−11 | Lbx1 | 16814 | ladybird homeobox 1 |
| ENSMUSG00000025348.9 | 1740.723 | 1.964564 | 0.27309 | 7.193833 | 6.30E−13 | 5.85E−11 | Itga7 | 16404 | integrin alpha 7 |
| ENSMUSG00000073878.3 | 35.41776 | 22.96937 | 3.194786 | 7.189641 | 6.50E−13 | 6.01E−11 | Gm13304 | 1.01E+08 | predicted gene 13304 |
| ENSMUSG00000026027.13 | 1336.055 | 1.844541 | 0.256659 | 7.186729 | 6.64E−13 | 6.12E−11 | Stradb | 227154 | STE20-related kinase adaptor beta |
| ENSMUSG00000102676.1 | 18.15951 | 5.088069 | 0.708763 | 7.178797 | 7.03E−13 | 6.46E−11 | NA | NA | NA |
| ENSMUSG00000114407.1 | 272.6848 | −2.44738 | 0.34106 | −7.1758 | 7.19E−13 | 6.58E−11 | NA | NA | NA |
| ENSMUSG00000055493.4 | 1201.267 | 2.230303 | 0.310862 | 7.17458 | 7.25E−13 | 6.61E−11 | Epm2a | 13853 | epilepsy, progressive myoclonic epilepsy, type 2 |
| | | | | | | | | | gene alpha |
| ENSMUSG00000067719.5 | 32.24336 | 22.8291 | 3.194911 | 7.145456 | 8.97E−13 | 8.14E−11 | NA | NA | NA |
| ENSMUSG00000041476.12 | 8660.952 | 2.181109 | 0.305535 | 7.13866 | 9.42E−13 | 8.52E−11 | Smpx | 66106 | small muscle protein, X-linked |
| ENSMUSG00000055116.8 | 638.0438 | −1.77094 | 0.248367 | −7.13032 | 1.00E−12 | 9.02E−11 | Arntl | 11865 | aryl hydrocarbon receptor nuclear translocator-like |
| ENSMUSG00000019797.11 | 1053.253 | 1.635139 | 0.229401 | 7.127876 | 1.02E−12 | 9.14E−11 | Mtres1 | 67851 | mitochondrial transcription rescue factor 1 |
| ENSMUSG00000020326.7 | 13388.34 | 2.160632 | 0.303372 | 7.122045 | 1.06E−12 | 9.50E−11 | Ccng1 | 12450 | cyclin G1 |
| ENSMUSG00000102531.1 | 34.86132 | 3.520809 | 0.49447 | 7.120376 | 1.08E−12 | 9.58E−11 | NA | NA | NA |
| ENSMUSG00000030399.2 | 245518.4 | 3.062915 | 0.430426 | 7.116008 | 1.11E−12 | 9.85E−11 | Ckm | 12715 | creatine kinase, muscle |
| ENSMUSG00000027961.7 | 1301.101 | 2.633309 | 0.370271 | 7.111844 | 1.15E−12 | 1.01E−10 | Lrrc39 | 109245 | leucine rich repeat containing 39 |
| ENSMUSG00000040694.4 | 26225.84 | 3.03529 | 0.427167 | 7.105621 | 1.20E−12 | 1.05E−10 | Apobec2 | 11811 | apolipoprotein B mRNA editing enzyme, catalytic |
| | | | | | | | | | polypeptide 2 |
| ENSMUSG00000081094.4 | 43.67079 | −22.9256 | 3.226629 | −7.10513 | 1.20E−12 | 1.05E−10 | Rpl19-ps11 | 1E+08 | ribosomal protein L19, pseudogene 11 |
| ENSMUSG00000042476.12 | 1368.47 | 3.110979 | 0.438369 | 7.096717 | 1.28E−12 | 1.12E−10 | Abcb4 | 18670 | ATP-binding cassette, sub-family B (MDR/TAP), |
| | | | | | | | | | member 4 |
| ENSMUSG00000087523.1 | 161.6609 | 2.875275 | 0.405448 | 7.091601 | 1.33E−12 | 1.15E−10 | NA | NA | NA |
| ENSMUSG00000005674.9 | 1277.43 | 0.926245 | 0.130624 | 7.090935 | 1.33E−12 | 1.16E−10 | Tomm40l | 641376 | translocase of outer mitochondrial membrane 40-like |
| ENSMUSG00000038670.11 | 36451.43 | 3.019984 | 0.42615 | 7.086676 | 1.37E−12 | 1.18E−10 | Mybpc2 | 233199 | myosin binding protein C, fast-type |
| ENSMUSG00000095338.1 | 41.8135 | −22.8663 | 3.226661 | −7.08668 | 1.37E−12 | 1.18E−10 | Igkv3-9 | 667928 | immunoglobulin kappa variable 3-9 |
| ENSMUSG00000050821.14 | 630.2236 | 1.856195 | 0.262883 | 7.060925 | 1.65E−12 | 1.42E−10 | Fam131a | 78408 | family with sequence similarity 131, member A |
| ENSMUSG00000001604.14 | 2371.705 | 2.942845 | 0.416895 | 7.058954 | 1.68E−12 | 1.43E−10 | Tcea3 | 21401 | transcription elongation factor A (SII), 3 |
| ENSMUSG00000028496.17 | 2046.712 | 2.459098 | 0.348502 | 7.056199 | 1.71E−12 | 1.46E−10 | Mllt3 | 70122 | myeloid/lymphoid or mixed-lineage leukemia; |
| | | | | | | | | | translocated to, 3 |
| ENSMUSG00000080895.1 | 12.62844 | 7.514961 | 1.067335 | 7.040863 | 1.91E−12 | 1.62E−10 | NA | NA | NA |
| ENSMUSG00000025511.15 | 1050.992 | 1.799744 | 0.255743 | 7.037315 | 1.96E−12 | 1.66E−10 | Tspan4 | 64540 | tetraspanin 4 |
| ENSMUSG00000024049.15 | 16515.27 | 2.5093 | 0.356837 | 7.032059 | 2.04E−12 | 1.71E−10 | Myom1 | 17929 | myomesin 1 |
| ENSMUSG00000048175.13 | 2081.75 | 1.75658 | 0.250171 | 7.021519 | 2.19E−12 | 1.84E−10 | Asb8 | 78541 | ankyrin repeat and SOCS box-containing 8 |
| ENSMUSG00000085888.7 | 960.2303 | 2.836049 | 0.404195 | 7.016534 | 2.27E−12 | 1.90E−10 | Gm12224 | 1.08E+08 | predicted gene 12224 |
| ENSMUSG00000026208.9 | 61777.09 | 3.161995 | 0.451201 | 7.007949 | 2.42E−12 | 2.01E−10 | Des | 13346 | desmin |
| ENSMUSG00000076552.3 | 34.29945 | −22.6001 | 3.226822 | −7.00383 | 2.49E−12 | 2.07E−10 | Igkv4-61 | 546244 | immunoglobulin kappa chain variable 4-61 |
| ENSMUSG00000028420.13 | 1701.872 | 1.940063 | 0.277203 | 6.998698 | 2.58E−12 | 2.13E−10 | Tmem38b | 52076 | transmembrane protein 38B |
| ENSMUSG00000031782.15 | 3796.617 | 1.605171 | 0.229354 | 6.998655 | 2.58E−12 | 2.13E−10 | Coq9 | 67914 | coenzyme Q9 |
| ENSMUSG00000046312.4 | 1245.314 | 2.32354 | 0.33205 | 6.997557 | 2.60E−12 | 2.14E−10 | Myorg | 329828 | myogenesis regulating glycosidase (putative) |
| ENSMUSG00000007097.14 | 23544.44 | 2.465089 | 0.35263 | 6.990581 | 2.74E−12 | 2.24E−10 | Atp1a2 | 98660 | ATPase, Na+/K+ transporting, alpha 2 polypeptide |
| ENSMUSG00000028684.14 | 1808.014 | 1.882644 | 0.269574 | 6.983773 | 2.87E−12 | 2.34E−10 | Urod | 22275 | uroporphyrinogen decarboxylase |
| ENSMUSG00000031147.8 | 470.2173 | 3.381717 | 0.484195 | 6.984209 | 2.86E−12 | 2.34E−10 | Magix | 54634 | MAGI family member, X-linked |
| ENSMUSG00000020925.16 | 1149.682 | 1.280388 | 0.183431 | 6.980202 | 2.95E−12 | 2.39E−10 | Ccdc43 | 52715 | coiled-coil domain containing 43 |
| ENSMUSG00000024247.14 | 798.9727 | 1.812348 | 0.260055 | 6.969101 | 3.19E−12 | 2.57E−10 | Pkdcc | 106522 | protein kinase domain containing, cytoplasmic |
| ENSMUSG00000020722.5 | 2485.093 | 2.902191 | 0.416491 | 6.968187 | 3.21E−12 | 2.58E−10 | Cacng1 | 12299 | calcium channel, voltage-dependent, gamma subunit 1 |
| ENSMUSG00000098814.2 | 77.36414 | 21.8778 | 3.146186 | 6.95374 | 3.56E−12 | 2.85E−10 | Igkv19-93 | 692161 | immunoglobulin kappa chain variable 19-93 |
| ENSMUSG00000093954.8 | 72.80968 | −8.5549 | 1.231529 | −6.94657 | 3.74E−12 | 2.99E−10 | Gm16867 | 1.01E+08 | predicted gene, 16867 |
| ENSMUSG00000060600.15 | 77180 | 3.003258 | 0.432416 | 6.94529 | 3.78E−12 | 3.01E−10 | Eno3 | 13808 | enolase 3, beta muscle |
| ENSMUSG00000028150.14 | 2186.546 | 2.231853 | 0.321526 | 6.941439 | 3.88E−12 | 3.08E−10 | Rorc | 19885 | RAR-related orphan receptor gamma |
| ENSMUSG00000022747.17 | 2086.402 | 2.266267 | 0.326517 | 6.940732 | 3.90E−12 | 3.08E−10 | St3gal6 | 54613 | ST3 beta-galactoside alpha-2,3-sialyltransferase 6 |
| ENSMUSG00000020836.15 | 1937.086 | 2.452708 | 0.353741 | 6.933629 | 4.10E−12 | 3.23E−10 | Coro6 | 216961 | coronin 6 |
| ENSMUSG00000103254.1 | 28.78692 | −22.3641 | 3.226993 | −6.93031 | 4.20E−12 | 3.30E−10 | NA | NA | NA |
| ENSMUSG00000095260.1 | 19.26571 | 22.12233 | 3.195864 | 6.922175 | 4.45E−12 | 3.48E−10 | NA | NA | NA |
| ENSMUSG00000093843.1 | 19.27326 | 22.1201 | 3.195863 | 6.921479 | 4.47E−12 | 3.49E−10 | NA | NA | NA |
| ENSMUSG00000020623.11 | 304.0646 | 1.415555 | 0.204616 | 6.918106 | 4.58E−12 | 3.56E−10 | Map2k6 | 26399 | mitogen-activated protein kinase kinase 6 |
| ENSMUSG00000116879.1 | 106.8637 | 1.525238 | 0.220564 | 6.915175 | 4.67E−12 | 3.62E−10 | NA | NA | NA |
| ENSMUSG00000055912.8 | 574.366 | 1.730574 | 0.250479 | 6.909052 | 4.88E−12 | 3.77E−10 | Tmem150a | 232086 | transmembrane protein 150A |
| ENSMUSG00000032355.16 | 2570.634 | 2.777834 | 0.402103 | 6.908266 | 4.91E−12 | 3.78E−10 | Mlip | 69642 | muscular LMNA-interacting protein |
| ENSMUSG00000035606.8 | 2677.306 | 3.250067 | 0.471232 | 6.89696 | 5.31E−12 | 4.08E−10 | Ky | 16716 | kyphoscoliosis peptidase |
| ENSMUSG00000095700.2 | 26.52019 | −22.252 | 3.227085 | −6.89539 | 5.37E−12 | 4.11E−10 | Ighv10-3 | 380809 | immunoglobulin heavy variable V10-3 |
| ENSMUSG00000104769.1 | 24.6387 | −22.1506 | 3.227174 | −6.86378 | 6.71E−12 | 5.11E−10 | Igkv8-34 | 620126 | immunoglobulin kappa variable 8-34 |
| ENSMUSG00000031633.4 | 59468.28 | 2.837582 | 0.413514 | 6.862113 | 6.78E−12 | 5.16E−10 | Slc25a4 | 11739 | solute carrier family 25 (mitochondrial carrier, adenine |
| | | | | | | | | | nucleotide translocator), member 4 |
| ENSMUSG00000069939.8 | 718.2112 | 3.451335 | 0.503123 | 6.859828 | 6.89E−12 | 5.22E−10 | NA | NA | NA |
| ENSMUSG00000083773.4 | 28.79443 | 8.686819 | 1.267151 | 6.855393 | 7.11E−12 | 5.37E−10 | Gm13394 | 1E+08 | glyceraldehyde-3-phosphate dehydrogenase pseudogene |
| ENSMUSG00000019088.14 | 1961.732 | 1.581967 | 0.231488 | 6.833911 | 8.26E−12 | 6.22E−10 | Dnase1l1 | 69537 | deoxyribonuclease 1-like 1 |
| ENSMUSG00000057003.12 | 160700.8 | 3.013963 | 0.441385 | 6.82842 | 8.59E−12 | 6.44E−10 | Myh4 | 17884 | myosin, heavy polypeptide 4, skeletal muscle |
| ENSMUSG00000028957.12 | 1768.47 | 1.443753 | 0.211482 | 6.826853 | 8.68E−12 | 6.49E−10 | Per3 | 18628 | period circadian clock 3 |
| ENSMUSG00000094787.2 | 22.54216 | −22.025 | 3.227289 | −6.8246 | 8.82E−12 | 6.57E−10 | NA | NA | NA |
| ENSMUSG00000036099.16 | 1475.669 | 1.458557 | 0.21385 | 6.820456 | 9.08E−12 | 6.73E−10 | Vezt | 215008 | vezatin, adherens junctions transmembrane protein |
| ENSMUSG00000082029.1 | 22.18272 | −22.0112 | 3.227311 | −6.82029 | 9.09E−12 | 6.73E−10 | NA | NA | NA |
| ENSMUSG00000064370.1 | 318237.9 | 1.778175 | 0.261047 | 6.811696 | 9.65E−12 | 7.12E−10 | CYTB | 17711 | cytochrome b |
| ENSMUSG00000013419.7 | 1557.726 | 1.908352 | 0.28031 | 6.808003 | 9.90E−12 | 7.28E−10 | Zfp651 | 270210 | zinc finger protein 651 |
| ENSMUSG00000030695.16 | 308771.2 | 3.068523 | 0.450961 | 6.804412 | 1.01E−11 | 7.44E−10 | Aldoa | 11674 | aldolase A, fructose-bisphosphate |
| ENSMUSG00000100783.1 | 128.6221 | 2.812656 | 0.413452 | 6.802867 | 1.03E−11 | 7.50E−10 | NA | NA | NA |
| ENSMUSG00000087408.10 | 120.3444 | 2.800718 | 0.411814 | 6.80093 | 1.04E−11 | 7.58E−10 | Gdf1 | 14559 | growth differentiation factor 1 |
| ENSMUSG00000002104.11 | 417.6794 | 2.751876 | 0.404851 | 6.797262 | 1.07E−11 | 7.75E−10 | Rapsn | 19400 | receptor-associated protein of the synapse |
| ENSMUSG00000096410.2 | 20.82038 | −21.9256 | 3.227402 | −6.79359 | 1.09E−11 | 7.92E−10 | NA | NA | NA |
| ENSMUSG00000042529.14 | 419.1761 | 2.769993 | 0.407793 | 6.792647 | 1.10E−11 | 7.95E−10 | Kcnj12 | 16515 | potassium inwardly-rectifying channel, subfamily J, |
| | | | | | | | | | member 12 |
| ENSMUSG00000036199.9 | 7762.181 | 2.146262 | 0.316017 | 6.791609 | 1.11E−11 | 7.98E−10 | Ndufa13 | 67184 | NADH:ubiquinone oxidoreductase subunit A13 |
| ENSMUSG00000064346.1 | 42.09662 | 2.591091 | 0.382228 | 6.778919 | 1.21E−11 | 8.69E−10 | NA | NA | NA |
| ENSMUSG00000039103.12 | 3596.534 | 2.777482 | 0.410086 | 6.772925 | 1.26E−11 | 9.03E−10 | Nexn | 68810 | nexilin |

TABLE 11
(Supplementary Table S6a. Ranked genes by differential methylation
(Old + OSKM vs Old-1): RANK comp., p-value comp., q-value comp.)

| | RANK | p-value | q-value | p-value | p-value | p-value | p-value | p-value |
|---|---|---|---|---|---|---|---|---|
| Gene | comp. | comp. | comp. | comp. TR1 | comp. TR2 | comp. TR3 | comp. TR4 | comp. TR5 |
| Peg12 | 1 | 2.22E−16 | 1.23E−12 | 0.000135 | 1.36E−05 | 1.25E−05 | 2.24E−05 | 0.000302 |
| Hoxa9 | 1 | 2.22E−16 | 1.23E−12 | 0 | 5.86E−05 | 0.003868 | 9.77E−06 | 5.86E−05 |
| Hoxaas3 | 3 | 2.22E−16 | 1.23E−12 | 0 | 0.000401 | 0.00074 | 0.109089 | 0.004308 |
| Fam83g | 4 | 2.22E−16 | 1.23E−12 | 0.178204 | 0 | 0.070355 | 0.296622 | 0.002159 |
| Tbx3 | 5 | 4.20E−15 | 1.86E−11 | 4.88E−05 | 0.000205 | 0.000166 | 5.86E−05 | 0.000225 |
| Hoxa2 | 6 | 7.57E−12 | 2.70E−08 | 1.95E−05 | 0.000283 | 0.000381 | 0.145488 | 0.000283 |
| Hoxd3 | 7 | 9.27E−12 | 2.70E−08 | 0.001036 | 0.001876 | 4.88E−05 | 0.002452 | 0.000469 |
| Gm10377 | 8 | 9.77E−12 | 2.70E−08 | 0.000787 | 0.000478 | 0.000802 | 0.000436 | 0.000881 |
| Tbx5 | 9 | 2.66E−11 | 5.51E−08 | 0.006995 | 0.005002 | 0.000166 | 0.000625 | 9.77E−05 |
| Hoxb3 | 10 | 2.73E−11 | 5.51E−08 | 0.002911 | 0.000469 | 7.82E−05 | 0.001407 | 0.002432 |
| Gm3772 | 11 | 2.91E−11 | 5.51E−08 | 3.36E−05 | 2.15E−05 | 0.002843 | 0.001993 | 0.095627 |
| Rbfox1 | 12 | 2.98E−11 | 5.51E−08 | 5.82E−07 | 0.060674 | 0.099965 | 6.57E−06 | 0.017428 |
| Hoxa3 | 13 | 3.87E−11 | 6.59E−08 | 1.95E−05 | 0.000234 | 0.000518 | 0.220366 | 0.001036 |
| Oxct2a | 14 | 1.12E−10 | 1.78E−07 | 0.000284 | 0.003004 | 0.010304 | 7.92E−05 | 0.002577 |
| Nfix | 15 | 2.22E−10 | 3.27E−07 | 0.000371 | 3.91E−05 | 0.002767 | 0.025868 | 0.003732 |
| Emilin2 | 16 | 2.76E−10 | 3.70E−07 | 0.000645 | 0.002931 | 0.003966 | 0.000166 | 0.003986 |
| 4933406F0 | 17 | 2.84E−10 | 3.70E−07 | 8.80E−05 | 0.006854 | 0.022477 | 0.000555 | 0.00068 |
| 9Rik | | | | | | | | |
| Adcy2 | 18 | 6.83E−10 | 8.41E−07 | 0.004279 | 0.000283 | 0.002315 | 0.000635 | 0.007805 |
| Hic1 | 19 | 8.63E−10 | 1.00E−06 | 0.033292 | 2.93E−05 | 0.001641 | 0.001036 | 0.010941 |
| Tescl | 20 | 9.05E−10 | 1.00E−06 | 6.39E−06 | 0.007646 | 0.017199 | 4.84E−05 | 0.470825 |
| Gemin5 | 21 | 1.77E−09 | 1.82E−06 | 0.000674 | 0.000164 | 0.014272 | 5.86E−05 | 0.444894 |
| Gm45351 | 22 | 1.81E−09 | 1.82E−06 | 0.00029 | 0.001571 | 0.008921 | 0.001055 | 0.009869 |
| Vmn2r47 | 23 | 1.90E−09 | 1.83E−06 | 0.00029 | 0.001646 | 0.009146 | 0.001055 | 0.009705 |
| Tent4a | 24 | 2.21E−09 | 2.04E−06 | 0.003868 | 0.001133 | 0.000635 | 0.002738 | 0.006965 |
| Mir196b | 25 | 2.45E−09 | 2.17E−06 | 0.001858 | 0.001776 | 0.009845 | 0.001698 | 0.001081 |
| Ly6l | 26 | 3.07E−09 | 2.62E−06 | 0.00161 | 0.002748 | 0.001696 | 0.004449 | 0.002319 |
| Defa23 | 27 | 7.30E−09 | 5.99E−06 | 0.004233 | 0.00044 | 0.002814 | 0.001387 | 0.02876 |
| Mup2 | 28 | 8.11E−09 | 6.42E−06 | 0.00821 | 0.002765 | 0.001001 | 0.003354 | 0.003101 |
| Skint5 | 29 | 9.19E−09 | 7.02E−06 | 0.002748 | 0.019535 | 0.002391 | 0.001578 | 0.001347 |
| Tmem94 | 30 | 1.00E−08 | 7.39E−06 | 0.001631 | 0.002364 | 0.003116 | 0.003878 | 0.006457 |
| Fktn | 31 | 1.08E−08 | 7.74E−06 | 0.001436 | 0.006721 | 0.000557 | 0.002159 | 0.028427 |
| Pitx2 | 32 | 1.44E−08 | 9.95E−06 | 0.014302 | 0.001895 | 0.001221 | 0.001631 | 0.008479 |
| Septin9 | 33 | 1.89E−08 | 1.27E−05 | 0.00295 | 0.011713 | 0.001299 | 0.000615 | 0.022683 |
| Mir6991 | 34 | 2.02E−08 | 1.31E−05 | 0.026264 | 0.011133 | 0.005376 | 0.00258 | 0.000167 |
| Gltp | 35 | 2.15E−08 | 1.36E−05 | 0.005383 | 0.002237 | 0.034015 | 0.003888 | 0.000459 |
| Fxyd1 | 36 | 2.47E−08 | 1.51E−05 | 0.003116 | 0.001075 | 0.164049 | 0.000225 | 0.006946 |
| Ebf1 | 37 | 2.53E−08 | 1.51E−05 | 0.011644 | 0.000195 | 0.018805 | 0.00169 | 0.012182 |
| Col28a1 | 38 | 2.69E−08 | 1.57E−05 | 0.000224 | 0.012303 | 0.0002 | 0.069403 | 0.024734 |
| Rnf39 | 39 | 3.30E−08 | 1.88E−05 | 0.010453 | 0.000674 | 0.000801 | 0.011508 | 0.018502 |
| Galnt10 | 40 | 4.17E−08 | 2.31E−05 | 0.000308 | 0.001622 | 0.155911 | 8.73E−05 | 0.232138 |
| Gal3st2c | 41 | 5.37E−08 | 2.90E−05 | 0.008948 | 0.001983 | 0.018913 | 0.013178 | 0.000479 |
| Pax1 | 42 | 5.63E−08 | 2.97E−05 | 0.002071 | 0.032472 | 0.000391 | 0.009046 | 0.009398 |
| Mapk4 | 43 | 6.12E−08 | 3.15E−05 | 0.005402 | 0.00295 | 0.014282 | 0.006721 | 0.001612 |
| Rnf165 | 44 | 6.53E−08 | 3.24E−05 | 0.004259 | 0.007307 | 0.003995 | 0.005392 | 0.003966 |
| Prxl2c | 45 | 6.58E−08 | 3.24E−05 | 0.038181 | 0.002354 | 0.00083 | 0.012094 | 0.00297 |
| Ahdc1 | 46 | 7.44E−08 | 3.58E−05 | 0.003947 | 0.003585 | 0.023465 | 0.003048 | 0.003058 |
| Esyt2 | 47 | 8.25E−08 | 3.89E−05 | 0.000469 | 0.000625 | 0.00887 | 0.007971 | 0.168533 |
| Skint6 | 48 | 9.06E−08 | 4.18E−05 | 0.004241 | 0.016457 | 0.008331 | 0.002772 | 0.002417 |
| Zfta | 49 | 1.05E−07 | 4.71E−05 | 0.050632 | 0.045806 | 0.009867 | 0.003458 | 5.86E−05 |
| Tango6 | 50 | 1.06E−07 | 4.71E−05 | 0.011216 | 0.056254 | 0.002384 | 0.006672 | 0.000469 |
| Cldn8 | 51 | 1.15E−07 | 5.01E−05 | 0.00202 | 0.005182 | 0.004003 | 0.034432 | 0.003591 |
| Stx1b | 52 | 1.31E−07 | 5.56E−05 | 0.004464 | 0.008948 | 0.002159 | 0.009329 | 0.007434 |
| Adcy7 | 53 | 1.42E−07 | 5.94E−05 | 0.002091 | 0.000899 | 0.012641 | 0.053768 | 0.005178 |
| Hoxa7 | 54 | 1.46E−07 | 6.00E−05 | 0.00382 | 0.005207 | 0.011713 | 0.008206 | 0.003575 |
| Nod2 | 55 | 1.52E−07 | 6.11E−05 | 0.00126 | 0.001641 | 0.007141 | 0.010589 | 0.04566 |
| 3110070M | 56 | 1.70E−07 | 6.65E−05 | 0.006737 | 0.00614 | 0.004695 | 0.001617 | 0.02599 |
| 22Rik | | | | | | | | |
| Col16a1 | 57 | 1.71E−07 | 6.65E−05 | 0.004591 | 0.001232 | 0.021443 | 0.004142 | 0.016363 |
| Irx3 | 58 | 1.76E−07 | 6.72E−05 | 0.001172 | 0.000371 | 0.025956 | 0.554286 | 0.001358 |
| Tdpoz5 | 59 | 1.80E−07 | 6.74E−05 | 0.004851 | 0.00616 | 0.020272 | 0.003267 | 0.004399 |
| Tmem267 | 60 | 2.25E−07 | 8.30E−05 | 0.01816 | 0.000303 | 0.017564 | 0.001143 | 0.1027 |
| Atp6v1c2 | 61 | 2.31E−07 | 8.39E−05 | 0.07014 | 0.012582 | 0.03172 | 1.95E−05 | 0.021404 |
| Hspa1a | 62 | 2.68E−07 | 9.59E−05 | 0.003105 | 0.000128 | 0.001022 | 0.289874 | ND |
| Gna14 | 63 | 2.80E−07 | 9.85E−05 | 0.006496 | 0.005783 | 0.019333 | 0.003888 | 0.005207 |
| Cyp3a41b | 64 | 3.11E−07 | 0.000107 | 0.019842 | 0.011093 | 0.008999 | 0.00539 | 0.001559 |
| Jak3 | 65 | 3.14E−07 | 0.000107 | 0.01564 | 0.000127 | 0.114784 | 0.006106 | 0.012094 |
| 4930578G1 | 66 | 3.25E−07 | 0.000109 | 0.024874 | 0.00797 | 0.002662 | 0.004779 | 0.006958 |
| 0Rik | | | | | | | | |
| Man1c1 | 67 | 3.42E−07 | 0.000113 | 0.004308 | 0.002999 | 0.007258 | 0.028926 | 0.006868 |
| Ubash3b | 68 | 3.50E−07 | 0.000114 | 0.013081 | 0.008733 | 0.002257 | 0.002618 | 0.028379 |
| Mir196a-1 | 69 | 3.87E−07 | 0.000124 | 0.009254 | 0.005347 | 0.007478 | 0.004925 | 0.01181 |
| Caskin1 | 70 | 4.13E−07 | 0.000131 | 0.032276 | 0.008274 | 0.025702 | 0.008678 | 0.000391 |
| Zfp617 | 71 | 4.18E−07 | 0.000131 | 0.025077 | 0.003204 | 0.037776 | 9.77E−05 | 0.079694 |
| Zfp111 | 72 | 4.41E−07 | 0.000134 | 0.00043 | 0.048395 | 0.014663 | 0.000166 | 0.496571 |
| Rapgefl1 | 73 | 4.42E−07 | 0.000134 | 0.049899 | 0.007923 | 0.002989 | 0.003146 | 0.00678 |
| 4930558F1 | 74 | 4.63E−07 | 0.000139 | 0.012852 | 0.003434 | 0.029956 | 0.002518 | 0.008001 |
| 7Rik | | | | | | | | |
| Zfp46 | 75 | 4.96E−07 | 0.000147 | 0.00804 | 0.003566 | 0.036506 | 0.000691 | 0.040013 |
| Gm16513 | 76 | 5.03E−07 | 0.000147 | 0.006721 | 0.019437 | 0.002742 | 0.014223 | 0.005777 |
| Zfp872 | 77 | 6.32E−07 | 0.000182 | 0.003204 | 0.00591 | 0.007346 | 0.041948 | 0.006604 |
| Umad1 | 78 | 6.65E−07 | 0.000188 | 0.000948 | 0.035627 | 0.001192 | 0.017945 | 0.056796 |
| Marveld2 | 79 | 6.70E−07 | 0.000188 | 0.003947 | 0.004611 | 0.005324 | 0.004797 | 0.088975 |
| Col1a1 | 80 | 7.32E−07 | 0.000203 | 0.152316 | 0.000801 | 0.003956 | 0.032911 | 0.002892 |
| Dock1 | 81 | 8.07E−07 | 0.000221 | 0.007082 | 0.027861 | 0.008831 | 0.004308 | 0.006877 |
| Rassf3 | 82 | 8.37E−07 | 0.000226 | 0.017076 | 0.00126 | 0.007805 | 0.013149 | 0.024403 |
| Zbtb11os1 | 83 | 8.55E−07 | 0.000228 | 0.006841 | 0.005566 | 0.022979 | 0.00552 | 0.011451 |
| Dchs1 | 84 | 8.81E−07 | 0.000232 | 0.102261 | 0.00213 | 0.000684 | 0.001065 | 0.361109 |
| Smad7 | 85 | 8.94E−07 | 0.000233 | 0.004687 | 0.010433 | 0.011762 | 0.00844 | 0.012006 |
| Atp6v1b2 | 86 | 9.39E−07 | 0.000241 | 0.00211 | 0.017818 | 0.017135 | 0.023719 | 0.004044 |
| Gm38499 | 87 | 9.47E−07 | 0.000241 | 0.000205 | 0.192769 | 0.032862 | 5.86E−05 | 0.819813 |
| Ptprd | 88 | 1.00E−06 | 0.000252 | 0.009222 | 0.302767 | 1.42E−05 | 0.164088 | 0.010277 |
| Crybb3 | 89 | 1.04E−06 | 0.000259 | 0.00929 | 0.00718 | 0.00889 | 0.01268 | 0.00929 |
| Kcnn4 | 90 | 1.17E−06 | 0.000287 | 0.006683 | 0.006037 | 0.022372 | 0.001576 | 0.056288 |
| Lsp1 | 91 | 1.30E−06 | 0.000316 | 0.036164 | 0.000742 | 0.013452 | 0.007444 | 0.033908 |
| Osr2 | 92 | 1.36E−06 | 0.000329 | 0.004611 | 0.017916 | 0.007805 | 0.007249 | 0.020681 |
| Ctnnd2 | 93 | 1.55E−06 | 0.000368 | 0.001172 | 0.001915 | 0.000742 | 0.359397 | 0.187611 |
| 4930521O1 | 94 | 1.56E−06 | 0.000368 | 0.070314 | 0.0048 | 0.01719 | 0.003369 | 0.005808 |
| 1Rik | | | | | | | | |
| Bmp8a | 95 | 1.59E−06 | 0.000372 | 0.002882 | 0.022195 | 0.044829 | 0.001876 | 0.021658 |
| Clmp | 96 | 1.67E−06 | 0.000381 | 0.069379 | 0.005822 | 0.023611 | 0.006184 | 0.002091 |
| Syt14 | 97 | 1.69E−06 | 0.000381 | 0.000205 | 0.419026 | 0.001817 | 0.009603 | 0.083387 |
| Gm53 | 98 | 1.70E−06 | 0.000381 | 0.015249 | 0.006565 | 0.020886 | 0.003868 | 0.015581 |
| Tbc1d30 | 99 | 1.70E−06 | 0.000381 | 0.115458 | 0.000225 | 0.000528 | 0.074341 | 0.123928 |
| Ptprf | 100 | 1.81E−06 | 0.000401 | 0.01478 | 0.006877 | 0.003888 | 0.028503 | 0.012035 |
| Dap | 101 | 1.86E−06 | 0.000409 | 0.00297 | 0.003419 | 0.004865 | 0.0262 | 0.10864 |
| Mmp2 | 102 | 1.93E−06 | 0.000419 | 0.004162 | 0.002257 | 0.085018 | 0.014722 | 0.012475 |
| Edn2 | 103 | 2.00E−06 | 0.00043 | 0.005783 | 0.011557 | 0.057246 | 0.006545 | 0.006106 |
| Gm4793 | 104 | 2.07E−06 | 0.000441 | 0.009769 | 0.058916 | 0.004992 | 0.022087 | 0.002511 |
| Gm14326 | 105 | 2.09E−06 | 0.000441 | 0.004083 | 0.005969 | 0.01988 | 0.036985 | 0.008997 |
| Zfhx2os | 106 | 2.30E−06 | 0.000481 | 0.020222 | 0.031964 | 0.006115 | 0.00382 | 0.011986 |
| Phldb1 | 107 | 2.33E−06 | 0.000483 | 0.032296 | 0.002882 | 0.020153 | 0.010609 | 0.009261 |
| Adamts18 | 108 | 2.40E−06 | 0.000491 | 0.000801 | 0.004005 | 0.036155 | 0.050788 | 0.032267 |
| Herc6 | 109 | 2.46E−06 | 0.000501 | 0.00801 | 0.006555 | 0.124573 | 0.002696 | 0.011156 |
| Gm3250 | 110 | 2.52E−06 | 0.000502 | 0.002842 | 0.003927 | 0.031707 | 0.036362 | 0.015693 |
| Asap3 | 111 | 2.54E−06 | 0.000502 | 0.021345 | 0.021902 | 0.033165 | 0.000193 | 0.06802 |
| Dtx3 | 112 | 2.54E−06 | 0.000502 | 0.008782 | 0.000925 | 0.034064 | 0.125432 | 0.005871 |
| Mir6899 | 113 | 2.57E−06 | 0.000503 | 0.005149 | 0.014742 | 0.007132 | 0.027741 | 0.013764 |
| Snx20 | 114 | 2.62E−06 | 0.000506 | 0.012592 | 0.012485 | 0.015894 | 0.024256 | 0.003487 |
| Cdc14b | 115 | 2.64E−06 | 0.000506 | 0.024872 | 0.044028 | 0.009446 | 0.000469 | 0.044146 |
| Cep85 | 116 | 2.65E−06 | 0.000506 | 0.132036 | 0.04566 | 0.000205 | 0.023924 | 0.007258 |
| Hoxb3os | 117 | 2.67E−06 | 0.000506 | 0.00801 | 0.050056 | 0.00041 | 0.006496 | 0.202948 |
| Mab21l2 | 118 | 2.76E−06 | 0.000517 | 0.003473 | 0.001568 | 0.014278 | 0.054184 | 0.053447 |
| Gpc2 | 119 | 2.78E−06 | 0.000517 | 0.006897 | 0.004884 | 0.004308 | 0.003263 | 0.479818 |
| Defa34 | 120 | 3.02E−06 | 0.000557 | 0.01069 | 0.000718 | ND | 0.007026 | 0.035649 |
| Mmp16 | 121 | 3.06E−06 | 0.00056 | 0.000586 | 0.075806 | 0.009261 | 0.097024 | 0.006389 |
| Aldoart1 | 122 | 3.28E−06 | 0.000595 | 0.007593 | 0.010728 | 0.007094 | 0.01933 | 0.024845 |
| Gm3002 | 123 | 3.38E−06 | 0.000605 | 0.021309 | 0.016342 | 0.001314 | 0.04126 | 0.015267 |
| E330014E1 | 124 | 3.39E−06 | 0.000605 | 0.006498 | 0.006114 | 0.066748 | 0.008798 | 0.012376 |
| 0Rik | | | | | | | | |
| Fto | 125 | 3.42E−06 | 0.000606 | 0.001114 | 0.00592 | 0.079724 | 0.041508 | 0.013393 |
| Tox3 | 126 | 3.47E−06 | 0.000606 | 0.00888 | 0.024932 | 0.018825 | 0.016695 | 0.004279 |
| Skint3 | 127 | 3.51E−06 | 0.000606 | 0.001915 | 0.100961 | 0.004308 | 0.014106 | 0.025673 |
| Cmbl | 128 | 3.52E−06 | 0.000606 | 0.000283 | 0.002696 | 0.008518 | 0.268243 | 0.17329 |
| Ephb4 | 129 | 3.53E−06 | 0.000606 | 0.050173 | 0.054999 | 0.007131 | 0.004992 | 0.003087 |
| Podnl1 | 130 | 3.56E−06 | 0.000606 | 0.000694 | 0.003702 | 0.007112 | 0.048815 | 0.343874 |
| Mdk | 131 | 3.62E−06 | 0.000613 | 0.142405 | 0.002769 | 0.022818 | 0.1125 | 0.000309 |
| Hoxd4 | 132 | 3.81E−06 | 0.000633 | 0.052625 | 0.010316 | 0.005246 | 0.012162 | 0.009613 |
| Fam241b | 133 | 3.82E−06 | 0.000633 | 0.001524 | 0.01266 | 0.034719 | 0.008626 | 0.057734 |
| Nr4a1 | 134 | 3.84E−06 | 0.000633 | 0.01099 | 0.00254 | 0.026855 | 0.030967 | 0.014497 |
| Glcci1 | 135 | 3.86E−06 | 0.000633 | 0.002813 | 0.037366 | 0.001856 | 0.022234 | 0.077956 |
| Fyb | 136 | 3.89E−06 | 0.000633 | 0.020505 | 0.004787 | 0.044663 | 0.042123 | 0.001846 |
| Dera | 137 | 4.21E−06 | 0.00068 | 0.002589 | 0.022752 | 0.007581 | 0.021482 | 0.039144 |
| Lgals4 | 138 | 4.27E−06 | 0.000685 | 0.006125 | 0.003087 | 0.106051 | 0.039417 | 0.004836 |
| Lpcat2 | 139 | 4.36E−06 | 0.000694 | 0.006281 | 0.004484 | 0.009085 | 0.078004 | 0.019626 |
| Sema4b | 140 | 4.40E−06 | 0.000696 | 0.037102 | 0.014497 | 0.028418 | 0.008196 | 0.003165 |
| Skint11 | 141 | 4.61E−06 | 0.000724 | 0.007138 | 0.019319 | 0.000705 | 0.156496 | 0.02753 |
| Rgl2 | 142 | 4.72E−06 | 0.000737 | 0.00126 | 0.033947 | 0.013129 | 0.021482 | 0.035793 |
| Lgals6 | 143 | 4.76E−06 | 0.000738 | 0.00776 | 0.002481 | 0.105841 | 0.06109 | 0.003505 |
| Vmn1r252 | 144 | 4.87E−06 | 0.000748 | ND | 0.002693 | 0.002127 | ND | 0.005032 |
| Chd9 | 145 | 4.89E−06 | 0.000748 | 0.000488 | 0.006965 | 0.019284 | 0.126468 | 0.054364 |
| Slc7a11 | 146 | 4.94E−06 | 0.00075 | 0.013081 | 0.056982 | 0.067376 | 0.00421 | 0.002159 |
| Mir142b | 147 | 4.98E−06 | 0.00075 | 0.026291 | 0.00913 | 0.011994 | 0.001259 | 0.1272 |
| 5830417I10 | 148 | 5.01E−06 | 0.00075 | 0.004757 | 0.034113 | 0.005969 | 0.044345 | 0.010804 |
| Rik | | | | | | | | |
| Morf4l1- | 149 | 5.20E−06 | 0.000773 | 0.002098 | 0.007419 | 0.017631 | 0.01323 | ND |
| ps1 | | | | | | | | |
| Ctif | 150 | 5.32E−06 | 0.000786 | 0.106188 | 0.048835 | 0.015357 | 0.002432 | 0.002579 |
| Oxct2b | 151 | 5.37E−06 | 0.000787 | 0.028802 | 0.000735 | 0.014285 | 0.297347 | 0.005607 |
| Fam110a | 152 | 5.46E−06 | 0.000796 | 0.022068 | 0.023123 | 0.00382 | 0.004132 | 0.063976 |
| Skint4 | 153 | 6.11E−06 | 0.000879 | 0.006062 | 0.042001 | 0.010874 | 0.002177 | 0.097964 |
| Serpina3n | 154 | 6.11E−06 | 0.000879 | 0.007532 | 0.000948 | 0.021599 | 0.073227 | 0.052371 |
| Fam171a2 | 155 | 6.28E−06 | 0.000898 | 0.000147 | 0.051384 | 0.003546 | 0.145234 | 0.15766 |
| Csf2ra | 156 | 6.37E−06 | 0.000904 | 0.00043 | 0.001133 | 0.111004 | 0.096438 | 0.119122 |
| Spata22 | 157 | 6.42E−06 | 0.000905 | 0.008186 | 0.164488 | 0.004425 | 0.026659 | 0.003947 |
| Atp1a3 | 158 | 6.74E−06 | 0.000941 | 0.001543 | 0.184876 | 0.048825 | 0.008479 | 0.005637 |
| Colgalt1 | 159 | 6.76E−06 | 0.000941 | 6.84E−05 | 0.308647 | 0.003107 | 0.065256 | 0.156067 |
| Zfp791 | 160 | 6.90E−06 | 0.000956 | 0.003355 | 0.122726 | 0.116845 | 0.005676 | 0.002511 |
| D930048N | 161 | 6.96E−06 | 0.000958 | 0.196012 | 0.221314 | 0.004611 | 0.001885 | 0.001837 |
| 14Rik | | | | | | | | |
| Itgb4 | 162 | 7.51E−06 | 0.001027 | 0.057363 | 0.071791 | 0.003546 | 0.010609 | 0.004904 |
| Ttc14 | 163 | 7.68E−06 | 0.00104 | 0.028418 | 0.008851 | 0.013286 | 0.014263 | 0.016392 |
| Aif1 | 164 | 7.70E−06 | 0.00104 | 0.05368 | 0.013598 | 0.00549 | 0.011166 | 0.017506 |
| Ctsl | 165 | 7.80E−06 | 0.001047 | 0.024168 | 0.015055 | 0.019538 | 0.02113 | 0.005295 |
| Mir142 | 166 | 7.86E−06 | 0.001048 | 0.029317 | 0.010178 | 0.013607 | 0.001447 | 0.136598 |
| Odf3l1 | 167 | 8.29E−06 | 0.00109 | 0.006496 | 0.047545 | 0.00337 | 0.035754 | 0.023035 |
| Gvin2 | 168 | 8.31E−06 | 0.00109 | 0.001176 | 0.097528 | 0.0013 | 0.014529 | 0.39716 |
| Gvin-ps2 | 168 | 8.31E−06 | 0.00109 | 0.001176 | 0.097528 | 0.0013 | 0.014529 | 0.39716 |
| Irgc1 | 170 | 8.66E−06 | 0.001128 | 0.01414 | 0.002012 | 0.001358 | 0.031662 | 0.738868 |
| 5730596B2 | 171 | 8.74E−06 | 0.001129 | 0.001693 | 0.01429 | 0.010749 | 0.080747 | 0.043545 |
| 0Rik | | | | | | | | |
| Stag3 | 172 | 8.79E−06 | 0.001129 | 0.006975 | 0.001543 | 0.011264 | 0.013266 | 0.571997 |
| Nyap1 | 173 | 8.82E−06 | 0.001129 | 0.065608 | 0.043169 | 0.001016 | 0.234697 | 0.001369 |
| Gdnf | 174 | 8.87E−06 | 0.001129 | 0.009827 | 0.021013 | 0.051609 | 0.010609 | 0.008235 |
| Dppa1 | 175 | 9.08E−06 | 0.001148 | 0.011371 | 0.001666 | 0.172645 | 0.001899 | 0.15426 |
| Cdkn1c | 176 | 9.12E−06 | 0.001148 | 0.111836 | 0.016179 | 0.001623 | 0.00239 | ND |
| Tecr | 177 | 9.18E−06 | 0.001148 | 0.00847 | 0.000703 | 0.143202 | 0.05916 | 0.019225 |
| Fignl2 | 178 | 9.53E−06 | 0.001185 | 0.005754 | 0.000469 | 0.001192 | 0.47336 | 0.66737 |
| Mef2b | 179 | 9.60E−06 | 0.001188 | 0.017326 | 0.607323 | 0.025643 | 0.043247 | 8.79E−05 |
| E230032D2 | 180 | 1.01E−05 | 0.001246 | 0.009261 | 0.031065 | 0.057519 | 0.01098 | 0.006027 |
| 3Rik | | | | | | | | |
| Trim47 | 181 | 1.02E−05 | 0.001253 | 0.114257 | 0.002091 | 0.005764 | 0.007288 | 0.110593 |
| Adamts12 | 182 | 1.06E−05 | 0.001282 | 0.317547 | 0.002227 | 0.111238 | 0.00593 | 0.002472 |
| Pnma8b | 183 | 1.06E−05 | 0.001282 | 0.020906 | 0.006838 | 0.043843 | 0.002169 | 0.085077 |
| Skint7 | 184 | 1.08E−05 | 0.001296 | 0.004111 | 0.008954 | 0.044075 | 0.007112 | 0.10223 |
| Mdga1 | 185 | 1.09E−05 | 0.001299 | 0.006487 | 0.001162 | 0.300363 | 0.030176 | 0.017437 |
| Zcchc24 | 186 | 1.12E−05 | 0.001327 | 0.050648 | 0.021824 | 0.001299 | 0.075758 | 0.011381 |
| 5730507C0 | 187 | 1.12E−05 | 0.001327 | 0.01163 | 0.004425 | 0.012758 | ND | 0.013628 |
| 1Rik | | | | | | | | |
| Cbx8 | 188 | 1.15E−05 | 0.001351 | 0.001299 | 0.006965 | 0.021833 | 0.071791 | 0.089962 |
| Ankrd33b | 189 | 1.15E−05 | 0.001351 | 0.002472 | 0.037747 | 0.002774 | 0.062218 | 0.079792 |
| Psg16 | 190 | 1.16E−05 | 0.001351 | 0.010873 | 0.05495 | 0.012289 | 0.006193 | 0.028398 |
| Tcap | 191 | 1.18E−05 | 0.00136 | 0.046707 | ND | 0.025292 | 0.004875 | 0.001651 |
| Nim1k | 192 | 1.18E−05 | 0.00136 | 0.013139 | 0.00464 | 0.098167 | 0.007903 | 0.028046 |
| 1700001C1 | 193 | 1.18E−05 | 0.00136 | 0.327126 | 0.002022 | 0.002384 | 0.531876 | 0.001583 |
| 9Rik | | | | | | | | |
| Khk | 194 | 1.20E−05 | 0.001365 | 0.008079 | 0.009339 | 0.025145 | 0.004992 | 0.141697 |
| Flrt1 | 195 | 1.23E−05 | 0.001394 | 0.015376 | 0.005256 | 0.044458 | 0.174697 | 0.002208 |
| Cachd1 | 196 | 1.32E−05 | 0.001489 | 0.010531 | 0.045171 | 0.003527 | 0.113182 | 0.007962 |
| Htr3a | 197 | 1.35E−05 | 0.001519 | 0.146494 | 0.000801 | 0.096067 | 0.003839 | 0.036037 |
| Exoc3l2 | 198 | 1.39E−05 | 0.001552 | 0.005955 | 0.03678 | 0.011644 | 0.070518 | 0.008958 |

TABLE 12
(Supplementary Table S6b. Ranked genes by differential methylation
(Old + OSKM vs Old-1): RANK prom., p-value prom., q-value prom.)

| | RANK | p-value | q-value | p-value | p-value | p-value | p-value | p-value |
|---|---|---|---|---|---|---|---|---|
| Gene | prom. | prom. | prom. | prom. TR1 | prom. TR2 | prom. TR3 | prom. TR4 | prom. TR5 |
| Peg12 | 1 | 2.22E−16 | 4.75E−12 | 0.000135 | 1.36E−05 | 1.25E−05 | 2.24E−05 | 0.000302 |
| Hoxa9 | 14 | 3.54E−08 | 5.23E−05 | 0.00333 | 0.002488 | 0.017079 | 0.002946 | 0.003121 |
| Hoxaas3 | 1023 | 0.006224 | 0.130122 | 0.014599 | 0.082486 | ND | 0.093258 | 0.203683 |
| Fam83g | 2261 | 0.035823 | 0.338689 | 0.489041 | 0.006001 | 0.217817 | 0.635503 | 0.153257 |
| Tbx3 | 266 | 0.000198 | 0.015958 | 0.019169 | 0.041567 | 0.035974 | 0.01813 | 0.087367 |
| Hoxa2 | 212 | 0.000119 | 0.011949 | 0.027295 | 0.015107 | 0.011051 | 0.335996 | 0.015379 |
| Hoxd3 | 171 | 6.19E−05 | 0.007729 | 0.049694 | 0.037873 | 0.01677 | 0.008151 | 0.040208 |
| Gm10377 | NA | ND | ND | ND | ND | ND | ND | ND |
| Tbx5 | 439 | 0.000786 | 0.03831 | 0.314318 | 0.160092 | 0.015731 | 0.037799 | 0.009127 |
| Hoxb3 | 1814 | 0.022927 | 0.270068 | 0.243049 | 0.020277 | 0.072162 | 0.158859 | 0.553097 |
| Gm3772 | 6901 | 0.289947 | 0.898277 | ND | ND | ND | ND | 0.289947 |
| Rbfox1 | 6465 | 0.260306 | 0.860673 | ND | 0.228163 | 0.663254 | ND | 0.140075 |
| Hoxa3 | 213 | 0.000119 | 0.011949 | 0.027231 | 0.015166 | 0.01105 | 0.335888 | 0.015412 |
| Oxct2a | 3 | 1.12E−10 | 8.01E−07 | 0.000284 | 0.003004 | 0.010304 | 7.92E−05 | 0.002577 |
| Nfix | 661 | 0.002118 | 0.068538 | 0.024459 | 0.003016 | ND | 0.631223 | 0.119164 |
| Emilin2 | 329 | 0.000354 | 0.02304 | 0.030736 | 0.041776 | 0.157989 | 0.010549 | 0.04487 |
| 4933406F09Rik | NA | ND | ND | ND | ND | ND | ND | ND |
| Adcy2 | 334 | 0.000378 | 0.024215 | 0.370407 | 0.010647 | 0.092164 | 0.023501 | 0.012229 |
| Hic1 | 268 | 0.000203 | 0.01622 | 0.086893 | 0.0186 | 0.054619 | 0.006802 | 0.077964 |
| Tescl | 7 | 9.05E−10 | 2.69E−06 | 6.39E−06 | 0.007646 | 0.017199 | 4.84E−05 | 0.470825 |
| Gemin5 | 698 | 0.002558 | 0.078156 | 0.020087 | ND | 0.053834 | 0.008004 | 0.818518 |
| Gm45351 | NA | ND | ND | ND | ND | ND | ND | ND |
| Vmn2r47 | NA | ND | ND | ND | ND | ND | ND | ND |
| Tent4a | 517 | 0.001179 | 0.048784 | 0.054174 | ND | 0.010054 | ND | 0.029711 |
| Mir196b | 9 | 2.45E−09 | 5.81E−06 | 0.001858 | 0.001776 | 0.009845 | 0.001698 | 0.001081 |
| Ly6l | 10 | 3.07E−09 | 6.57E−06 | 0.00161 | 0.002748 | 0.001696 | 0.004449 | 0.002319 |
| Defa23 | NA | ND | ND | ND | ND | ND | ND | ND |
| Mup2 | 4428 | 0.133202 | 0.64302 | ND | 0.133202 | ND | ND | ND |
| Skint5 | NA | ND | ND | ND | ND | ND | ND | ND |
| Tmem94 | 98 | 1.71E−05 | 0.00371 | 0.013085 | 0.014448 | 0.026087 | 0.071336 | 0.005939 |
| Fktn | 2 | 5.35E−13 | 5.72E−09 | 9.51E−05 | 0.000896 | 4.85E−05 | 0.000163 | 0.006772 |
| Pitx2 | 14354 | 0.788041 | 1 | 0.502222 | 0.822841 | 0.904154 | 0.837374 | 0.135862 |
| Septin9 | 91 | 1.19E−05 | 0.002785 | 0.005373 | 0.169131 | 0.00851 | 0.001021 | 0.169698 |
| Mir6991 | 13 | 2.02E−08 | 3.32E−05 | 0.026264 | 0.011133 | 0.005376 | 0.00258 | 0.000167 |
| Gltp | 811 | 0.003715 | 0.097913 | 0.16562 | 0.036836 | 0.087732 | 0.072208 | 0.057935 |
| Fxyd1 | 245 | 0.000162 | 0.014173 | 0.061175 | 0.038325 | 0.383311 | 0.003589 | 0.010884 |
| Ebf1 | 12 | 9.65E−09 | 1.72E−05 | 0.020324 | 0.00036 | 0.003852 | 0.002335 | 0.00439 |
| Col28a1 | NA | ND | ND | ND | ND | ND | ND | ND |
| Rnf39 | 1407 | 0.012808 | 0.194484 | 0.271331 | 0.035685 | 0.049494 | 0.085418 | 0.319859 |
| Galnt10 | 10111 | 0.51984 | 1 | ND | 0.085196 | 0.999603 | ND | 0.877058 |
| Gal3st2c | 354 | 0.000454 | 0.027414 | 0.027203 | 0.010515 | 0.113429 | 0.121781 | 0.033529 |
| Pax1 | 4140 | 0.117076 | 0.604486 | 0.087342 | 0.416466 | 0.100699 | 0.294892 | 0.412488 |
| Mapk4 | 149 | 4.50E−05 | 0.00646 | 0.045422 | 0.010249 | 0.057476 | 0.013089 | 0.019796 |
| Rnf165 | 25 | 2.62E−07 | 0.000217 | 0.003986 | 0.021258 | 0.003564 | 0.009951 | 0.004514 |
| Prxl2c | 281 | 0.000221 | 0.01681 | ND | 0.009572 | 0.007387 | 0.101361 | 0.045056 |
| Ahdc1 | 564 | 0.00152 | 0.057636 | 0.214457 | 0.029522 | 0.077928 | 0.02626 | 0.050858 |
| Esyt2 | 4952 | 0.162679 | 0.702171 | 0.157672 | 0.054133 | 0.631405 | 0.392944 | 0.383487 |
| Skint6 | NA | ND | ND | ND | ND | ND | ND | ND |
| Zfta | 5127 | 0.174517 | 0.727672 | 0.307032 | 0.929916 | 0.352795 | 0.217717 | 0.04228 |
| Tango6 | 83 | 8.70E−06 | 0.002221 | ND | ND | 0.00643 | 0.01176 | 0.000731 |
| Cldn8 | 21 | 1.15E−07 | 0.000118 | 0.00202 | 0.005182 | 0.004003 | 0.034432 | 0.003591 |
| Stx1b | 1033 | 0.006386 | 0.132211 | 0.173113 | 0.102943 | 0.019061 | 0.371597 | 0.037977 |
| Adcy7 | 425 | 0.000722 | 0.03632 | 0.006703 | 0.026857 | 0.163069 | 0.240252 | 0.034567 |
| Hoxa7 | 18 | 6.61E−08 | 7.85E−05 | 0.001326 | 0.022107 | 0.006438 | 0.00408 | 0.003502 |
| Nod2 | 649 | 0.002037 | 0.066951 | 0.216369 | 0.114161 | 0.108149 | 0.005351 | 0.068543 |
| 3110070M22Rik | 22 | 1.70E−07 | 0.000165 | 0.006737 | 0.00614 | 0.004695 | 0.001617 | 0.02599 |
| Col16a1 | 2519 | 0.044329 | 0.376197 | 0.052976 | ND | 0.425864 | 0.117798 | 0.13493 |
| Irx3 | 221 | 0.000124 | 0.012024 | 0.06135 | 0.004677 | 0.006792 | 0.504831 | 0.025401 |
| Tdpoz5 | NA | ND | ND | ND | ND | ND | ND | ND |
| Tmem267 | 23 | 2.21E−07 | 0.000205 | 0.006755 | 0.007132 | 0.004475 | 0.001636 | 0.031506 |
| Atp6v1c2 | 1055 | 0.006774 | 0.137123 | 0.569854 | 0.325331 | 0.155556 | 0.004909 | 0.03682 |
| Hspa1a | 26 | 2.68E−07 | 0.000217 | 0.003105 | 0.000128 | 0.001022 | 0.289874 | ND |
| Gna14 | 6 | 7.36E−10 | 2.62E−06 | 0.001949 | 0.001997 | 0.010677 | 0.000453 | 0.000803 |
| Cyp3a41b | NA | ND | ND | ND | ND | ND | ND | ND |
| Jak3 | 7847 | 0.357886 | 0.975123 | 0.809316 | 0.364253 | 0.212668 | 0.124902 | 0.523132 |
| 4930578G10Rik | NA | ND | ND | ND | ND | ND | ND | ND |
| Man1c1 | 507 | 0.001115 | 0.047044 | 0.036725 | 0.014753 | 0.081177 | 0.181228 | 0.054544 |
| Ubash3b | 230 | 0.000132 | 0.012268 | 0.033594 | 0.047598 | 0.008611 | 0.008294 | 0.235866 |
| Mir196a-1 | 29 | 3.87E−07 | 0.000285 | 0.009254 | 0.005347 | 0.007478 | 0.004925 | 0.01181 |
| Caskin1 | 624 | 0.001851 | 0.063429 | 0.647983 | 0.10656 | 0.044478 | ND | 0.001518 |
| Zfp617 | 317 | 0.000314 | 0.02116 | 0.072729 | 0.046207 | 0.149105 | 0.00341 | 0.047948 |
| Zfp111 | 73 | 7.00E−06 | 0.002052 | 0.005561 | 0.019056 | 0.00741 | 0.001915 | 0.463999 |
| Rapgefl1 | 196 | 9.34E−05 | 0.010145 | 0.18344 | 0.010974 | 0.023448 | 0.020305 | 0.018134 |
| 4930558F17Rik | NA | ND | ND | ND | ND | ND | ND | ND |
| Zfp46 | 2342 | 0.038668 | 0.352949 | 0.043971 | 0.087442 | 0.174409 | ND | 0.436974 |
| Gm16513 | NA | ND | ND | ND | ND | ND | ND | ND |
| Zfp872 | 4 | 3.32E−10 | 1.78E−06 | 0.000276 | 0.000656 | 0.00212 | 0.008906 | 0.001794 |
| Umad1 | 5648 | 0.206616 | 0.782057 | 0.292812 | 0.417233 | 0.319509 | 0.114759 | 0.286787 |
| Marveld2 | 8 | 1.01E−09 | 2.69E−06 | 0.000545 | 0.000749 | 0.001486 | 0.000499 | 0.071249 |
| Col1a1 | 751 | 0.003083 | 0.087793 | 0.755767 | 0.003198 | 0.157247 | 0.424185 | 0.010726 |
| Dock1 | 5 | 5.09E−10 | 2.18E−06 | 0.001313 | 0.007189 | 0.001089 | 0.000554 | 0.001748 |
| Rassf3 | 9693 | 0.489348 | 1 | 0.517958 | 0.26603 | 0.554174 | 0.170205 | 0.680129 |
| Zbtb11os1 | 38 | 8.55E−07 | 0.000481 | 0.006841 | 0.005566 | 0.022979 | 0.00552 | 0.011451 |
| Dchs1 | 104 | 2.05E−05 | 0.004219 | 0.509078 | 0.004044 | 0.001463 | 0.006081 | ND |
| Smad7 | 1363 | 0.011927 | 0.186999 | ND | 0.047681 | 0.125155 | 0.046857 | ND |
| Atp6v1b2 | 88 | 9.87E−06 | 0.002398 | 0.025748 | 0.065633 | 0.005445 | 0.011293 | 0.010205 |
| Gm38499 | 2753 | 0.053006 | 0.411611 | 0.066276 | 0.238822 | 0.352216 | 0.033184 | 0.628855 |
| Ptprd | 2972 | 0.062731 | 0.451212 | 0.050573 | 0.827852 | ND | 0.511074 | 0.028276 |
| Crybb3 | 4973 | 0.164452 | 0.706931 | 0.263551 | 0.28107 | 0.213196 | 0.340661 | 0.154051 |
| Kcnn4 | 5296 | 0.185044 | 0.746952 | ND | 0.087963 | ND | ND | 0.513391 |
| Lsp1 | 4727 | 0.151112 | 0.682858 | 0.367665 | 0.346211 | 0.26485 | 0.170483 | 0.123156 |
| Osr2 | 17077 | 0.910217 | 1 | 0.22013 | 0.776853 | 0.810677 | 0.930272 | 0.739008 |
| Ctnnd2 | 11064 | 0.589279 | 1 | 0.381379 | 0.569011 | 0.37526 | 0.403503 | 0.455073 |
| 4930521O11Rik | NA | ND | ND | ND | ND | ND | ND | ND |
| Bmp8a | 6089 | 0.235092 | 0.825423 | 0.188821 | 0.22082 | 0.650684 | 0.301959 | 0.202857 |
| Clmp | 2282 | 0.036735 | 0.344111 | 0.939554 | 0.201497 | 0.293923 | 0.025539 | 0.045571 |
| Syt14 | 738 | 0.002946 | 0.085372 | 0.001095 | 0.65682 | 0.007193 | 0.366865 | 0.855815 |
| Gm53 | 10612 | 0.558367 | 1 | 0.197949 | 0.631766 | 0.21653 | 0.766667 | 0.613922 |
| Tbc1d30 | 7406 | 0.327999 | 0.946667 | 0.430992 | 0.151332 | 0.648016 | 0.339675 | 0.234306 |
| Ptprf | 1329 | 0.011111 | 0.178655 | 0.300449 | 0.052586 | 0.044598 | ND | 0.071128 |
| Dap | 396 | 0.000576 | 0.031035 | 0.099585 | 0.022844 | 0.026086 | 0.068753 | 0.044368 |
| Mmp2 | 521 | 0.001208 | 0.049604 | 0.054327 | 0.033268 | 0.102872 | 0.040699 | 0.063969 |
| Edn2 | 127 | 3.41E−05 | 0.005748 | 0.014661 | 0.0259 | 0.088633 | 0.008061 | 0.018096 |
| Gm4793 | 19 | 1.04E−07 | 0.000117 | 0.002338 | 0.04174 | 0.002082 | 0.032168 | 0.000702 |
| Gm14326 | 233 | 0.000137 | 0.012555 | 0.015928 | 0.01523 | 0.057569 | 0.070229 | 0.028754 |
| Zfhx2os | 72 | 6.67E−06 | 0.001983 | 0.029295 | 0.015893 | 0.010999 | 0.002621 | 0.049007 |
| Phldb1 | 4351 | 0.129048 | 0.634004 | 0.135338 | 0.1242 | 0.418822 | 0.304486 | 0.247472 |
| Adamts18 | 11108 | 0.591154 | 1 | 0.386307 | 0.535105 | 0.699257 | 0.302819 | 0.344934 |
| Herc6 | 130 | 3.77E−05 | 0.006135 | 0.042185 | 0.007752 | 0.165071 | 0.012758 | 0.008064 |
| Gm3250 | 51 | 2.52E−06 | 0.001056 | 0.002842 | 0.003927 | 0.031707 | 0.036362 | 0.015693 |
| Asap3 | 16278 | 0.879097 | 1 | 0.854093 | 0.436131 | 0.80713 | ND | ND |
| Dtx3 | 1002 | 0.005883 | 0.125569 | 0.077472 | ND | 0.063567 | 0.050696 | 0.084969 |
| Mir6899 | 52 | 2.57E−06 | 0.001056 | 0.005149 | 0.014742 | 0.007132 | 0.027741 | 0.013764 |
| Snx20 | 619 | 0.001832 | 0.063307 | 0.167281 | 0.056901 | 0.005905 | 0.228039 | 0.066196 |
| Cdc14b | 107 | 2.27E−05 | 0.004516 | 0.084736 | 0.123998 | 0.014641 | 0.000302 | 0.063594 |
| Cep85 | 14524 | 0.797153 | 1 | 0.6904 | 0.708054 | 0.131785 | 0.789796 | 0.880125 |
| Hoxb3os | 385 | 0.000556 | 0.030873 | 0.01799 | 0.014197 | 0.121486 | 0.027728 | 0.20096 |
| Mab21l2 | 54 | 2.76E−06 | 0.001081 | 0.003473 | 0.001568 | 0.014278 | 0.054184 | 0.053447 |
| Gpc2 | 41 | 1.22E−06 | 0.000639 | 0.002293 | 0.002459 | 0.002804 | 0.007479 | 0.718595 |
| Defa34 | 57 | 3.02E−06 | 0.001121 | 0.01069 | 0.000718 | ND | 0.007026 | 0.035649 |
| Mmp16 | 36 | 5.53E−07 | 0.000329 | 0.000443 | 0.065985 | 0.007804 | 0.034708 | 0.004157 |
| Aldoart1 | 60 | 3.28E−06 | 0.001168 | 0.007593 | 0.010728 | 0.007094 | 0.01933 | 0.024845 |
| Gm3002 | NA | ND | ND | ND | ND | ND | ND | ND |
| E330014E10Rik | NA | ND | ND | ND | ND | ND | ND | ND |
| Fto | 5450 | 0.194066 | 0.76098 | ND | 0.395007 | 0.688811 | 0.190001 | 0.073826 |
| Tox3 | 93 | 1.29E−05 | 0.00297 | 0.042134 | ND | 0.011532 | 0.009242 | 0.002357 |
| Skint3 | 2779 | 0.054088 | 0.415876 | 0.063126 | 0.222844 | 0.141867 | 0.297696 | 0.20236 |
| Cmbl | 2751 | 0.052713 | 0.409635 | 0.008268 | 0.311553 | 0.21728 | 0.255729 | 0.8055 |
| Ephb4 | 361 | 0.000481 | 0.028507 | 0.281651 | 0.122964 | 0.006725 | 0.032936 | 0.018649 |
| Podnl1 | 568 | 0.001557 | 0.058611 | 0.014363 | 0.109908 | 0.086526 | 0.010553 | 0.47211 |
| Mdk | 61 | 3.62E−06 | 0.001258 | 0.142405 | 0.002769 | 0.022818 | 0.1125 | 0.000309 |
| Hoxd4 | 172 | 6.22E−05 | 0.007729 | 0.049721 | 0.037894 | 0.016762 | 0.008187 | 0.040197 |
| Fam241b | 167 | 5.91E−05 | 0.007566 | 0.003376 | 0.022428 | 0.0833 | 0.010471 | 0.147629 |
| Nr4a1 | 6423 | 0.258143 | 0.859244 | 0.324424 | 0.049788 | 0.742519 | 0.881708 | 0.190327 |
| Glcci1 | 5212 | 0.180263 | 0.739255 | 0.292854 | 0.405959 | 0.323907 | 0.098571 | 0.259822 |
| Fyb | 30 | 4.22E−07 | 0.0003 | 0.020364 | 0.004052 | 0.01399 | 0.051652 | 0.0004 |
| Dera | 3923 | 0.106124 | 0.578237 | 0.064153 | 0.455969 | 0.292926 | 0.283973 | 0.153945 |
| Lgals4 | 210 | 0.000116 | 0.011847 | 0.020347 | 0.006061 | 0.325966 | 0.086815 | 0.006576 |
| Lpcat2 | 6386 | 0.255262 | 0.854435 | 0.251801 | 0.158474 | 0.619972 | 0.233414 | 0.340546 |
| Sema4b | 9190 | 0.453491 | 1 | 0.634559 | 0.659221 | 0.609409 | 0.124411 | 0.228663 |
| Skint11 | NA | ND | ND | ND | ND | ND | ND | ND |
| Rgl2 | 28 | 3.23E−07 | 0.000247 | 0.003031 | 0.008279 | 0.009668 | 0.01078 | 0.006653 |
| Lgals6 | 64 | 4.76E−06 | 0.001591 | 0.00776 | 0.002481 | 0.105841 | 0.06109 | 0.003505 |
| Vmn1r252 | NA | ND | ND | ND | ND | ND | ND | ND |
| Chd9 | 833 | 0.003856 | 0.098891 | 0.021602 | 0.044497 | 0.077937 | 0.180337 | 0.174543 |
| Slc7a11 | 92 | 1.20E−05 | 0.002785 | 0.018785 | 0.143751 | 0.134189 | 0.001715 | 0.002165 |
| Mir142b | 65 | 4.98E−06 | 0.001639 | 0.026291 | 0.00913 | 0.011994 | 0.001259 | 0.1272 |
| 5830417I10Rik | 541 | 0.001377 | 0.054442 | 0.015883 | 0.221828 | 0.039454 | ND | 0.02296 |
| Morf4l1-ps1 | 66 | 5.20E−06 | 0.001685 | 0.002098 | 0.007419 | 0.017631 | 0.01323 | ND |
| Ctif | 182 | 7.22E−05 | 0.008487 | 0.06121 | 0.348353 | 0.055625 | 0.002574 | 0.004112 |
| Oxct2b | 67 | 5.37E−06 | 0.001713 | 0.028802 | 0.000735 | 0.014285 | 0.297347 | 0.005607 |
| Fam110a | 1623 | 0.017538 | 0.230946 | 0.152886 | 0.170019 | 0.174856 | 0.042769 | 0.107318 |
| Skint4 | NA | ND | ND | ND | ND | ND | ND | ND |
| Serpina3n | 2216 | 0.034397 | 0.331802 | 0.111904 | 0.057666 | 0.249244 | 0.486824 | 0.074598 |
| Fam171a2 | 15632 | 0.85158 | 1 | 0.649652 | 0.758728 | 0.227531 | 0.570326 | 0.974967 |
| Csf2ra | 1575 | 0.0165 | 0.223902 | 0.009992 | 0.030232 | 0.364249 | 0.276242 | 0.626579 |
| Spata22 | 53 | 2.66E−06 | 0.001075 | 0.002079 | 0.398572 | 0.004739 | 0.044686 | 0.001231 |
| Atp1a3 | 989 | 0.00568 | 0.122761 | 0.021135 | 0.316465 | 0.031376 | 0.120491 | 0.160558 |
| Colgalt1 | 1681 | 0.0188 | 0.239 | 0.001008 | 0.818671 | 0.06768 | 0.748609 | 0.553731 |
| Zfp791 | 301 | 0.000256 | 0.018079 | 0.003355 | 0.358541 | 0.37111 | 0.010537 | 0.013382 |
| D930048N14Rik | 134 | 3.88E−05 | 0.006139 | 0.158229 | 0.632783 | 0.002444 | 0.008515 | 0.002763 |
| Itgb4 | 7298 | 0.319994 | 0.937368 | 0.843552 | 0.396773 | 0.243522 | 0.115776 | 0.337475 |
| Ttc14 | 11 | 5.12E−09 | 9.95E−06 | 0.004916 | 0.00171 | 0.002648 | 0.002835 | 0.002203 |
| Aif1 | 353 | 0.000452 | 0.02737 | 0.072675 | 0.046224 | 0.036477 | 0.017622 | 0.060995 |
| Ctsl | 81 | 8.23E−06 | 0.002172 | 0.024168 | 0.015055 | 0.007341 | 0.033608 | 0.009458 |
| Mir142 | 79 | 7.86E−06 | 0.002122 | 0.029317 | 0.010178 | 0.013607 | 0.001447 | 0.136598 |
| Odf3l1 | 491 | 0.00104 | 0.045157 | 0.039765 | 0.113966 | 0.019571 | 0.186098 | 0.024005 |
| Gvin2 | NA | ND | ND | ND | ND | ND | ND | ND |
| Gvin-ps2 | NA | ND | ND | ND | ND | ND | ND | ND |
| Irgc1 | 6027 | 0.231022 | 0.819477 | ND | 0.146831 | 0.183627 | ND | 0.646874 |
| 5730596B20Rik | 84 | 8.74E−06 | 0.002221 | 0.001693 | 0.01429 | 0.010749 | 0.080747 | 0.043545 |
| Stag3 | 46 | 1.54E−06 | 0.000715 | 0.002428 | 0.002431 | 0.003076 | 0.008863 | 0.693271 |
| Nyap1 | 1368 | 0.012044 | 0.188154 | 0.082993 | 0.273453 | 0.009369 | 0.263331 | ND |
| Gdnf | 166 | 5.90E−05 | 0.007566 | 0.008877 | 0.023446 | 0.089035 | 0.01844 | 0.028457 |
| Dppa1 | 7146 | 0.307954 | 0.921247 | 0.649641 | ND | 0.313264 | ND | 0.138243 |
| Cdkn1c | 86 | 9.12E−06 | 0.002268 | 0.111836 | 0.016179 | 0.001623 | 0.00239 | ND |
| Tecr | 6342 | 0.252399 | 0.850765 | 0.360123 | 0.082213 | 0.397051 | 0.435124 | 0.375609 |
| Fignl2 | 49 | 1.91E−06 | 0.000836 | 0.001876 | 0.000328 | 0.00051 | 0.845751 | 0.547391 |
| Mef2b | 179 | 7.07E−05 | 0.008412 | ND | ND | 0.017459 | 0.021957 | 0.001563 |
| E230032D23Rik | 101 | 1.96E−05 | 0.004144 | 0.007348 | 0.020903 | 0.181549 | 0.015361 | 0.005749 |
| Trim47 | 3561 | 0.089196 | 0.535375 | 0.331513 | 0.184767 | 0.372599 | 0.017427 | 0.696657 |
| Adamts12 | 16 | 5.45E−08 | 7.28E−05 | 0.364032 | 0.000301 | 0.031956 | 0.001085 | 0.000566 |
| Pnma8b | 188 | 8.25E−05 | 0.009387 | 0.020906 | 0.047905 | 0.085528 | 0.002169 | 0.079928 |
| Skint7 | NA | ND | ND | ND | ND | ND | ND | ND |
| Mdga1 | 4953 | 0.162716 | 0.702171 | 0.284298 | 0.0716 | 0.569905 | 0.429449 | 0.163075 |
| Zcchc24 | 584 | 0.001642 | 0.060131 | ND | 0.083387 | 0.008555 | 0.504166 | 0.011118 |
| 5730507C01Rik | 291 | 0.000237 | 0.017401 | ND | 0.013271 | 0.007527 | ND | 0.024313 |
| Cbx8 | 13308 | 0.73035 | 1 | 0.361484 | 0.837456 | 0.437437 | 0.663717 | 0.352633 |
| Ankrd33b | 4080 | 0.113723 | 0.595804 | 0.195016 | 0.111928 | 0.057585 | 0.526492 | 0.639426 |
| Psg16 | 1721 | 0.020386 | 0.253176 | 0.351358 | 0.052812 | 0.128062 | 0.135291 | 0.081346 |
| Tcap | 90 | 1.18E−05 | 0.002785 | 0.046707 | ND | 0.025292 | 0.004875 | 0.001651 |
| Nim1k | 859 | 0.004065 | 0.101132 | 0.05102 | 0.064336 | 0.129946 | 0.146468 | 0.040634 |
| 1700001C19Rik | 117 | 2.77E−05 | 0.005062 | ND | 0.00106 | 0.01009 | 0.375827 | 0.006515 |
| Khk | 24 | 2.47E−07 | 0.000217 | 0.002415 | 0.004163 | 0.012775 | 0.001152 | 0.085612 |
| Flrt1 | 70 | 6.05E−06 | 0.001848 | 0.025494 | 0.009981 | 0.028243 | 0.061144 | 0.001328 |
| Cachd1 | 328 | 0.000353 | 0.023014 | 0.074465 | 0.145404 | 0.002546 | 0.155388 | 0.022294 |
| Htr3a | 1117 | 0.007733 | 0.147933 | 0.088735 | 0.0188 | 0.269265 | 0.083924 | 0.16706 |
| Exoc3l2 | 3111 | 0.069287 | 0.475866 | ND | 0.238523 | 0.061637 | ND | 0.197007 |

TABLE 13
(Supplementary Table Soc. Ranked genes by differential methylation (Old
+ OSKM vs Old-1): Rank_body, p-value body, q-value body.)

| | | p-value | q-value | p-value | p-value | p-value | p-value | p-value |
|---|---|---|---|---|---|---|---|---|
| Gene | Rank_body | body | body | body TR1 | body TR2 | body TR3 | body TR4 | body TR5 |
| Peg12 | NA | ND | ND | ND | ND | ND | ND | ND |
| Hoxa9 | 3 | 1.10E−12 | 6.60E−09 | 2.61E−05 | 0.000603 | 0.017667 | 7.44E−05 | 0.000492 |
| Hoxaas3 | 10 | 5.98E−11 | 1.07E−07 | 1.28E−05 | 0.00022 | 0.00074 | 0.240161 | 0.001757 |
| Fam83g | 62 | 2.11E−07 | 6.01E−05 | 0.087835 | 1.59E−05 | 0.057446 | 0.137701 | 0.000952 |
| Tbx3 | 1 | 2.06E−15 | 3.69E−11 | 7.78E−05 | 0.000149 | 0.00013 | 8.69E−05 | 7.67E−05 |
| Hoxa2 | 9 | 4.90E−11 | 9.76E−08 | 1.21E−05 | 0.000674 | 0.001573 | 0.097434 | 0.000565 |
| Hoxd3 | 11 | 1.63E−10 | 2.66E−07 | 0.001197 | 0.003195 | 6.39E−05 | 0.020529 | 0.000545 |
| Gm10377 | 6 | 9.77E−12 | 2.92E−08 | 0.000787 | 0.000478 | 0.000802 | 0.000436 | 0.000881 |
| Tbx5 | 8 | 3.92E−11 | 8.79E−08 | 0.002251 | 0.002793 | 0.000296 | 0.000872 | 0.000339 |
| Hoxb3 | 2 | 5.26E−13 | 4.72E−09 | 0.000873 | 0.001141 | 2.86E−05 | 0.00052 | 0.000302 |
| Gm3772 | 7 | 2.06E−11 | 5.28E−08 | 3.36E−05 | 2.15E−05 | 0.002843 | 0.001993 | 0.064928 |
| Rbfox1 | 5 | 7.05E−12 | 2.53E−08 | 5.82E−07 | 0.045169 | 0.030052 | 6.57E−06 | 0.015492 |
| Hoxa3 | 14 | 4.33E−10 | 5.55E−07 | 1.11E−05 | 0.00047 | 0.002379 | 0.171254 | 0.003894 |
| Oxct2a | NA | ND | ND | ND | ND | ND | ND | ND |
| Nfix | 13 | 2.86E−10 | 3.94E−07 | 0.000591 | 0.000235 | 0.002767 | 0.005598 | 0.002401 |
| Emilin2 | 18 | 1.41E−09 | 1.33E−06 | 0.001109 | 0.005129 | 0.002016 | 0.000389 | 0.00711 |
| 4933406F09Rik | 12 | 2.84E−10 | 3.94E−07 | 8.80E−05 | 0.006854 | 0.022477 | 0.000555 | 0.00068 |
| Adcy2 | 26 | 4.62E−09 | 3.12E−06 | 0.000946 | 0.000817 | 0.001704 | 0.001418 | 0.066201 |
| Hic1 | 38 | 1.53E−08 | 7.24E−06 | 0.056051 | 3.40E−05 | 0.001888 | 0.008653 | 0.015829 |
| Tescl | NA | ND | ND | ND | ND | ND | ND | ND |
| Gemin5 | 35 | 1.22E−08 | 6.26E−06 | 0.001872 | 0.000164 | 0.03229 | 0.000193 | 0.198155 |
| Gm45351 | 21 | 1.81E−09 | 1.55E−06 | 0.00029 | 0.001571 | 0.008921 | 0.001055 | 0.009869 |
| Vmn2r47 | 22 | 1.90E−09 | 1.55E−06 | 0.00029 | 0.001646 | 0.009146 | 0.001055 | 0.009705 |
| Tent4a | 47 | 3.71E−08 | 1.39E−05 | 0.005589 | 0.001133 | 0.003377 | 0.002738 | 0.023481 |
| Mir196b | NA | ND | ND | ND | ND | ND | ND | ND |
| Ly6l | NA | ND | ND | ND | ND | ND | ND | ND |
| Defa23 | 31 | 7.30E−09 | 4.10E−06 | 0.004233 | 0.00044 | 0.002814 | 0.001387 | 0.02876 |
| Mup2 | 27 | 4.72E−09 | 3.12E−06 | 0.00821 | 0.001481 | 0.001001 | 0.003354 | 0.003101 |
| Skint5 | 33 | 9.19E−09 | 5.00E−06 | 0.002748 | 0.019535 | 0.002391 | 0.001578 | 0.001347 |
| Tmem94 | 120 | 4.01E−06 | 0.000594 | 0.007798 | 0.011223 | 0.008974 | 0.004269 | 0.105524 |
| Fktn | 16514 | 0.979521 | 1 | 0.913315 | 0.734033 | 0.608286 | 0.895858 | 0.58732 |
| Pitx2 | 4 | 3.14E−12 | 1.41E−08 | 0.003474 | 0.00015 | 7.78E−05 | 0.000122 | 0.006593 |
| Septin9 | 183 | 1.36E−05 | 0.001323 | 0.040314 | 0.007941 | 0.008773 | 0.031486 | 0.017829 |
| Mir6991 | NA | ND | ND | ND | ND | ND | ND | ND |
| Gltp | 43 | 2.88E−08 | 1.18E−05 | 0.002961 | 0.004101 | 0.056847 | 0.004232 | 0.000351 |
| Fxyd1 | 91 | 1.08E−06 | 0.000211 | 0.003824 | 0.001617 | 0.100159 | 0.001845 | 0.06395 |
| Ebf1 | 1619 | 0.010354 | 0.114588 | 0.065517 | 0.016273 | 0.617719 | 0.044899 | 0.324534 |
| Col28a1 | 42 | 2.69E−08 | 1.12E−05 | 0.000224 | 0.012303 | 0.0002 | 0.069403 | 0.024734 |
| Rnf39 | 37 | 1.49E−08 | 7.22E−06 | 0.00427 | 0.001053 | 0.000951 | 0.015341 | 0.007264 |
| Galnt10 | 25 | 3.19E−09 | 2.29E−06 | 0.000308 | 0.001193 | 0.035879 | 8.73E−05 | 0.070357 |
| Gal3st2c | 92 | 1.23E−06 | 0.000238 | 0.035436 | 0.012263 | 0.02119 | 0.012945 | 0.000718 |
| Pax1 | 16 | 1.16E−09 | 1.30E−06 | 0.00156 | 0.011326 | 0.000173 | 0.003334 | 0.002488 |
| Mapk4 | 191 | 1.48E−05 | 0.00138 | 0.010857 | 0.021076 | 0.030324 | 0.050271 | 0.004986 |
| Rnf165 | 900 | 0.002018 | 0.04016 | 0.08718 | 0.035581 | 0.089577 | 0.049375 | 0.070523 |
| Prxl2c | 113 | 3.35E−06 | 0.000523 | 0.038181 | 0.016827 | 0.006602 | 0.013919 | 0.004826 |
| Ahdc1 | 74 | 3.76E−07 | 8.99E−05 | 0.001475 | 0.009268 | 0.040228 | 0.008542 | 0.004432 |
| Esyt2 | 17 | 1.28E−09 | 1.33E−06 | 0.000141 | 0.00061 | 0.0015 | 0.002116 | 0.103781 |
| Skint6 | 52 | 9.06E−08 | 3.07E−05 | 0.004241 | 0.016457 | 0.008331 | 0.002772 | 0.002417 |
| Zfta | 20 | 1.48E−09 | 1.33E−06 | 0.026622 | 0.007747 | 0.003079 | 0.001198 | 4.40E−05 |
| Tango6 | 379 | 0.000133 | 0.006269 | 0.011216 | 0.056254 | 0.025643 | 0.055434 | 0.030265 |
| Cldn8 | NA | ND | ND | ND | ND | ND | ND | ND |
| Stx1b | 59 | 1.81E−07 | 5.41E−05 | 0.002199 | 0.009375 | 0.007665 | 0.002735 | 0.020293 |
| Adcy7 | 104 | 2.32E−06 | 0.000397 | 0.020983 | 0.001935 | 0.009133 | 0.036742 | 0.013433 |
| Hoxa7 | 1815 | 0.014703 | 0.14521 | 0.224099 | 0.021204 | 0.208438 | 0.209483 | 0.077349 |
| Nod2 | 84 | 7.25E−07 | 0.000152 | 0.000331 | 0.0009 | 0.006642 | 0.21932 | 0.104501 |
| 3110070M22Rik | NA | ND | ND | ND | ND | ND | ND | ND |
| Col16a1 | 50 | 6.33E−08 | 2.23E−05 | 0.007468 | 0.001232 | 0.00657 | 0.002836 | 0.014951 |
| Irx3 | 211 | 2.03E−05 | 0.001618 | 0.001102 | 0.003125 | 0.522614 | 0.458868 | 0.003124 |
| Tdpoz5 | 58 | 1.80E−07 | 5.41E−05 | 0.004851 | 0.00616 | 0.020272 | 0.003267 | 0.004399 |
| Tmem267 | 1480 | 0.008283 | 0.100131 | 0.335987 | 0.001591 | 0.489829 | 0.040498 | 0.655518 |
| Atp6v1c2 | 65 | 2.49E−07 | 6.78E−05 | 0.021895 | 0.004541 | 0.029435 | 5.77E−05 | 0.075896 |
| Hspala | NA | ND | ND | ND | ND | ND | ND | ND |
| Gna14 | 8772 | 0.470298 | 0.960954 | 0.325501 | 0.269487 | 0.23102 | 0.673768 | 0.58328 |
| Cyp3a41b | 68 | 3.11E−07 | 8.09E−05 | 0.019842 | 0.011093 | 0.008999 | 0.00539 | 0.001559 |
| Jak3 | 19 | 1.44E−09 | 1.33E−06 | 0.002371 | 9.62E−06 | 0.112664 | 0.004666 | 0.002704 |
| 4930578G10Rik | 70 | 3.25E−07 | 8.22E−05 | 0.024874 | 0.00797 | 0.002662 | 0.004779 | 0.006958 |
| Manlcl | 122 | 4.14E−06 | 0.000604 | 0.009688 | 0.014841 | 0.009192 | 0.022475 | 0.012385 |
| Ubash3b | 270 | 3.99E−05 | 0.002645 | 0.046547 | 0.019541 | 0.017664 | 0.022187 | 0.016754 |
| Mir196a-1 | NA | ND | ND | ND | ND | ND | ND | ND |
| Caskin1 | 131 | 5.02E−06 | 0.000683 | 0.007227 | 0.008096 | 0.078909 | 0.008678 | 0.011619 |
| Zfp617 | 212 | 2.03E−05 | 0.001618 | 0.046816 | 0.005238 | 0.038426 | 0.000893 | 0.306887 |
| Zfp111 | 666 | 0.000911 | 0.024502 | 0.003514 | 0.403195 | 0.240935 | 0.002335 | 0.416355 |
| Rapgefl1 | 319 | 7.46E−05 | 0.004184 | 0.043588 | 0.07484 | 0.009318 | 0.011655 | 0.036924 |
| 4930558F17Rik | 77 | 4.63E−07 | 0.000106 | 0.012852 | 0.003434 | 0.029956 | 0.002518 | 0.008001 |
| Zfp46 | 71 | 3.31E−07 | 8.26E−05 | 0.01901 | 0.003087 | 0.031468 | 0.000691 | 0.014037 |
| Gm16513 | 80 | 5.03E−07 | 0.000112 | 0.006721 | 0.019437 | 0.002742 | 0.014223 | 0.005777 |
| Zfp872 | 13923 | 0.866944 | 1 | 0.873979 | 0.839594 | 0.360692 | 0.728215 | 0.358339 |
| Umad1 | 34 | 1.08E−08 | 5.71E−06 | 0.000184 | 0.012698 | 0.000219 | 0.019541 | 0.032926 |
| Marveld2 | 10917 | 0.648669 | 1 | 0.581147 | 0.532859 | 0.326618 | 0.836667 | 0.239572 |
| Col1a1 | 118 | 3.53E−06 | 0.000533 | 0.046166 | 0.014754 | 0.002017 | 0.011298 | 0.019569 |
| Dock1 | 13686 | 0.853868 | 1 | 0.543664 | 0.534686 | 0.867018 | 0.647587 | 0.387826 |
| Rassf3 | 24 | 2.88E−09 | 2.16E−06 | 0.004072 | 0.000271 | 0.001462 | 0.009247 | 0.00482 |
| Zbtb11os1 | NA | ND | ND | ND | ND | ND | ND | ND |
| Dchs1 | 532 | 0.000462 | 0.015554 | 0.040276 | 0.035462 | 0.026046 | 0.010108 | 0.361109 |
| Smad7 | 110 | 3.28E−06 | 0.000523 | 0.004687 | 0.024204 | 0.010796 | 0.018882 | 0.012006 |
| Atp6v1b2 | 799 | 0.001508 | 0.033835 | 0.005497 | 0.033885 | 0.390613 | 0.280876 | 0.031914 |
| Gm38499 | 61 | 2.08E−07 | 6.01E−05 | 9.18E−05 | 0.199724 | 0.013576 | 5.39E−05 | 0.772285 |
| Ptprd | 94 | 1.31E−06 | 0.000248 | 0.019793 | 0.108885 | 1.42E−05 | 0.075157 | 0.040112 |
| Crybb3 | 46 | 3.48E−08 | 1.33E−05 | 0.003838 | 0.002579 | 0.004458 | 0.004406 | 0.006562 |
| Kcnn4 | 79 | 4.82E−07 | 0.000108 | 0.006683 | 0.006533 | 0.022372 | 0.001576 | 0.018174 |
| Lsp1 | 49 | 5.04E−08 | 1.81E−05 | 0.014778 | 0.000119 | 0.006074 | 0.004548 | 0.040315 |
| Osr2 | 15 | 7.34E−10 | 8.78E−07 | 0.00181 | 0.002883 | 0.000999 | 0.000794 | 0.003645 |
| Ctnnd2 | 28 | 4.86E−09 | 3.12E−06 | 0.000179 | 0.000222 | 0.000113 | 0.289496 | 0.101159 |
| 4930521O11Rik | 99 | 1.56E−06 | 0.00028 | 0.070314 | 0.0048 | 0.01719 | 0.003369 | 0.005808 |
| Bmp8a | 41 | 2.55E−08 | 1.09E−05 | 0.001108 | 0.013305 | 0.010787 | 0.000399 | 0.014016 |
| Clmp | 78 | 4.66E−07 | 0.000106 | 0.013114 | 0.002694 | 0.010754 | 0.023201 | 0.003047 |
| Syt14 | 168 | 9.86E−06 | 0.001047 | 0.005484 | 0.224899 | 0.016333 | 0.002873 | 0.018307 |
| Gm53 | 29 | 5.36E−09 | 3.32E−06 | 0.009424 | 0.001013 | 0.012548 | 0.000394 | 0.00311 |
| Tbc1d30 | 36 | 1.48E−08 | 7.22E−06 | 0.056065 | 4.42E−05 | 4.27E−05 | 0.039628 | 0.112835 |
| Ptprf | 117 | 3.52E−06 | 0.000533 | 0.006021 | 0.012942 | 0.006908 | 0.028503 | 0.019701 |
| Dap | 306 | 6.30E−05 | 0.003682 | 0.002179 | 0.011292 | 0.016343 | 0.052335 | 0.502222 |
| Mmp2 | 254 | 3.17E−05 | 0.002229 | 0.006213 | 0.004569 | 0.15618 | 0.044224 | 0.022846 |
| Edn2 | 739 | 0.001256 | 0.03047 | 0.036719 | 0.050976 | 0.107326 | 0.078898 | 0.032178 |
| Gm4793 | 4529 | 0.129053 | 0.510889 | 0.459398 | 0.237089 | 0.214317 | 0.090739 | 0.250464 |
| Gm14326 | 494 | 0.000339 | 0.012291 | 0.020504 | 0.037036 | 0.044346 | 0.079559 | 0.033874 |
| Zfhx2os | 1311 | 0.006055 | 0.082756 | 0.089371 | 0.290778 | 0.05308 | 0.113352 | 0.028427 |
| Phldb1 | 55 | 1.25E−07 | 4.01E−05 | 0.034629 | 0.001682 | 0.006187 | 0.003894 | 0.004062 |
| Adamts18 | 32 | 7.31E−09 | 4.10E−06 | 0.000122 | 0.000601 | 0.007768 | 0.027136 | 0.01357 |
| Herc6 | 790 | 0.001449 | 0.032871 | 0.01977 | 0.08237 | 0.161376 | 0.015106 | 0.155604 |
| Gm3250 | NA | ND | ND | ND | ND | ND | ND | ND |
| Asap3 | 48 | 4.45E−08 | 1.63E−05 | 0.00326 | 0.006613 | 0.006007 | 0.000193 | 0.06802 |
| Dtx3 | 235 | 2.31E−05 | 0.001751 | 0.012141 | 0.000925 | 0.078567 | 0.530229 | 0.00645 |
| Mir6899 | NA | ND | ND | ND | ND | ND | ND | ND |
| Snx20 | 251 | 3.05E−05 | 0.002173 | 0.008853 | 0.025759 | 0.330832 | 0.014239 | 0.003974 |
| Cdc14b | 953 | 0.0024 | 0.045152 | 0.039642 | 0.055142 | 0.070422 | 0.073811 | 0.107899 |
| Cep85 | 23 | 2.70E−09 | 2.11E−06 | 0.041526 | 0.010115 | 4.67E−05 | 0.004047 | 0.000841 |
| Hoxb3os | 370 | 0.000118 | 0.005694 | 0.046398 | 0.564946 | 0.000153 | 0.022824 | 0.254554 |
| Mab21l2 | NA | ND | ND | ND | ND | ND | ND | ND |
| Gpc2 | 2536 | 0.033246 | 0.234976 | 0.299297 | 0.173895 | 0.126974 | 0.033008 | 0.253912 |
| Defa34 | NA | ND | ND | ND | ND | ND | ND | ND |
| Mmp16 | 3265 | 0.062201 | 0.341401 | 0.067798 | 0.209225 | 0.128859 | 0.553966 | 0.149272 |
| Aldoart1 | NA | ND | ND | ND | ND | ND | ND | ND |
| Gm3002 | 114 | 3.38E−06 | 0.000523 | 0.021309 | 0.016342 | 0.001314 | 0.04126 | 0.015267 |
| E330014E10Rik | 115 | 3.39E−06 | 0.000523 | 0.006498 | 0.006114 | 0.066748 | 0.008798 | 0.012376 |
| Fto | 76 | 4.28E−07 | 9.98E−05 | 0.001114 | 0.0014 | 0.021373 | 0.033602 | 0.021689 |
| Tox3 | 1266 | 0.005476 | 0.077552 | 0.022527 | 0.024932 | 0.206902 | 0.22276 | 0.148937 |
| Skint3 | 85 | 7.26E−07 | 0.000152 | 0.001999 | 0.090472 | 0.002514 | 0.005782 | 0.017314 |
| Cmbl | 83 | 7.21E−07 | 0.000152 | 0.001156 | 0.000618 | 0.004163 | 0.295665 | 0.051335 |
| Ephb4 | 411 | 0.00017 | 0.007405 | 0.028553 | 0.073952 | 0.106447 | 0.013485 | 0.012284 |
| Podnl1 | 299 | 5.86E−05 | 0.003502 | 0.002664 | 0.002569 | 0.00826 | 0.736316 | 0.231639 |
| Mdk | NA | ND | ND | ND | ND | ND | ND | ND |
| Hoxd4 | 802 | 0.001546 | 0.034494 | 0.172543 | 0.030184 | 0.028413 | 0.173673 | 0.026235 |
| Fam241b | 796 | 0.001485 | 0.033437 | 0.027262 | 0.066643 | 0.061257 | 0.08788 | 0.065293 |
| Nr4a1 | 51 | 7.67E−08 | 2.65E−05 | 0.003827 | 0.003598 | 0.004983 | 0.005036 | 0.009288 |
| Glcci1 | 56 | 1.29E−07 | 4.06E−05 | 0.000693 | 0.013931 | 0.000371 | 0.029873 | 0.055194 |
| Fyb | 3729 | 0.084538 | 0.406295 | 0.130816 | 0.10283 | 0.498983 | 0.126094 | 0.298671 |
| Dera | 72 | 3.57E−07 | 8.77E−05 | 0.002855 | 0.006658 | 0.002675 | 0.009889 | 0.038915 |
| Lgals4 | 657 | 0.000887 | 0.024196 | 0.028715 | 0.037819 | 0.066296 | 0.069438 | 0.064107 |
| Lpcat2 | 53 | 9.69E−08 | 3.22E−05 | 0.002421 | 0.002418 | 0.001591 | 0.06146 | 0.007375 |
| Sema4b | 45 | 3.06E−08 | 1.19E−05 | 0.008825 | 0.002684 | 0.006513 | 0.006859 | 0.001039 |
| Skint11 | 126 | 4.61E−06 | 0.000651 | 0.007138 | 0.019319 | 0.000705 | 0.156496 | 0.02753 |
| Rgl2 | 4417 | 0.12253 | 0.497279 | 0.023662 | 0.601169 | 0.162492 | 0.260782 | 0.801756 |
| Lgals6 | NA | ND | ND | ND | ND | ND | ND | ND |
| Vmn1r252 | 130 | 4.87E−06 | 0.000668 | ND | 0.002693 | 0.002127 | ND | 0.005032 |
| Chd9 | 252 | 3.09E−05 | 0.002192 | 0.001129 | 0.015755 | 0.031512 | 0.150805 | 0.051326 |
| Slc7a11 | 1451 | 0.008011 | 0.098874 | 0.0833 | 0.06591 | 0.088517 | 0.20132 | 0.067721 |
| Mir142b | NA | ND | ND | ND | ND | ND | ND | ND |
| 5830417I10Rik | 349 | 0.000103 | 0.005294 | 0.026032 | 0.022552 | 0.014327 | 0.044345 | 0.05292 |
| Morf4l1-ps1 | NA | ND | ND | ND | ND | ND | ND | ND |
| Ctif | 832 | 0.001697 | 0.036529 | 0.353771 | 0.022323 | 0.033871 | 0.064764 | 0.04415 |
| Oxct2b | NA | ND | ND | ND | ND | ND | ND | ND |
| Fam110a | 132 | 5.10E−06 | 0.000689 | 0.019079 | 0.018196 | 0.001698 | 0.007791 | 0.103317 |
| Skint4 | 142 | 6.11E−06 | 0.000766 | 0.006062 | 0.042001 | 0.010874 | 0.002177 | 0.097964 |
| Serpina3n | 108 | 2.80E−06 | 0.000462 | 0.006963 | 0.000932 | 0.011373 | 0.027194 | 0.114526 |
| Fam171a2 | 30 | 6.09E−09 | 3.64E−06 | 6.16E−06 | 0.010986 | 0.001174 | 0.057302 | 0.037326 |
| Csf2ra | 150 | 7.39E−06 | 0.000879 | 0.001974 | 0.002167 | 0.062884 | 0.06906 | 0.040111 |
| Spata22 | 2638 | 0.037296 | 0.253421 | 0.409593 | 0.096664 | 0.079596 | 0.08213 | 0.256261 |
| Atp1a3 | 245 | 2.72E−05 | 0.001983 | 0.004431 | 0.142608 | 0.247655 | 0.007424 | 0.003184 |
| Colgalt1 | 149 | 7.33E−06 | 0.000877 | 0.001924 | 0.113223 | 0.003431 | 0.015209 | 0.064853 |
| Zfp791 | 763 | 0.001353 | 0.031778 | ND | 0.072706 | 0.066058 | 0.049247 | 0.013191 |
| D930048N14Rik | 1156 | 0.004379 | 0.067876 | 0.308052 | 0.091358 | 0.163087 | 0.01433 | 0.042824 |
| Itgb4 | 54 | 1.17E−07 | 3.81E−05 | 0.011299 | 0.032397 | 0.001099 | 0.010219 | 0.001277 |
| Ttc14 | 15825 | 0.956742 | 1 | 0.807266 | 0.553364 | 0.600459 | 0.612863 | 0.919171 |
| Aif1 | 551 | 0.000499 | 0.016206 | 0.121118 | 0.035269 | 0.013718 | 0.071366 | 0.035847 |
| Ctsl | 2747 | 0.041379 | 0.270017 | ND | ND | 0.339808 | 0.082082 | 0.051112 |
| Mir142 | NA | ND | ND | ND | ND | ND | ND | ND |
| Odf3l1 | 442 | 0.000214 | 0.008663 | 0.015856 | 0.066107 | 0.012999 | 0.028613 | 0.128173 |
| Gvin2 | 157 | 8.31E−06 | 0.000938 | 0.001176 | 0.097528 | 0.0013 | 0.014529 | 0.39716 |
| Gvin-ps2 | 157 | 8.31E−06 | 0.000938 | 0.001176 | 0.097528 | 0.0013 | 0.014529 | 0.39716 |
| Irgc1 | 96 | 1.46E−06 | 0.000269 | 0.01414 | 0.000898 | 0.000431 | 0.031662 | 0.603438 |
| 5730596B20Rik | NA | ND | ND | ND | ND | ND | ND | ND |
| Stag3 | 3890 | 0.09269 | 0.427151 | 0.290415 | 0.039129 | 0.412883 | 0.179056 | 0.352351 |
| Nyap1 | 278 | 4.45E−05 | 0.002863 | 0.138423 | 0.024475 | 0.006189 | 0.23831 | 0.001369 |
| Gdnf | 1132 | 0.004108 | 0.065072 | 0.121713 | 0.116514 | 0.094121 | 0.064038 | 0.03014 |
| Dppa1 | 106 | 2.61E−06 | 0.000438 | 0.001983 | 0.001666 | 0.13141 | 0.001899 | 0.255639 |
| Cdkn1c | NA | ND | ND | ND | ND | ND | ND | ND |
| Tecr | 66 | 2.73E−07 | 7.28E−05 | 0.00247 | 0.000479 | 0.080861 | 0.022854 | 0.006513 |
| Fignl2 | 4021 | 0.099875 | 0.445204 | 0.282656 | 0.070813 | 0.135599 | 0.211055 | 0.588105 |
| Mef2b | 922 | 0.002145 | 0.041701 | 0.017326 | 0.607323 | 0.200226 | 0.305497 | 0.001634 |
| E230032D23Rik | 1719 | 0.012332 | 0.128567 | 0.136919 | 0.213283 | 0.052797 | 0.080684 | 0.099568 |
| Trim47 | 98 | 1.51E−06 | 0.000273 | 0.071736 | 0.000752 | 0.00143 | 0.043108 | 0.032734 |
| Adamts12 | 10195 | 0.586389 | 1 | 0.265598 | 0.500308 | 0.718756 | 0.511326 | 0.301673 |
| Pnma8b | 1597 | 0.010031 | 0.112572 | ND | 0.014042 | 0.079448 | ND | 0.201143 |
| Skint7 | 172 | 1.08E−05 | 0.001116 | 0.004111 | 0.008954 | 0.044075 | 0.007112 | 0.10223 |
| Mdga1 | 82 | 6.96E−07 | 0.00015 | 0.002211 | 0.00094 | 0.156296 | 0.009992 | 0.01332 |
| Zcchc24 | 438 | 0.000211 | 0.00863 | 0.050648 | 0.034354 | 0.008887 | 0.027354 | 0.116203 |
| 5730507C01Rik | 865 | 0.001779 | 0.036865 | 0.01163 | 0.028174 | 0.201349 | ND | 0.067168 |
| Cbx8 | 44 | 3.05E−08 | 1.19E−05 | 0.00021 | 0.000836 | 0.006554 | 0.019366 | 0.049142 |
| Ankrd33b | 90 | 1.07E−06 | 0.00021 | 0.000876 | 0.051123 | 0.003441 | 0.020246 | 0.023052 |
| Psg16 | 179 | 1.26E−05 | 0.00126 | 0.003479 | 0.17198 | 0.011212 | 0.004402 | 0.048658 |
| Tcap | NA | ND | ND | ND | ND | ND | ND | ND |
| Nim1k | 329 | 8.50E−05 | 0.004624 | 0.030799 | 0.006254 | 0.150062 | 0.005588 | 0.095488 |
| 1700001C19Rik | 1393 | 0.007204 | 0.092615 | 0.327126 | 0.124722 | 0.016193 | 0.575893 | 0.014959 |
| Khk | 7012 | 0.322601 | 0.824945 | 0.349295 | 0.244613 | 0.267415 | 0.383724 | 0.369798 |
| Flrt1 | 2644 | 0.037651 | 0.255247 | 0.074023 | 0.047968 | 0.245906 | 0.684237 | 0.112685 |
| Cachd1 | 715 | 0.001137 | 0.028493 | 0.0157 | 0.048676 | 0.103813 | 0.151048 | 0.037221 |
| Htr3a | 283 | 4.70E−05 | 0.002969 | 0.372386 | 0.002409 | 0.070427 | 0.003587 | 0.032295 |
| Exoc3l2 | 167 | 9.70E−06 | 0.001036 | 0.005955 | 0.023276 | 0.0216 | 0.070518 | 0.004918 |

TABLE 14
(Supplementary Table S7a. Ranked genes by differential methylation
(Old + OSKM vs Old-3): RANK comp., p-value comp., q-value comp.)

| | RANK | p-value | q-value | p-value | p-value | p-value | p-value | p-value |
|---|---|---|---|---|---|---|---|---|
| Gene | comp. | comp. | comp. | comp. TR1 | comp. TR2 | comp. TR3 | comp. TR4 | comp. TR5 |
| Nfix | 1 | 2.22E−16 | 1.64E−12 | 7.82E−05 | 0 | 0.00257 | 0.005041 | 0.000361 |
| Nlrp5-ps | 2 | 2.22E−16 | 1.64E−12 | 0.140977 | 0 | 0.290766 | 0.048207 | 0.000381 |
| Atp6v1c2 | 3 | 2.22E−16 | 1.64E−12 | 0.011811 | 0.033664 | 0.372555 | 0 | 0.031739 |
| Hoxb3 | 4 | 2.35E−13 | 1.30E−09 | 4.88E−05 | 0.000186 | 2.93E−05 | 0.001641 | 0.00421 |
| Mup2 | 5 | 1.78E−12 | 7.51E−09 | 0.000931 | 0.000361 | 0.000318 | 0.000217 | 0.000746 |
| Rbfox1 | 6 | 2.04E−12 | 7.51E−09 | 1.76E−07 | 0.052146 | 0.17245 | 3.73E−07 | 0.033966 |
| Gm10377 | 7 | 6.15E−12 | 1.95E−08 | 0.000634 | 0.000373 | 0.000673 | 0.000352 | 0.001228 |
| Lncppara | 8 | 8.45E−12 | 2.33E−08 | 0.000567 | 0.000244 | 0.005637 | 0.000645 | 0.000195 |
| Vmn2r47 | 9 | 1.02E−11 | 2.33E−08 | 7.51E−05 | 0.00118 | 0.003396 | 0.000129 | 0.003129 |
| Gm45351 | 10 | 1.05E−11 | 2.33E−08 | 7.51E−05 | 0.001151 | 0.003794 | 0.000129 | 0.002962 |
| Irx3 | 11 | 2.05E−11 | 4.12E−08 | 0.000283 | 8.79E−05 | 0.005852 | 0.00465 | 0.000391 |
| Otx1 | 12 | 5.83E−11 | 1.08E−07 | 0.000225 | 0.003116 | 0.002911 | 0.001485 | 0.000283 |
| Smco2 | 13 | 7.54E−11 | 1.28E−07 | 0.018873 | 0.003566 | 0.000205 | 0.000283 | 0.000293 |
| Gria4 | 14 | 1.01E−10 | 1.57E−07 | 8.79E−05 | 0.000801 | 0.003067 | 0.000928 | 0.007942 |
| 4930558F1 | 15 | 1.06E−10 | 1.57E−07 | 0.00031 | 0.000452 | 0.000643 | 0.000934 | 0.020045 |
| 7Rik | | | | | | | | |
| Ptprd | 16 | 1.20E−10 | 1.66E−07 | 0.004953 | 0.003087 | 1.30E−05 | 0.00295 | 0.003292 |
| Defa23 | 17 | 1.32E−10 | 1.72E−07 | 0.003051 | 0.000221 | 0.000614 | 0.000381 | 0.01361 |
| Skint6 | 18 | 3.74E−10 | 4.60E−07 | 0.001798 | 0.002031 | 0.001656 | 0.000833 | 0.001391 |
| Emilin2 | 19 | 4.38E−10 | 5.11E−07 | 0.003478 | 0.000762 | 0.003058 | 0.000586 | 0.001768 |
| Gucy1a2 | 20 | 4.83E−10 | 5.35E−07 | 0.002667 | 0.002696 | 0.002774 | 0.000225 | 0.002091 |
| Obscn | 21 | 6.83E−10 | 7.20E−07 | 0.000166 | 0.000186 | 0.002794 | 0.013852 | 0.011644 |
| Skint5 | 22 | 8.54E−10 | 8.60E−07 | 0.002136 | 0.005779 | 0.001824 | 0.000738 | 0.001078 |
| Gm3002 | 23 | 9.17E−10 | 8.83E−07 | 0.014151 | 0.00014 | 0.001266 | 0.011594 | 0.000667 |
| Irx5 | 24 | 1.30E−09 | 1.20E−06 | 0.045621 | 0.000342 | 0.00379 | 0.002384 | 0.000205 |
| Lrrc4b | 25 | 1.66E−09 | 1.47E−06 | 0.000274 | 0.008381 | 0.003322 | 0.001911 | 0.002618 |
| Gm16513 | 26 | 1.82E−09 | 1.55E−06 | 0.001709 | 0.000143 | 0.036228 | 0.001232 | 0.003919 |
| Xkr4 | 27 | 2.38E−09 | 1.91E−06 | 0.01058 | 0.004406 | 0.001172 | 0.000469 | 0.002257 |
| Galnt10 | 28 | 2.42E−09 | 1.91E−06 | 0.00206 | 0.000381 | 0.026044 | 0.000195 | 0.014771 |
| Hoxa9 | 29 | 2.96E−09 | 2.26E−06 | 0.000225 | 0.002774 | 0.110134 | 0.000391 | 0.002765 |
| Snhg14 | 30 | 4.58E−09 | 3.38E−06 | 0.002335 | 0.000635 | 0.012065 | 0.004611 | 0.001485 |
| Raet1d | 31 | 4.76E−09 | 3.40E−06 | 0.01556 | 0.02055 | 4.88E−05 | 0.003888 | 0.00211 |
| Dnah6 | 32 | 4.92E−09 | 3.41E−06 | 0.002911 | 0.002364 | 0.004005 | 0.00043 | 0.011224 |
| Ankrd33b | 33 | 5.16E−09 | 3.46E−06 | 0.000244 | 0.00804 | 0.004367 | 0.003575 | 0.004582 |
| Rims2 | 34 | 7.88E−09 | 5.03E−06 | 0.00931 | 0.000469 | 0.031495 | 0.001299 | 0.00128 |
| Hoxd3 | 35 | 7.95E−09 | 5.03E−06 | 0.084618 | 1.95E−05 | 0.093947 | 0.03044 | 4.88E−05 |
| Shmt2 | 36 | 8.73E−09 | 5.37E−06 | 0.007942 | 0.000381 | 0.000528 | 0.00043 | 0.375007 |
| Cd84 | 37 | 1.09E−08 | 6.55E−06 | 0.00043 | 0.007288 | 0.000801 | 0.002091 | 0.063693 |
| Atp6v1c1 | 38 | 1.17E−08 | 6.68E−06 | 0.000782 | 0.000664 | 0.011908 | 0.003058 | 0.019137 |
| Gm9962 | 39 | 1.18E−08 | 6.68E−06 | 0.000889 | 0.001475 | 0.001612 | 0.003556 | 0.048288 |
| Sparc | 40 | 1.29E−08 | 7.15E−06 | 0.000234 | 0.001329 | 0.007571 | 0.005011 | 0.034318 |
| Trim52 | 41 | 1.32E−08 | 7.15E−06 | 0.002452 | 0.002208 | 0.003888 | 0.011771 | 0.00168 |
| Rfx8 | 42 | 1.39E−08 | 7.32E−06 | 0.002667 | 0.011908 | 0.003097 | 0.001612 | 0.002774 |
| Serpina3n | 43 | 1.57E−08 | 7.97E−06 | 0.00043 | 0.000322 | 0.003087 | 0.047389 | 0.025018 |
| Nr4a2 | 44 | 1.59E−08 | 7.97E−06 | 0.009935 | 0.001065 | 0.003868 | 0.002335 | 0.005383 |
| Hoxa7 | 45 | 1.62E−08 | 7.97E−06 | 0.002159 | 0.003429 | 0.004601 | 0.004953 | 0.003116 |
| Jak3 | 46 | 1.67E−08 | 8.02E−06 | 0.000352 | 0.000664 | 0.003614 | 0.017086 | 0.03761 |
| Mrgpra2b | 47 | 1.83E−08 | 8.56E−06 | 0.001634 | 0.001498 | 0.054981 | 0.000378 | 0.011923 |
| 4930509J0 | 48 | 1.85E−08 | 8.56E−06 | 0.005011 | 0.010589 | 0.000977 | 0.001612 | 0.007356 |
| 9Rik | | | | | | | | |
| Mical3 | 49 | 1.99E−08 | 8.98E−06 | 0.007102 | 0.003116 | 0.002931 | 0.001006 | 0.010199 |
| Vmn2r- | 50 | 2.12E−08 | 9.25E−06 | 0.00966 | 0.011556 | 0.007297 | 0.000667 | 0.001322 |
| ps158 | | | | | | | | |
| 4930578G1 | 51 | 2.13E−08 | 9.25E−06 | 0.021507 | 0.002198 | 0.003067 | 0.00136 | 0.003659 |
| 0Rik | | | | | | | | |
| Lrrc47 | 52 | 2.31E−08 | 9.84E−06 | 0.001612 | 0.004162 | 0.003868 | 0.006389 | 0.004787 |
| Gm3772 | 53 | 2.39E−08 | 1.00E−05 | 0.000117 | 0.000401 | 0.029895 | 0.001598 | 0.367925 |
| Tamalin | 54 | 2.64E−08 | 1.08E−05 | 0.001797 | 0.001133 | 0.010414 | 0.000635 | 0.068871 |
| Erbb4 | 55 | 2.73E−08 | 1.10E−05 | 0.005568 | 0.004367 | 0.005627 | 0.00339 | 0.002071 |
| Zfp61 | 56 | 3.07E−08 | 1.21E−05 | 0.000127 | 0.059688 | 0.040687 | 8.79E−05 | 0.040619 |
| Pacsin1 | 57 | 3.16E−08 | 1.21E−05 | 0.013442 | 0.006076 | 0.000596 | 0.005949 | 0.003947 |
| Zfp46 | 58 | 3.21E−08 | 1.21E−05 | 0.00382 | 0.006193 | 0.025487 | 0.000106 | 0.01819 |
| E4f1 | 59 | 3.22E−08 | 1.21E−05 | 0.00337 | 0.024793 | 0.000967 | 0.008948 | 0.001612 |
| Cbx8 | 60 | 3.31E−08 | 1.22E−05 | 0.001407 | 0.000381 | 0.002638 | 0.011215 | 0.075943 |
| Tbc1d9 | 61 | 3.45E−08 | 1.22E−05 | 0.001612 | 0.017838 | 0.004025 | 0.001813 | 0.006018 |
| Ctnnd2 | 62 | 3.46E−08 | 1.22E−05 | 0.003194 | 0.002178 | 0.000322 | 0.03 | 0.018864 |
| Aktip | 63 | 3.48E−08 | 1.22E−05 | 0.000195 | 6.84E−05 | 0.015904 | 0.27728 | 0.021628 |
| Ripor2 | 64 | 4.08E−08 | 1.41E−05 | 0.003702 | 0.001807 | 0.003654 | 0.002091 | 0.030059 |
| Dnah7c | 65 | 4.54E−08 | 1.52E−05 | 0.051462 | 0.001504 | 0.000244 | 0.005939 | 0.015474 |
| Hoxa2 | 66 | 4.54E−08 | 1.52E−05 | 0.001543 | 0.001768 | 0.042495 | 0.000703 | 0.021345 |
| Mrgprx2 | 67 | 4.63E−08 | 1.53E−05 | 0.00069 | 0.000605 | ND | 0.0004 | ND |
| 1500004A1 | 68 | 4.84E−08 | 1.57E−05 | 0.009776 | 0.000801 | 0.001446 | 0.012133 | 0.013657 |
| 3Rik | | | | | | | | |
| Gon4l | 69 | 4.90E−08 | 1.57E−05 | 0.01478 | 0.000283 | 0.002813 | 0.001768 | 0.091358 |
| Gabrg3 | 70 | 5.27E−08 | 1.67E−05 | 0.001749 | 0.002384 | 0.003575 | 0.002853 | 0.048708 |
| 1700021N2 | 71 | 6.72E−08 | 2.10E−05 | 0.001476 | 0.004139 | 0.004927 | 0.001059 | 0.086288 |
| 1Rik | | | | | | | | |
| Camk2b | 72 | 6.88E−08 | 2.11E−05 | 0.001612 | 0.008518 | 0.017437 | 0.011176 | 0.001055 |
| Syngap1 | 73 | 6.98E−08 | 2.11E−05 | 0.008392 | 0.004328 | 0.001749 | 0.015574 | 0.002903 |
| Gpr137b-ps | 74 | 7.05E−08 | 2.11E−05 | 0.000568 | 0.000245 | 0.028771 | 0.008624 | 0.084356 |
| Slc22a27 | 75 | 7.93E−08 | 2.34E−05 | 0.004324 | 0.005464 | 0.039565 | 0.00195 | 0.00183 |
| Skint2 | 76 | 8.20E−08 | 2.39E−05 | 0.0042 | 0.012131 | 0.003151 | 0.003363 | 0.00643 |
| Zbtb7a | 77 | 8.40E−08 | 2.39E−05 | 0.235537 | 0.000225 | 0.000469 | 0.061104 | 0.002354 |
| Paqr5 | 78 | 8.41E−08 | 2.39E−05 | 0.012162 | 0.000967 | 0.009622 | 0.004425 | 0.007131 |
| 1700101I11 | 79 | 8.86E−08 | 2.45E−05 | 0.051927 | 0.005295 | 0.002418 | 0.003771 | 0.001515 |
| Rik | | | | | | | | |
| Cacna1a | 80 | 8.89E−08 | 2.45E−05 | 0.002341 | 0.001188 | 0.004914 | 0.006174 | 0.04523 |
| Sema4b | 81 | 8.97E−08 | 2.45E−05 | 0.048239 | 0.021111 | 0.006262 | 0.000469 | 0.001289 |
| Esyt2 | 82 | 9.96E−08 | 2.69E−05 | 0.000205 | 0.003575 | 0.047867 | 0.008509 | 0.014585 |
| Nr2f2 | 83 | 1.01E−07 | 2.69E−05 | 0.006946 | 0.016138 | 0.002335 | 0.091622 | 0.000186 |
| Adcy2 | 84 | 1.02E−07 | 2.69E−05 | 0.000655 | 0.00591 | 0.000977 | 0.010951 | 0.108376 |
| Galnt6 | 85 | 1.08E−07 | 2.81E−05 | 0.004142 | 0.007219 | 0.000733 | 0.01563 | 0.013979 |
| Wnt3a | 86 | 1.10E−07 | 2.84E−05 | 0.004279 | 0.001573 | 0.004435 | 0.022459 | 0.007336 |
| Sprn | 87 | 1.16E−07 | 2.94E−05 | 0.00429 | 0.011022 | 0.003621 | 0.021623 | 0.001408 |
| Rgs22 | 88 | 1.18E−07 | 2.94E−05 | 0.011898 | 0.019303 | 0.004425 | 0.002911 | 0.001797 |
| Col28a1 | 89 | 1.18E−07 | 2.94E−05 | 6.82E−05 | 0.042567 | 0.000151 | 0.096835 | 0.125218 |
| Gm38499 | 90 | 1.20E−07 | 2.95E−05 | 0.000361 | 0.108796 | 0.021472 | 4.88E−05 | 0.131518 |
| Ribc2 | 91 | 1.22E−07 | 2.96E−05 | 0.000537 | 0.005998 | 0.015249 | 0.023142 | 0.004845 |
| Hoxaas3 | 92 | 1.29E−07 | 3.10E−05 | 0.006252 | 0.00464 | 0.021015 | 0.000127 | 0.076207 |
| Pde9a | 93 | 1.37E−07 | 3.25E−05 | 0.006672 | 0.003107 | 0.016715 | 0.004367 | 0.004171 |
| Skint8 | 94 | 1.46E−07 | 3.45E−05 | 0.005283 | 0.002113 | 0.034209 | 0.000979 | 0.018328 |
| Arhgap35 | 95 | 1.50E−07 | 3.50E−05 | 0.001029 | 0.026689 | 0.0169 | 0.000159 | 0.095557 |
| Gm5796 | 96 | 1.62E−07 | 3.73E−05 | 0.002774 | 0.080183 | 0.005246 | 0.004562 | 0.001446 |
| Lingo2 | 97 | 1.74E−07 | 3.98E−05 | 0.001543 | 0.001729 | 0.007698 | 0.027705 | 0.01478 |
| Gm2042 | 98 | 1.77E−07 | 4.00E−05 | 0.002551 | 0.003354 | 0.008974 | 0.014766 | 0.007558 |
| Raet1c | 99 | 1.82E−07 | 4.06E−05 | 0.012929 | 0.008089 | 0.008797 | 0.001254 | 0.007648 |
| Gm1968 | 100 | 1.86E−07 | 4.13E−05 | 0.004484 | 0.007141 | 0.177295 | 0.003155 | 0.000508 |
| Tdpoz5 | 101 | 1.92E−07 | 4.20E−05 | 0.010389 | 0.004823 | 0.00029 | 0.002903 | 0.223518 |
| Vmn2r- | 102 | 1.94E−07 | 4.20E−05 | 0.001351 | 0.003748 | 0.0117 | 0.008976 | 0.017919 |
| ps11 | | | | | | | | |
| Usp13 | 103 | 1.95E−07 | 4.20E−05 | 0.002159 | 0.001534 | 0.013081 | 0.005236 | 0.042348 |
| Fxyd1 | 104 | 2.02E−07 | 4.28E−05 | 0.004328 | 0.008743 | 0.009222 | 0.000293 | 0.097493 |
| Gm14486 | 105 | 2.03E−07 | 4.28E−05 | 0.020554 | 0.005187 | 0.011254 | 0.001895 | 0.004425 |
| Lrp1b | 106 | 2.11E−07 | 4.40E−05 | 0.005998 | 0.007805 | 0.004308 | 0.002774 | 0.018795 |
| 1700030N0 | 107 | 2.15E−07 | 4.44E−05 | 0.036897 | 0.001426 | 0.006047 | 0.01266 | 0.002667 |
| 3Rik | | | | | | | | |
| Has1 | 108 | 2.23E−07 | 4.56E−05 | 0.140779 | 0.001319 | 0.001631 | 0.00253 | 0.014624 |
| Yars | 109 | 2.30E−07 | 4.68E−05 | 0.00171 | 0.002882 | 0.016754 | 0.006701 | 0.021091 |
| Crtc1 | 110 | 2.50E−07 | 5.03E−05 | 0.003556 | 0.00253 | 0.00297 | 0.010873 | 0.044233 |
| Ephb4 | 111 | 2.75E−07 | 5.48E−05 | 0.020935 | 0.009788 | 0.00253 | 0.007815 | 0.003546 |
| Mir7649 | 112 | 2.95E−07 | 5.83E−05 | 0.002575 | 0.005792 | 0.002526 | 0.054925 | 0.007552 |
| Kcnk9 | 113 | 3.06E−07 | 5.99E−05 | 0.003204 | 0.138171 | 0.002618 | 0.000801 | 0.017574 |
| Apba2 | 114 | 3.14E−07 | 6.10E−05 | 0.00845 | 0.006545 | 0.002774 | 0.000283 | 0.38702 |
| Lrrc25 | 115 | 3.22E−07 | 6.19E−05 | 0.004757 | 0.005558 | 0.00295 | 0.00211 | 0.105172 |
| Mmp2 | 116 | 3.40E−07 | 6.48E−05 | 0.000283 | 0.003732 | 0.317879 | 0.003116 | 0.017633 |
| Tmem181c- | 117 | 3.51E−07 | 6.63E−05 | 0.026386 | 0.002853 | 0.021531 | 0.001016 | 0.011644 |
| ps | | | | | | | | |
| Fbxl7 | 118 | 3.58E−07 | 6.71E−05 | 0.102251 | 0.000283 | 0.003194 | 0.002901 | 0.07311 |
| Klra14-ps | 119 | 3.76E−07 | 6.99E−05 | 0.003344 | 0.00847 | 0.013882 | 0.005825 | 0.009085 |
| Kcnh7 | 120 | 3.86E−07 | 7.07E−05 | 0.001973 | 0.017862 | 0.124866 | 0.000283 | 0.017213 |
| Ccm2l | 121 | 3.86E−07 | 7.07E−05 | 0.001319 | 0.004308 | 0.180402 | 0.00211 | 0.009945 |
| Qrfp | 122 | 4.03E−07 | 7.31E−05 | 0.006916 | 0.002696 | 0.007258 | 0.004523 | 0.036907 |
| Hoxa3 | 123 | 4.18E−07 | 7.52E−05 | 0.003409 | 0.004259 | 0.030997 | 0.000977 | 0.05366 |
| Ugt8a | 124 | 4.45E−07 | 7.89E−05 | 0.007288 | 0.012416 | 0.003292 | 0.01224 | 0.006965 |
| Prkd2 | 125 | 4.45E−07 | 7.89E−05 | 0.002335 | 0.209943 | 0.005246 | 0.000114 | 0.086748 |
| Mrgpra2a | 126 | 4.66E−07 | 8.19E−05 | 0.035901 | 0.003762 | 0.001461 | 0.00291 | 0.046814 |
| Rex2 | 127 | 4.72E−07 | 8.22E−05 | 0.000899 | 0.002027 | 0.005832 | 0.010404 | 0.246596 |
| Rnf17 | 128 | 5.03E−07 | 8.70E−05 | 0.002188 | 0.011517 | 0.038694 | 0.004621 | 0.006526 |
| Pcdhga12 | 129 | 5.29E−07 | 9.08E−05 | 0.089307 | 0.005715 | 0.003927 | 0.001289 | 0.012084 |
| Mfap2 | 130 | 5.50E−07 | 9.36E−05 | 0.025985 | 0.023055 | 0.000918 | 0.002931 | 0.02027 |
| Fam171a2 | 131 | 5.61E−07 | 9.48E−05 | 0.00254 | 0.006106 | 0.021365 | 0.011557 | 0.008743 |
| Tc2n | 132 | 5.69E−07 | 9.54E−05 | 0.039769 | 0.004386 | 0.004328 | 0.006438 | 0.007004 |
| Egr3 | 133 | 5.89E−07 | 9.80E−05 | 0.003194 | 0.058232 | 0.001915 | 0.005324 | 0.018698 |
| Nox4 | 134 | 6.21E−07 | 0.000103 | 0.038802 | 0.006233 | 0.012348 | 0.00336 | 0.003761 |
| 4930433N1 | 135 | 6.26E−07 | 0.000103 | 0.000381 | 0.001895 | 0.02154 | 0.055839 | 0.04395 |
| 2Rik | | | | | | | | |
| 5830417I10 | 136 | 6.70E−07 | 0.000109 | 0.007082 | 0.001573 | 0.007669 | 0.00854 | 0.05664 |
| Rik | | | | | | | | |
| Gm14326 | 137 | 6.79E−07 | 0.000109 | 0.004679 | 0.027519 | 0.001719 | 0.012094 | 0.015689 |
| Ranbp17 | 138 | 6.86E−07 | 0.000109 | 0.009847 | 0.013149 | 0.012025 | 0.037776 | 0.000723 |
| Asic4 | 139 | 6.87E−07 | 0.000109 | 0.001368 | 0.005666 | 0.011557 | 0.06046 | 0.007864 |
| Cntnap5c | 140 | 7.10E−07 | 0.000112 | 0.015277 | 0.00127 | 0.003173 | 0.014653 | 0.049147 |
| Gemin5 | 141 | 7.30E−07 | 0.000115 | 0.006281 | 0.002036 | 0.020485 | 0.001104 | 0.158256 |
| Cacng7 | 142 | 7.37E−07 | 0.000115 | 0.002081 | 0.066966 | 0.000469 | 0.018414 | 0.038499 |
| Samd14 | 143 | 7.94E−07 | 0.000123 | 0.001993 | 0.003116 | 0.013129 | 0.001172 | 0.529297 |
| Tmc4 | 144 | 8.12E−07 | 0.000125 | 0.000283 | 0.000508 | 0.063673 | 0.160385 | 0.035393 |
| Pnma1 | 145 | 8.18E−07 | 0.000125 | 0.095962 | 0.006037 | 0.001008 | 0.002719 | 0.033021 |
| Sirpb1c | 146 | 8.30E−07 | 0.000125 | 0.028728 | 0.00148 | 0.005127 | 0.004768 | 0.051351 |
| Tescl | 147 | 8.40E−07 | 0.000125 | 3.63E−05 | 0.099978 | 0.252425 | 0.000457 | 0.129281 |
| Fxyd7 | 148 | 8.40E−07 | 0.000125 | 0.002911 | 0.005764 | 0.002169 | 0.003546 | 0.419719 |
| Gm28453 | 149 | 8.44E−07 | 0.000125 | 0.018825 | 0.001036 | 0.018424 | 0.006877 | 0.022039 |
| Tango6 | 150 | 8.82E−07 | 0.00013 | 0.0172 | 0.010604 | 0.043413 | 0.002296 | 0.003155 |
| Cntnap5b | 151 | 8.84E−07 | 0.00013 | 0.001592 | 0.061515 | 0.001104 | 0.041049 | 0.012954 |
| Foxg1 | 152 | 9.21E−07 | 0.000134 | 0.050573 | 0.005998 | 0.046881 | 0.011156 | 0.000381 |
| Abi3 | 153 | 9.28E−07 | 0.000134 | 0.001172 | 0.003761 | 0.006047 | 0.036985 | 0.061798 |
| Csmd1 | 154 | 9.32E−07 | 0.000134 | 0.004689 | 0.00295 | 0.025018 | 0.011605 | 0.015249 |
| Hk1 | 155 | 9.51E−07 | 0.000136 | 0.079782 | 0.004279 | 0.01309 | 0.007854 | 0.001788 |
| D830030K | 156 | 9.87E−07 | 0.00014 | 0.032906 | 0.000547 | 0.009326 | 0.014227 | 0.027487 |
| 20Rik | | | | | | | | |
| Mir23a | 157 | 1.01E−06 | 0.000143 | ND | 0.005251 | 0.014235 | 0.003384 | 0.002139 |
| Yap1 | 158 | 1.03E−06 | 0.000144 | 0.002579 | 0.0203 | 0.008391 | 0.002774 | 0.056347 |
| Rsph9 | 159 | 1.06E−06 | 0.000148 | 0.0186 | 0.042416 | 0.014526 | 0.006291 | 0.000996 |
| Mark4 | 160 | 1.08E−06 | 0.000149 | 0.003758 | 0.007378 | 0.056789 | 0.008219 | 0.005629 |
| Skint10 | 161 | 1.09E−06 | 0.000149 | 0.004378 | 0.012916 | 0.076106 | 0.001075 | 0.015995 |
| Hoxc5 | 162 | 1.09E−06 | 0.000149 | 0.022537 | 0.024881 | 0.035148 | 0.000547 | 0.006868 |
| Tmem267 | 163 | 1.10E−06 | 0.000149 | 0.177061 | 0.000615 | 0.122697 | 0.008284 | 0.000674 |
| Mir196a-1 | 164 | 1.13E−06 | 0.000153 | 0.016553 | 0.006832 | 0.016678 | 0.003005 | 0.013611 |
| Galnt13 | 165 | 1.17E−06 | 0.000156 | 0.002022 | 0.004836 | 0.03127 | 0.022586 | 0.011596 |
| Rab44 | 166 | 1.19E−06 | 0.000159 | 0.002766 | 0.079275 | 0.002095 | 0.037758 | 0.004736 |
| Srcin1 | 167 | 1.22E−06 | 0.000162 | 0.092482 | 0.005979 | 0.000878 | 0.006909 | 0.025247 |
| Dync2i1 | 168 | 1.24E−06 | 0.000164 | 0.000133 | 0.003909 | 0.006043 | 0.197927 | 0.138708 |
| Spag | 169 | 1.34E−06 | 0.000174 | 0.004279 | 0.056728 | 0.014243 | 0.005988 | 0.004562 |
| Prxl2c | 170 | 1.34E−06 | 0.000174 | 0.020313 | 0.012348 | 0.006076 | 0.007864 | 0.007893 |
| Umad1 | 171 | 1.35E−06 | 0.000174 | 0.001446 | 0.03845 | 0.00043 | 0.057627 | 0.069066 |
| Mroh7 | 172 | 1.44E−06 | 0.000185 | 0.006487 | 0.006076 | 0.010199 | 0.012133 | 0.021091 |
| Espn | 173 | 1.45E−06 | 0.000185 | 0.031778 | 0.000234 | 0.023123 | 0.020935 | 0.028711 |
| Kcnk12 | 174 | 1.47E−06 | 0.000187 | 0.017848 | 0.009837 | 0.018346 | 0.003761 | 0.008743 |
| Ptprf | 175 | 1.56E−06 | 0.000198 | 0.01424 | 0.004558 | 0.006331 | 0.01914 | 0.01446 |
| Inpp4b | 176 | 1.58E−06 | 0.000198 | 0.002003 | 0.047907 | 0.021872 | 0.002745 | 0.019948 |
| Mlkl | 177 | 1.65E−06 | 0.000205 | 0.007932 | 0.00719 | 0.007903 | 0.029121 | 0.009232 |
| Pitx2 | 178 | 1.65E−06 | 0.000205 | 0.006184 | 0.004601 | 0.042309 | 0.025018 | 0.004025 |
| Pcyox1l | 179 | 1.75E−06 | 0.000216 | 0.026591 | 0.081521 | 0.003585 | 0.025604 | 0.000655 |
| Prss32 | 180 | 1.84E−06 | 0.000227 | 0.020075 | 0.006496 | 0.038773 | 0.05199 | 0.000528 |
| Ajm1 | 181 | 1.86E−06 | 0.000228 | 0.004706 | 0.035338 | 0.022994 | 0.010578 | 0.003468 |
| Tox3 | 182 | 1.88E−06 | 0.000229 | 0.00424 | 0.011178 | 0.025809 | 0.018815 | 0.006174 |
| Bcat1 | 183 | 1.94E−06 | 0.000234 | 0.004836 | 0.005402 | 0.028242 | 0.016343 | 0.012211 |
| Rtn4rl2 | 184 | 1.99E−06 | 0.00024 | 0.004005 | 0.008997 | 0.041059 | 0.008743 | 0.011762 |
| Rassf3 | 185 | 2.02E−06 | 0.000241 | 0.035246 | 0.000537 | 0.002569 | 0.006164 | 0.516597 |
| 4933438B1 | 186 | 2.02E−06 | 0.000241 | 0.001436 | 0.01564 | 0.011918 | 0.00802 | 0.072172 |
| 7Rik | | | | | | | | |
| Asap2 | 187 | 2.04E−06 | 0.000242 | 0.063148 | 0.071286 | 0.123596 | 0.000602 | 0.000469 |
| Mir141 | 188 | 2.18E−06 | 0.000257 | 0.005555 | 0.024731 | 0.006873 | 0.035109 | 0.005118 |
| Ceacam20 | 189 | 2.21E−06 | 0.000259 | 0.005295 | 0.080349 | 0.035754 | 0.000166 | 0.068245 |
| Nbea | 190 | 2.29E−06 | 0.000266 | 0.001036 | 0.004992 | 0.004953 | 0.043765 | 0.160288 |
| Mylk3 | 191 | 2.39E−06 | 0.000277 | 0.015738 | 0.014878 | 0.005988 | 0.016382 | 0.008255 |
| Adgrb3 | 192 | 2.44E−06 | 0.000281 | 0.010873 | 0.003888 | 0.058369 | 0.013764 | 0.005725 |
| Obox3 | 193 | 2.46E−06 | 0.000281 | 0.007269 | 0.006142 | 0.003745 | 0.016583 | 0.070856 |
| Rtkn2 | 194 | 2.47E−06 | 0.000281 | 0.070971 | 0.000586 | 0.000361 | 0.049421 | 0.266016 |
| Pex5l | 195 | 2.48E−06 | 0.000281 | 0.008518 | 0.011322 | 0.029707 | 0.005822 | 0.011869 |
| Rnaset2a | 196 | 2.54E−06 | 0.000287 | 0.005803 | 0.006846 | 0.034777 | 0.008642 | 0.017076 |
| Pals2 | 197 | 2.56E−06 | 0.000287 | 0.036194 | 0.001612 | 0.028984 | 0.028418 | 0.004289 |

TABLE 15
(Supplementary Table S7b. Ranked genes by differential methylation
(Old + OSKM vs Old-3): RANK prom., p-value prom., q-value prom.)

| | RANK | p-value | q-value | p-value | p-value | p-value | p-value | p-value |
|---|---|---|---|---|---|---|---|---|
| Gene | prom. | prom. | prom. | prom. TR1 | prom. TR2 | prom. TR3 | prom. TR4 | prom. TR5 |
| Nfix | 467 | 0.000408 | 0.018611 | 0.008277 | 0.004036 | ND | 0.391114 | 0.05272 |
| Nlrp5-ps | 15 | 4.93E−08 | 6.13E−05 | ND | 3.55E−05 | ND | ND | 6.65E−05 |
| Atp6v1c2 | 1513 | 0.009344 | 0.131582 | 0.04847 | 0.470887 | 0.590701 | 0.002457 | 0.249685 |
| Hoxb3 | 784 | 0.00184 | 0.050012 | 0.023456 | 0.010329 | 0.040094 | 0.203797 | 0.430928 |
| Mup2 | 3248 | 0.054834 | 0.35971 | ND | 0.054834 | ND | ND | ND |
| Rbfox1 | 10994 | 0.503706 | 0.976451 | ND | 0.396429 | 0.926297 | ND | 0.190665 |
| Gm10377 | NA | ND | ND | ND | ND | ND | ND | ND |
| Lncppara | 950 | 0.003027 | 0.067933 | 0.056315 | 0.029515 | 0.026931 | 0.619913 | 0.060768 |
| Vmn2r47 | NA | ND | ND | ND | ND | ND | ND | ND |
| Gm45351 | NA | ND | ND | ND | ND | ND | ND | ND |
| Irx3 | 829 | 0.002129 | 0.054763 | 0.115427 | 0.016084 | 0.058091 | 0.083957 | 0.114953 |
| Otx1 | 3940 | 0.080932 | 0.437716 | 0.01995 | 0.593235 | 0.136475 | 0.691809 | 0.210059 |
| Smco2 | 641 | 0.001033 | 0.03433 | 0.414748 | 0.109836 | 0.029589 | 0.012033 | 0.024184 |
| Gria4 | 10 | 3.41E−08 | 6.13E−05 | 0.000267 | 0.002184 | 0.00831 | 0.012447 | 0.020708 |
| 4930558F17Rik | NA | ND | ND | ND | ND | ND | ND | ND |
| Ptprd | 7236 | 0.253171 | 0.745707 | 0.341787 | 0.466479 | ND | 0.28087 | 0.138076 |
| Defa23 | NA | ND | ND | ND | ND | ND | ND | ND |
| Skint6 | NA | ND | ND | ND | ND | ND | ND | ND |
| Emilin2 | 252 | 7.43E−05 | 0.006285 | 0.055426 | 0.009499 | 0.204766 | 0.012574 | 0.009594 |
| Gucy1a2 | 90 | 3.82E−06 | 0.000905 | 0.047282 | 0.013072 | 0.027986 | 0.004345 | 0.004449 |
| Obscn | 2485 | 0.029974 | 0.257057 | 0.070027 | 0.091178 | 0.113455 | 0.453818 | 0.143406 |
| Skint5 | NA | ND | ND | ND | ND | ND | ND | ND |
| Gm3002 | NA | ND | ND | ND | ND | ND | ND | ND |
| Irx5 | 1200 | 0.005405 | 0.096033 | 0.322613 | 0.028673 | 0.17098 | 0.079625 | 0.030059 |
| Lrrc4b | NA | ND | ND | ND | ND | ND | ND | ND |
| Gm16513 | NA | ND | ND | ND | ND | ND | ND | ND |
| Xkr4 | 77 | 2.73E−06 | 0.000756 | 0.042032 | 0.049247 | 0.010763 | 0.001004 | 0.009956 |
| Galnt10 | 1154 | 0.005 | 0.092181 | ND | 0.011153 | 0.199645 | ND | 0.042145 |
| Hoxa9 | 234 | 5.89E−05 | 0.00537 | 0.018336 | 0.013868 | 0.138818 | 0.012345 | 0.022309 |
| Snhg14 | 242 | 6.47E−05 | 0.005699 | 0.010412 | 0.017072 | 0.213069 | 0.02489 | 0.011592 |
| Raet1d | 32 | 1.48E−07 | 9.65E−05 | ND | ND | 0.000722 | 0.001286 | 0.000644 |
| Dnah6 | 6 | 1.31E−08 | 4.67E−05 | 0.00229 | 0.010052 | 0.003101 | 0.00028 | 0.020607 |
| Ankrd33b | 3217 | 0.054058 | 0.357936 | 0.102707 | 0.198016 | 0.077995 | 0.355839 | 0.212805 |
| Rims2 | 463 | 0.000403 | 0.018544 | 0.268597 | 0.006271 | 0.716792 | 0.014302 | 0.006566 |
| Hoxd3 | 613 | 0.00093 | 0.032273 | 0.279759 | 0.003738 | 0.185318 | 0.420047 | 0.004192 |
| Shmt2 | 159 | 1.79E−05 | 0.002403 | 0.037566 | 0.006437 | 0.008756 | 0.004036 | 0.258499 |
| Cd84 | 71 | 2.28E−06 | 0.000672 | 0.005647 | 0.005591 | 0.004513 | 0.008965 | 0.140464 |
| Atp6v1c1 | 824 | 0.002079 | 0.053795 | 0.017941 | 0.045171 | 0.172765 | 0.032194 | 0.223527 |
| Gm9962 | 59 | 1.31E−06 | 0.000472 | 0.003091 | 0.010656 | 0.007339 | 0.007334 | 0.051746 |
| Sparc | 2822 | 0.040687 | 0.307089 | 0.262976 | 0.055714 | 0.072506 | 0.115176 | 0.622022 |
| Trim52 | 9 | 2.97E−08 | 6.13E−05 | 0.004248 | 0.004004 | 0.002697 | 0.014675 | 0.001576 |
| Rfx8 | 30 | 1.44E−07 | 9.65E−05 | 0.004249 | 0.008108 | 0.004523 | 0.004677 | 0.009228 |
| Serpina3n | 188 | 2.67E−05 | 0.003027 | 0.008614 | 0.0079 | 0.024074 | 0.176081 | 0.012534 |
| Nr4a2 | 11544 | 0.542148 | 1 | 0.569837 | 0.448666 | 0.623605 | 0.165412 | 0.444058 |
| Hoxa7 | 82 | 3.13E−06 | 0.000813 | 0.002154 | 0.056872 | 0.012647 | 0.014666 | 0.011546 |
| Jak3 | 5443 | 0.151648 | 0.59378 | 0.098334 | 0.450839 | 0.517054 | 0.10113 | 0.307351 |
| Mrgpra2b | NA | ND | ND | ND | ND | ND | ND | ND |
| 4930509J09Rik | 19 | 6.48E−08 | 6.83E−05 | 0.004145 | 0.016363 | 0.002101 | 0.004952 | 0.003737 |
| Mical3 | 8 | 2.90E−08 | 6.13E−05 | 0.007363 | 0.003547 | 0.002354 | 0.002033 | 0.008264 |
| Vmn2r-ps158 | NA | ND | ND | ND | ND | ND | ND | ND |
| 4930578G10Rik | NA | ND | ND | ND | ND | ND | ND | ND |
| Lrrc47 | 448 | 0.000369 | 0.017577 | 0.011988 | 0.079527 | 0.050048 | 0.039879 | 0.053231 |
| Gm3772 | 14912 | 0.767828 | 1 | ND | ND | ND | ND | 0.767828 |
| Tamalin | 955 | 0.003056 | 0.068165 | 0.019538 | 0.061024 | 0.058082 | 0.060272 | 0.409357 |
| Erbb4 | 2931 | 0.044219 | 0.321429 | 0.306631 | 0.437209 | 0.174013 | 0.173234 | 0.021501 |
| Zfp61 | 63 | 1.70E−06 | 0.000576 | 0.000139 | 0.247516 | 0.128577 | 0.00056 | 0.050969 |
| Pacsin1 | 741 | 0.001544 | 0.044321 | 0.059413 | 0.052736 | 0.062285 | 0.018398 | 0.1875 |
| Zfp46 | 9282 | 0.390592 | 0.896869 | 0.191153 | 0.348471 | 0.333636 | ND | 0.65738 |
| E4f1 | 3823 | 0.07672 | 0.427625 | 0.47218 | 0.593084 | 0.0138 | 0.536309 | 0.103462 |
| Cbx8 | 1145 | 0.004924 | 0.091566 | 0.427195 | 0.013414 | 0.042894 | 0.072418 | 0.186498 |
| Tbc1d9 | 9167 | 0.383139 | 0.890738 | 0.511015 | 0.432232 | 0.227715 | ND | 0.278854 |
| Ctnnd2 | 2680 | 0.036428 | 0.289552 | 0.227946 | 0.154413 | 0.035313 | 0.418182 | 0.122963 |
| Aktip | 393 | 0.000265 | 0.014345 | 0.006632 | 0.006096 | 0.026845 | 0.571759 | 0.106049 |
| Ripor2 | 37 | 2.82E−07 | 0.000161 | 0.006832 | 0.005838 | 0.003145 | 0.002042 | 0.057929 |
| Dnah7c | 387 | 0.000259 | 0.014275 | 0.640566 | 0.011232 | 0.001075 | 0.103184 | 0.080183 |
| Hoxa2 | 8826 | 0.360499 | 0.870523 | 0.68601 | 0.172607 | 0.149477 | 0.440877 | 0.533811 |
| Mrgprx2 | NA | ND | ND | ND | ND | ND | ND | ND |
| 1500004A13Rik | 1230 | 0.00568 | 0.09845 | ND | 0.00651 | 0.100322 | 0.180448 | 0.171733 |
| Gon4l | 293 | 0.00011 | 0.008039 | 0.321926 | 0.00105 | 0.02184 | 0.009554 | 0.30474 |
| Gabrg3 | 58 | 1.28E−06 | 0.000469 | 0.003105 | 0.010554 | 0.007213 | 0.007305 | 0.051686 |
| 1700021N21Rik | 21 | 6.72E−08 | 6.83E−05 | 0.001476 | 0.004139 | 0.004927 | 0.001059 | 0.086288 |
| Camk2b | 4 | 4.63E−09 | 2.47E−05 | 0.003567 | 0.003309 | 0.005554 | 0.006857 | 0.000276 |
| Syngap1 | 15512 | 0.804166 | 1 | ND | 0.482918 | 0.918567 | ND | ND |
| Gpr137b-ps | NA | ND | ND | ND | ND | ND | ND | ND |
| Slc22a27 | NA | ND | ND | ND | ND | ND | ND | ND |
| Skint2 | NA | ND | ND | ND | ND | ND | ND | ND |
| Zbtb7a | 17872 | 0.918707 | 1 | 0.765397 | 0.409352 | 0.685998 | 0.673757 | 0.707158 |
| Paqr5 | 185 | 2.55E−05 | 0.002923 | 0.020456 | 0.003601 | 0.065944 | 0.036545 | 0.019231 |
| 1700101I11Rik | 27 | 8.86E−08 | 7.00E−05 | 0.051927 | 0.005295 | 0.002418 | 0.003771 | 0.001515 |
| Cacna1a | 2337 | 0.025851 | 0.23573 | ND | ND | 0.027393 | 0.122119 | 0.227545 |
| Sema4b | 709 | 0.001388 | 0.041709 | 0.097724 | 0.114668 | 0.211994 | 0.014513 | 0.016907 |
| Esyt2 | 1280 | 0.006164 | 0.10267 | 0.025251 | 0.061106 | 0.527932 | 0.060806 | 0.092034 |
| Nr2f2 | 1525 | 0.009464 | 0.132192 | 0.166656 | 0.734269 | 0.043049 | 0.109337 | 0.014625 |
| Adcy2 | 4183 | 0.091792 | 0.467575 | 0.017125 | 0.727199 | 0.045547 | 0.623557 | 0.822997 |
| Galnt6 | 1981 | 0.017406 | 0.187237 | 0.082425 | 0.040678 | 0.113736 | 0.580745 | 0.093144 |
| Wnt3a | 2149 | 0.020869 | 0.206896 | 0.192483 | 0.395848 | 0.007133 | 0.093774 | 0.531644 |
| Sprn | 29 | 1.16E−07 | 8.53E−05 | 0.00429 | 0.011022 | 0.003621 | 0.021623 | 0.001408 |
| Rgs22 | 453 | 0.00039 | 0.018354 | 0.035039 | 0.087387 | 0.0216 | 0.049651 | 0.033109 |
| Col28a1 | NA | ND | ND | ND | ND | ND | ND | ND |
| Gm38499 | 1895 | 0.015626 | 0.17571 | 0.111638 | 0.421674 | 0.062525 | 0.02511 | 0.237648 |
| Ribc2 | 57 | 1.24E−06 | 0.000464 | 0.006058 | 0.005832 | 0.016332 | 0.017552 | 0.008507 |
| Hoxaas3 | 2556 | 0.032429 | 0.270284 | 0.709245 | 0.188379 | ND | 0.004248 | 0.399179 |
| Pde9a | 928 | 0.002841 | 0.065277 | 0.084659 | 0.238371 | 0.09403 | 0.035304 | 0.023067 |
| Skint8 | NA | ND | ND | ND | ND | ND | ND | ND |
| Arhgap35 | NA | ND | ND | ND | ND | ND | ND | ND |
| Gm5796 | 93 | 3.99E−06 | 0.000914 | 0.056778 | 0.057223 | 0.010624 | 0.015308 | 0.000666 |
| Lingo2 | 736 | 0.001524 | 0.044144 | 0.009654 | 0.020345 | 0.122105 | 0.369996 | 0.074517 |
| Gm2042 | NA | ND | ND | ND | ND | ND | ND | ND |
| Raet1c | NA | ND | ND | ND | ND | ND | ND | ND |
| Gm1968 | 933 | 0.002896 | 0.066165 | 0.069547 | 0.048345 | 0.482971 | 0.117437 | 0.008319 |
| Tdpoz5 | NA | ND | ND | ND | ND | ND | ND | ND |
| Vmn2r-ps11 | NA | ND | ND | ND | ND | ND | ND | ND |
| Usp13 | 114 | 7.68E−06 | 0.001436 | 0.005426 | 0.012284 | 0.023719 | 0.003555 | 0.138913 |
| Fxyd1 | 1141 | 0.004866 | 0.090917 | 0.296577 | 0.666777 | 0.034872 | 0.00794 | 0.059626 |
| Gm14486 | 20 | 6.69E−08 | 6.83E−05 | 0.016159 | 0.000791 | 0.004422 | 0.008053 | 0.00601 |
| Lrp1b | 5423 | 0.150243 | 0.590363 | 0.202151 | 0.515868 | 0.214895 | 0.341333 | 0.091554 |
| 1700030N03Rik | 4985 | 0.128917 | 0.550776 | 0.400254 | 0.209111 | 0.098018 | 0.615302 | 0.104897 |
| Has1 | 1215 | 0.005536 | 0.097144 | 0.403283 | 0.011897 | 0.106496 | 0.038135 | 0.200949 |
| Yars | 150 | 1.59E−05 | 0.002263 | 0.007456 | 0.02029 | 0.026169 | 0.033816 | 0.014273 |
| Crtc1 | 24 | 7.89E−08 | 6.95E−05 | 0.004248 | 0.001072 | 0.004721 | 0.012108 | 0.012744 |
| Ephb4 | 2172 | 0.021367 | 0.209642 | 0.101456 | 0.354044 | 0.008641 | 0.148267 | 0.610142 |
| Mir7649 | NA | ND | ND | ND | ND | ND | ND | ND |
| Kcnk9 | 28 | 1.03E−07 | 7.86E−05 | 0.005905 | 0.148414 | 0.002187 | 0.000463 | 0.005122 |
| Apba2 | 1 | 2.46E−09 | 2.47E−05 | 0.002388 | 0.002656 | 0.001495 | 6.26E−05 | ND |
| Lrrc25 | 862 | 0.002391 | 0.059133 | 0.005267 | 0.2718 | 0.113367 | 0.022209 | 0.338362 |
| Mmp2 | 36 | 2.06E−07 | 0.000122 | 0.001074 | 0.011256 | 0.341137 | 0.000871 | 0.002857 |
| Tmem181c-ps | 1585 | 0.010337 | 0.138951 | 0.295388 | 0.010454 | 0.102417 | 0.196138 | 0.15432 |
| Fbxl7 | 7 | 2.17E−08 | 6.13E−05 | 0.29539 | 0.000119 | 0.001593 | 0.000679 | 0.019355 |
| Klra14-ps | NA | ND | ND | ND | ND | ND | ND | ND |
| Kcnh7 | 214 | 3.91E−05 | 0.00389 | 0.00215 | ND | 0.569754 | 0.001209 | 0.02671 |
| Ccm2l | 3374 | 0.059359 | 0.374864 | 0.023637 | 0.070697 | 0.490498 | 0.58428 | 0.29224 |
| Qrfp | 406 | 0.000285 | 0.014986 | 0.03529 | 0.018976 | 0.047185 | 0.018485 | 0.124115 |
| Hoxa3 | 8856 | 0.362116 | 0.871258 | 0.685884 | 0.173372 | 0.149595 | 0.441318 | 0.5361 |
| Ugt8a | 5 | 1.14E−08 | 4.67E−05 | 0.00118 | 0.018265 | 0.000767 | 0.009723 | 0.002178 |
| Prkd2 | 4981 | 0.128868 | 0.550776 | 0.150819 | 0.142186 | 0.269287 | ND | 0.328438 |
| Mrgpra2a | NA | ND | ND | ND | ND | ND | ND | ND |
| Rex2 | 483 | 0.000468 | 0.020679 | 0.011429 | ND | 0.030162 | 0.01568 | ND |
| Rnf17 | 66 | 1.91E−06 | 0.000611 | 0.002873 | 0.011245 | 0.06685 | 0.012983 | 0.005152 |
| Pcdhga12 | 12 | 4.31E−08 | 6.13E−05 | 0.076172 | 0.002013 | 0.002199 | 0.001007 | 0.004816 |
| Mfap2 | 39 | 3.36E−07 | 0.000182 | 0.01915 | 0.022444 | 0.0005 | 0.002445 | 0.034676 |
| Fam171a2 | 10877 | 0.496084 | 0.97189 | 0.463029 | 0.847549 | 0.093033 | 0.341244 | 0.735873 |
| Tc2n | 89 | 3.80E−06 | 0.000905 | 0.049615 | 0.00313 | 0.017294 | 0.011819 | 0.010469 |
| Egr3 | 16444 | 0.853847 | 1 | 0.215045 | 0.970186 | 0.764904 | 0.678991 | 0.584088 |
| Nox4 | 35 | 1.87E−07 | 0.000114 | 0.054062 | 0.00477 | 0.017752 | 0.002661 | 0.000749 |
| 4930433N12Rik | 235 | 5.93E−05 | 0.005378 | 0.000978 | 0.020596 | 0.061162 | 0.080316 | 0.098987 |
| 5830417I10Rik | 4056 | 0.085775 | 0.450647 | 0.044614 | 0.043955 | 0.61614 | ND | 0.813721 |
| Gm14326 | 162 | 1.99E−05 | 0.00256 | 0.013336 | 0.02629 | 0.024111 | 0.012649 | 0.023436 |
| Ranbp17 | 2 | 2.97E−09 | 2.47E−05 | 0.003091 | 0.005334 | 0.002156 | 0.021223 | 9.87E−05 |
| Asic4 | 1490 | 0.008987 | 0.128384 | 0.007015 | 0.044873 | 0.107191 | 0.752219 | 0.30803 |
| Cntnap5c | 5341 | 0.146649 | 0.584787 | ND | 0.02094 | ND | 0.524032 | 0.782797 |
| Gemin5 | 2998 | 0.046803 | 0.332307 | 0.120893 | ND | 0.204373 | 0.019815 | 0.794137 |
| Cacng7 | 1397 | 0.007665 | 0.116981 | 0.008723 | 0.260966 | 0.053039 | 0.148018 | 0.348 |
| Samd14 | 1343 | 0.006927 | 0.109965 | 0.128313 | 0.006147 | 0.220282 | 0.033468 | 0.9255 |
| Tmc4 | 43 | 4.70E−07 | 0.000233 | 0.001133 | 0.000398 | 0.051154 | 0.084522 | 0.013913 |
| Pnma1 | 45 | 8.18E−07 | 0.000387 | 0.095962 | 0.006037 | 0.001008 | 0.002719 | 0.033021 |
| Sirpb1c | NA | ND | ND | ND | ND | ND | ND | ND |
| Tescl | 46 | 8.40E−07 | 0.000389 | 3.63E−05 | 0.099978 | 0.252425 | 0.000457 | 0.129281 |
| Fxyd7 | 3 | 4.14E−09 | 2.47E−05 | 0.000656 | 0.000728 | 0.001339 | 0.000988 | 0.17261 |
| Gm28453 | 1774 | 0.01331 | 0.159821 | 0.741742 | 0.018299 | 0.200554 | 0.054944 | 0.092635 |
| Tango6 | 1265 | 0.006047 | 0.101914 | ND | ND | 0.141636 | 0.039254 | 0.021378 |
| Cntnap5b | 478 | 0.00045 | 0.020076 | 0.001815 | 0.597423 | 0.001978 | 0.788163 | 0.077576 |
| Foxg1 | 482 | 0.000456 | 0.020153 | 0.113798 | 0.039799 | 0.118377 | 0.04118 | 0.006032 |
| Abi3 | 64 | 1.86E−06 | 0.00061 | 0.011499 | 0.018215 | 0.00229 | 0.015933 | 0.018306 |
| Csmd1 | 225 | 5.03E−05 | 0.004765 | 0.009341 | 0.017638 | 0.05982 | 0.055661 | 0.014522 |
| Hk1 | 356 | 0.000203 | 0.012166 | 0.132388 | 0.037443 | 0.143782 | 0.006519 | 0.010067 |
| D830030K20Rik | NA | ND | ND | ND | ND | ND | ND | ND |
| Mir23a | 51 | 1.01E−06 | 0.000423 | ND | 0.005251 | 0.014235 | 0.003384 | 0.002139 |
| Yap1 | 33 | 1.49E−07 | 9.65E−05 | 0.00104 | 0.014957 | 0.003901 | 0.007218 | 0.015999 |
| Rsph9 | 50 | 9.90E−07 | 0.000422 | 0.013546 | 0.032777 | 0.004374 | 0.012575 | 0.002696 |
| Mark4 | NA | ND | ND | ND | ND | ND | ND | ND |
| Skint10 | NA | ND | ND | ND | ND | ND | ND | ND |
| Hoxc5 | 3018 | 0.04724 | 0.333493 | 0.12969 | 0.326211 | 0.352037 | 0.030044 | 0.215875 |
| Tmem267 | 189 | 2.70E−05 | 0.003046 | 0.070145 | 0.007252 | 0.536121 | 0.033129 | 0.000406 |
| Mir196a-1 | 52 | 1.13E−06 | 0.000456 | 0.016553 | 0.006832 | 0.016678 | 0.003005 | 0.013611 |
| Galnt13 | 343 | 0.000179 | 0.01112 | 0.004725 | 0.017881 | 0.118289 | 0.223621 | 0.017784 |
| Rab44 | NA | ND | ND | ND | ND | ND | ND | ND |
| Srcin1 | 11739 | 0.557174 | 1 | 0.592638 | 0.37577 | ND | ND | ND |
| Dync2i1 | 10284 | 0.457841 | 0.94876 | ND | ND | ND | 0.436853 | 0.372062 |
| Spag1 | 1155 | 0.005004 | 0.092181 | 0.364325 | 0.065072 | 0.024182 | 0.060937 | 0.097193 |
| Prxl2c | 1340 | 0.006905 | 0.109862 | ND | 0.052448 | 0.049993 | 0.170658 | 0.058749 |
| Umad1 | 12203 | 0.58847 | 1 | 0.99999 | 0.392618 | 0.385575 | 0.305642 | 0.321843 |
| Mroh7 | 5620 | 0.162062 | 0.614558 | 0.156361 | 0.157715 | 0.176493 | 0.407168 | 0.455003 |
| Espn | 5877 | 0.175437 | 0.636142 | 0.085975 | 0.137974 | 0.265652 | 0.711549 | 0.417661 |
| Kcnk12 | 4073 | 0.086823 | 0.45425 | 0.191619 | 0.093067 | 0.715831 | 0.095872 | 0.216173 |
| Ptprf | NA | ND | ND | ND | ND | ND | ND | ND |
| Inpp4b | 4377 | 0.101054 | 0.491984 | 0.366254 | 0.649636 | 0.190931 | 0.029028 | 0.260749 |
| Mlkl | 41 | 3.50E−07 | 0.000182 | 0.006928 | 0.008065 | 0.004707 | 0.010776 | 0.006742 |
| Pitx2 | 47 | 9.20E−07 | 0.000408 | 0.005116 | 0.014481 | 0.036589 | 0.00736 | 0.003024 |
| Pcyox1l | 23 | 7.49E−08 | 6.94E−05 | 0.003952 | 0.035638 | 0.006099 | 0.006727 | 0.00054 |
| Prss32 | 690 | 0.001254 | 0.038754 | 0.11911 | 0.005129 | 0.283092 | 0.085602 | 0.03437 |
| Ajm1 | 65 | 1.86E−06 | 0.00061 | 0.004706 | 0.035338 | 0.022994 | 0.010578 | 0.003468 |
| Tox3 | 212 | 3.90E−05 | 0.00389 | 0.06432 | ND | 0.016757 | 0.01227 | 0.002981 |
| Bcat1 | 720 | 0.001437 | 0.042554 | 0.059079 | 0.067531 | 0.064246 | 0.15478 | 0.015403 |
| Rtn4rl2 | 5299 | 0.144966 | 0.582976 | 0.546465 | 0.042348 | 0.436997 | 0.466606 | 0.138946 |
| Rassf3 | 1478 | 0.008823 | 0.127179 | 0.161032 | 0.033274 | 0.069776 | 0.021468 | 0.948426 |
| 4933438B17Rik | 14205 | 0.72454 | 1 | 0.27031 | 0.312573 | 0.83449 | 0.438566 | 0.97189 |
| Asap2 | 388 | 0.000261 | 0.01432 | ND | ND | 0.148633 | ND | 0.00015 |
| Mir141 | 69 | 2.18E−06 | 0.000672 | 0.005555 | 0.024731 | 0.006873 | 0.035109 | 0.005118 |
| Ceacam20 | 3321 | 0.057499 | 0.368906 | 0.17589 | 0.368171 | 0.177944 | 0.028466 | 0.40497 |
| Nbea | 165 | 2.01E−05 | 0.00256 | 0.000926 | 0.00214 | 0.042841 | 0.049814 | 0.601219 |
| Mylk3 | 2151 | 0.020885 | 0.206904 | 0.301084 | 0.05173 | 0.142658 | 0.116557 | 0.10474 |
| Adgrb3 | 31 | 1.45E−07 | 9.65E−05 | 0.003739 | 0.003242 | 0.042801 | 0.009558 | 0.001369 |
| Obox3 | NA | ND | ND | ND | ND | ND | ND | ND |
| Rtkn2 | 16783 | 0.871648 | 1 | 0.463466 | 0.953184 | 0.288687 | 0.653352 | 0.856087 |
| Pex5l | 17 | 5.17E−08 | 6.13E−05 | 0.002717 | 0.005734 | 0.026749 | 0.002465 | 0.001971 |
| Rnaset2a | 5959 | 0.179107 | 0.640436 | 0.047037 | ND | 0.449644 | ND | 0.551351 |
| Pals2 | 38 | 2.88E−07 | 0.000161 | 0.010506 | 0.002228 | 0.009647 | 0.024936 | 0.002694 |

TABLE 16
(Supplementary Table S7c. Ranked genes by differential methylation
(Old + OSKM vs Old-3): Rank_body, p-value body, q-value body)

| | | p-value | q-value | p-value | p-value | p-value | p-value | p-value |
|---|---|---|---|---|---|---|---|---|
| Gene | Rank_body | body | body | body TR1 | body TR2 | body TR3 | body TR4 | body TR5 |
| Nfix | 4 | 4.19E−13 | 1.88E−09 | 0.000245 | 1.84E−05 | 0.00257 | 0.00116 | 0.000258 |
| Nlrp5-ps | 1748 | 0.00292 | 0.029889 | 0.140977 | 0.003209 | 0.290766 | 0.048207 | 0.252995 |
| Atp6v1c2 | 109 | 2.97E−07 | 4.82E−05 | 0.028049 | 0.010443 | 0.209071 | 1.40E−05 | 0.018352 |
| Hoxb3 | 1 | 2.37E−14 | 4.25E−10 | 5.85E−05 | 0.000492 | 1.26E−05 | 0.000503 | 0.000801 |
| Mup2 | 6 | 1.23E−12 | 3.68E−09 | 0.000931 | 0.000239 | 0.000318 | 0.000217 | 0.000746 |
| Rbfox1 | 3 | 2.12E−13 | 1.27E−09 | 1.76E−07 | 0.02143 | 0.044377 | 3.73E−07 | 0.02612 |
| Gm10377 | 8 | 6.15E−12 | 1.38E−08 | 0.000634 | 0.000373 | 0.000673 | 0.000352 | 0.001228 |
| Lncppara | 7 | 1.50E−12 | 3.86E−09 | 0.000526 | 0.000269 | 0.01899 | 5.52E−05 | 9.68E−05 |
| Vmn2r47 | 10 | 1.02E−11 | 1.69E−08 | 7.51E−05 | 0.00118 | 0.003396 | 0.000129 | 0.003129 |
| Gm45351 | 11 | 1.05E−11 | 1.69E−08 | 7.51E−05 | 0.001151 | 0.003794 | 0.000129 | 0.002962 |
| Irx3 | 9 | 7.47E−12 | 1.49E−08 | 7.75E−05 | 0.000158 | 0.009391 | 0.004806 | 0.000155 |
| Otx1 | 2 | 1.48E−13 | 1.27E−09 | 0.000341 | 0.000395 | 0.001555 | 0.000129 | 4.06E−05 |
| Smco2 | 15 | 6.68E−11 | 7.99E−08 | 0.005777 | 0.00245 | 0.000213 | 0.000748 | 0.000443 |
| Gria4 | 302 | 8.64E−06 | 0.000512 | 0.008933 | 0.02151 | 0.027452 | 0.004294 | 0.039817 |
| 4930558F17Rik | 16 | 1.06E−10 | 1.18E−07 | 0.00031 | 0.000452 | 0.000643 | 0.000934 | 0.020045 |
| Ptprd | 5 | 1.21E−12 | 3.68E−09 | 0.001277 | 0.000491 | 1.30E−05 | 0.000768 | 0.0018 |
| Defa23 | 18 | 1.32E−10 | 1.24E−07 | 0.003051 | 0.000221 | 0.000614 | 0.000381 | 0.01361 |
| Skint6 | 23 | 3.74E−10 | 2.82E−07 | 0.001798 | 0.002031 | 0.001656 | 0.000833 | 0.001391 |
| Emilin2 | 51 | 1.96E−08 | 6.77E−06 | 0.004727 | 0.004531 | 0.001102 | 0.002388 | 0.011639 |
| Gucy1a2 | 119 | 3.76E−07 | 5.61E−05 | 0.003996 | 0.014773 | 0.007061 | 0.001585 | 0.031525 |
| Obscn | 14 | 1.70E−11 | 2.18E−08 | 6.11E−05 | 5.83E−05 | 0.001765 | 0.003681 | 0.009273 |
| Skint5 | 27 | 8.54E−10 | 5.68E−07 | 0.002136 | 0.005779 | 0.001824 | 0.000738 | 0.001078 |
| Gm3002 | 28 | 9.17E−10 | 5.87E−07 | 0.014151 | 0.00014 | 0.001266 | 0.011594 | 0.000667 |
| Irx5 | 24 | 3.77E−10 | 2.82E−07 | 0.022165 | 0.000428 | 0.001726 | 0.002058 | 0.00021 |
| Lrrc4b | 32 | 1.66E−09 | 8.94E−07 | 0.000274 | 0.008381 | 0.003322 | 0.001911 | 0.002618 |
| Gm16513 | 34 | 1.82E−09 | 9.13E−07 | 0.001709 | 0.000143 | 0.036228 | 0.001232 | 0.003919 |
| Xkr4 | 252 | 5.16E−06 | 0.000363 | 0.027892 | 0.007534 | 0.006343 | 0.023603 | 0.015276 |
| Galnt10 | 48 | 1.45E−08 | 5.33E−06 | 0.00206 | 0.001506 | 0.017901 | 0.000195 | 0.042871 |
| Hoxa9 | 106 | 2.15E−07 | 3.61E−05 | 0.000367 | 0.014352 | 0.16343 | 0.001415 | 0.008845 |
| Snhg14 | 125 | 4.31E−07 | 6.07E−05 | 0.015199 | 0.001976 | 0.0066 | 0.016034 | 0.007711 |
| Raet1d | 649 | 0.000149 | 0.004105 | 0.01556 | 0.02055 | 0.001869 | 0.238557 | 0.220454 |
| Dnah6 | 1410 | 0.001546 | 0.01964 | 0.092424 | 0.016092 | 0.103491 | 0.071234 | 0.061464 |
| Ankrd33b | 20 | 1.39E−10 | 1.24E−07 | 8.08E−05 | 0.004237 | 0.004697 | 0.000763 | 0.001856 |
| Rims2 | 87 | 1.17E−07 | 2.38E−05 | 0.003775 | 0.003734 | 0.006335 | 0.005281 | 0.011148 |
| Hoxd3 | 62 | 3.72E−08 | 1.06E−05 | 0.057051 | 0.000118 | 0.09922 | 0.010333 | 0.000199 |
| Shmt2 | 221 | 4.32E−06 | 0.000347 | 0.021916 | 0.002506 | 0.003046 | 0.004805 | 0.482854 |
| Cd84 | 448 | 3.91E−05 | 0.001562 | 0.003506 | 0.134515 | 0.010079 | 0.015609 | 0.078325 |
| Atp6v1c1 | 58 | 3.36E−08 | 1.01E−05 | 0.002461 | 0.00082 | 0.007983 | 0.006996 | 0.010883 |
| Gm9962 | 506 | 6.20E−05 | 0.002194 | 0.016673 | 0.008355 | 0.01369 | 0.036641 | 0.148205 |
| Sparc | 26 | 6.78E−10 | 4.68E−07 | 2.87E−05 | 0.00141 | 0.010802 | 0.003886 | 0.008095 |
| Trim52 | 1566 | 0.002115 | 0.024202 | 0.03954 | 0.037323 | 0.114096 | 0.092209 | 0.066423 |
| Rfx8 | 1017 | 0.000559 | 0.00985 | 0.044429 | 0.170213 | 0.050877 | 0.021088 | 0.021478 |
| Serpina3n | 217 | 4.22E−06 | 0.000347 | 0.002256 | 0.001522 | 0.009518 | 0.042548 | 0.270665 |
| Nr4a2 | 12 | 1.13E−11 | 1.69E−08 | 0.00193 | 0.000138 | 0.000487 | 0.000956 | 0.001105 |
| Hoxa7 | 449 | 3.92E−05 | 0.001563 | 0.0677 | 0.004556 | 0.031306 | 0.029911 | 0.020198 |
| Jak3 | 19 | 1.35E−10 | 1.24E−07 | 0.000131 | 8.16E−05 | 0.000534 | 0.020925 | 0.018542 |
| Mrgpra2b | 50 | 1.83E−08 | 6.45E−06 | 0.001634 | 0.001498 | 0.054981 | 0.000378 | 0.011923 |
| 4930509J09Rik | 1494 | 0.001838 | 0.022049 | 0.108317 | 0.071756 | 0.02653 | 0.020129 | 0.205326 |
| Mical3 | 1895 | 0.003705 | 0.035026 | 0.097038 | 0.065793 | 0.090833 | 0.028246 | 0.136191 |
| Vmn2r-ps158 | 52 | 2.12E−08 | 7.08E−06 | 0.00966 | 0.011556 | 0.007297 | 0.000667 | 0.001322 |
| 4930578G10Rik | 53 | 2.13E−08 | 7.08E−06 | 0.021507 | 0.002198 | 0.003067 | 0.00136 | 0.003659 |
| Lrrc47 | 130 | 4.59E−07 | 6.29E−05 | 0.008463 | 0.004251 | 0.006033 | 0.015528 | 0.007832 |
| Gm3772 | 45 | 1.15E−08 | 4.45E−06 | 0.000117 | 0.000401 | 0.029895 | 0.001598 | 0.157694 |
| Tamalin | 68 | 5.25E−08 | 1.36E−05 | 0.005869 | 0.001075 | 0.019833 | 0.000553 | 0.029778 |
| Erbb4 | 31 | 1.65E−09 | 8.94E−07 | 0.001652 | 0.000838 | 0.002935 | 0.001471 | 0.006347 |
| Zfp61 | 632 | 0.000139 | 0.003914 | 0.026353 | 0.040672 | 0.048616 | 0.004503 | 0.122298 |
| Pacsin1 | 97 | 1.59E−07 | 2.91E−05 | 0.027069 | 0.011003 | 0.00049 | 0.030547 | 0.001688 |
| Zfp46 | 25 | 5.24E−10 | 3.76E−07 | 0.001573 | 0.001715 | 0.01039 | 0.000106 | 0.003459 |
| E4f1 | 29 | 9.70E−10 | 6.00E−07 | 0.000536 | 0.005639 | 0.003952 | 0.001801 | 0.000962 |
| Cbx8 | 59 | 3.39E−08 | 1.01E−05 | 0.000195 | 0.00113 | 0.004336 | 0.017482 | 0.074175 |
| Tbc1d9 | 22 | 2.98E−10 | 2.43E−07 | 0.000198 | 0.005164 | 0.001425 | 0.001813 | 0.002051 |
| Ctnnd2 | 37 | 2.74E−09 | 1.29E−06 | 0.001055 | 0.000956 | 0.00034 | 0.010193 | 0.019466 |
| Aktip | 160 | 1.10E−06 | 0.000122 | 0.00089 | 0.00031 | 0.072904 | 0.138572 | 0.026776 |
| Ripor2 | 1208 | 0.000989 | 0.014656 | 0.041416 | 0.01976 | 0.088951 | 0.069026 | 0.073707 |
| Dnah7c | 170 | 1.43E−06 | 0.00015 | 0.013033 | 0.008049 | 0.007656 | 0.005388 | 0.023662 |
| Hoxa2 | 17 | 1.12E−10 | 1.18E−07 | 0.000137 | 0.000649 | 0.043959 | 8.82E−05 | 0.005215 |
| Mrgprx2 | 65 | 4.63E−08 | 1.25E−05 | 0.00069 | 0.000605 | ND | 0.0004 | ND |
| 1500004A13Rik | 84 | 1.07E−07 | 2.26E−05 | 0.009776 | 0.007295 | 0.000885 | 0.007856 | 0.009567 |
| Gon4l | 207 | 3.43E−06 | 0.000296 | 0.005621 | 0.008249 | 0.009289 | 0.011752 | 0.057952 |
| Gabrg3 | 830 | 0.000327 | 0.007054 | 0.035168 | 0.015446 | 0.037732 | 0.028186 | 0.149737 |
| 1700021N21Rik | NA | ND | ND | ND | ND | ND | ND | ND |
| Camk2b | 4742 | 0.055001 | 0.20794 | 0.028132 | 0.274002 | 0.39139 | 0.184097 | 0.222445 |
| Syngap1 | 30 | 1.47E−09 | 8.52E−07 | 0.008392 | 0.000746 | 0.000118 | 0.015574 | 0.002903 |
| Gpr137b-ps | 73 | 7.05E−08 | 1.71E−05 | 0.000568 | 0.000245 | 0.028771 | 0.008624 | 0.084356 |
| Slc22a27 | 74 | 7.93E−08 | 1.90E−05 | 0.004324 | 0.005464 | 0.039565 | 0.00195 | 0.00183 |
| Skint2 | 75 | 8.20E−08 | 1.94E−05 | 0.0042 | 0.012131 | 0.003151 | 0.003363 | 0.00643 |
| Zbtb7a | 13 | 1.33E−11 | 1.84E−08 | 0.082386 | 1.67E−05 | 3.38E−05 | 0.015438 | 0.000228 |
| Paqr5 | 457 | 4.28E−05 | 0.001676 | 0.069549 | 0.015292 | 0.016009 | 0.010265 | 0.037262 |
| 1700101I11Rik | NA | ND | ND | ND | ND | ND | ND | ND |
| Cacna1a | 92 | 1.42E−07 | 2.72E−05 | 0.002341 | 0.001188 | 0.015812 | 0.004818 | 0.031178 |
| Sema4b | 140 | 6.17E−07 | 7.85E−05 | 0.07834 | 0.024009 | 0.002864 | 0.001594 | 0.004367 |
| Esyt2 | 83 | 1.03E−07 | 2.21E−05 | 0.000248 | 0.004453 | 0.01436 | 0.014827 | 0.019307 |
| Nr2f2 | 69 | 5.71E−08 | 1.45E−05 | 0.004171 | 0.002714 | 0.003674 | 0.16208 | 0.000337 |
| Adcy2 | 40 | 3.86E−09 | 1.69E−06 | 0.002027 | 0.000759 | 0.001222 | 0.001983 | 0.027008 |
| Galnt6 | 64 | 4.17E−08 | 1.15E−05 | 0.004051 | 0.01806 | 0.00036 | 0.0033 | 0.018151 |
| Wnt3a | 55 | 2.98E−08 | 9.43E−06 | 0.001816 | 0.000244 | 0.053002 | 0.031739 | 0.001433 |
| Sprn | NA | ND | ND | ND | ND | ND | ND | ND |
| Rgs22 | 203 | 3.35E−06 | 0.000294 | 0.039313 | 0.028155 | 0.017491 | 0.004261 | 0.003457 |
| Col28a1 | 88 | 1.18E−07 | 2.39E−05 | 6.82E−05 | 0.042567 | 0.000151 | 0.096835 | 0.125218 |
| Gm38499 | 56 | 3.03E−08 | 9.43E−06 | 0.000119 | 0.052933 | 0.044863 | 3.20E−05 | 0.119956 |
| Ribc2 | 1282 | 0.001158 | 0.016182 | 0.004676 | 0.097212 | 0.114244 | 0.176427 | 0.049902 |
| Hoxaas3 | 57 | 3.05E−08 | 9.43E−06 | 0.000851 | 0.002133 | 0.021015 | 0.000824 | 0.034814 |
| Pde9a | 129 | 4.40E−07 | 6.07E−05 | 0.007696 | 0.000973 | 0.021924 | 0.01035 | 0.014753 |
| Skint8 | 94 | 1.46E−07 | 2.77E−05 | 0.005283 | 0.002113 | 0.034209 | 0.000979 | 0.018328 |
| Arhgap35 | 96 | 1.50E−07 | 2.78E−05 | 0.001029 | 0.026689 | 0.0169 | 0.000159 | 0.095557 |
| Gm5796 | 962 | 0.000467 | 0.008688 | 0.003503 | 0.259235 | 0.044678 | 0.025624 | 0.132235 |
| Lingo2 | 165 | 1.21E−06 | 0.000131 | 0.009723 | 0.005248 | 0.006532 | 0.010322 | 0.024274 |
| Gm2042 | 99 | 1.77E−07 | 3.15E−05 | 0.002551 | 0.003354 | 0.008974 | 0.014766 | 0.007558 |
| Raet1c | 101 | 1.82E−07 | 3.20E−05 | 0.012929 | 0.008089 | 0.008797 | 0.001254 | 0.007648 |
| Gm1968 | 144 | 7.01E−07 | 8.67E−05 | 0.005523 | 0.014912 | 0.088378 | 0.002018 | 0.002969 |
| Tdpoz5 | 102 | 1.92E−07 | 3.35E−05 | 0.010389 | 0.004823 | 0.00029 | 0.002903 | 0.223518 |
| Vmn2r-ps11 | 103 | 1.94E−07 | 3.35E−05 | 0.001351 | 0.003748 | 0.0117 | 0.008976 | 0.017919 |
| Usp13 | 824 | 0.000319 | 0.006935 | 0.026904 | 0.007565 | 0.06592 | 0.132668 | 0.047092 |
| Fxyd1 | 123 | 3.96E−07 | 5.73E−05 | 0.001218 | 0.001403 | 0.028692 | 0.001391 | 0.324431 |
| Gm14486 | 3299 | 0.020801 | 0.112996 | 0.16586 | 0.587418 | 0.286963 | 0.015368 | 0.062748 |
| Lrp1b | 41 | 4.21E−09 | 1.80E−06 | 0.002817 | 0.00157 | 0.001658 | 0.000583 | 0.025961 |
| 1700030N03Rik | 42 | 5.29E−09 | 2.21E−06 | 0.013915 | 0.000402 | 0.0059 | 0.002431 | 0.0018 |
| Has1 | 117 | 3.67E−07 | 5.58E−05 | 0.077706 | 0.006558 | 0.000958 | 0.004677 | 0.008855 |
| Yars | 712 | 0.000192 | 0.004815 | 0.014266 | 0.01033 | 0.079136 | 0.019369 | 0.19209 |
| Crtc1 | 3285 | 0.020456 | 0.111596 | 0.063134 | 0.16584 | 0.045974 | 0.101157 | 0.539885 |
| Ephb4 | 79 | 9.30E−08 | 2.08E−05 | 0.026845 | 0.00304 | 0.020536 | 0.005468 | 0.000438 |
| Mir7649 | 108 | 2.95E−07 | 4.82E−05 | 0.002575 | 0.005792 | 0.002526 | 0.054925 | 0.007552 |
| Kcnk9 | 3401 | 0.02252 | 0.118672 | 0.040977 | 0.205816 | 0.08424 | 0.099899 | 0.428434 |
| Apba2 | 6762 | 0.147151 | 0.390177 | 0.370799 | 0.239222 | 0.132782 | 0.147846 | 0.38702 |
| Lrrc25 | 172 | 1.53E−06 | 0.000159 | 0.078202 | 0.001857 | 0.001902 | 0.006371 | 0.063097 |
| Mmp2 | 2825 | 0.01335 | 0.08467 | 0.009385 | 0.025253 | 0.283847 | 0.268188 | 0.771323 |
| Tmem181c-ps | 114 | 3.16E−07 | 4.93E−05 | 0.012256 | 0.019727 | 0.027535 | 0.000295 | 0.008626 |
| Fbxl7 | 5238 | 0.071634 | 0.245199 | 0.069403 | 0.086209 | 0.151336 | 0.30929 | 0.682368 |
| Klra14-ps | 118 | 3.76E−07 | 5.61E−05 | 0.003344 | 0.00847 | 0.013882 | 0.005825 | 0.009085 |
| Kcnh7 | 646 | 0.000144 | 0.004003 | 0.059683 | 0.017862 | 0.046872 | 0.007573 | 0.079832 |
| Ccm21 | 61 | 3.67E−08 | 1.06E−05 | 0.003302 | 0.005047 | 0.089045 | 0.000243 | 0.003769 |
| Qrfp | 379 | 2.26E−05 | 0.001067 | 0.019499 | 0.010119 | 0.015744 | 0.021102 | 0.044883 |
| Hoxa3 | 36 | 1.96E−09 | 9.52E−07 | 0.000374 | 0.002009 | 0.029706 | 0.000127 | 0.016418 |
| Ugt8a | 6976 | 0.1589 | 0.408396 | 0.637551 | 0.079386 | 0.32344 | 0.14717 | 0.322576 |
| Prkd2 | 78 | 9.23E−08 | 2.08E−05 | 0.001048 | 0.376824 | 0.001761 | 0.000114 | 0.050224 |
| Mrgpra2a | 131 | 4.66E−07 | 6.34E−05 | 0.035901 | 0.003762 | 0.001461 | 0.00291 | 0.046814 |
| Rex2 | 381 | 2.31E−05 | 0.001083 | 0.004575 | 0.002027 | 0.01805 | 0.073336 | 0.246596 |
| Rnf17 | 1853 | 0.00345 | 0.033354 | 0.051507 | 0.116701 | 0.088308 | 0.030885 | 0.123254 |
| Pcdhga12 | 5131 | 0.068592 | 0.239676 | 0.224887 | 0.260528 | 0.141441 | 0.073346 | 0.292425 |
| Mfap2 | 3013 | 0.015944 | 0.094811 | 0.185964 | 0.137383 | 0.106575 | 0.087696 | 0.075791 |
| Fam171a2 | 33 | 1.69E−09 | 8.94E−07 | 0.000386 | 0.000688 | 0.029978 | 0.00387 | 0.00127 |
| Tc2n | 1589 | 0.002195 | 0.02476 | 0.123045 | 0.117944 | 0.020942 | 0.052831 | 0.067586 |
| Egr3 | 21 | 2.97E−10 | 2.43E−07 | 0.001117 | 0.010059 | 0.000166 | 0.000717 | 0.004041 |
| Nox4 | 3646 | 0.027502 | 0.135195 | 0.109553 | 0.12587 | 0.081272 | 0.094824 | 0.388407 |
| 4930433N12Rik | 679 | 0.000165 | 0.004336 | 0.01636 | 0.005964 | 0.046119 | 0.115359 | 0.068916 |
| 5830417I10Rik | 82 | 1.01E−07 | 2.19E−05 | 0.015976 | 0.002189 | 0.001289 | 0.00854 | 0.011559 |
| Gm14326 | 1019 | 0.000567 | 0.009957 | 0.030412 | 0.143785 | 0.004415 | 0.111726 | 0.082221 |
| Ranbp17 | 10965 | 0.457151 | 0.747736 | 0.350968 | 0.294785 | 0.648503 | 0.269973 | 0.408674 |
| Asic4 | 150 | 8.95E−07 | 0.000106 | 0.011376 | 0.011554 | 0.01232 | 0.013622 | 0.002646 |
| Cntnap5c | 90 | 1.26E−07 | 2.47E−05 | 0.015277 | 0.003466 | 0.003173 | 0.003403 | 0.010008 |
| Gemin5 | 116 | 3.62E−07 | 5.55E−05 | 0.005042 | 0.002036 | 0.013006 | 0.003238 | 0.046087 |
| Cacng7 | 162 | 1.12E−06 | 0.000124 | 0.015802 | 0.045089 | 0.000408 | 0.015606 | 0.016857 |
| Samd14 | 178 | 1.73E−06 | 0.000174 | 0.00101 | 0.037966 | 0.007123 | 0.002031 | 0.232073 |
| Tmc4 | 3078 | 0.016941 | 0.098626 | 0.008631 | 0.064275 | 0.215018 | 0.4409 | 0.376673 |
| Pnma1 | NA | ND | ND | ND | ND | ND | ND | ND |
| Sirpb1c | 148 | 8.30E−07 | 9.99E−05 | 0.028728 | 0.00148 | 0.005127 | 0.004768 | 0.051351 |
| Tescl | NA | ND | ND | ND | ND | ND | ND | ND |
| Fxyd7 | 10544 | 0.422151 | 0.717925 | 0.324551 | 0.731448 | 0.109721 | 0.271162 | 0.858316 |
| Gm28453 | 147 | 7.59E−07 | 9.20E−05 | 0.003219 | 0.003301 | 0.011531 | 0.012471 | 0.031388 |
| Tango6 | 219 | 4.26E−06 | 0.000347 | 0.0172 | 0.010604 | 0.047582 | 0.003959 | 0.011083 |
| Cntnap5b | 432 | 3.41E−05 | 0.001413 | 0.054016 | 0.017596 | 0.03242 | 0.007997 | 0.019892 |
| Foxg1 | 428 | 3.38E−05 | 0.001412 | 0.071704 | 0.014275 | 0.06242 | 0.030481 | 0.002491 |
| Abi3 | 2262 | 0.006401 | 0.05069 | 0.005911 | 0.015935 | 0.252158 | 0.350791 | 0.577244 |
| Csmd1 | 853 | 0.000345 | 0.007234 | 0.043553 | 0.012262 | 0.056724 | 0.023802 | 0.1285 |
| Hk1 | 555 | 8.75E−05 | 0.002823 | 0.111309 | 0.009338 | 0.010885 | 0.124959 | 0.011309 |
| D830030K20Rik | 154 | 9.87E−07 | 0.000114 | 0.032906 | 0.000547 | 0.009326 | 0.014227 | 0.027487 |
| Mir23a | NA | ND | ND | ND | ND | ND | ND | ND |
| Yap1 | 4628 | 0.051574 | 0.199783 | 0.175294 | 0.17609 | 0.224098 | 0.027541 | 0.584155 |
| Rsph9 | 2837 | 0.013588 | 0.085819 | 0.172576 | 0.200138 | 0.40521 | 0.048806 | 0.020911 |
| Mark4 | 157 | 1.08E−06 | 0.000122 | 0.003758 | 0.007378 | 0.056789 | 0.008219 | 0.005629 |
| Skint10 | 159 | 1.09E−06 | 0.000122 | 0.004378 | 0.012916 | 0.076106 | 0.001075 | 0.015995 |
| Hoxc5 | 105 | 2.14E−07 | 3.61E−05 | 0.023056 | 0.010299 | 0.01474 | 0.000974 | 0.003133 |
| Tmem267 | 1177 | 0.000893 | 0.013586 | 0.607179 | 0.004488 | 0.048619 | 0.026077 | 0.093565 |
| Mir196a-1 | NA | ND | ND | ND | ND | ND | ND | ND |
| Galnt13 | 612 | 0.000125 | 0.003666 | 0.028029 | 0.023699 | 0.038032 | 0.013432 | 0.074342 |
| Rab44 | 163 | 1.19E−06 | 0.00013 | 0.002766 | 0.079275 | 0.002095 | 0.037758 | 0.004736 |
| Srcin1 | 95 | 1.49E−07 | 2.78E−05 | 0.030355 | 0.001505 | 0.000878 | 0.006909 | 0.025247 |
| Dync2i1 | 133 | 5.03E−07 | 6.66E−05 | 0.000133 | 0.003909 | 0.006043 | 0.112892 | 0.082573 |
| Spag1 | 215 | 4.20E−06 | 0.000347 | 0.000961 | 0.144923 | 0.071722 | 0.009295 | 0.004039 |
| Prxl2c | 242 | 4.71E−06 | 0.000347 | 0.020313 | 0.027494 | 0.011588 | 0.004778 | 0.01392 |
| Umad1 | 38 | 3.20E−09 | 1.47E−06 | 8.80E−05 | 0.01492 | 5.17E−05 | 0.031452 | 0.03805 |
| Mroh7 | 67 | 5.08E−08 | 1.34E−05 | 0.004024 | 0.003675 | 0.006388 | 0.00348 | 0.006031 |
| Espn | 60 | 3.52E−08 | 1.04E−05 | 0.053417 | 5.63E−05 | 0.011646 | 0.003828 | 0.009653 |
| Kcnk12 | 89 | 1.26E−07 | 2.47E−05 | 0.011657 | 0.011622 | 0.00321 | 0.003034 | 0.004333 |
| Ptprf | 173 | 1.56E−06 | 0.000161 | 0.01424 | 0.004558 | 0.006331 | 0.01914 | 0.01446 |
| Inpp4b | 80 | 9.70E−08 | 2.15E−05 | 0.000359 | 0.011686 | 0.015069 | 0.006771 | 0.009856 |
| Mlkl | 4579 | 0.049944 | 0.195537 | 0.118665 | 0.090245 | 0.173957 | 0.381564 | 0.148643 |
| Pitx2 | 3329 | 0.021313 | 0.114735 | 0.115544 | 0.027408 | 0.178634 | 0.460914 | 0.107271 |
| Pcyox11 | 6571 | 0.136016 | 0.371046 | 0.92538 | 0.425386 | 0.044865 | 0.518747 | 0.063692 |
| Prss32 | 414 | 3.12E−05 | 0.001344 | 0.021676 | 0.123388 | 0.020902 | 0.098705 | 0.000796 |
| Ajm1 | NA | ND | ND | ND | ND | ND | ND | ND |
| Tox3 | 1301 | 0.001211 | 0.016676 | 0.005389 | 0.011178 | 0.210376 | 0.194181 | 0.197254 |
| Bcat1 | 401 | 2.81E−05 | 0.001253 | 0.007166 | 0.007287 | 0.061074 | 0.013024 | 0.092659 |
| Rtn4rl2 | 76 | 8.94E−08 | 2.07E−05 | 0.000592 | 0.023029 | 0.014426 | 0.002004 | 0.009736 |
| Rassf3 | 224 | 4.61E−06 | 0.000347 | 0.032364 | 0.000841 | 0.002595 | 0.027325 | 0.217176 |
| 4933438B17Rik | 39 | 3.27E−09 | 1.47E−06 | 0.000322 | 0.006143 | 0.001657 | 0.001903 | 0.01332 |
| Asap2 | 796 | 0.000283 | 0.006352 | 0.063148 | 0.071286 | 0.17726 | 0.000602 | 0.14899 |
| Mir141 | NA | ND | ND | ND | ND | ND | ND | ND |
| Ceacam20 | 107 | 2.89E−07 | 4.80E−05 | 0.002747 | 0.04041 | 0.029919 | 0.000154 | 0.029743 |
| Nbea | 1611 | 0.002289 | 0.025462 | 0.064285 | 0.207465 | 0.0102 | 0.136336 | 0.061941 |
| Mylk3 | 177 | 1.72E−06 | 0.000173 | 0.006418 | 0.035141 | 0.00397 | 0.017358 | 0.008202 |
| Adgrb3 | 6306 | 0.120881 | 0.34368 | 0.327781 | 0.094593 | 0.22862 | 0.1733 | 0.383988 |
| Obox3 | 191 | 2.46E−06 | 0.00023 | 0.007269 | 0.006142 | 0.003745 | 0.016583 | 0.070856 |
| Rtkn2 | 35 | 1.83E−09 | 9.13E−07 | 0.027316 | 3.14E−05 | 4.73E−05 | 0.012077 | 0.08724 |
| Pex5l | 8293 | 0.243897 | 0.527349 | 0.333358 | 0.223025 | 0.157402 | 0.220147 | 0.695138 |
| Rnaset2a | 135 | 5.05E−07 | 6.66E−05 | 0.011479 | 0.006846 | 0.011365 | 0.008642 | 0.003825 |
| Pals2 | 5294 | 0.074226 | 0.251385 | 0.518051 | 0.044489 | 0.423818 | 0.159153 | 0.130474 |

TABLE 17
(Supplementary Table S8a. Ranked genes by differential methylation
(Old + OSKM vs Old-4): RANK comp., p-value comp., q-value comp.)

| | RANK | p-value | q-value | p-value | p-value | p-value | p-value | p-value |
|---|---|---|---|---|---|---|---|---|
| Gene | comp. | comp. | comp. | comp. TR1 | comp. TR2 | comp. TR3 | comp. TR4 | comp. TR5 |
| Nfix | 1 | 2.22E−16 | 1.23E−12 | 0 | 0 | 0.000191 | 0.001319 | 0.000166 |
| Serpina1b | 2 | 2.22E−16 | 1.23E−12 | 0.001299 | 0.003575 | 0.00083 | 0 | 0.009779 |
| Ciapin1 | 3 | 2.22E−16 | 1.23E−12 | 0 | 0.001368 | 0.022654 | 0.05577 | 0.004025 |
| Atp6v1c2 | 4 | 2.22E−16 | 1.23E−12 | 0.084344 | 0.038411 | 0.503468 | 0 | 0.104576 |
| Def8 | 5 | 8.75E−15 | 3.87E−11 | 9.77E−05 | 0.000283 | 4.88E−05 | 9.77E−05 | 0.000371 |
| Rbfox1 | 6 | 2.18E−13 | 8.04E−10 | 3.48E−08 | 0.037122 | 0.170301 | 3.96E−07 | 0.019391 |
| Vmn2r47 | 7 | 1.03E−12 | 3.25E−09 | 8.96E−05 | 0.000263 | 0.001326 | 0.000119 | 0.002522 |
| Irgc1 | 8 | 1.32E−12 | 3.52E−09 | 0.000135 | 0.000381 | 5.86E−05 | 0.005147 | 0.000801 |
| Gm45351 | 9 | 1.43E−12 | 3.52E−09 | 8.96E−05 | 0.000265 | 0.00169 | 0.000119 | 0.00284 |
| Mmp16 | 10 | 1.82E−12 | 4.03E−09 | 4.88E−05 | 0.000762 | 0.000733 | 0.002667 | 0.000244 |
| Hoxa7 | 11 | 3.25E−12 | 6.53E−09 | 6.84E−05 | 0.000928 | 0.000742 | 0.000459 | 0.001563 |
| Gm53 | 12 | 1.91E−11 | 3.52E−08 | 0.00464 | 0.000186 | 0.001368 | 0.000283 | 0.000733 |
| Gm10377 | 13 | 4.04E−11 | 6.41E−08 | 0.001623 | 0.000799 | 0.000861 | 0.000544 | 0.000937 |
| Mup2 | 14 | 4.06E−11 | 6.41E−08 | 0.000828 | 0.000244 | 0.002707 | 0.000994 | 0.001049 |
| Hoxa2 | 15 | 5.56E−11 | 8.20E−08 | 0.000293 | 0.000361 | 0.002022 | 0.0072 | 0.000528 |
| Skint8 | 16 | 7.60E−11 | 1.05E−07 | 0.024848 | 0.000588 | 0.00037 | 0.000599 | 0.000356 |
| Skint5 | 17 | 8.79E−11 | 1.14E−07 | 0.000899 | 0.003666 | 0.001097 | 0.000421 | 0.000895 |
| Fam171a2 | 18 | 1.24E−10 | 1.52E−07 | 0.035168 | 0.000234 | 0.006721 | 0.000186 | 0.000195 |
| Mir196a-1 | 19 | 4.47E−10 | 5.20E−07 | 0.004113 | 0.000901 | 0.004629 | 0.000414 | 0.001209 |
| Defa23 | 20 | 5.18E−10 | 5.73E−07 | 0.000726 | 0.00027 | 0.000869 | 0.002206 | 0.026984 |
| Gnas | 21 | 6.48E−10 | 6.55E−07 | 0.001299 | 0.000313 | 0.00295 | 0.029453 | 0.000371 |
| Skint6 | 22 | 6.51E−10 | 6.55E−07 | 0.003955 | 0.001434 | 0.001082 | 0.000455 | 0.004716 |
| Hic1 | 23 | 9.94E−10 | 9.56E−07 | 0.037122 | 8.79E−05 | 0.000596 | 0.000723 | 0.015161 |
| Skint10 | 24 | 1.26E−09 | 1.16E−06 | 0.002298 | 0.001838 | 0.007845 | 0.000139 | 0.006048 |
| Irx5 | 25 | 1.83E−09 | 1.60E−06 | 0.02873 | 0.00211 | 0.00466 | 0.003097 | 4.88E−05 |
| Hoxaas3 | 26 | 1.88E−09 | 1.60E−06 | 0.000472 | 0.001142 | 0.008222 | 0.003302 | 0.00301 |
| Acer2 | 27 | 1.98E−09 | 1.60E−06 | 0.001084 | 0.007356 | 8.79E−05 | 0.004914 | 0.013559 |
| Abi3 | 28 | 2.06E−09 | 1.60E−06 | 0.000234 | 0.000655 | 0.005431 | 0.004884 | 0.012045 |
| Rpl391 | 29 | 2.15E−09 | 1.60E−06 | 0.025216 | 0.000237 | 0.006901 | 0.006322 | 0.000197 |
| Hoxa9 | 30 | 2.17E−09 | 1.60E−06 | 0.000498 | 0.002081 | 0.027841 | 0.000899 | 0.002003 |
| Hoxa3 | 31 | 3.07E−09 | 2.19E−06 | 0.000664 | 0.000674 | 0.004924 | 0.01266 | 0.002774 |
| Chst5 | 32 | 3.28E−09 | 2.24E−06 | 0.004171 | 0.000322 | 0.008743 | 0.002403 | 0.00295 |
| Gse1 | 33 | 3.34E−09 | 2.24E−06 | 0.002071 | 0.008948 | 0.004748 | 0.000615 | 0.001573 |
| Hoxb3 | 34 | 3.51E−09 | 2.28E−06 | 0.001768 | 0.000655 | 0.001729 | 0.0448 | 0.001006 |
| Kansl1 | 35 | 4.29E−09 | 2.71E−06 | 0.002774 | 0.002491 | 0.001514 | 0.004484 | 0.002423 |
| Bcl91 | 36 | 4.78E−09 | 2.94E−06 | 0.004328 | 0.000405 | 0.00255 | 0.014429 | 0.001993 |
| Mir467h | 37 | 5.63E−09 | 3.37E−06 | 0.004885 | 0.002002 | 0.002159 | 0.005377 | 0.001367 |
| Col28a1 | 38 | 6.26E−09 | 3.65E−06 | 0.000181 | 0.004048 | 0.000175 | 0.029203 | 0.046763 |
| Prxl2c | 39 | 7.14E−09 | 4.05E−06 | 0.007819 | 0.00255 | 0.001192 | 0.001993 | 0.004308 |
| Gm2042 | 40 | 7.33E−09 | 4.05E−06 | 0.002781 | 0.000699 | 0.007059 | 0.002685 | 0.005708 |
| Syngap1 | 41 | 7.62E−09 | 4.11E−06 | 0.025122 | 0.002606 | 0.003709 | 0.00024 | 0.00377 |
| Zfp61 | 42 | 8.02E−09 | 4.23E−06 | 0.000508 | 0.02242 | 0.020935 | 9.77E−06 | 0.100229 |
| Septin9 | 43 | 9.03E−09 | 4.65E−06 | 0.001143 | 0.003868 | 0.004836 | 0.001075 | 0.011644 |
| 1700021N21Rik | 44 | 9.62E−09 | 4.84E−06 | 0.001029 | 0.00288 | 0.005521 | 0.001153 | 0.015268 |
| Hoxd3 | 45 | 1.08E−08 | 5.31E−06 | 0.004181 | 0.002081 | 0.005011 | 0.007805 | 0.000967 |
| Cbx8 | 46 | 1.17E−08 | 5.64E−06 | 0.001036 | 0.000234 | 0.002345 | 0.019215 | 0.033058 |
| Vdr | 47 | 1.24E−08 | 5.82E−06 | 0.005803 | 0.000166 | 0.004181 | 0.011977 | 0.007971 |
| Gja4 | 48 | 1.57E−08 | 7.26E−06 | 0.012914 | 0.011149 | 0.00353 | 0.000122 | 0.008167 |
| Slc7a11 | 49 | 1.70E−08 | 7.68E−06 | 0.004171 | 0.004083 | 0.011605 | 0.00297 | 0.000948 |
| Gm16513 | 50 | 1.76E−08 | 7.70E−06 | 0.006422 | 0.000311 | 0.032325 | 0.000581 | 0.015437 |
| 4930558F17Rik | 51 | 1.81E−08 | 7.70E−06 | 0.005846 | 0.002015 | 0.000685 | 0.008233 | 0.008997 |
| Ubash3b | 52 | 1.85E−08 | 7.70E−06 | 0.00675 | 0.007297 | 0.003947 | 0.000762 | 0.004132 |
| Phldb1 | 53 | 1.85E−08 | 7.70E−06 | 0.020847 | 0.000166 | 0.004171 | 0.007473 | 0.005676 |
| Prrt2 | 54 | 1.88E−08 | 7.70E−06 | 0.00804 | 0.011508 | 0.002237 | 0.000518 | 0.005822 |
| Zfhx2os | 55 | 2.21E−08 | 8.91E−06 | 0.002462 | 0.009105 | 0.00423 | 0.001807 | 0.004406 |
| Gvin2 | 56 | 2.29E−08 | 8.91E−06 | 0.001394 | 0.002401 | 0.007693 | 0.018323 | 0.001668 |
| Gvin-ps2 | 56 | 2.29E−08 | 8.91E−06 | 0.001394 | 0.002401 | 0.007693 | 0.018323 | 0.001668 |
| Ptprd | 58 | 2.53E−08 | 9.47E−06 | 0.031739 | 0.003888 | 4.71E−05 | 0.002462 | 0.061485 |
| Proc | 59 | 2.57E−08 | 9.47E−06 | 0.001661 | 0.002774 | 0.011176 | 0.001973 | 0.008851 |
| Lgals6 | 60 | 2.60E−08 | 9.47E−06 | 0.016576 | 0.000278 | 0.005143 | 0.116256 | 0.00033 |
| Mmp2 | 61 | 2.61E−08 | 9.47E−06 | 0.001797 | 0.003732 | 0.029824 | 0.000381 | 0.011986 |
| Ptk7 | 62 | 3.06E−08 | 1.09E−05 | 0.001426 | 0.010253 | 0.002706 | 0.004865 | 0.005719 |
| Dnah7c | 63 | 3.32E−08 | 1.17E−05 | 0.048415 | 0.000469 | 0.000479 | 0.052732 | 0.00211 |
| Zfp791 | 64 | 3.79E−08 | 1.31E−05 | 0.005296 | 0.006096 | 0.057519 | 0.000948 | 0.000801 |
| Rps4l | 65 | 4.11E−08 | 1.39E−05 | 0.078857 | 0.00196 | 0.00248 | 8.69E−05 | 0.046437 |
| Ggt5 | 66 | 4.14E−08 | 1.39E−05 | 0.000596 | 0.058193 | 0.001993 | 0.007942 | 0.002843 |
| Ptprf | 67 | 4.39E−08 | 1.45E−05 | 0.027578 | 0.00168 | 0.000938 | 0.049212 | 0.000782 |
| Galnt10 | 68 | 4.48E−08 | 1.46E−05 | 0.001061 | 0.006682 | 0.038255 | 3.26E−05 | 0.193658 |
| Marveld2 | 69 | 4.63E−08 | 1.48E−05 | 0.0034 | 0.00083 | 0.007678 | 0.002051 | 0.040013 |
| 2310043O21Rik | 70 | 5.18E−08 | 1.64E−05 | 0.007766 | 0.003575 | 0.003575 | 0.00382 | 0.005353 |
| Thtpa | 71 | 5.91E−08 | 1.84E−05 | 0.004318 | 0.006975 | 0.00382 | 0.000967 | 0.021247 |
| Gon4l | 72 | 6.13E−08 | 1.86E−05 | 0.001289 | 0.017457 | 0.033312 | 0.000782 | 0.00421 |
| Tmem267 | 73 | 6.14E−08 | 1.86E−05 | 0.011078 | 0.000225 | 0.065168 | 0.028916 | 0.000528 |
| Lsr | 74 | 6.52E−08 | 1.92E−05 | 0.008391 | 0.004972 | 0.012035 | 0.00043 | 0.012289 |
| Gata3 | 75 | 6.52E−08 | 1.92E−05 | 0.067005 | 0.000303 | 0.005949 | 0.003566 | 0.006164 |
| Tiam1 | 76 | 6.64E−08 | 1.93E−05 | 0.008157 | 0.002012 | 0.003898 | 0.006877 | 0.006164 |
| Adad1 | 77 | 6.83E−08 | 1.96E−05 | 0.000596 | 0.001788 | 0.013813 | 0.015738 | 0.012094 |
| Skint11 | 78 | 7.33E−08 | 2.08E−05 | 0.006943 | 0.011878 | 0.000949 | 0.009528 | 0.004078 |
| Obscn | 79 | 7.53E−08 | 2.11E−05 | 0.002491 | 0.001036 | 0.011635 | 0.006174 | 0.016939 |
| Pxn | 80 | 8.06E−08 | 2.21E−05 | 0.016793 | 0.016334 | 0.001123 | 0.000469 | 0.023514 |
| Med15 | 81 | 8.09E−08 | 2.21E−05 | 0.001583 | 0.022654 | 0.005588 | 0.004083 | 0.004171 |
| Ebf1 | 82 | 8.70E−08 | 2.35E−05 | 0.010736 | 0.00043 | 0.024608 | 0.002706 | 0.012094 |
| Tcp10a | 83 | 9.12E−08 | 2.43E−05 | 0.019948 | 0.007874 | 0.006027 | 0.000821 | 0.00506 |
| BC021767 | 84 | 9.23E−08 | 2.43E−05 | 0.004601 | 0.01352 | 0.033165 | 0.010404 | 0.000186 |
| Znrf1 | 85 | 9.73E−08 | 2.53E−05 | 0.001065 | 0.006814 | 0.043462 | 0.005637 | 0.002384 |
| Ckm | 86 | 1.01E−07 | 2.56E−05 | 0.012494 | 0.019225 | 0.006965 | 0.00126 | 0.002091 |
| Evpl | 87 | 1.01E−07 | 2.56E−05 | 0.001612 | 0.013969 | 0.001592 | 0.023094 | 0.005324 |
| Mir1966 | 88 | 1.02E−07 | 2.57E−05 | 0.224916 | 0.000634 | 0.001937 | 0.006306 | 0.002577 |
| Espn | 89 | 1.28E−07 | 3.17E−05 | 0.019772 | 8.79E−05 | 0.112938 | 0.001368 | 0.021843 |
| Ctps | 90 | 1.29E−07 | 3.17E−05 | 0.003019 | 0.008391 | 0.028212 | 0.000996 | 0.008313 |
| Asap2 | 91 | 1.30E−07 | 3.17E−05 | 0.032984 | 0.084154 | 0.00805 | 0.000702 | 0.000381 |
| Ephb4 | 92 | 1.33E−07 | 3.19E−05 | 0.003927 | 0.120831 | 0.000869 | 0.002794 | 0.005285 |
| D2hgdh | 93 | 1.38E−07 | 3.28E−05 | 0.082479 | 0.003546 | 0.000742 | 0.006555 | 0.004484 |
| Cdca7l | 94 | 1.41E−07 | 3.32E−05 | 0.018151 | 0.000186 | 0.070688 | 0.004836 | 0.005695 |
| Adamts18 | 95 | 1.46E−07 | 3.41E−05 | 0.000528 | 0.001172 | 0.010296 | 0.05665 | 0.018961 |
| Chd9 | 96 | 1.55E−07 | 3.58E−05 | 0.000215 | 0.012358 | 0.025155 | 0.022195 | 0.004953 |
| Efemp2 | 97 | 1.69E−07 | 3.84E−05 | 0.014272 | 0.003878 | 0.002696 | 0.005207 | 0.010394 |
| Kbtbd7 | 98 | 1.74E−07 | 3.91E−05 | 0.001681 | 0.022992 | 0.002196 | 0.046864 | 0.002108 |
| Larp1 | 99 | 1.75E−07 | 3.91E−05 | 0.002407 | 0.004308 | 0.024979 | 0.006965 | 0.004679 |
| Ccdc152 | 100 | 1.83E−07 | 4.06E−05 | 0.001837 | 0.015738 | 0.003868 | 0.009661 | 0.008255 |
| 1500004A13Rik | 101 | 1.90E−07 | 4.09E−05 | 0.012462 | 0.006194 | 0.000796 | 0.026866 | 0.005626 |
| Ccnjl | 102 | 1.90E−07 | 4.09E−05 | 0.004484 | 0.000283 | 0.037884 | 0.007727 | 0.025077 |
| Spred2 | 103 | 1.92E−07 | 4.09E−05 | 0.006721 | 0.002325 | 0.014497 | 0.006389 | 0.006496 |
| Plekhm2 | 104 | 1.92E−07 | 4.09E−05 | 0.027128 | 0.011371 | 0.001749 | 0.001768 | 0.009886 |
| Mrpl40 | 105 | 2.12E−07 | 4.43E−05 | 0.002833 | 0.009505 | 0.003194 | 0.005178 | 0.023719 |
| Unc80 | 106 | 2.12E−07 | 4.43E−05 | 0.00549 | 0.0072 | 0.010736 | 0.002511 | 0.009935 |
| Aktip | 107 | 2.14E−07 | 4.43E−05 | 0.000518 | 0.000283 | 0.037034 | 0.058652 | 0.033624 |
| A430093F15Rik | 108 | 2.47E−07 | 5.06E−05 | 0.011166 | 0.005558 | 0.007776 | 0.003097 | 0.008489 |
| Vmn1r252 | 109 | 2.56E−07 | 5.15E−05 | ND | 0.000724 | 0.000735 | ND | 0.00205 |
| Prkd2 | 110 | 2.59E−07 | 5.15E−05 | 0.037806 | 0.022918 | 0.002354 | 0.00161 | 0.004074 |
| Kcnk12 | 111 | 2.59E−07 | 5.15E−05 | 0.001788 | 0.001993 | 0.026786 | 0.003732 | 0.037649 |
| Tcea3 | 112 | 2.64E−07 | 5.15E−05 | 0.053485 | 0.004865 | 0.015943 | 0.000823 | 0.004005 |
| Gm3002 | 113 | 2.65E−07 | 5.15E−05 | 0.024036 | 0.00149 | 0.018116 | 0.005245 | 0.004048 |
| AI661453 | 114 | 2.66E−07 | 5.15E−05 | 0.006794 | 0.000313 | 0.012758 | 0.0917 | 0.005558 |
| Plbd1 | 115 | 2.68E−07 | 5.15E−05 | 0.02072 | 0.000742 | 0.002384 | 0.015161 | 0.025135 |
| Rerg | 116 | 2.72E−07 | 5.15E−05 | 0.004005 | 0.002384 | 0.019538 | 0.018424 | 0.004132 |
| Pnma8b | 117 | 2.72E−07 | 5.15E−05 | 0.009268 | 0.000147 | 0.004181 | 0.032279 | 0.077633 |
| Sptan1 | 118 | 2.97E−07 | 5.52E−05 | 0.034621 | 0.011537 | 0.001172 | 0.002579 | 0.013041 |
| Gm14327 | 119 | 2.97E−07 | 5.52E−05 | 0.017652 | 0.028253 | 0.001289 | 0.001036 | 0.023677 |
| Hdac5 | 120 | 3.02E−07 | 5.52E−05 | 0.012016 | 0.003067 | 0.007678 | 0.006184 | 0.009173 |
| Smarca5-ps | 121 | 3.19E−07 | 5.52E−05 | ND | 0.003257 | 0.001777 | 0.008197 | 0.003028 |
| Vmn1r-ps79 | 122 | 3.37E−07 | 5.52E−05 | ND | 0.000935 | 0.000504 | 0.101492 | 0.003198 |
| Vmn1r101 | 123 | 3.37E−07 | 5.52E−05 | ND | 0.000935 | 0.000504 | 0.101492 | 0.003198 |
| Vmn1r250 | 124 | 3.37E−07 | 5.52E−05 | ND | 0.000935 | 0.000504 | 0.101492 | 0.003198 |
| Gm10665 | 125 | 3.37E−07 | 5.52E−05 | ND | 0.000935 | 0.000504 | 0.101492 | 0.003198 |
| Vmn1r256 | 126 | 3.37E−07 | 5.52E−05 | ND | 0.000935 | 0.000504 | 0.101492 | 0.003198 |
| Vmn1r100 | 127 | 3.37E−07 | 5.52E−05 | ND | 0.000935 | 0.000504 | 0.101492 | 0.003198 |
| Gm10668 | 128 | 3.37E−07 | 5.52E−05 | ND | 0.000935 | 0.000504 | 0.101492 | 0.003198 |
| Gm4513 | 129 | 3.37E−07 | 5.52E−05 | ND | 0.000935 | 0.000504 | 0.101492 | 0.003198 |
| Vmn1r142 | 130 | 3.37E−07 | 5.52E−05 | ND | 0.000935 | 0.000504 | 0.101492 | 0.003198 |
| Vmn1r143 | 131 | 3.37E−07 | 5.52E−05 | ND | 0.000935 | 0.000504 | 0.101492 | 0.003198 |
| Vmn1r251 | 132 | 3.37E−07 | 5.52E−05 | ND | 0.000935 | 0.000504 | 0.101492 | 0.003198 |
| Vmn1r254 | 133 | 3.37E−07 | 5.52E−05 | ND | 0.000935 | 0.000504 | 0.101492 | 0.003198 |
| Vmn1r152 | 134 | 3.37E−07 | 5.52E−05 | ND | 0.000935 | 0.000504 | 0.101492 | 0.003198 |
| Vmn1r255 | 135 | 3.37E−07 | 5.52E−05 | ND | 0.000935 | 0.000504 | 0.101492 | 0.003198 |
| Tent4a | 136 | 3.51E−07 | 5.70E−05 | 0.005764 | 0.001501 | 0.003732 | 0.019421 | 0.030586 |
| Ddr1 | 137 | 3.58E−07 | 5.75E−05 | 0.003683 | 0.000283 | 0.090948 | 0.006193 | 0.033458 |
| Mrgpra2b | 138 | 3.59E−07 | 5.75E−05 | 0.005806 | 0.006427 | 0.00721 | 0.001307 | 0.056003 |
| Tdpoz5 | 139 | 3.62E−07 | 5.76E−05 | 0.007658 | 0.001486 | 0.002008 | 0.008727 | 0.099744 |
| Abcg1 | 140 | 3.73E−07 | 5.90E−05 | 0.105494 | 0.00255 | 0.002169 | 0.001446 | 0.024491 |
| 5830428M24Rik | 141 | 3.88E−07 | 6.10E−05 | 0.012914 | 0.001162 | 0.055712 | 0.003614 | 0.007161 |
| Slc29a1 | 142 | 3.96E−07 | 6.15E−05 | 0.004386 | 0.007717 | 0.014653 | 0.037493 | 0.001192 |
| Gm38499 | 143 | 3.97E−07 | 6.15E−05 | 0.00043 | 0.109441 | 0.034113 | 0.001358 | 0.010199 |
| Crtc1 | 144 | 4.04E−07 | 6.20E−05 | 0.00168 | 0.017193 | 0.012025 | 0.010824 | 0.006027 |
| Cdcp1 | 145 | 4.49E−07 | 6.84E−05 | 0.013764 | 0.000772 | 0.003419 | 0.010892 | 0.064885 |
| Emilin2 | 146 | 4.58E−07 | 6.94E−05 | 0.000244 | 0.042719 | 0.030235 | 0.003761 | 0.022195 |
| Ddx4 | 147 | 4.63E−07 | 6.96E−05 | 0.001377 | 0.001583 | 0.00677 | 0.081668 | 0.022097 |
| Mir7649 | 148 | 4.68E−07 | 6.97E−05 | 0.010151 | 0.002029 | 0.00472 | 0.011442 | 0.024298 |
| Shisal1 | 149 | 4.70E−07 | 6.97E−05 | 0.011762 | 0.006174 | 0.014771 | 0.001426 | 0.017721 |
| Ripor3 | 150 | 4.83E−07 | 7.12E−05 | 0.027148 | 0.000977 | 0.004425 | 0.034357 | 0.006946 |
| Capn1 | 151 | 4.96E−07 | 7.26E−05 | 0.005744 | 0.014839 | 0.00424 | 0.01521 | 0.005256 |
| Traf3ip3 | 152 | 5.25E−07 | 7.63E−05 | 0.004328 | 0.004122 | 0.003956 | 0.011801 | 0.037112 |
| Mdga1 | 153 | 5.28E−07 | 7.63E−05 | 0.014458 | 0.005578 | 0.011762 | 0.004122 | 0.007962 |
| Guca1b | 154 | 5.41E−07 | 7.77E−05 | 0.002296 | 0.000469 | 0.017789 | 0.295332 | 0.005666 |
| Lgals4 | 155 | 5.61E−07 | 8.01E−05 | 0.032276 | 0.000234 | 0.009798 | 0.204501 | 0.002208 |
| 4930554H23Rik | 156 | 6.11E−07 | 8.65E−05 | 0.007346 | 0.005011 | 0.033087 | 0.02663 | 0.001143 |
| Wnt10b | 157 | 6.14E−07 | 8.65E−05 | 0.005383 | 0.002159 | 0.012778 | 0.013667 | 0.018365 |
| Gm33301 | 158 | 6.61E−07 | 9.26E−05 | 0.001075 | 0.01816 | 0.011742 | 0.007356 | 0.024149 |
| Slc27a1 | 159 | 6.79E−07 | 9.45E−05 | 0.002472 | 0.005246 | 0.003028 | 0.160366 | 0.006672 |
| Mark4 | 160 | 6.99E−07 | 9.63E−05 | 0.029993 | 0.008921 | 0.043663 | 0.00095 | 0.003917 |
| Dapk1 | 161 | 7.01E−07 | 9.63E−05 | 0.016238 | 0.006451 | 0.008908 | 0.004298 | 0.010873 |
| Esyt2 | 162 | 7.07E−07 | 9.66E−05 | 0.000469 | 0.000723 | 0.135133 | 0.009788 | 0.098382 |
| Usp29 | 163 | 7.24E−07 | 9.83E−05 | 0.006877 | 0.014976 | 0.034846 | 0.002081 | 0.006076 |
| Nkpd1 | 164 | 7.38E−07 | 9.96E−05 | 0.048385 | 0.006604 | 0.002862 | 0.002491 | 0.020368 |
| 5830417I10Rik | 165 | 7.57E−07 | 0.000101 | 0.028339 | 0.002091 | 0.003419 | 0.022461 | 0.010502 |
| Col1a2 | 166 | 7.61E−07 | 0.000101 | 0.005246 | 0.007717 | 0.004562 | 0.032999 | 0.007893 |
| Vmn1r107 | 167 | 7.61E−07 | 0.000101 | ND | 0.000876 | 0.003279 | 0.027866 | 0.004871 |
| Oxct2b | 168 | 7.93E−07 | 0.000104 | 0.012056 | 0.013256 | 0.002493 | 0.004852 | 0.026138 |
| Ccdc8 | 169 | 8.32E−07 | 0.000109 | 0.006672 | 0.001094 | 0.175127 | 0.005822 | 0.00719 |
| Fxyd1 | 170 | 8.49E−07 | 0.00011 | 0.001426 | 0.001192 | 0.012025 | 0.007913 | 0.338794 |
| Cpne5 | 171 | 8.99E−07 | 0.000116 | 0.006233 | 0.002472 | 0.011684 | 0.026161 | 0.012475 |
| Ctnnd2 | 172 | 9.19E−07 | 0.000118 | 0.00464 | 0.004884 | 0.000684 | 0.047379 | 0.082098 |
| Nr4a2 | 173 | 9.41E−07 | 0.00012 | 0.021081 | 0.004425 | 0.004914 | 0.002071 | 0.065285 |
| 1700010N08Rik | 174 | 9.57E−07 | 0.000122 | 0.001006 | 0.008777 | 0.008786 | 0.056602 | 0.014414 |
| Gm12185 | 175 | 9.66E−07 | 0.000122 | 0.060607 | 0.001244 | 0.0063 | 0.007792 | 0.017272 |
| Tmem94 | 176 | 9.68E−07 | 0.000122 | 0.017828 | 0.006447 | 0.006623 | 0.011664 | 0.007219 |
| Exoc3l2 | 177 | 1.00E−06 | 0.000125 | 0.060608 | 0.00295 | 0.016724 | 0.015445 | 0.001446 |
| Capn11 | 178 | 1.01E−06 | 0.000125 | 0.016871 | 0.076207 | 0.001153 | 0.000166 | 0.273313 |
| Skint3 | 179 | 1.03E−06 | 0.000127 | 0.004484 | 0.016715 | 0.001504 | 0.031085 | 0.019606 |
| Zbtb11os1 | 180 | 1.04E−06 | 0.000128 | 0.00915 | 0.012066 | 0.004167 | 0.016322 | 0.009317 |
| 1700029B22Rik | 181 | 1.12E−06 | 0.000137 | 0.04536 | 0.034804 | 0.004003 | 0.001746 | 0.006928 |
| Clmp | 182 | 1.13E−06 | 0.000138 | 0.132134 | 0.004904 | 0.00086 | 0.011811 | 0.011762 |
| Dync2i1 | 183 | 1.18E−06 | 0.000143 | 0.000185 | 0.020621 | 0.003459 | 0.09677 | 0.063976 |
| Rims2 | 184 | 1.23E−06 | 0.000148 | 0.009564 | 0.007971 | 0.035266 | 0.023406 | 0.001358 |
| Col6a3 | 185 | 1.24E−06 | 0.000148 | 0.001543 | 0.004298 | 0.007805 | 0.048444 | 0.034406 |
| Cacna1a | 186 | 1.28E−06 | 0.000152 | 0.004253 | 0.001798 | 0.030586 | 0.012152 | 0.031407 |
| St6gal2 | 187 | 1.29E−06 | 0.000153 | 0.003214 | 0.103286 | 0.004015 | 0.008997 | 0.007551 |
| Nkd1 | 188 | 1.33E−06 | 0.000156 | 0.009587 | 0.004781 | 0.00171 | 0.012755 | 0.09386 |
| Pagr1a | 189 | 1.33E−06 | 0.000156 | 0.039525 | 0.04017 | 0.006672 | 0.001133 | 0.007815 |
| Fign | 190 | 1.36E−06 | 0.000158 | 0.026855 | 0.01563 | 0.004406 | 0.002774 | 0.018707 |
| Zcchc24 | 191 | 1.40E−06 | 0.000163 | 0.06204 | 0.008479 | 0.002403 | 0.016128 | 0.004904 |
| Snx31 | 192 | 1.44E−06 | 0.000166 | 0.003802 | 0.00506 | 0.154075 | 0.011644 | 0.002999 |
| Zbtb7a | 193 | 1.52E−06 | 0.000173 | 0.18427 | 0.000508 | 0.000596 | 0.312936 | 0.006291 |
| Sik1 | 194 | 1.52E−06 | 0.000173 | 0.141375 | 0.002051 | 0.077389 | 0.005637 | 0.000869 |
| Hif3a | 195 | 1.56E−06 | 0.000176 | 0.042739 | 0.034123 | 0.00677 | 0.002501 | 0.004582 |
| Atp4b | 196 | 1.56E−06 | 0.000176 | 0.005939 | 0.001309 | 0.032061 | 0.011459 | 0.039662 |
| Erbb4 | 197 | 1.60E−06 | 0.00018 | 0.010941 | 0.006018 | 0.047369 | 0.024315 | 0.001543 |
| Tmc4 | 198 | 1.61E−06 | 0.00018 | 0.000606 | 0.001895 | 0.027206 | 0.140173 | 0.026943 |

TABLE 18
(Supplementary Table S8b. Ranked genes by differential methylation
(Old + OSKM vs Old-4): RANK prom., p-value prom., q-value prom.)

| | RANK | p-value | q-value | p-value | p-value | p-value | p-value | p-value |
|---|---|---|---|---|---|---|---|---|
| Gene | prom. | prom. | prom. | prom. TR1 | prom. TR2 | prom. TR3 | prom. TR4 | prom. TR5 |
| Nfix | 284 | 9.70E−05 | 0.007229 | 0.008571 | 0.007079 | ND | 0.117776 | 0.016546 |
| Serpina1b | 246 | 6.40E−05 | 0.00553 | 0.044667 | 0.071802 | 0.014929 | 0.004707 | 0.047855 |
| Ciapin1 | 483 | 0.000463 | 0.020346 | 0.00027 | 0.08842 | 0.70037 | 0.192026 | 0.042428 |
| Atp6v1c2 | 2008 | 0.016454 | 0.174121 | 0.183918 | 0.589667 | 0.656522 | 0.002073 | 0.128496 |
| Def8 | 5363 | 0.139911 | 0.554 | 0.559119 | 0.50555 | 0.020494 | 0.16737 | 0.633648 |
| Rbfox1 | 6042 | 0.178843 | 0.628605 | ND | 0.161153 | 0.544068 | ND | 0.132694 |
| Vmn2r47 | NA | ND | ND | ND | ND | ND | ND | ND |
| Irgc1 | 1587 | 0.009117 | 0.12207 | ND | 0.36339 | 0.020252 | ND | 0.027024 |
| Gm45351 | NA | ND | ND | ND | ND | ND | ND | ND |
| Mmp16 | 1 | 2.76E−13 | 5.86E−09 | 4.75E−05 | 0.001178 | 0.000228 | 0.001064 | 0.000161 |
| Hoxa7 | 2 | 4.33E−12 | 4.60E−08 | 0.000158 | 0.002167 | 0.000617 | 0.000513 | 0.000429 |
| Gm53 | 391 | 0.000263 | 0.014311 | 0.080446 | 0.017908 | 0.027765 | 0.063141 | 0.02587 |
| Gm10377 | NA | ND | ND | ND | ND | ND | ND | ND |
| Mup2 | 1926 | 0.01503 | 0.165817 | ND | 0.01503 | ND | ND | ND |
| Hoxa2 | 585 | 0.000736 | 0.026659 | 0.036029 | 0.034333 | 0.01239 | 0.454948 | 0.035878 |
| Skint8 | NA | ND | ND | ND | ND | ND | ND | ND |
| Skint5 | NA | ND | ND | ND | ND | ND | ND | ND |
| Fam171a2 | 15677 | 0.814967 | 1 | 0.408359 | 0.709057 | 0.712262 | 0.243974 | 0.987746 |
| Mir196a-1 | 5 | 4.47E−10 | 1.90E−06 | 0.004113 | 0.000901 | 0.004629 | 0.000414 | 0.001209 |
| Defa23 | NA | ND | ND | ND | ND | ND | ND | ND |
| Gnas | 242 | 6.34E−05 | 0.00553 | 0.035071 | 0.004281 | 0.019963 | 0.083549 | 0.042518 |
| Skint6 | NA | ND | ND | ND | ND | ND | ND | ND |
| Hic1 | 134 | 1.44E−05 | 0.002278 | 0.023906 | 0.035835 | 0.027456 | 0.001692 | 0.042251 |
| Skint10 | NA | ND | ND | ND | ND | ND | ND | ND |
| Irx5 | 3195 | 0.048462 | 0.322106 | 0.423593 | 0.087915 | 0.57929 | 0.054386 | 0.085784 |
| Hoxaas3 | NA | ND | ND | ND | ND | ND | ND | ND |
| Acer2 | 197 | 3.87E−05 | 0.004173 | 0.036547 | 0.007985 | 0.006213 | 0.113487 | 0.027885 |
| Abi3 | 1234 | 0.005165 | 0.088947 | 0.062735 | 0.301806 | 0.253008 | 0.093502 | 0.007928 |
| Rpl39l | 8 | 2.15E−09 | 5.72E−06 | 0.025216 | 0.000237 | 0.006901 | 0.006322 | 0.000197 |
| Hoxa9 | 13064 | 0.656305 | 1 | 0.841163 | 0.375565 | 0.230527 | 0.820454 | 0.352852 |
| Hoxa3 | 587 | 0.000738 | 0.026659 | 0.036054 | 0.034322 | 0.012379 | 0.457694 | 0.035788 |
| Chst5 | 487 | 0.000469 | 0.020485 | 0.093124 | 0.068341 | 0.078289 | 0.031096 | 0.00894 |
| Gse1 | 380 | 0.000238 | 0.013279 | 0.020269 | 0.318944 | 0.084059 | 0.013711 | 0.007696 |
| Hoxb3 | 5513 | 0.147705 | 0.568997 | 0.420781 | 0.038691 | 0.673123 | 0.387067 | 0.159987 |
| Kansl1 | 23 | 6.37E−08 | 5.88E−05 | 0.00485 | 0.004782 | 0.003011 | 0.006317 | 0.005853 |
| Bcl9l | 11709 | 0.566066 | 1 | 0.163893 | ND | 0.424303 | 0.6486 | 0.766425 |
| Mir467h | 11 | 5.63E−09 | 1.03E−05 | 0.004885 | 0.002002 | 0.002159 | 0.005377 | 0.001367 |
| Col28a1 | NA | ND | ND | ND | ND | ND | ND | ND |
| Prxl2c | 287 | 9.76E−05 | 0.007229 | ND | 0.018494 | 0.012699 | 0.030473 | 0.01665 |
| Gm2042 | NA | ND | ND | ND | ND | ND | ND | ND |
| Syngap1 | NA | ND | ND | ND | ND | ND | ND | ND |
| Zfp61 | 52 | 7.89E−07 | 0.000318 | 0.0012 | 0.039824 | 0.032828 | 0.000771 | 0.041544 |
| Septin9 | 498 | 0.000501 | 0.021247 | 0.015005 | 0.097767 | 0.075453 | 0.007063 | 0.192909 |
| 1700021N21Rik | 16 | 9.62E−09 | 1.28E−05 | 0.001029 | 0.00288 | 0.005521 | 0.001153 | 0.015268 |
| Hoxd3 | 1370 | 0.006593 | 0.102135 | 0.047819 | 0.340621 | 0.04199 | 0.016486 | 0.444779 |
| Cbx8 | 203 | 4.10E−05 | 0.004291 | 0.092921 | 0.004239 | 0.013042 | 0.018866 | 0.063648 |
| Vdr | 4170 | 0.083529 | 0.425333 | 0.362589 | 0.031901 | 0.142206 | 0.371896 | 0.404863 |
| Gja4 | 17 | 1.57E−08 | 1.97E−05 | 0.012914 | 0.011149 | 0.00353 | 0.000122 | 0.008167 |
| Slc7a11 | 45 | 3.71E−07 | 0.000175 | 0.010325 | 0.007849 | 0.034245 | 0.002471 | 0.002993 |
| Gm16513 | NA | ND | ND | ND | ND | ND | ND | ND |
| 4930558F17Rik | NA | ND | ND | ND | ND | ND | ND | ND |
| Ubash3b | 4789 | 0.110317 | 0.489175 | 0.161276 | 0.254516 | 0.43107 | 0.078382 | 0.289141 |
| Phldb1 | 491 | 0.000477 | 0.020628 | 0.121062 | 0.003586 | 0.034913 | 0.062664 | 0.148737 |
| Prrt2 | 80 | 3.49E−06 | 0.000927 | 0.005755 | 0.047241 | 0.008078 | 0.003087 | 0.04415 |
| Zfhx2os | 21 | 4.12E−08 | 4.17E−05 | 0.007385 | 0.002593 | 0.006633 | 0.001254 | 0.009745 |
| Gvin2 | NA | ND | ND | ND | ND | ND | ND | ND |
| Gvin-ps2 | NA | ND | ND | ND | ND | ND | ND | ND |
| Ptprd | 1592 | 0.009166 | 0.122348 | 0.432645 | 0.012741 | ND | 0.009729 | 0.718749 |
| Proc | 116 | 1.01E−05 | 0.001846 | 0.006867 | 0.009983 | 0.019031 | 0.011247 | 0.074148 |
| Lgals6 | 19 | 2.60E−08 | 2.91E−05 | 0.016576 | 0.000278 | 0.005143 | 0.116256 | 0.00033 |
| Mmp2 | 290 | 0.000102 | 0.007458 | 0.005565 | 0.230172 | 0.123159 | 0.007652 | 0.016044 |
| Ptk7 | 657 | 0.000991 | 0.032019 | 0.00415 | ND | 0.078774 | 0.040225 | ND |
| Dnah7c | 198 | 3.92E−05 | 0.004204 | 0.28398 | 0.002278 | 0.005175 | 0.361165 | 0.004821 |
| Zfp791 | 406 | 0.000287 | 0.014999 | 0.005296 | 0.194567 | 0.131812 | 0.033788 | 0.015884 |
| Rps4l | 20 | 4.11E−08 | 4.17E−05 | 0.078857 | 0.00196 | 0.00248 | 8.69E−05 | 0.046437 |
| Ggt5 | 54 | 8.73E−07 | 0.000342 | 0.001744 | 0.118051 | 0.003348 | 0.00923 | 0.008906 |
| Ptprf | 180 | 2.80E−05 | 0.00329 | 0.026531 | 0.008081 | 0.014171 | ND | 0.008742 |
| Galnt10 | 16327 | 0.848545 | 1 | ND | 0.518293 | 0.524475 | ND | 0.966305 |
| Marveld2 | 4 | 4.37E−11 | 2.32E−07 | 0.000321 | 0.000407 | 0.000892 | 0.000277 | 0.019245 |
| 2310043O21Rik | 660 | 0.001002 | 0.03225 | 0.301164 | 0.048928 | 0.017369 | 0.032017 | 0.045967 |
| Thtpa | 24 | 7.50E−08 | 6.64E−05 | 0.021707 | 0.002128 | 0.003791 | 0.000735 | 0.024277 |
| Gon4l | 1846 | 0.013591 | 0.156444 | 0.126997 | 0.241034 | 0.449228 | 0.028604 | 0.036326 |
| Tmem267 | 556 | 0.000648 | 0.02477 | 0.017462 | 0.031705 | 0.810689 | 0.751755 | 0.000627 |
| Lsr | 6 | 8.92E−10 | 3.16E−06 | 0.001735 | 0.009602 | 0.003222 | 0.000151 | 0.002326 |
| Gata3 | 8173 | 0.315534 | 0.819917 | 0.718495 | 0.40911 | 0.185041 | 0.121566 | 0.466969 |
| Tiam1 | 190 | 3.48E−05 | 0.003894 | 0.037811 | 0.015931 | 0.013929 | 0.018074 | 0.033181 |
| Adad1 | 3 | 1.20E−11 | 8.51E−08 | 7.32E−05 | 0.000213 | 0.002053 | 0.002344 | 0.001943 |
| Skint11 | NA | ND | ND | ND | ND | ND | ND | ND |
| Obscn | 8692 | 0.349224 | 0.853244 | 0.389444 | 0.645883 | 0.234532 | 0.180278 | 0.364208 |
| Pxn | 3885 | 0.07336 | 0.401037 | 0.415105 | 0.701613 | 0.04958 | 0.022087 | 0.623567 |
| Med15 | 9 | 2.84E−09 | 6.71E−06 | 0.0007 | 0.022522 | 0.002282 | 0.00216 | 0.000911 |
| Ebfl | 27 | 1.08E−07 | 8.46E−05 | 0.022508 | 0.000871 | 0.009794 | 0.003188 | 0.007791 |
| Tcp10a | 226 | 5.35E−05 | 0.005026 | 0.019959 | 0.027581 | 0.030475 | 0.012927 | 0.039648 |
| BC021767 | 353 | 0.000195 | 0.011759 | 0.064668 | 0.045414 | 0.176702 | 0.040353 | 0.002124 |
| Znrfl | 194 | 3.67E−05 | 0.004014 | 0.007077 | ND | 0.029954 | 0.012802 | 0.013485 |
| Ckm | 7543 | 0.273324 | 0.76966 | 0.671626 | 0.881465 | 0.08347 | 0.277774 | 0.165211 |
| Evpl | 61 | 1.44E−06 | 0.000503 | 0.011518 | 0.028367 | 0.004264 | 0.007932 | 0.009359 |
| Mir1966 | 26 | 1.02E−07 | 8.35E−05 | 0.224916 | 0.000634 | 0.001937 | 0.006306 | 0.002577 |
| Espn | 10672 | 0.491261 | 0.977643 | 0.429822 | 0.139813 | 0.445792 | 0.372873 | 0.894154 |
| Ctps | 694 | 0.001132 | 0.034648 | 0.06217 | 0.040649 | 0.058098 | 0.077527 | 0.038944 |
| Asap2 | 207 | 4.36E−05 | 0.004472 | ND | ND | 0.005047 | ND | 0.000632 |
| Ephb4 | 461 | 0.000409 | 0.018859 | 0.134597 | 0.217326 | 0.011882 | 0.012699 | 0.026222 |
| D2hgdh | 750 | 0.001372 | 0.038872 | 0.187808 | 0.051533 | 0.013325 | 0.033405 | 0.133242 |
| Cdca7l | 756 | 0.001404 | 0.039469 | 0.021254 | 0.006534 | 0.981708 | 0.084363 | 0.051492 |
| Adamts18 | 4215 | 0.084809 | 0.427244 | 0.051791 | 0.142727 | 0.256179 | 0.507506 | 0.264476 |
| Chd9 | 530 | 0.000559 | 0.022402 | 0.020032 | 0.160234 | 0.111149 | 0.050694 | 0.009621 |
| Efemp2 | 13 | 8.61E−09 | 1.28E−05 | 0.012339 | 0.002574 | 0.002441 | 0.002161 | 0.00151 |
| Kbtbd7 | 32 | 1.74E−07 | 0.000114 | 0.001681 | 0.022992 | 0.002196 | 0.046864 | 0.002108 |
| Larp1 | 11265 | 0.535713 | 1 | ND | 0.248299 | 0.354328 | 0.550234 | 0.621141 |
| Ccdc152 | 15 | 9.15E−09 | 1.28E−05 | 0.005681 | 0.003889 | 0.001356 | 0.004388 | 0.002065 |
| 1500004A13Rik | NA | ND | ND | ND | ND | ND | ND | ND |
| Ccnjl | 12281 | 0.60385 | 1 | 0.439763 | 0.355958 | 0.442238 | 0.645144 | 0.360835 |
| Spred2 | 196 | 3.82E−05 | 0.004147 | 0.029055 | 0.003601 | 0.038275 | 0.039845 | 0.035456 |
| Plekhm2 | 486 | 0.000469 | 0.020485 | 0.033236 | 0.241111 | 0.043123 | 0.018874 | 0.0212 |
| Mrpl40 | 969 | 0.002652 | 0.058133 | 0.027986 | 0.068913 | 0.067573 | 0.049791 | 0.216615 |
| Unc80 | 1677 | 0.01053 | 0.133419 | 0.184638 | 0.152087 | 0.05974 | 0.043411 | 0.135037 |
| Aktip | 463 | 0.000415 | 0.019047 | 0.009274 | 0.005235 | 0.028502 | 0.485244 | 0.175731 |
| A430093F15Rik | 39 | 2.46E−07 | 0.000134 | 0.010551 | 0.006046 | 0.010812 | 0.001827 | 0.01003 |
| Vmn1r252 | NA | ND | ND | ND | ND | ND | ND | ND |
| Prkd2 | 1520 | 0.008275 | 0.115683 | 0.302676 | 0.13255 | 0.128352 | ND | 0.006516 |
| Kcnk12 | 511 | 0.000524 | 0.021737 | 0.027471 | 0.008046 | 0.144925 | 0.017467 | 0.286126 |
| Tcea3 | 1106 | 0.003861 | 0.074131 | 0.09433 | 0.063474 | 0.132042 | ND | 0.015353 |
| Gm3002 | NA | ND | ND | ND | ND | ND | ND | ND |
| AI661453 | 475 | 0.000448 | 0.020047 | ND | 0.003403 | 0.0198 | 0.782454 | 0.014696 |
| Plbd1 | 2510 | 0.028412 | 0.240429 | 0.417702 | 0.051277 | 0.085941 | 0.116418 | 0.20252 |
| Rerg | 468 | 0.000425 | 0.019236 | 0.023773 | 0.017081 | 0.205694 | 0.044075 | 0.03302 |
| Pnma8b | 179 | 2.80E−05 | 0.00329 | 0.009268 | 0.003267 | 0.08362 | 0.032279 | 0.046966 |
| Sptan1 | 67 | 1.97E−06 | 0.000625 | 0.063237 | 0.019487 | 0.000781 | 0.00516 | 0.030238 |
| Gm14327 | 146 | 1.75E−05 | 0.002547 | 0.117309 | ND | 0.005784 | 0.00018 | ND |
| Hdac5 | 1412 | 0.007061 | 0.106261 | 0.352638 | 0.092794 | 0.148926 | 0.059715 | 0.019005 |
| Smarca5-ps | 43 | 3.19E−07 | 0.000157 | ND | 0.003257 | 0.001777 | 0.008197 | 0.003028 |
| Vmn1r-ps79 | NA | ND | ND | ND | ND | ND | ND | ND |
| Vmn1r101 | NA | ND | ND | ND | ND | ND | ND | ND |
| Vmn1r250 | NA | ND | ND | ND | ND | ND | ND | ND |
| Gm10665 | NA | ND | ND | ND | ND | ND | ND | ND |
| Vmn1r256 | NA | ND | ND | ND | ND | ND | ND | ND |
| Vmnlr100 | NA | ND | ND | ND | ND | ND | ND | ND |
| Gm10668 | NA | ND | ND | ND | ND | ND | ND | ND |
| Gm4513 | NA | ND | ND | ND | ND | ND | ND | ND |
| Vmn1r142 | NA | ND | ND | ND | ND | ND | ND | ND |
| Vmn1r143 | NA | ND | ND | ND | ND | ND | ND | ND |
| Vmn1r251 | NA | ND | ND | ND | ND | ND | ND | ND |
| Vmn1r254 | NA | ND | ND | ND | ND | ND | ND | ND |
| Vmn1r152 | NA | ND | ND | ND | ND | ND | ND | ND |
| Vmn1r255 | NA | ND | ND | ND | ND | ND | ND | ND |
| Tent4a | 5082 | 0.124559 | 0.520502 | 0.091244 | ND | 0.163353 | ND | 0.45156 |
| Ddr1 | 90 | 5.67E−06 | 0.00131 | 0.001703 | 0.004966 | 0.576112 | 0.003579 | 0.030921 |
| Mrgpra2b | NA | ND | ND | ND | ND | ND | ND | ND |
| Tdpoz5 | NA | ND | ND | ND | ND | ND | ND | ND |
| Abcg1 | 312 | 0.000126 | 0.008586 | 0.402176 | 0.038976 | 0.010457 | 0.004665 | 0.033244 |
| 5830428M24Rik | 502 | 0.000505 | 0.021247 | 0.030786 | 0.008917 | 0.141574 | 0.023852 | 0.164188 |
| Slc29a1 | 154 | 1.96E−05 | 0.002702 | 0.004554 | 0.032195 | 0.031072 | 0.038195 | 0.01416 |
| Gm38499 | 1985 | 0.016069 | 0.172012 | 0.02596 | 0.138876 | 0.230422 | 0.158352 | 0.139185 |
| Crtc1 | 2507 | 0.028292 | 0.239659 | 0.103853 | 0.195732 | 0.343376 | 0.390199 | 0.015831 |
| Cdcp1 | 41 | 3.07E−07 | 0.000157 | 0.004183 | 0.001587 | 0.005154 | 0.002545 | 0.188423 |
| Emilin2 | 869 | 0.002044 | 0.049941 | 0.001952 | 0.376685 | 0.086518 | 0.04507 | 0.343346 |
| Ddx4 | 10 | 4.57E−09 | 9.71E−06 | 0.001294 | 0.000478 | 0.001136 | 0.035418 | 0.004903 |
| Mir7649 | NA | ND | ND | ND | ND | ND | ND | ND |
| Shisal1 | 35 | 2.03E−07 | 0.000123 | 0.009133 | 0.008051 | 0.009108 | 0.002666 | 0.005622 |
| Ripor3 | 523 | 0.00054 | 0.021928 | 0.239642 | 0.007008 | 0.03834 | 0.021683 | 0.119091 |
| Capn1 | 10701 | 0.493028 | 0.978551 | 0.144535 | 0.436105 | 0.253488 | 0.975955 | 0.578298 |
| Traf3ip3 | 50 | 7.70E−07 | 0.000318 | 0.002339 | 0.005774 | 0.007926 | 0.009156 | 0.049805 |
| Mdga1 | 4467 | 0.096511 | 0.458785 | 0.511526 | 0.136743 | 0.171931 | 0.128332 | 0.205673 |
| Guca1b | 55 | 8.86E−07 | 0.000342 | 0.001559 | 0.000594 | 0.008188 | 0.554301 | 0.013731 |
| Lgals4 | 74 | 2.64E−06 | 0.000758 | 0.106913 | 0.000817 | 0.026153 | 0.109168 | 0.000857 |
| 4930554H23Rik | 188 | 3.19E−05 | 0.003601 | 0.066188 | 0.008025 | 0.051186 | 0.099025 | 0.001674 |
| Wnt10b | 82 | 3.92E−06 | 0.00101 | 0.033334 | 0.005459 | 0.016192 | 0.012355 | 0.009468 |
| Gm33301 | 12 | 5.81E−09 | 1.03E−05 | 0.000195 | 0.009994 | 0.003451 | 0.00157 | 0.015267 |
| Slc27a1 | 8341 | 0.327331 | 0.833488 | 0.139811 | 0.176654 | 0.147993 | 0.928849 | 0.986308 |
| Mark4 | NA | ND | ND | ND | ND | ND | ND | ND |
| Dapk1 | 3585 | 0.062013 | 0.367358 | ND | ND | ND | 0.084973 | 0.133129 |
| Esyt2 | 4543 | 0.099646 | 0.465722 | 0.072552 | 0.047204 | 0.920865 | 0.18443 | 0.576899 |
| Usp29 | 378 | 0.000234 | 0.01317 | 0.053299 | 0.057747 | 0.126819 | 0.01042 | 0.013816 |
| Nkpd1 | 3054 | 0.043515 | 0.302665 | 0.158886 | 0.130302 | 0.208632 | 0.051951 | 0.377424 |
| 5830417I10Rik | 5347 | 0.138583 | 0.550329 | 0.252165 | 0.08674 | 0.428638 | ND | 0.228306 |
| Col1a2 | 3492 | 0.058549 | 0.355954 | 0.205249 | 0.122175 | 0.125115 | 0.419542 | 0.103956 |
| Vmn1r107 | NA | ND | ND | ND | ND | ND | ND | ND |
| Oxct2b | 53 | 7.93E−07 | 0.000318 | 0.012056 | 0.013256 | 0.002493 | 0.004852 | 0.026138 |
| Ccdc8 | 177 | 2.69E−05 | 0.003231 | 0.015199 | 0.014607 | 0.081044 | 0.005812 | 0.034927 |
| Fxyd1 | 1006 | 0.002978 | 0.0629 | 0.032288 | 0.035828 | 0.01709 | 0.362331 | 0.230138 |
| Cpne5 | 2036 | 0.017006 | 0.17749 | 0.338172 | 0.068512 | 0.223309 | 0.053745 | 0.071652 |
| Ctnnd2 | 8736 | 0.352545 | 0.856883 | 0.518324 | 0.473538 | 0.09755 | 0.404227 | 0.408929 |
| Nr4a2 | 15200 | 0.788415 | 1 | 0.503696 | 0.8201 | 0.601908 | 0.432115 | 0.396486 |
| 1700010N08Rik | NA | ND | ND | ND | ND | ND | ND | ND |
| Gm12185 | NA | ND | ND | ND | ND | ND | ND | ND |
| Tmem94 | 2617 | 0.031307 | 0.254101 | 0.297934 | 0.072631 | 0.33666 | 0.204782 | 0.033808 |
| Exoc3l2 | 2598 | 0.030679 | 0.250823 | ND | 0.032364 | 0.13509 | ND | 0.218365 |
| Capn11 | 3374 | 0.054486 | 0.342792 | 0.320024 | 0.055069 | 0.312845 | 0.025111 | 0.878811 |
| Skint3 | 1823 | 0.01308 | 0.152464 | 0.069408 | 0.175387 | 0.041653 | 0.125479 | 0.212258 |
| Zbtb11os1 | 56 | 1.04E−06 | 0.000389 | 0.00915 | 0.012066 | 0.004167 | 0.016322 | 0.009317 |
| 1700029B22Rik | 59 | 1.12E−06 | 0.000404 | 0.04536 | 0.034804 | 0.004003 | 0.001746 | 0.006928 |
| Clmp | 2410 | 0.026033 | 0.229408 | 0.575613 | 0.301581 | 0.04824 | 0.039536 | 0.114584 |
| Dync2i1 | 9414 | 0.397792 | 0.897408 | ND | ND | ND | 0.757753 | 0.173222 |
| Rims2 | 481 | 0.000463 | 0.020346 | 0.03697 | 0.052883 | 0.169135 | 0.172409 | 0.002383 |
| Col6a3 | 1566 | 0.00879 | 0.119272 | 0.017664 | 0.038824 | 0.128638 | 0.551289 | 0.155691 |
| Cacna1a | 5273 | 0.134893 | 0.543279 | ND | ND | 0.268806 | 0.085158 | 0.331011 |
| St6gal2 | 214 | 4.81E−05 | 0.004747 | 0.021168 | 0.08262 | 0.017258 | 0.011201 | 0.022311 |
| Nkd1 | 1837 | 0.01337 | 0.154624 | ND | ND | 0.01337 | ND | ND |
| Pagr1a | 100 | 6.86E−06 | 0.001458 | 0.00775 | 0.071651 | 0.014129 | 0.002723 | 0.031855 |
| Fign | 18 | 1.94E−08 | 2.29E−05 | 0.01036 | 0.006031 | 0.001157 | 0.001122 | 0.007986 |
| Zcchc24 | 1597 | 0.009237 | 0.122879 | ND | 0.17699 | 0.03251 | 0.32401 | 0.020891 |
| Snx31 | 1920 | 0.014962 | 0.165589 | ND | ND | 0.658532 | 0.064368 | 0.008817 |
| Zbtb7a | 18890 | 0.956553 | 1 | 0.964095 | 0.311313 | 0.635661 | 0.874583 | 0.903456 |
| Sik1 | 4373 | 0.09259 | 0.449496 | 0.096674 | 0.031873 | 0.687416 | 0.387923 | 0.359583 |
| Hif3a | 816 | 0.001742 | 0.045371 | 0.015319 | 0.203326 | 0.23469 | 0.071732 | 0.015114 |
| Atp4b | 11113 | 0.525179 | 1 | 0.33677 | 0.251751 | 0.361852 | 0.417581 | 0.836032 |
| Erbb4 | 5772 | 0.163485 | 0.601539 | 0.338876 | 0.196168 | 0.901672 | 0.637602 | 0.021447 |
| Tmc4 | 153 | 1.94E−05 | 0.00269 | 0.002212 | 0.00214 | 0.021894 | 0.43251 | 0.054227 |

TABLE 19
(Supplementary Table S8c. Ranked genes by differential methylation
(Old + OSKM vs Old-4): Rank_body, p-value body, q-value body)

| | | p-value | q-value | p-value | p-value | p-value | p-value | p-value |
|---|---|---|---|---|---|---|---|---|
| Gene | Rank_body | body | body | body TR1 | body TR2 | body TR3 | body TR4 | body TR5 |
| Nfix | 1 | 2.22E−16 | 1.99E−12 | 6.91E−06 | 1.67E−06 | 0.000191 | 0.000658 | 0.000274 |
| Serpina1b | 29 | 9.19E−10 | 5.49E−07 | 0.00167 | 0.003774 | 0.003287 | 4.18E−05 | 0.022477 |
| Ciapin1 | 58 | 2.71E−08 | 8.25E−06 | 0.000667 | 0.000903 | 0.004312 | 0.048172 | 0.007642 |
| Atp6v1c2 | 269 | 6.17E−06 | 0.000409 | 0.086392 | 0.009923 | 0.300856 | 1.41E−05 | 0.164791 |
| Def8 | 1 | 2.22E−16 | 1.99E−12 | 5.53E−06 | 1.85E−05 | 5.18E−05 | 1.80E−05 | 2.30E−05 |
| Rbfox1 | 4 | 9.38E−14 | 4.20E−10 | 3.48E−08 | 0.034777 | 0.07434 | 3.96E−07 | 0.018667 |
| Vmn2r47 | 7 | 1.03E−12 | 2.63E−09 | 8.96E−05 | 0.000263 | 0.001326 | 0.000119 | 0.002522 |
| Irgc1 | 6 | 5.07E−13 | 1.51E−09 | 0.000135 | 4.47E−05 | 7.94E−05 | 0.005147 | 0.001739 |
| Gm45351 | 8 | 1.43E−12 | 3.21E−09 | 8.96E−05 | 0.000265 | 0.00169 | 0.000119 | 0.00284 |
| Mmp16 | 1603 | 0.002433 | 0.027185 | 0.021519 | 0.036886 | 0.178934 | 0.177623 | 0.049489 |
| Hoxa7 | 975 | 0.000585 | 0.01074 | 0.011975 | 0.024624 | 0.068129 | 0.041231 | 0.223041 |
| Gm53 | 13 | 7.37E−11 | 9.73E−08 | 0.005008 | 0.000297 | 0.002918 | 0.000163 | 0.00158 |
| Gm10377 | 12 | 4.04E−11 | 6.04E−08 | 0.001623 | 0.000799 | 0.000861 | 0.000544 | 0.000937 |
| Mup2 | 15 | 8.42E−11 | 9.85E−08 | 0.000828 | 0.000556 | 0.002707 | 0.000994 | 0.001049 |
| Hoxa2 | 17 | 9.43E−11 | 9.95E−08 | 0.00029 | 0.000381 | 0.01068 | 0.001608 | 0.000776 |
| Skint8 | 14 | 7.60E−11 | 9.73E−08 | 0.024848 | 0.000588 | 0.00037 | 0.000599 | 0.000356 |
| Skint5 | 16 | 8.79E−11 | 9.85E−08 | 0.000899 | 0.003666 | 0.001097 | 0.000421 | 0.000895 |
| Fam171a2 | 3 | 3.04E−15 | 1.82E−11 | 0.012721 | 1.00E−05 | 0.000922 | 2.24E−05 | 5.85E−06 |
| Mir196a-1 | NA | ND | ND | ND | ND | ND | ND | ND |
| Defa23 | 24 | 5.18E−10 | 3.71E−07 | 0.000726 | 0.00027 | 0.000869 | 0.002206 | 0.026984 |
| Gnas | 60 | 2.91E−08 | 8.44E−06 | 0.002129 | 0.002692 | 0.010798 | 0.049876 | 0.000336 |
| Skint6 | 26 | 6.51E−10 | 4.28E−07 | 0.003955 | 0.001434 | 0.001082 | 0.000455 | 0.004716 |
| Hic1 | 124 | 3.44E−07 | 4.94E−05 | 0.234482 | 6.82E−05 | 0.001129 | 0.023682 | 0.043873 |
| Skint10 | 33 | 1.26E−09 | 6.64E−07 | 0.002298 | 0.001838 | 0.007845 | 0.000139 | 0.006048 |
| Irx5 | 11 | 3.17E−11 | 5.17E−08 | 0.009549 | 0.001609 | 0.000699 | 0.00425 | 9.48E−06 |
| Hoxaas3 | 36 | 1.88E−09 | 9.11E−07 | 0.000472 | 0.001142 | 0.008222 | 0.003302 | 0.00301 |
| Acer2 | 104 | 3.04E−07 | 4.88E−05 | 0.001721 | 0.09607 | 0.00044 | 0.003816 | 0.058339 |
| Abi3 | 30 | 9.85E−10 | 5.70E−07 | 0.000113 | 0.000116 | 0.001968 | 0.004581 | 0.177022 |
| Rpl39l | NA | ND | ND | ND | ND | ND | ND | ND |
| Hoxa9 | 5 | 4.94E−13 | 1.51E−09 | 2.91E−05 | 0.000364 | 0.016653 | 6.34E−05 | 0.000373 |
| Hoxa3 | 53 | 2.10E−08 | 6.99E−06 | 0.001018 | 0.001092 | 0.035134 | 0.003269 | 0.005574 |
| Chst5 | 59 | 2.76E−08 | 8.26E−06 | 0.003658 | 0.000174 | 0.011936 | 0.005324 | 0.024086 |
| Gse1 | 72 | 7.04E−08 | 1.73E−05 | 0.006724 | 0.003029 | 0.004889 | 0.002327 | 0.012523 |
| Hoxb3 | 10 | 1.93E−11 | 3.47E−08 | 0.000266 | 0.000912 | 0.000158 | 0.01811 | 0.000357 |
| Kansl1 | 799 | 0.000301 | 0.006743 | 0.041012 | 0.036162 | 0.03026 | 0.060779 | 0.028442 |
| Bcl9l | 9 | 1.40E−11 | 2.80E−08 | 0.002201 | 0.000405 | 0.000425 | 0.002715 | 0.000169 |
| Mir467h | NA | ND | ND | ND | ND | ND | ND | ND |
| Col28a1 | 42 | 6.26E−09 | 2.61E−06 | 0.000181 | 0.004048 | 0.000175 | 0.029203 | 0.046763 |
| Prxl2c | 145 | 6.16E−07 | 7.53E−05 | 0.007819 | 0.009707 | 0.005413 | 0.004263 | 0.021366 |
| Gm2042 | 46 | 7.33E−09 | 2.80E−06 | 0.002781 | 0.000699 | 0.007059 | 0.002685 | 0.005708 |
| Syngap1 | 47 | 7.62E−09 | 2.85E−06 | 0.025122 | 0.002606 | 0.003709 | 0.00024 | 0.00377 |
| Zfp61 | 577 | 9.81E−05 | 0.003044 | 0.020673 | 0.074564 | 0.082973 | 0.0003 | 0.481225 |
| Septin9 | 81 | 1.05E−07 | 2.29E−05 | 0.004403 | 0.003093 | 0.005607 | 0.008791 | 0.006879 |
| 1700021N21Rik | NA | ND | ND | ND | ND | ND | ND | ND |
| Hoxd3 | 44 | 6.62E−09 | 2.62E−06 | 0.007187 | 0.000408 | 0.01066 | 0.049 | 0.000122 |
| Cbx8 | 194 | 1.74E−06 | 0.000159 | 0.000638 | 0.001687 | 0.012235 | 0.12946 | 0.075699 |
| Vdr | 19 | 2.13E−10 | 1.88E−07 | 0.001488 | 0.000134 | 0.002417 | 0.003744 | 0.002052 |
| Gja4 | NA | ND | ND | ND | ND | ND | ND | ND |
| Slc7a11 | 813 | 0.000318 | 0.007012 | 0.032822 | 0.041672 | 0.038668 | 0.087797 | 0.017989 |
| Gm16513 | 50 | 1.76E−08 | 6.19E−06 | 0.006422 | 0.000311 | 0.032325 | 0.000581 | 0.015437 |
| 4930558F17Rik | 51 | 1.81E−08 | 6.25E−06 | 0.005846 | 0.002015 | 0.000685 | 0.008233 | 0.008997 |
| Ubash3b | 23 | 3.07E−10 | 2.29E−07 | 0.004096 | 0.002956 | 0.000735 | 0.000546 | 0.001152 |
| Phldb1 | 96 | 2.28E−07 | 4.21E−05 | 0.022418 | 0.001223 | 0.009709 | 0.012387 | 0.00349 |
| Prrt2 | 469 | 4.97E−05 | 0.001897 | 0.145625 | 0.027728 | 0.018761 | 0.008442 | 0.012276 |
| Zfhx2os | 1678 | 0.002908 | 0.031012 | 0.022864 | 0.381689 | 0.052203 | 0.092125 | 0.038008 |
| Gvin2 | 54 | 2.29E−08 | 7.35E−06 | 0.001394 | 0.002401 | 0.007693 | 0.018323 | 0.001668 |
| Gvin-ps2 | 54 | 2.29E−08 | 7.35E−06 | 0.001394 | 0.002401 | 0.007693 | 0.018323 | 0.001668 |
| Ptprd | 74 | 7.36E−08 | 1.75E−05 | 0.010587 | 0.024079 | 4.71E−05 | 0.01741 | 0.014619 |
| Proc | 368 | 2.22E−05 | 0.00108 | 0.015074 | 0.01982 | 0.066271 | 0.011408 | 0.012755 |
| Lgals6 | NA | ND | ND | ND | ND | ND | ND | ND |
| Mmp2 | 205 | 2.01E−06 | 0.000175 | 0.020602 | 0.001237 | 0.034291 | 0.002023 | 0.086828 |
| Ptk7 | 141 | 5.31E−07 | 6.68E−05 | 0.020499 | 0.010253 | 0.002457 | 0.010612 | 0.005719 |
| Dnah7c | 283 | 8.28E−06 | 0.000522 | 0.027088 | 0.009655 | 0.004674 | 0.023831 | 0.029376 |
| Zfp791 | 195 | 1.74E−06 | 0.000159 | ND | 0.002987 | 0.072663 | 0.001596 | 0.002921 |
| Rps4l | NA | ND | ND | ND | ND | ND | ND | ND |
| Ggt5 | 865 | 0.000411 | 0.008503 | 0.017563 | 0.082577 | 0.038873 | 0.089285 | 0.023095 |
| Ptprf | 333 | 1.48E−05 | 0.000795 | 0.143259 | 0.01296 | 0.003788 | 0.049212 | 0.005041 |
| Galnt10 | 31 | 1.11E−09 | 6.19E−07 | 0.001061 | 0.001261 | 0.011097 | 3.26E−05 | 0.049642 |
| Marveld2 | 11887 | 0.564943 | 0.852046 | 0.795132 | 0.12029 | 0.891402 | 0.485371 | 0.318683 |
| 2310043O21Rik | 132 | 4.10E−07 | 5.53E−05 | 0.00267 | 0.005571 | 0.01571 | 0.009309 | 0.010617 |
| Thtpa | 2032 | 0.005319 | 0.046887 | 0.016549 | 0.331992 | 0.078832 | 0.074788 | 0.114259 |
| Gon4l | 56 | 2.41E−08 | 7.59E−06 | 0.000578 | 0.009032 | 0.010849 | 0.00155 | 0.009505 |
| Tmem267 | 159 | 7.97E−07 | 8.94E−05 | 0.071497 | 0.000218 | 0.014003 | 0.005414 | 0.043058 |
| Lsr | 6960 | 0.163355 | 0.420602 | 0.503865 | 0.045845 | 0.435089 | 0.131805 | 0.617828 |
| Gata3 | 20 | 2.21E−10 | 1.88E−07 | 0.016409 | 2.79E−05 | 0.003027 | 0.002213 | 0.001257 |
| Tiam1 | 366 | 2.13E−05 | 0.001043 | 0.022466 | 0.008279 | 0.022211 | 0.037509 | 0.017677 |
| Adad1 | 16106 | 0.925044 | 1 | 0.41783 | 0.534079 | 0.81064 | 0.825245 | 0.725684 |
| Skint11 | 73 | 7.33E−08 | 1.75E−05 | 0.006943 | 0.011878 | 0.000949 | 0.009528 | 0.004078 |
| Obscn | 21 | 2.49E−10 | 1.98E−07 | 0.000447 | 9.31E−05 | 0.005655 | 0.003262 | 0.00575 |
| Pxn | 38 | 3.86E−09 | 1.78E−06 | 0.005 | 0.002871 | 0.001302 | 0.001067 | 0.005047 |
| Med15 | 5337 | 0.081099 | 0.272287 | 0.139366 | 0.134102 | 0.22232 | 0.151139 | 0.375087 |
| Ebf1 | 2152 | 0.006064 | 0.05047 | 0.053424 | 0.022319 | 0.338295 | 0.060921 | 0.181233 |
| Tcp10a | 373 | 2.34E−05 | 0.001123 | 0.128712 | 0.029577 | 0.018839 | 0.003741 | 0.011456 |
| BC021767 | 245 | 4.78E−06 | 0.000349 | 0.00613 | 0.03573 | 0.027445 | 0.028505 | 0.002561 |
| Znrf1 | 452 | 4.28E−05 | 0.001696 | 0.008701 | 0.006814 | 0.22534 | 0.040049 | 0.012179 |
| Ckm | 25 | 5.59E−10 | 3.86E−07 | 0.002183 | 0.002774 | 0.008417 | 0.000258 | 0.000844 |
| Evpl | 1044 | 0.000709 | 0.01215 | 0.008601 | 0.059337 | 0.023032 | 0.389434 | 0.05198 |
| Mir1966 | NA | ND | ND | ND | ND | ND | ND | ND |
| Espn | 22 | 2.54E−10 | 1.98E−07 | 0.005898 | 2.11E−05 | 0.05254 | 0.000216 | 0.003207 |
| Ctps | 181 | 1.20E−06 | 0.000119 | 0.00354 | 0.021582 | 0.06747 | 0.000724 | 0.022278 |
| Asap2 | 468 | 4.90E−05 | 0.001876 | 0.032984 | 0.084154 | 0.166319 | 0.000702 | 0.023811 |
| Ephb4 | 237 | 3.87E−06 | 0.000292 | 0.002313 | 0.11766 | 0.004282 | 0.015858 | 0.018386 |
| D2hgdh | 173 | 1.06E−06 | 0.000109 | 0.082162 | 0.005145 | 0.003092 | 0.019107 | 0.00287 |
| Cdca7l | 162 | 8.23E−07 | 9.06E−05 | 0.106618 | 0.000763 | 0.012833 | 0.004989 | 0.010144 |
| Adamts18 | 43 | 6.50E−09 | 2.62E−06 | 0.000514 | 0.000475 | 0.004445 | 0.01854 | 0.009113 |
| Chd9 | 227 | 2.92E−06 | 0.00023 | 0.00033 | 0.009013 | 0.03074 | 0.057951 | 0.04553 |
| Efemp2 | 5235 | 0.076897 | 0.263226 | 0.140861 | 0.118451 | 0.078364 | 0.216416 | 0.760774 |
| Kbtbd7 | NA | ND | ND | ND | ND | ND | ND | ND |
| Larp1 | 32 | 1.24E−09 | 6.64E−07 | 0.002407 | 0.001437 | 0.009533 | 0.001276 | 0.000654 |
| Ccdc152 | 5320 | 0.08069 | 0.271764 | 0.020828 | 0.497231 | 0.22297 | 0.242311 | 0.417368 |
| 1500004A13Rik | 90 | 1.90E−07 | 3.71E−05 | 0.012462 | 0.006194 | 0.000796 | 0.026866 | 0.005626 |
| Ccnjl | 18 | 1.91E−10 | 1.81E−07 | 0.000869 | 2.47E−05 | 0.013028 | 0.001241 | 0.00943 |
| Spred2 | 528 | 7.28E−05 | 0.002468 | 0.022654 | 0.043733 | 0.046192 | 0.015563 | 0.017801 |
| Plekhm2 | 251 | 4.98E−06 | 0.000354 | 0.112296 | 0.005342 | 0.002513 | 0.005939 | 0.051507 |
| Mrpl40 | 163 | 8.31E−07 | 9.09E−05 | 0.007328 | 0.015092 | 0.00355 | 0.009295 | 0.014652 |
| Unc80 | 88 | 1.79E−07 | 3.61E−05 | 0.002712 | 0.004805 | 0.020155 | 0.00405 | 0.008146 |
| Aktip | 263 | 5.71E−06 | 0.000388 | 0.002833 | 0.001725 | 0.19627 | 0.020276 | 0.027959 |
| A430093F15Rik | 2477 | 0.009368 | 0.067732 | 0.119017 | 0.083341 | 0.074389 | 0.125841 | 0.089389 |
| Vmn1r252 | 99 | 2.56E−07 | 4.58E−05 | ND | 0.000724 | 0.000735 | ND | 0.00205 |
| Prkd2 | 154 | 7.09E−07 | 8.20E−05 | 0.018942 | 0.023074 | 0.001255 | 0.00161 | 0.050041 |
| Kcnk12 | 264 | 5.75E−06 | 0.000389 | 0.004149 | 0.01607 | 0.02545 | 0.016203 | 0.019942 |
| Tcea3 | 208 | 2.09E−06 | 0.000179 | 0.092754 | 0.006712 | 0.014893 | 0.000823 | 0.021075 |
| Gm3002 | 101 | 2.65E−07 | 4.66E−05 | 0.024036 | 0.00149 | 0.018116 | 0.005245 | 0.004048 |
| AI661453 | 315 | 1.21E−05 | 0.000687 | 0.006794 | 0.003373 | 0.07659 | 0.022677 | 0.034226 |
| Plbd1 | 69 | 6.61E−08 | 1.69E−05 | 0.006463 | 0.000809 | 0.001921 | 0.01592 | 0.016851 |
| Rerg | 282 | 8.19E−06 | 0.000519 | 0.013578 | 0.009683 | 0.012132 | 0.052459 | 0.010089 |
| Pnma8b | 640 | 0.000155 | 0.004347 | ND | 0.001193 | 0.00411 | ND | 0.303576 |
| Sptan1 | 1445 | 0.001842 | 0.022838 | 0.080539 | 0.067545 | 0.086332 | 0.035307 | 0.051532 |
| Gm14327 | 717 | 0.000227 | 0.005678 | 0.018794 | 0.028253 | 0.012681 | 0.33902 | 0.023677 |
| Hdac5 | 136 | 4.68E−07 | 6.09E−05 | 0.00396 | 0.002445 | 0.00534 | 0.009949 | 0.052534 |
| Smarca5-ps | NA | ND | ND | ND | ND | ND | ND | ND |
| Vmn1r-ps79 | 110 | 3.37E−07 | 4.88E−05 | ND | 0.000935 | 0.000504 | 0.101492 | 0.003198 |
| Vmn1r101 | 111 | 3.37E−07 | 4.88E−05 | ND | 0.000935 | 0.000504 | 0.101492 | 0.003198 |
| Vmn1r250 | 112 | 3.37E−07 | 4.88E−05 | ND | 0.000935 | 0.000504 | 0.101492 | 0.003198 |
| Gm10665 | 113 | 3.37E−07 | 4.88E−05 | ND | 0.000935 | 0.000504 | 0.101492 | 0.003198 |
| Vmn1r256 | 114 | 3.37E−07 | 4.88E−05 | ND | 0.000935 | 0.000504 | 0.101492 | 0.003198 |
| Vmn1r100 | 115 | 3.37E−07 | 4.88E−05 | ND | 0.000935 | 0.000504 | 0.101492 | 0.003198 |
| Gm10668 | 116 | 3.37E−07 | 4.88E−05 | ND | 0.000935 | 0.000504 | 0.101492 | 0.003198 |
| Gm4513 | 117 | 3.37E−07 | 4.88E−05 | ND | 0.000935 | 0.000504 | 0.101492 | 0.003198 |
| Vmn1r142 | 118 | 3.37E−07 | 4.88E−05 | ND | 0.000935 | 0.000504 | 0.101492 | 0.003198 |
| Vmn1r143 | 119 | 3.37E−07 | 4.88E−05 | ND | 0.000935 | 0.000504 | 0.101492 | 0.003198 |
| Vmn1r251 | 120 | 3.37E−07 | 4.88E−05 | ND | 0.000935 | 0.000504 | 0.101492 | 0.003198 |
| Vmn1r254 | 121 | 3.37E−07 | 4.88E−05 | ND | 0.000935 | 0.000504 | 0.101492 | 0.003198 |
| Vmn1r152 | 122 | 3.37E−07 | 4.88E−05 | ND | 0.000935 | 0.000504 | 0.101492 | 0.003198 |
| Vmn1r255 | 123 | 3.37E−07 | 4.88E−05 | ND | 0.000935 | 0.000504 | 0.101492 | 0.003198 |
| Tent4a | 71 | 6.98E−08 | 1.73E−05 | 0.005846 | 0.001501 | 0.001745 | 0.019421 | 0.009668 |
| Ddr1 | 1052 | 0.00072 | 0.012272 | 0.165076 | 0.001838 | 0.030494 | 0.166109 | 0.158231 |
| Mrgpra2b | 126 | 3.59E−07 | 5.04E−05 | 0.005806 | 0.006427 | 0.00721 | 0.001307 | 0.056003 |
| Tdpoz5 | 127 | 3.62E−07 | 5.04E−05 | 0.007658 | 0.001486 | 0.002008 | 0.008727 | 0.099744 |
| Abcg1 | 449 | 4.24E−05 | 0.001685 | 0.053344 | 0.004606 | 0.014056 | 0.018812 | 0.099067 |
| 5830428M24Rik | 311 | 1.14E−05 | 0.000653 | 0.049977 | 0.007532 | 0.065287 | 0.01166 | 0.00441 |
| Slc29a1 | 814 | 0.000319 | 0.007012 | 0.08109 | 0.024804 | 0.057409 | 0.148569 | 0.004873 |
| Gm38499 | 93 | 2.13E−07 | 4.06E−05 | 0.000747 | 0.1618 | 0.021713 | 0.000503 | 0.008069 |
| Crtc1 | 83 | 1.13E−07 | 2.41E−05 | 0.001007 | 0.010884 | 0.004073 | 0.00312 | 0.036211 |
| Cdcp1 | 2881 | 0.014507 | 0.090195 | 0.395312 | 0.02755 | 0.050043 | 0.482214 | 0.059864 |
| Emilin2 | 213 | 2.44E−06 | 0.000205 | 0.004032 | 0.017586 | 0.049763 | 0.006432 | 0.008563 |
| Ddx4 | 8327 | 0.252421 | 0.543409 | 0.063184 | 0.202915 | 0.585412 | 0.42976 | 0.595739 |
| Mir7649 | 137 | 4.68E−07 | 6.09E−05 | 0.010151 | 0.002029 | 0.00472 | 0.011442 | 0.024298 |
| Shisal1 | 3270 | 0.020786 | 0.11388 | 0.147906 | 0.07301 | 0.198395 | 0.031883 | 0.394295 |
| Ripor3 | 328 | 1.41E−05 | 0.000767 | 0.015588 | 0.007948 | 0.009764 | 0.232652 | 0.005823 |
| Capn1 | 34 | 1.45E−09 | 7.44E−07 | 0.003664 | 0.004158 | 0.001369 | 0.001906 | 0.000826 |
| Traf3ip3 | 2291 | 0.007448 | 0.05824 | 0.155118 | 0.057606 | 0.040055 | 0.148246 | 0.112498 |
| Mdga1 | 61 | 2.92E−08 | 8.44E−06 | 0.003446 | 0.003705 | 0.00787 | 0.002567 | 0.004031 |
| Guca1b | 2306 | 0.007625 | 0.059231 | 0.099678 | 0.038547 | 0.271426 | 0.156712 | 0.037766 |
| Lgals4 | 1617 | 0.002508 | 0.02778 | 0.043801 | 0.008743 | 0.041255 | 0.473167 | 0.174156 |
| 4930554H23Rik | 828 | 0.000336 | 0.007259 | 0.01158 | 0.05601 | 0.094353 | 0.037005 | 0.039489 |
| Wnt10b | 1579 | 0.002351 | 0.026676 | 0.014691 | 0.026715 | 0.093751 | 0.133216 | 0.243145 |
| Gm33301 | 9026 | 0.307126 | 0.610001 | 0.317586 | 0.226934 | 0.390355 | 0.488619 | 0.21173 |
| Slc27a1 | 39 | 4.84E−09 | 2.17E−06 | 0.001214 | 0.002683 | 0.001507 | 0.040112 | 0.000662 |
| Mark4 | 152 | 6.99E−07 | 8.19E−05 | 0.029993 | 0.008921 | 0.043663 | 0.00095 | 0.003917 |
| Dapk1 | 143 | 5.92E−07 | 7.36E−05 | 0.016238 | 0.006451 | 0.008908 | 0.004178 | 0.00916 |
| Esyt2 | 65 | 4.62E−08 | 1.25E−05 | 0.000326 | 0.000853 | 0.032194 | 0.005844 | 0.033901 |
| Usp29 | 494 | 5.62E−05 | 0.002035 | 0.012836 | 0.031754 | 0.040385 | 0.013254 | 0.041944 |
| Nkpd1 | 85 | 1.23E−07 | 2.57E−05 | 0.048343 | 0.004943 | 0.000992 | 0.003368 | 0.006999 |
| 5830417I10Rik | 64 | 4.54E−08 | 1.25E−05 | 0.015636 | 0.001619 | 0.000599 | 0.022461 | 0.005103 |
| Col1a2 | 80 | 9.79E−08 | 2.17E−05 | 0.002309 | 0.006538 | 0.003136 | 0.011456 | 0.007869 |
| Vmn1r107 | 158 | 7.61E−07 | 8.59E−05 | ND | 0.000876 | 0.003279 | 0.027866 | 0.004871 |
| Oxct2b | NA | ND | ND | ND | ND | ND | ND | ND |
| Ccdc8 | 978 | 0.000592 | 0.010845 | 0.04298 | 0.004334 | 0.518064 | 0.093318 | 0.020843 |
| Fxyd1 | 243 | 4.71E−06 | 0.000346 | 0.002585 | 0.001934 | 0.081948 | 0.002266 | 0.463614 |
| Cpne5 | 144 | 5.95E−07 | 7.36E−05 | 0.001776 | 0.002476 | 0.005997 | 0.066826 | 0.020387 |
| Ctnnd2 | 45 | 6.72E−09 | 2.62E−06 | 0.000776 | 0.000905 | 0.00039 | 0.018529 | 0.037455 |
| Nr4a2 | 28 | 7.82E−10 | 4.83E−07 | 0.005431 | 0.00046 | 0.00072 | 0.000313 | 0.028744 |
| 1700010N08Rik | 169 | 9.57E−07 | 0.000101 | 0.001006 | 0.008777 | 0.008786 | 0.056602 | 0.014414 |
| Gm12185 | 170 | 9.66E−07 | 0.000101 | 0.060607 | 0.001244 | 0.0063 | 0.007792 | 0.017272 |
| Tmem94 | 107 | 3.26E−07 | 4.88E−05 | 0.007479 | 0.008618 | 0.001926 | 0.006519 | 0.021737 |
| Exoc3l2 | 148 | 6.26E−07 | 7.53E−05 | 0.060608 | 0.006655 | 0.015268 | 0.015445 | 0.000401 |
| Capn11 | 87 | 1.26E−07 | 2.58E−05 | 0.00652 | 0.252353 | 0.000213 | 0.000186 | 0.088359 |
| Skint3 | 164 | 9.04E−07 | 9.82E−05 | 0.005512 | 0.011757 | 0.002174 | 0.035568 | 0.011799 |
| Zbtb11os1 | NA | ND | ND | ND | ND | ND | ND | ND |
| 1700029B22Rik | NA | ND | ND | ND | ND | ND | ND | ND |
| Clmp | 140 | 5.08E−07 | 6.46E−05 | 0.049859 | 0.001432 | 0.001027 | 0.034387 | 0.01179 |
| Dync2i1 | 131 | 3.84E−07 | 5.21E−05 | 0.000185 | 0.020621 | 0.003459 | 0.025276 | 0.064005 |
| Rims2 | 478 | 5.19E−05 | 0.001944 | 0.028366 | 0.015718 | 0.030823 | 0.018115 | 0.033315 |
| Col6a3 | 199 | 1.83E−06 | 0.000164 | 0.005288 | 0.009133 | 0.006288 | 0.013978 | 0.03246 |
| Cacna1a | 139 | 4.87E−07 | 6.24E−05 | 0.004253 | 0.001798 | 0.016246 | 0.016689 | 0.013661 |
| St6gal2 | 954 | 0.000541 | 0.010153 | 0.011502 | 0.252309 | 0.018781 | 0.08742 | 0.034964 |
| Nkd1 | 24 | 4.73E−06 | 0.000347 | 0.009587 | 0.004781 | 0.007895 | 0.012755 | 0.09386 |
| Pagr1a | 1758 | 0.003304 | 0.033656 | 0.780047 | 0.086035 | 0.046069 | 0.024162 | 0.025478 |
| Fign | 8296 | 0.250571 | 0.541348 | 0.357078 | 0.317836 | 0.31963 | 0.176382 | 0.295814 |
| Zcchc24 | 218 | 2.53E−06 | 0.000207 | 0.06204 | 0.00506 | 0.005092 | 0.006148 | 0.020695 |
| Snx31 | 260 | 5.57E−06 | 0.000383 | 0.003802 | 0.00506 | 0.053601 | 0.020624 | 0.024828 |
| Zbtb7a | 27 | 6.69E−10 | 4.28E−07 | 0.046583 | 8.00E−05 | 4.96E−05 | 0.108336 | 0.000678 |
| Sik1 | 84 | 1.18E−07 | 2.48E−05 | 0.326353 | 0.004234 | 0.020639 | 0.001323 | 0.00014 |
| Hif3a | 347 | 1.61E−05 | 0.000827 | 0.432566 | 0.02461 | 0.002844 | 0.002448 | 0.026019 |
| Atp4b | 40 | 5.36E−09 | 2.35E−06 | 0.00165 | 0.000305 | 0.012812 | 0.003128 | 0.007283 |
| Erbb4 | 66 | 5.08E−08 | 1.36E−05 | 0.00364 | 0.002911 | 0.008304 | 0.005118 | 0.004399 |
| Tmc4 | 1357 | 0.001583 | 0.020886 | 0.014295 | 0.057666 | 0.171016 | 0.072101 | 0.068455 |

Although the invention has been described with reference to the present embodiment, it should be understood that various modifications can be made without departing from the spirit of the invention. Accordingly, the invention is limited only by the following claims.

1. A method of identifying a compound comprising:
analyzing a PRC2-associated epigenomic change in a sample before and after contacting the sample with the compound,
wherein the PRC2-associated epigenomic change comprises a decrease in mean methylation level (MML) or a reduction in normalized methylation entropy (NME) in one or more PRC2 gene targets in the sample after contacting with the compound.

2. The method of claim 1, wherein the PRC2-associated epigenomic change comprises a decrease in MML or NME in one or more of:
a) a promoter of the PRC2 gene target;
b) a H3K27me3 methylation site of a PRC2 gene target and/or a
c) a binding site of EZH2, SUZ12, EED, JARID2, MTF2 or a combination thereof in the PRC2 gene target.

3. The method of claim 2, wherein the promoter comprises H3K27me3, H3K4me3 or a combination thereof.

4. The method of claim 1, wherein the compound increases gene expression of one or more PRC2 gene targets.

5. The method of claim 1, wherein the PRC2 gene target is CRTC1, SMAD3, WNT3A, NR4A2, HMBOX1, FOXA1, CAMK2B, EGR, NGF, PIM1, TBX3, TWIST1, MSRB3, ETV6 or a combination thereof.

6. A compound for PRC2 gene target inhibition identified by the method of claim 1.

7. A method of rejuvenating a population of aging cells comprising contacting the population of cells with a compound of claim 6.

8. A method of improving proliferative capacity of a population of cells comprising contacting the population of cells with a compound of claim 6.

9. A method of reducing fibrosis comprising contacting a compound of claim 6 with cells of a subject.

10. A method of tissue regeneration comprising contacting tissue with a compound of claim 6.

11. A method of identifying a genomic region of differential DNA methylation comprising:
a) obtaining a DNA sample from one or more of a young cohort, an old cohort and an Oct4, Sox2, Klf4 and c-Myc (OSKM) treated old cohort;
b) performing whole-genome bisulfite sequencing (WGBS) on the DNA sample;
c) calculating DNA methylation potential energy landscape across the genome of the DNA sample;
d) measuring a mean methylation level (MML) and a normalized methylation entropy (NME) in an analysis region of the DNA sample; and
e) identifying the genomic region of differential DNA methylation in the young cohort and the OSKM treated old cohort as compared to the DNA sample from the old cohort.

12. The method of claim 11, wherein identifying the genomic region of differential DNA methylation is by a Jensen-Shannon distance (JSD) of information theory.

13. The method of claim 1, wherein aging is associated with an increase in MML or NME in one or more PRC2 gene targets.
